(19) United States
(12) Patent Application Publication
(10) Pub. No.: US 2025/0354145 A1
(43) Pub. Date: Nov. 20, 2025

(54) MODIFIED U7 SNRNA CONSTRUCT

(71) Applicants: UCL BUSINESS LTD, London, Greater London (GB); KING'S COLLEGE LONDON, London, Greater London (GB)

(72) Inventors: Pietro FRATTA, London (GB); Christopher SHAW, London (GB); Marc-David RUEPP, London (GB)

(73) Assignees: UCL BUSINESS LTD, London, Greater London (GB); KING'S COLLEGE LONDON, London, Greater London (GB)

(21) Appl. No.: 18/872,530

(22) PCT Filed: Jun. 7, 2023
(86) PCT No.: PCT/EP2023/065308

§ 371 Date: Dec. 6, 2024

(30) Foreign Application Priority Data
Jun. 8, 2022 (GB) 2208387.7

(51) Int. Cl. C12N 15/113

(52) U.S. Cl. CPC C12N 15/113; C12N 2320/33

(57) ABSTRACT

A modified U7 snRNA construct, more particularly a U7 smOPT construct, is described having (i) an antisense sequence having between 16 to 30 nucleotides which is at least 90% complementary to a TDP-43 regulated cryptic exon sequence or flanking regions thereof, and (ii) a sequence comprising a binding domain for a hnRNP protein, wherein the construct is capable of modulating splicing of the TDP-43 regulated cryptic exon in a cell. Vectors and pharmaceutical compositions comprising the construct are also described, as well as constructs for use in the treatment of diseases associated with TDP-43 dysfunction. Example TDP-43 regulated cryptic exon sequences include TDP-43 regulated cryptic exons in UNC13A, STMN2 and INSR genes.

### BACKGROUND

Loss of nuclear TDP-43 is observed in a number of diseases or disorders including >95% of all Amyotrophic Lateral Sclerosis (ALS) and tau-negative Frontotemporal Dementia (FTD) cases. This results in the inclusion of cryptic exons (CE) with subsequent functional loss of important disease-modifying genes, due to the absence of TDP-43 repression of these cryptic exons. TDP-43 regulated cryptic exons in both STMN2 and UNC13A have been mechanistically linked to ALS and FTD: STMN2 and UNC13A encode an axonal and synaptic protein, respectively and are crucial for normal neuronal function. In both cases, loss of nuclear TDP-43 results in the incorporation of a CE during splicing resulting in the depletion of the full-length mRNA and reduction of functional protein expression. Loss of nuclear TDP-43 also results in aberrant RNA processing, with STMN2 being the most significantly affected. Its depletion results in impaired axonal regeneration, which is alleviated when STMN2 levels are restored. For UNC13A human genetic evidence supports its impact in disease aetiology: Intronic SNPs in UNC13A are the second strongest risk factor for sporadic ALS, are associated with reduced patient survival, and shown to directly enhance cryptic exon inclusion.

TDP-43 regulated cryptic exons (CEs) are also known to affect numerous other transcripts which have crucial neuronal functions. One such example is in the ELAVL3 gene which encodes for a neuronal-specific RNA binding protein. The ELAVL3 CE leads to protein loss, which has been documented in ALS post mortem neurons, and leads to alterations in neurite maturation, maintenance. Similarly, TDP-43 loss induces a CE and consequent loss of another neuronal-specific RNA binding protein, CELF5, loss of which is known to cause motor neuron degeneration in model systems. CEs also appears in the INSR transcript leading to its reduction, with insulin signalling having emerged as an important pathway for neuronal health and maintenance.

There is therefore a need to further understand the role of TDP-43 depletion in disease, and to generate new therapeutic approaches for alleviating diseases associated with TDP-pathology, including but not limited to neurodegeneration, particularly in ALS/FTD.

### SUMMARY OF INVENTION

According to a first aspect of the present invention, is provided a modified U7 snRNA construct comprising
(i) an antisense sequence having between 16 to 30 nucleotides which is at least 90% complementary to a TDP-43 regulated cryptic exon sequence or flanking regions thereof, and
(ii) a sequence comprising a binding domain for a hnRNP protein, wherein the construct is capable of modulating splicing of the TDP-43 regulated cryptic exon in a cell. In some embodiments, the flanking regions described herein may be defined as 150 nucleotides upstream and downstream of the TDP-43 regulated cryptic exon. In some embodiments, the cryptic exon sequence or flanking regions thereof may be defined by a defined sequence for a particular TDP-43 cryptic exon (e.g., SEQ ID NO: 1, 2, 3,4, 7 or 9). In some embodiments, the sequence comprising a binding domain for a hnRNP protein comprises a binding domain for a hnRNP A or hnRNP H protein, as may be defined herein.

The antisense sequence directs the construct to the TDP-43 regulated cryptic exon sequence or flanking regions thereof, while the sequence comprising a binding domain for hnRNP is capable of recruiting a hnRNP protein, and more particularly an endogenous hnRNP protein in a cell, to pre-mRNA containing the cryptic exon. Importantly, binding of the hnRNP protein acts to repress splicing of the cryptic exon, even in the absence of TDP-43 binding, or in cells depleted of TDP-43, such that the cryptic exon is at least partially excluded in the mature RNA of the cell transcript. This restores the functionality of genes containing TDP-43 regulated cryptic exons, e.g., in cells depleted of TDP-43. The constructs herein can therefore be used to further probe, understand, or treat diseases or disorders characterised by TDP-43 dysfunction or pathology.

According to a second aspect of the present invention, is provided a vector that comprises or encodes for the modified U7 snRNA construct of the first aspect. In some embodiments, the vector is a viral vector.

According to a third aspect of the present invention, is provided a pharmaceutical composition comprising one or more of the constructs according to the first aspect, and/or one or more of the vectors according to the second aspect.

According to a fourth aspect of the present invention, is provided the construct of the first aspect, the vector of the second aspect or the pharmaceutical composition of the third aspect for use in therapy. Also disclosed herein is the construct of the first aspect, the vector of the second aspect or the pharmaceutical composition of the third aspect for use as a medicament, for use in the manufacture of a medicament, or for use in a method of treatment (e.g., of a neurodegenerative or muscular disease or disorder).

According to a fifth aspect of the present invention, is provided the construct of the first aspect, the vector of the second aspect, or the pharmaceutical composition of the third aspect, for use in the treatment of a disease characterised by TDP-43 dysfunction. In some embodiments, the disease is a neurodegenerative or muscular disease. In some embodiments, the disease is selected from Amyotrophic lateral sclerosis (ALS) and frontotemporal dementia (FTD), Inclusion body myositis or myopathy (IBM), Alzheimer's disease, FOSMNN (Facial onset sensory and motor neuronopathy), Perry Syndrome, Limbic-Predominant Age-Related TDP-43 Encephalopathy (LATE) or a combination thereof.

According to a sixth aspect of the present invention, is a method of modulating splicing of a TDP-43 regulated cryptic exon, the method comprising delivering to a cell the construct of the first aspect, the vector of the second aspect, or the pharmaceutical composition of the third aspect, wherein the method comprises contacting the construct with a cell to modulate splicing of the TDP-43 regulated cryptic exon in the cell.

According to a sixth aspect of the invention, there is provided a combined vector comprising two or more of the constructs described herein or of the first aspect of the invention (i.e., in tandem, or one downstream of another, such that the combined vector comprises at least two constructs, each comprising one antisense sequence as defined herein and each comprising a sequence comprising a binding domains for a hnRNP protein as defined herein). In preferred embodiments, the two or more modified U7 snRNA constructs comprise different antisense sequences that are capable of binding to (i.e., they are at least 90%, or at least 95%, or 100% complementary to) different TDP-43 regulated cryptic exons described herein. In some embodiments, the combined vector may comprise three or more constructs as defined herein. In some embodiments, the combined construct comprises two or more antisense sequences that are complementary (i.e., at least 90% complementary, or at least 95% complementary, or 100% complementary) to two or more TDP-43 regulated cryptic exon sequences or flanking regions thereof. In some embodiments, the TDP-43 regulated cryptic exon is selected from one of the TDP-43 regulated cryptic exons defined herein. In some embodiments, each antisense sequence is a sequence that is complementary (i.e., 90%, 95% or 100% complementary) to SEQ ID NO: 1, 2, 3,4, 7, 9, or 448-453). In some embodiments, at least one of the antisense sequences, or each antisense sequences, is complementary to a TDP-43 binding region of the TDP-43 regulated cryptic exon, preferably wherein at least one of the antisense sequences, or each antisense sequence, is complementary (i.e., 90%, 95% or 100% complementary) to SEQ ID NO: 12, 23-26 or 32. In some embodiments, the combined vector comprises a construct as defined herein comprising an antisense sequence which is at least 90% complementary to a UNC13A TDP-43 regulated cryptic exon or flanking region thereof and a construct as defined herein comprising an antisense sequence which is at least 90% complementary to a STMN2 TDP-43 regulated cryptic exon or flanking region thereof. In some embodiments, the combined vector comprises a construct as defined herein comprising an antisense sequence which is at least 90% complementary to a UNC13A TDP-43 regulated cryptic exon or flanking region thereof and a construct as defined herein comprising an antisense sequence which is at least 90% complementary (or 95%, or 100% complementary) to a INSR TDP-43 regulated cryptic exon or flanking region thereof. In some embodiments, the combined vector comprises a construct as defined herein comprising an antisense sequence which is at least 90% complementary (or 95%, or 100% complementary) to a STMN2 TDP-43 regulated cryptic exon or flanking region thereof and a construct as defined herein comprising an antisense sequence which is at least 90% complementary (or 95%, or 100% complementary) to a INSR TDP-43 regulated cryptic exon or flanking region thereof. In some embodiments, the combined vector comprises a construct comprising an antisense sequence which is at least 90% complementary (or 95%, or 100% complementary) to a UNC13A TDP-43 regulated cryptic exon or flanking region thereof, a construct comprising an antisense sequence which is at least 90% complementary (or 95%, or 100% complementary) to a STMN2 TDP-43 regulated cryptic exon or flanking region thereof, and a construct comprising an antisense sequence which is at least 90% complementary (or 95%, or 100% complementary) to a INSR TDP-43 regulated cryptic exon or flanking region thereof.

In some embodiments, the combined vector comprises two or more constructs defined herein, wherein the two or more sequences comprising a binding domain for a hnRNP protein may be according to any sequence as described herein. In some embodiments, the two or more sequences comprising a binding domain for a hnRNP protein may be different or identical. In some embodiments, the two or more sequences comprising a binding domain for a hnRNP protein may be a binding domain for a hnRNP A or hnRNP H protein, and in some examples, a hnRNP A protein.

In some embodiments, the combined vector comprises two or more promoter sequences, wherein the two or more promoter sequences are upstream of each construct. The promoters may be any promoter sequence used in the art. In some embodiments, each of the two or more promoter sequences are the same or different. In some embodiments, the combined vector comprises two or more 3′ box sequences, wherein the two or more 3′ box sequences are downstream of each construct. The 3′ box sequences may be the same or different and may be any 3′ box sequence used in the art.

In some embodiments, the combined vector comprises two or more U7 cassettes, wherein each cassette comprises a promoter, a modified U7 snRNA construct as defined herein, and a 3′ box sequence, wherein the promoter is upstream of the modified U7 snRNA construct and the 3′ box sequence is downstream of the modified U7 snRNA construct. In some embodiments, the combined vector comprises a stuffer sequence between each of the two or more U7 cassettes. The stuffer sequences serve to space out the two promoters. The stuffer sequence may be any suitable stuffer sequence used in the art.

In some embodiments, the combined vector comprises (from upstream to downstream) at least a:
A first promoter,
A first modified U7 modified RNA construct as defined herein,
A first 3′ box sequence,
A stuffer sequence,
A second promoter,
A second modified U7 modified RNA construct as defined herein, and
A second 3′ box sequence.

The present inventors have developed tools that can target TDP-43 regulated cryptic exons and modulate their aberrant cryptic splicing in cells (e.g., upon depletion of TDP-43). The modulation of splicing means that splicing of the cryptic exon is at least partially repressed which in turn means that inclusion of the TDP-43 regulated cryptic exon in mature RNA is at least partially prevented, leading to the formation of a correctly spliced mature RNA transcript which can be translated into a fully functional protein. This therefore restores the production of functional proteins encoded by genes that contain TDP-43 regulated cryptic exons.

There are a number of TDP-43 regulated cryptic exons that are aberrantly spliced upon depletion of TDP-43 in the nucleus. TDP-43 depletion is associated with a number of diseases including neurodegenerative and muscular diseases, including ALS and FTD as described in the background section of this application. TDP-43 regulated cryptic exons are characterised by a TDP-43 binding region either within the cryptic exon or in close proximity to the cryptic exon (i.e., in the flanking regions of the cryptic exon), said TDP-43 binding region typically being UG rich. During normal splicing (i.e., in healthy cells), TDP-43, which is a transcriptional repressor protein, binds to the TDP-43 binding domain and represses splicing of the cryptic exon; this has the effect that the cryptic exon is not included in the mature mRNA of the transcript and a functional protein is produced. However, depletion of TDP-43 from the nucleus of cells means that the cryptic exon sequence is aberrantly spliced; this has the effect that the cryptic exon is included in the mature mRNA of the transcript meaning functional protein is not produced.

The constructs, vectors and pharmaceutical compositions disclosed herein can crucially be used to at least partially, or in some instances substantially completely or completely, restore correct splicing in the absence of TDP-43. The U7 constructs disclosed herein comprise both (i) an antisense sequence that guides the U7 snRNP to bind to the target cryptic exon (i.e., present in the pre-mRNA) and (ii) an hnRNP binding sequence for recruitment of an endogenous hnRNP protein. The tethering of hnRNPs substitutes for the loss of TDP-43 allowing for at least partial abolishment the cryptic splicing event. This restores the “normal” protein production which occurs in healthy cells (i.e., without TDP-43 depletion of dysfunction). This approach is particularly effective because hnRNPs are ubiquitously expressed and hence the constructs can be used in all cells that express them. It is particularly surprising that the tethering and recruitment of a hnRNP protein can almost completely substitute for the loss of TDP-43 function in repression of cryptic exons. Such an effect is found to be more pronounced and most effective with the highly endogenously expressed proteins such as hnRNP A1, with hnRNP H also showing good efficacy.

To the present inventor's knowledge, there has been no modified U7 snRNA constructs targeting TDP-43 regulated cryptic exons in the prior art, nor would it have been expected that recruiting a hnRNP A1 protein to a TDP-43 regulated cryptic exon (i.e., in the pre-mRNA) with a U7 construct would be sufficient to rescue the loss of TDP-43 binding in a TDP-43 depleted cell, given TDP-43's widespread binding in and/or across the suppressed cryptic exon. While other modified U7 constructs have been previously used in gene therapy, such constructs have a different target and a different mode of action. Instead, modified U7 snRNA constructs of the prior art seek to target standard constitutive exons or constitutive exons that are alternatively spliced due to mutations in the DNA, rather than cryptic exons, let alone constructs used to rescue splicing of TDP-43 regulated cryptic exons. The difference is that TDP-43 regulated cryptic exons are non-conserved intronic sequences that are erroneously included in mature RNA in cells depleted of TDP-43. These differ from typical constitutive exons which are instead supposed to be included in mature RNA. Previous U7 modified constructs therefore had a different aim, to promote exon inclusion and reduce gene expression of various genes. This is different to the constructs of the present invention which instead repress splicing of the cryptic exon to restore expression of TDP-43 regulated genes. The prior art constructs also have completely different targets and therefore completely different uses. No prior art constructs have been used to correct TDP-43 regulated cryptic exons, to rescue the correct splicing of genes which are depleted in the cell (e.g., due to TDP-43 pathology).

The construct in accordance with the present invention may be referred to as a “bifunctional construct”. This “bifunctional” approach provides a modified U7 snRNA construct which comprises both (i) an antisense sequence which binds to the TDP-43 regulated cryptic exon or flanking regions thereof, and (ii) a binding sequence for an hnRNP protein to recruit an endogenous hnRNP. This is demonstrated to be more effective than analogous U7 snRNA constructs which only comprise an antisense sequence (i.e., in the absence of a hnRNP binding sequence, which may be referred to as “single” target constructs herein). The design and approach of the present invention also allows for more flexibility as the antisense sequence need not be restricted to targeting core splice elements (e.g., splice sites) for reinstalling splicing repression. Indeed, example constructs described herein are found to effectively correct splicing, despite comprising antisense sequences that target different regions of TDP-43 regulated cryptic exons. In some examples, the antisense sequence binds to a TDP-43 binding region of a TDP-43 regulated cryptic exon, while correcting splicing. Since TDP-43 has as repressive role in healthy cells, and blocks splicing machinery from recognising the cryptic exon, constructs comprising antisense sequences that target the TDP-43 binding region serve to provide a steric block within this region, which contributes to blocking cryptic splicing. In alternative examples, the antisense sequence binds to a splice site of the TDP-43 regulated cryptic exon while correcting splicing. Constructs comprising antisense sequences that target the splice sites means that the splice sites are masked and less available for splicing by the splicing machinery within the cell. Further, it is also demonstrated that correct splicing is restored when the antisense sequence binds to an exonic splice enhancer (i.e., as identified by ESE finder 3.0) located within the TDP-43 regulated cryptic exon. Since ESEs are motifs within the cryptic exon sequence that promote or enhance splicing, blocking these motifs blocks cryptic splicing of the cryptic exon sequence. The present inventors demonstrate that the constructs can target a wide range of different target sequences within the TDP-43 regulated cryptic exon and flanking regions thereof, while still being effective at correcting splicing. Further, the present inventors demonstrate that this approach can be used to effectively correct splicing, at least partially, of various TDP-43 regulated cryptic exons.

There is also no prior example of a U7 construct that aims to target and correctly splice a TDP-43 regulated cryptic exon, which comprises both an antisense sequence which targets the TDP-43 regulated exon and a binding domain for a hnRNP protein. Crucially, different to prior approaches, the binding domain for the hnRNP protein seeks to recruit a hnRNP protein which takes over the repressive function (e.g., in cells depleted of TDP-43) that TDP-43 normally has in “healthy cells”. While previous U7 constructs have been described that couple antisense sequences with a binding sequence for a protein, these have been used against a different gene target. Additional U7 constructs of the prior art have a different aim, that is, to promote inclusion of a constitutive exon in the resultant mRNA (e.g., due to a mutation in a gene which alters splicing), rather than repress the inclusion of a cryptic exon in the resultant mRNA, let alone a TDP-43 regulated cryptic exon. Finally, in some instances, other U7 constructs in the art have instead aimed to recruit exonic splicing enhancers, such as SR proteins. SR proteins have the opposite effect to recruitment of a hnRNP protein as described in the present invention, since hnRNP proteins instead have a repressive effect.

A major advantage of a using a modified U7 snRNA approach is that snRNPs naturally reside in the nucleus where cryptic exon splicing happens. This results in localisation of the antisense containing U7 snRNA in the cellular compartment where splicing needs to be corrected. The use of antisense sequences in snRNPs also provides enhanced stability of the resultant RNA-protein complexes with the pre-mRNA (i.e., which contains the cryptic exon).

Another advantage is that modified U7 snRNAs can be packaged into vectors, such as viral vectors, which enable long lasting manufacture of the gene therapy following a single injection. This allows cells to produce their own therapeutic molecules as a single dose gene therapy, and is therefore improved as compared to ASO approaches. These constructs also provide a more stable therapeutic approach as compared to ASO targeting which are more sensitive to degradation. The small delivery of the U7 expression gene also allows their delivery in combination with other antisense or supplemental gene constructs in a single viral vector or ITR cassette. Finally, it is hypothesised that the larger size of the modified U7 snRNA construct as compared to an ASO approach could, in some instances, be more effective at correcting splicing due to steric effects; this is since the constructs may also provide a more effective steric block which contributes to the repression of the cryptic splicing event.

Since aspects of the invention are demonstrated to at least partially correct the splicing of TDP-43 regulated cryptic exons, aspects of the present invention can therefore be used to probe TDP-43 pathology and/or the role of TDP-43 pathology in disease. For example, as TDP-43 clearance is happening in >95% of ALS cases this approach is applicable and beneficial for the vast majority of ALS patients.

The present inventors have also uniquely demonstrated that a vector comprising two or more of the constructs of the invention (i.e., in tandem, or one after each other) suppresses TDP-43 cryptic exon inclusion in different genes. Different from any prior approach, this combined construct is able to target and rescue splicing for multiple TDP-43 regulated cryptic exons in different genes. The combined construct showed similar suppression of three TDP-43 regulated exons, UNC13A, INSR and STMN2, as compared to individual construct transfection. The result is unexpected considering the combined construct comprises multiple (and in some examples, identical promoters) and surprising in the context of promoter competition and promoter interference given three identical promoters were used to drive the expression of three different antisense sequences.

In some embodiments, constructs of the invention can be used to correct splicing of the TDP-43 regulated UNC13A cryptic exon. This cryptic exon is found to cause UNC13A downregulation at the transcript and protein level and is detected specifically in patient post-mortem brain regions affected by TDP-43 proteinopathy or dysfunction, including both ALS and FTD. Further, this cryptic exon is also found to overlap with the disease-associated variant rs12973192 previously identified in multiple genome-wide association studies linked to ALS/FTD risk, as well as disease aggressiveness. The UNC13A cryptic exon is therefore associated with TDP pathology, and disease aggressiveness. Correcting splicing of the UNC13A gene can therefore be used to further understand and/or treat diseases associated with ALS and FTD, and SNPs (e.g., rs12973192) in the UNC13A gene.

In some embodiments, constructs of the invention can be used to correct splicing of the TDP-43 regulated STMN2 cryptic exon 2a. This is important considering loss of nuclear TDP-43 results in the incorporation of this cryptic exon during splicing resulting in the depletion of the full-length mRNA and reduction of functional protein expression. This effect is most pronounced for STMN2, where aberrant RNA processing results in impaired axonal regeneration. Correcting splicing of the STMN2 gene can therefore be used to further understand and/or treat diseases associated with TDP-43.

Embodiments of the present invention are also used to correct splicing of the TDP-43 regulated INSR cryptic exon (between INSR exons 6 and 7). The INSR CE leads to loss of the protein, which normally acts as a receptor for insulin. Insulin signalling plays an important role in neuronal maintenance, and restoration of INSR levels would contribute to an amelioration of neuronal homeostasis.

Embodiments of the present invention are also used to correct splicing of other TDP-43 regulated cryptic exons, such as the ELAVL3 CE, the G3BP1 CE, the AARS1 CE, the CELF5 CE, the CAMK2B CE or the UNC13B CE. Preventing cryptic splicing and restoration of these proteins is considered to be therapeutically beneficial. In particular, the ELAVL3 CE leads to alterations in neurite maturation and is implicated in ALS, while the CELF5 CE leads to motor neuron degeneration in model systems.

Also described herein is a modified U7 snRNA construct comprising
(i) an antisense sequence having between 16 to 30 nucleotides which are at least 90% complementary to a TDP-43 regulated cryptic exon sequence in UNC13A and flanking regions thereof, preferably wherein the antisense sequence is at least 90% complementary to SEQ ID NO: 1 or 2, and
(ii) a sequence comprising a binding domain for a hnRNP protein.

In some embodiments, the antisense sequence is at least 90% complementary to SEQ ID NO: 3 or 4.

Also described herein is a modified U7 snRNA construct comprising (i) an antisense sequence having between 16 to 30 nucleotides which are at least 90% complementary to a TDP-43 regulated cryptic exon sequence in STMN2 and flanking regions thereof, preferably wherein the antisense sequence is at least 90% complementary to SEQ ID NO: 7, and
(ii) a sequence comprising a binding domain for a hnRNP protein.

Also described herein is a modified U7 snRNA construct comprising
(i) an antisense sequence having between 16 to 30 nucleotides which are at least 90% complementary to a TDP-43 regulated cryptic exon sequence in INSR and flanking regions thereof, preferably wherein the antisense sequence is at least 90% complementary to SEQ ID NO: 9, and
(ii) a sequence comprising a binding domain for a hnRNP protein.

Also described herein is a modified U7 snRNA construct comprising
(i) an antisense sequence having between 16 to 30 nucleotides which are at least 90% complementary to a TDP-43 regulated cryptic exon sequence or flanking regions thereof, wherein the flanking regions refer to the 150 nucleotides upstream and downstream of the cryptic exon, (or optionally the 100 nucleotides, or the 75 nucleotides, or up to 50 nucleotides, or up to 25 nucleotides upstream and downstream of the cryptic exon) and (ii) a sequence comprising a binding domain for a hnRNP protein.

Also described herein is a modified U7 snRNA construct comprising
(i) an antisense sequence having between 16 to 30 nucleotides which are at least 90% complementary to a TDP-43 binding region of a TDP-43 regulated cryptic exon sequence and
(ii) a sequence comprising a binding domain for a hnRNP protein.

Also described herein is a modified U7 snRNA construct comprising a modified Sm motif comprising
(i) an antisense sequence having between 16 to 30 nucleotides which are at least 90% complementary to a TDP-43 regulated cryptic exon sequence or flanking regions thereof, and
(ii) a sequence comprising a binding domain for a hnRNP protein.

Also disclosed herein is a modified U7 snRNA construct comprising
(i) an antisense sequence having between 16 to 30 nucleotides which is at least 90% complementary to a TDP-43 regulated cryptic exon sequence or flanking regions thereof, and
(ii) a sequence comprising a binding domain for hnRNP A or hnRNP H wherein the construct is capable of modulating splicing of the TDP-43 regulated cryptic exon in a cell. In some embodiments, the flanking regions described herein may be defined as 150 nucleotides upstream and downstream of the TDP-43 regulated cryptic exon. In some embodiments, the cryptic exon sequence or flanking regions thereof may be defined by a defined sequence for a particular TDP-43 cryptic exon (e.g., SEQ ID NO: 1, 2, 3,4, 7 or 9). The sequence comprising a binding domain for hnRNP A or hnRNP H may be defined in accordance with any definition defined elsewhere herein.

Also disclosed herein is a system comprising a construct, vector, or pharmaceutical composition and a cell, wherein said cell comprises or expresses a hnRNP protein. The cell may be as elsewhere defined herein.

For any sequence disclosed herein, the complementary sequence and reverse complement sequence is also disclosed. Also disclosed herein is a vector or construct with a complementary sequence to that described herein which may be used to encode for the constructs described herein.

### BRIEF DESCRIPTION OF FIGURES

FIG. 1A) shows a schematic of splicing in healthy cells (top) and “diseased” cells depleted of TDP-43 (bottom). In healthy cells, TDP-43 binds to a TDP-43 binding domain in close proximity to the cryptic exon and represses splicing of said cryptic exon in the pre-mRNA such that the cryptic exon sequence is not included in the mature mRNA of the cell transcript. In diseased cells, depletion of TDP-43 means that no such repression of the cryptic exon occurs such that the cryptic exon sequence is present in the mature RNA of the cell transcript; this minimizes or prevents production of a fully functional protein encoded by the gene in which the TDP-43 regulated cryptic exon is present; FIG. 1B) shows a schematic of how the modified U7 snRNA construct of the invention can restore correct splicing in diseased cells. The bifunctional U7 smOPT construct, aided by an antisense sequence which is specific to the TDP-43 regulated cryptic exon, is directed to the pre-mRNA containing the cryptic exon sequence; next, an endogenous hnRNP protein is recruited to the binding sequence of the hnRNP protein which is present in the construct. The presence of a hnRNP protein represses splicing of the cryptic exon, fulfilling the role of TDP-43 in healthy cells, and therefore prevents or minimizes inclusion of the cryptic exon in mature mRNA.

FIG. 2 shows the rescue of UNC13A splicing in TDP-43 depleted electroporated SH-SY5Y cells using an example modified U7 snRNA construct of the invention (i.e., Example 1). This is demonstrated by gel electrophoresis of the mature mRNA UNC13A transcripts, where a band is observed corresponding to correctly spliced UNC13A mature RNA.

FIG. 3 shows the RT-PCR product of the UNC13A mature RNA in TDP-43 knockdown SK-N-DZs cells transfected with the UNC13A minigene after treatment with an example modified U7 snRNA construct of the invention (i.e., Example 1). A band is observed corresponding to the correctly spliced product.

FIG. 4 shows the % differential splicing of the correctly spliced mature RNA (far left bar), mature RNA comprising the short UNC13A cryptic exon (middle bar) and mature RNA comprising the long UNC13A cryptic exon (far right bar) in TDP-43 knockdown SK-N-DZs cells transfected with a UNC13A minigene after treatment with either an example construct of the invention (i.e., corresponding to Example 1) or a control.

FIG. 5 shows RT-PCR product of the UNC13A mature RNA in TDP-43 depleted SH-SY5Y cells after treatment with an example construct of the invention (i.e., corresponding to Example 1). For the example construct of the invention, a band is observed corresponding to the correctly spliced product, with no band observed for controls.

FIG. 6 shows the % differential splicing of the correctly spliced mature RNA (far left bar), mature RNA comprising the short UNC13A cryptic exon (middle bar) and mature RNA comprising the long UNC13A cryptic exon (far right bar) deriving from endogenous UNC13A in electroporated TDP-43 depleted SH-SY5Y cells after treatment with an example construct of the invention (i.e., corresponding to Example 1).

FIG. 7A shows the ratio of cryptic exon containing to correctly spliced mRNA expressed from the UNC13A minigene in the presence of different modified U7 snRNA constructs of the invention comprising different antisense sequences which target the TDP-43 binding region of the UNC13A cryptic exon, (i.e., along with a binding sequence for hnRNP A1.

FIG. 7B shows the ratio of cryptic exon containing to correctly spliced mRNA expressed from the UNC13A minigene in the presence of a different example construct comprising an antisense sequence that targets the 3′-splice site of the UNC13A cryptic exon (i.e., along with a binding sequence for hnRNP A1).

FIG. 8 shows partial rescue of STMN2 cryptic splicing using an example construct of the invention (i.e., corresponding to Example 2) in TDP-43 depleted electroporated SH-SY5Y cells. For the example construct of the invention, a band is observed corresponding to the correctly spliced product.

FIG. 9 shows the differential splicing of the correctly spliced mature RNA (left bar) compared with mature RNA containing the STMN2 cryptic exon (right bar) using an example construct of the invention (i.e., corresponding to Example 2).

FIG. 10A shows the ratio of cryptic exon containing to correctly spliced mRNA expressed from the STMN2 minigene in the presence of different constructs of the invention comprising various antisense sequences that target the TDP-43 binding region of the STMN2 cryptic exon (i.e., along with a binding sequence for hnRNP A1). FIG. 10B shows the ratio of cryptic exon containing to correctly spliced mRNA expressed from the STMN2 minigene in the presence of a different example construct of comprising an antisense sequence that instead targets an ESE site in the STMN2 cryptic exon (i.e., along with a binding sequence for hnRNP A1).

FIG. 11 shows the RT-PCR product of the INSR mature RNA in TDP-43 knockdown SK-N-DZs cells transfected with the INSR minigene after treatment with Example constructs of the invention (i.e., corresponding to Examples 3A and 3B). For the example constructs of the invention, bands are observed corresponding to the correctly spliced product.

FIG. 12A shows the ratio of cryptic exon containing to correct spliced mRNA expressed from the UNC13A minigene in the presence of either (i) “bifunctional” constructs of the invention, i.e., comprising an antisense sequence that targets the TDP-43 binding region of the UNC13A cryptic exon and a binding sequence for hnRNP A1 or (ii) a comparative “single” construct comprising an analogous antisense sequence but which lacks the hnRNP A1 binding sequence. FIG. 12B shows the ratio of cryptic exon containing to correct spliced mRNA expressed from the UNC13A minigene in the presence of either (i) “bifunctional” constructs of the invention, i.e., comprising an antisense sequence that targets a 3′ splice site of the UNC13A cryptic exon and a binding sequence for hnRNP A1 or (ii) a comparative “single” construct comprising an analogous antisense sequence, but which lacks the hnRNP A1 binding sequence.

FIG. 13 shows the TDP-43 regulated UNC13A cryptic exon target and flanking regions thereof, annotated with splicing elements. The sequence corresponds to SEQ ID NO: 4

FIG. 14 shows the TDP-43 regulated SNTM2 cryptic exon target and flanking regions thereof, annotated with splicing elements. The sequence corresponds to SEQ ID NO: 7

FIG. 15 shows the TDP-43 regulated INSR cryptic exon target and flanking regions thereof, annotated with splicing elements. The sequence corresponds to SEQ ID NO: 9

FIG. 16 shows the ratio of cryptic exon included to correctly spliced RT-qPCR levels of STMN2 mRNA from bifunctional approach relative to the ratio obtained with monofunctional approach targeting either TDP-43 binding site (BS) or putative ESE (ESE) in 293T-2xTDP-shRNA cells containing STMN2 minigene and under TDP-43 knockdown.

FIG. 17 shows the ratio of cryptic exon included to correctly spliced RT-qPCR levels of UNC13A mRNA from bifunctional approach relative to the ratio obtained with monofunctional approach targeting either TDP-43 binding site (BS) or 3′ splice site (3′ss) in 293T-2xTDP-shRNA cells containing UNC13A minigene and under TDP-43 knockdown.

FIG. 18 shows the ratio of cryptic exon included to correctly spliced RT-qPCR levels of UNC13A mRNA comparing bifunctional approach targeting TDP-43 binding site (TDP-43 BS) or 5′ splice site/TDP-43 BS (5′ss/TDP-43 BS) to a 3′ splice site (3′ss). Data is shown relative to a ratio in non-targeting control (U7 Control) transfected 293T-2xTDP-shRNA cells containing UNC13A minigene and under TDP-43 knockdown normalized to GAPDH mRNA.

FIG. 19 shows ratio of cryptic exon included to correctly spliced RT-qPCR levels of STMN2 mRNA comparing bifunctional approach targeting TDP-43 binding site (TDP-43 BS) to putative ESE. Data shown relative to ratio in non-targeting control (U7 Control) transfected 293T-2xTDP-shRNA cells containing STMN2 minigene and under TDP-43 knockdown normalized to GAPDH mRNA.

FIG. 20 shows that STMN2 levels are rescued using vectorised U7 constructs targeting the STMN2 cryptic exon. STMN2 protein levels were assessed in Doxycycline (Dox)-inducible TDP-43 SH-SY5Y cells that were either non-transduced (Control), or transduced with either, a non-targeting U7SmOPT (U7 Control), a comparative monofunctional U7SmOPT targeting 3′ splice site (Ex. 2J) or a bifunctional U7SmOPT construct of the invention targeting TDP-43 binding site (Ex. 2C) expressing lentiviral vector in the presence (TDP-43 KD+) or absence (TDP-43 KD-) of a TDP-43 knockdown. GAPDH protein levels were assessed as loading control.

FIG. 21 shows that U7 snRNPs targeting the STMN2 cryptic exon suppress cryptic exon inclusion. FIG. 21 shows ratio of cryptic exon included to correctly spliced STMN2 mRNA assessed by RT-qPCR. Doxycycline (Dox)-inducible TDP-43 SH-SY5Y cells were either non-transduced (Control), or transduced with either, a non-targeting U7SmOPT (U7 Control), a comparative monofunctional U7SmOPT construct targeting 3′ splice site (Ex. 2J) or bifunctional U7SmOPT example construct of the invention targeting TDP-43 binding site (Ex. 2C) expressing lentiviral vector. SH-SY5Y cells were either uninduced (No KD) or were depleted from TDP-43 by the addition of Dox (TDP-43 KD) and RNA was isolated, reverse transcribed and subjected to RT-qPCR. Data are presented as mean±SD relative to U7 Control normalised to GAPDH and analyzed using ordinary one-way ANOVA with Tukey's multiple comparison test (*p<0.05, **p<0.01, ***p<0.001, ****p<0.0001).

FIG. 22 shows ratio of cryptic exon included to correctly spliced UNC13A mRNA assessed by RT-qPCR. Doxycycline (Dox)-inducible TDP-43 SH-SY5Y cells were either non-transduced (Control), or transduced with either, a non-targeting U7SmOPT (U7 Control), a comparative monofunctional U7SmOPT (Ex. 1R) or bifunctional U7SmOPT of the invention (Ex. 1O) expressing lentiviral vector. SH-SY5Y cells were either uninduced (No KD) or were depleted from TDP-43 by the addition of Dox (TDP-43 KD) and RNA was isolated, reverse transcribed and subjected to RT-qPCR. Data are presented as mean±SD relative to U7 Control normalised to GAPDH and analyzed using ordinary one-way ANOVA with Tukey's multiple comparison test (*p<0.05, **p<0.01, ***p<0.001, ****p<0.0001).

FIG. 23 shows that UNC13A levels are rescued using vectorised U7 snRNPs targeting the UNC13A cryptic exon. UNC13A protein levels were assessed in Doxycycline (Dox)-inducible TDP-43 SH-SY5Y cells that were either non-transduced (Control), or transduced with either, a non-targeting U7SmOPT (U7 Control), a comparative monofunctional U7SmOPT targeting TDP-43 binding site and 5′ splice site (Ex. 1R) or a bifunctional U7SmOPT construct of the invention targeting TDP-43 binding site and 5′ splice site (Ex. 10) expressing lentiviral vector in the presence (TDP-43 KD+) or absence (TDP-43 KD-) of a TDP-43 knockdown. GAPDH protein levels were assessed as loading control.

FIG. 24 shows that U7 constructs of the invention targeting the INSRa cryptic exon suppresses cryptic exon inclusion. The figure shows ratio of cryptic exon included to correctly spliced INSRa mRNA assessed by RT-qPCR. Doxycycline (Dox)-inducible TDP-43 SH-SY5Y cells were either non-transduced (Control), or transduced with either, a non-targeting U7SmOPT (U7 Control) or bifunctional U7SmOPT construct of the invention targeting TDP-43 binding site (Ex. 3B) expressing lentiviral vector. SH-SY5Y cells were either uninduced (No KD) or were depleted from TDP-43 by the addition of Dox (TDP-43 KD) and RNA was isolated, reverse transcribed and subjected to RT-qPCR. Data are presented as mean±SD relative to U7 Control normalised to GAPDH and analyzed using ordinary one-way ANOVA with Tukey's multiple comparison test (*p<0.05, **p<0.01, ***p<0.001, p<0.0001).

FIG. 25 shows that INSRa levels are rescued using constructs of the invention targeting the INSRa cryptic exon. INSRa protein levels were assessed in Doxycycline (Dox)-inducible TDP-43 SH-SY5Y cells that were either non-transduced (Control), or transduced with either, a non-targeting U7SmOPT (U7 Control) or a bifunctional U7SmOPT construct of the invention targeting TDP-43 binding site (Ex. 3B) expressing lentiviral vector in the presence (TDP-43 KD+) or absence (TDP-43 KD-) of a TDP-43 knockdown. GAPDH protein levels were assessed as loading control.

FIG. 26 shows RNA and protein rescue of (IN (13A mis-splicing using UNC′13A-targeting U7 Single (Ex. 1R) and Bifunctional (Ex. 1O) constructs. Human iPSC-derived cortical neurons (i3Neurons) expressing the U7 constructs were cultured. TDP-43 knockdown was achieved by treating the cells with Halo-Protac (300 nM). RNA and protein were harvested on day 11. Top) RT-PCR analysis of UN (13A splicing between exons 19 and 22 shows a rescue in splicing with U7 Bifunctional and Single constructs. Bottom) Western blot analysis of UNC13A levels following treatment with U7 Bifunctional and Single constructs shows a rescue of UNC13A protein.

FIG. 27 shows RNA and protein rescue of STMN2 mis-splicing using example bifunctional constructs of the invention (Ex. 2C). Human iPSC-derived cortical neurons (13Neurons) expressing the U7 constructs were cultured. TDP-43 knockdown was achieved by treating the cells with Halo-Protac (300 nM). RNA and protein were harvested on day 11. Top) Three-primer RT-PCR analysis of STMN2 splicing at between exons 1 and 2 shows a rescue in splicing with U7 Bifunctional and Single constructs. Bottom) Western blot analysis of STMN2 levels following treatment with the construct of the invention shows a rescue of STMN2 protein.

FIG. 28 shows RNA and protein rescue of INSR mis-splicing using an INSR-targeting U7 Bifunctional (Ex. 3B) construct. Human iPSC-derived cortical neurons (13Neurons) expressing the U7 construct were cultured. TDP-43 knockdown was achieved by treating the cells with Halo-Protac (300 nM). RNA and protein were harvested on day 11. Top) RT-PCR analysis of INSR splicing at between exons 6 and 7 shows a rescue in splicing with the U7 Bifunctional construct of the invention. Bottom) Western blot analysis of INSR levels following treatment with the U7 Bifunctional construct shows a rescue of INSR protein.

FIG. 29-33 shows the neurite outgrowth of i3 Neurons is impaired by TDP-43 depletion and rescued by a STMN2-targeting U7 Bifunctional construct of the invention (Ex. 2C). After three days of neuronal induction media, human iPSC-derived cortical neurons (13Neurons) expressing a non-targeting Control U7 construct and a STMN2-targeting Bifunctional U7 construct (Ex. 2C) were plated alongside wildtype i3Neurons in a 96-well plate. TDP-43 knockdown was achieved in the Control U7 and STMN2 Bifunctional U7 conditions by treating the cells with Halo-Protac (300 nM) from day 1 of induction media. The i3Neurons were longitudinally imaged for several days using an IncuCyte (Sartorius) imaging and analysis system, with eight technical replicates for each condition. Neurite outgrowth and cell body area were calculated. Five independent differentiations were performed and plotted on separate graphs. Neurite length, normalised for cell body area, is reduced in TDP-43 depleted i3 Neurons expressing the Control U7, but is rescued in those expressing the STMN2-targeting U7 Bifunctional construct of the invention (Ex. 2C).

FIG. 34 shows the ratio of cryptic exon included to correctly spliced or total RT-qPCR levels of STMN2 (A), UNC13A (B) and INSR (C) mRNA in 293T-2xTDP-shRNA cells transfected with an STMN2 and an UNC13A minigene upon transfection with non-targeting control (Uninduced and U7 Control) or a combined vector comprising multiple constructs pMA-3x-U7SmOPT (3x-tU7SmOPT). The 3x-tU7SmOPT construct contains three U7s in tandem (Ex. 2C, Ex. 10 and Ex. 3D) and is compared to CE/Correct ratios obtained upon transfection with an individual U7 construct Ex. 2C, Ex. 10 or Ex. 3B. Data is presented as mean±SD relative to the ratio in non-targeting control and analyzed using ordinary one-way ANOVA with Tukey's multiple comparison test (*p<0.05, **p<0.01, ***p<0.001, * p<0.0001).

FIG. 35 shows RNA rescue of (NC13A, STMN2, and INSR mis-splicing using a combined triple U7 Bifunctional construct (Ex. 10 for UNC13A, Ex. 2C for STMN2, and Ex. 3D for INSR) in SH-SY5Y neuronal cells. TDP-43 inducible shRNA knockdown SH-SY5Y cells were left untreated or treated with doxycycline 0.025 μg/mL for 5 days. The cells were then electroporated with 2 μg of U7 DNA constructs with Ingenio Electroporation Kit (Mirus) using the A-023 setting on an Amaxa II nucleofector (Lonza). The cells were then left untreated or treated with 1 μg/mL doxycycline for 5 further days before RNA extraction on day 10. RT-PCR analysis of STMN2, INSR, and UNC13A splicing shows a rescue in splicing of all three genes using the combined triple U7 construct. The positive control demonstrated good electroporation efficiency. PCR products were resolved on a TapeStation 4200 (Agilent).

FIG. 36 shows the ratio of cryptic exon included to total RT-qPCR levels of INSRa in cells treated with a bifunctional construct of the invention “Example 3D” which targets the 3′ splice site. Data is shown relative to ratio in non-targeting control (U7 Control) transfected 293T-2xTDP-shRNA cells containing INSRa minigene and under TDP-43 knockdown normalized to GAPDH mRNA.

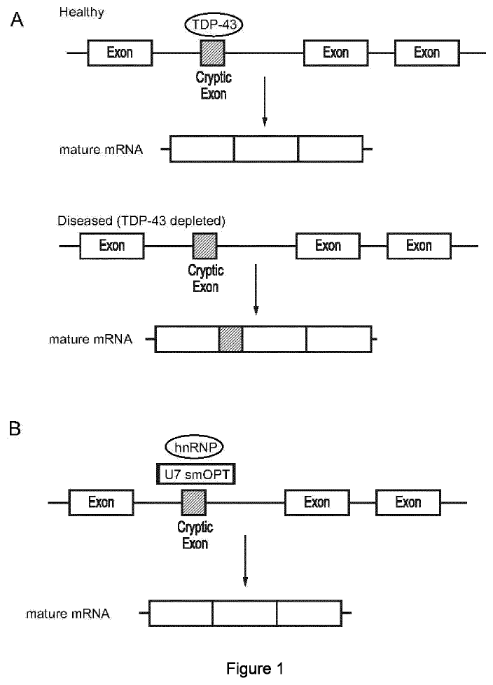

### DETAILED DESCRIPTION

The terms “treatment” and “treating” herein refer to an approach for obtaining beneficial or desired results in a subject, which includes a prophylactic benefit and a therapeutic benefit.

“Therapeutic benefit” refers to eradication, amelioration or slowing the progression of the underlying disorder being treated. Also, a therapeutic benefit is achieved with the eradication or amelioration of one or more of the physiological symptoms associated with the underlying disorder such that an improvement is observed in the subject, notwithstanding that the patient may still be afflicted with the underlying disorder.

“Prophylactic benefit” refers to delaying or eliminating the appearance of a disease or condition, delaying or eliminating the onset of symptoms of a disease or condition, slowing, halting, or reversing the progression of a disease or condition, or any combination thereof. In the context of the present invention, the prophylactic benefit or effect may involve the prevention of the condition or disease. The construct, vector, or pharmaceutical composition may be administered to a subject at risk of developing a particular disease, or to a subject reporting one or more of the physiological symptoms of a disease, even though a diagnosis of this disease may not have been made.

The term “effective amount” or “therapeutically effective amount” refers to the amount of the construct, vector, or pharmaceutical composition needed to bring about an acceptable outcome of the therapy as determined by reducing the likelihood of disease as measurable by clinical, biochemical or other indicators that are familiar to those trained in the art. The therapeutically effective amount may vary depending upon the condition, the severity of the condition, the subject, e.g., the weight and age of the subject and the mode of administration and the like, which can readily be determined by one of ordinary skill in the art.

The term “subject” refers to any suitable subject, including any animal, such as a mammal. In preferred embodiments described herein, the subject is a human.

The term “comprising” (and related terms such as “comprise” or “comprises” or “having” or “including”) includes those embodiments, for example, an embodiment of any composition of matter, composition, method, or process, or the like, that “consist of” or “consist essentially of” the described features. The term “comprises” or “comprising” can be used interchangeably with “includes”.

“Capable of binding” as described herein refers to any nucleotide sequence that binds to the stated target region (e.g., the pre-mRNA containing the TDP-43 regulated cryptic exon). This can be defined as any nucleotide sequence may be substantially complementary (e.g., at least 90% complementary, or at least 95%) or complementary (e.g., 100% complementary) to the target sequence and/or at least part of a splicing element which has the same number of nucleotides as the antisense sequence.

“Sequence identity” as described herein refers to the % degree of similarity between two nucleotide sequences of the same length.

“UNC13A” as defined herein is a gene that encodes for the UNC13A protein. UNC13 proteins play an important role in neurotransmitter release at synapses.

“STMN2” as defined herein is a gene that encodes for stathmin 2 protein. This protein plays a regulatory role in neuronal growth.

“INSR” as defined herein is a gene that encodes for an insulin receptor which is a member of the receptor tyrosine kinase family of proteins, where binding of insulin or other ligands to this receptor activates the insulin signalling pathway.

“ELAVL3” as defined herein refers to a gene that encodes for the neural-specific protein ELAV like RNA binding protein 3.

“CELF5” as defined herein refers to a gene that encodes for CUGBP Elav-Like Family Member 5 protein.

“TDP-43” as defined herein refers to TAR DNA Binding protein 43 (Transactive response DNA binding protein 43 kDa), which in humans is a protein encoded by the TARDBP gene. TDP-43 has been shown to bind both DNA and RNA and have multiple functions in transcriptional repression, pre-mRNA splicing and translational regulation, among other functions. Pathological TDP-43 may refer to a TDP-43 protein that is associated with a disease state. Pathological TDP-43 may be a hyper-phosphorylated, ubiquitinated or cleaved form of TDP-43, a TDP-43 form with decreased solubility, or a misfolded form of TDP-43, a mutant form of TDP-43, or a TDP-43 with altered cellular location.

A “construct” described herein has its normal meaning in the art and refers to a synthetic nucleic acid sequence that is used to incorporate genetic material into a target cell or tissue. A construct is intended not to be a complete naturally occurring nucleic acid sequence, i.e., as found in the genome of an organism (although the construct itself may comprise component parts that are derived from naturally occurring sequences). The construct may have a maximum length, i.e., the construct may comprise less than 50,000 nucleotides, or less than 40,000 nucleotides, or less than 30,000 nucleotides, or less than 20,000 nucleotides, or in some examples, less than 10,000 nucleotides or less than 5000 nucleotides, or less than 2500 nucleotides, or less than 2000 nucleotides.

A “U7 snRNA” described herein refers to a modified variant of U7 small nuclear RNA which can form a component of the small nuclear ribonucleoprotein complex (U7 snRNP). An unmodified or wildtype U7 snRNA is any U7 snRNA that is involved in processing of replication-dependent histone pre-mRNA. A modified version of U7 snRNA refers to any U7 snRNA variant with controlled changes in the wildtype U7 snRNA such that it is not involved in the processing of replication-dependent histone-dependent pre-mRNA. This is achieved by modifying the Sm binding site of U7 snRNA (i.e., corresponding to SEQ ID NO: 353 AUUUGUCUAG in the wildtype) and modifying the sequence in the wildtype or unmodified U7 snRNA which binds to histone-downstream element within replication-dependent histone pre-mRNAs in the wildtype (i.e., SEQ ID NO: 354 AAGUGUUACAGCUCUUUUAG). The modified U7 snRNA construct described herein instead comprises an antisense sequence that binds to a target sequence (e.g., the TDP-43 regulated cryptic exon or flanking regions thereof) in place of the histone-binding sequence (SEQ ID NO: 354) in unmodified or wildtype U7 snRNA, while also comprising a modified Sm sequence. An example of a modified U7 snRNA with a modified Sm sequence is a U7 smOPT. As defined herein, “U7 smOPT” refers to a modified U7 snRNA as described above but wherein the Sm sequence has been modified to SEQ ID NO 355: AAUUUUUGGAG for the same number of nucleotides.

“Nucleotides” described herein describe the constituent parts of a nucleic acid sequence. Nucleotides comprise a nucleobase (e.g., A, G, T and C in DNA, or A, G, U and C in RNA, however other nucleobases may be used), linked to a sugar (e.g., deoxyribose in DNA, and ribose in RNA, however, other sugars may be used). In DNA and RNA, the sugars are linked by a phosphodiester backbone to form a nucleic acid sequence, however other backbones may be used.

“Complementarity” or “complementary” disclosed herein refers to Watson-Crick base pairing in nucleic acids, e.g., wherein A binds with U (or T or modified variants thereof), and wherein C binds with G (or modified variants thereof).

Reverse complement as described herein refers to the complementary strand or antisense sequence of a sequence, shown from 5′ (left) to 3′ (right).

A cell with depletion (e.g., nuclear depletion) of TDP-43 as described herein may be referred to as a “diseased cell” herein. A cell without depletion (e.g., nuclear depletion) of TDP-43 may be referred to as “healthy cell” herein.

“Splicing” as defined herein refers to the process wherein pre-mRNAs are transformed into mature mRNAs, wherein introns are removed and exons are joined together.

A “cryptic exon” as defined herein refers to a splicing variant that is incorporated into a mature mRNA, introducing frameshifts or stop codons, among other changes in the resulting mRNA. Cryptic exons are typically absent or have much reduced inclusion in the “normal” or “healthy” form of mRNA, and are usually skipped by the spliceosome, but arise in an aberrant form. A cryptic exon may otherwise be referred to as “CE”, “cryptic” “cryptic event” or “cryptic splicing event” herein or elsewhere in the art. The cryptic exon refers to the sequence which is incorrectly incorporated into mature mRNA, defined by a cryptic acceptor splice site and a cryptic donor splice site.

As defined herein, sequences comprising or defined using “T” or thymine, are intended to refer to “U” or uracil, when referring to RNA molecules and sequences defined using “U” or uracil are intended to refer to “T” when referring to DNA molecules. Sequences comprising or defined using “A”, “G”, “C”, “T” or “U” are intended to encompass modified variants of nucleotides, including nucleotides with modified nucleobases and/or modified sugars. In some embodiments, the sequences comprise only unmodified bases.

As defined herein a “splicing factor” is a protein involved in splicing, i.e., the removal of introns from mRNA so that exons are bound together.

As defined herein “a splicing repressor” is a protein involved in repressing or preventing splicing.

As defined herein, splicing elements are any part of the pre-mRNA that is involved in cryptic exon splicing. Splicing elements encompass splice sites (i.e., splice acceptor site and/or splice donor sites defining the cryptic exon), exonic sequence enhancers (ESEs) (defined below), a TDP-43 binding region (or TDP-43 binding motif) (both defined below), or other splicing regulatory elements (i.e., site or sequences where RNA-binding proteins bind and promote splicing events).

An “exonic splice enhancer” or “ESE” defined herein may refer to an ESE that is identified by ESE finder 3.0 (http://krainer01.cshl.edu/cgi-bin/tools/ESE3/esefinder.cgi?process-home). In some embodiments, the ESE is a binding site for an SR protein, for example, a binding site or binding motif for SRSF1, SRSF2, SRSF5 or SRSF6.

A splice site, as understood in the art, is the boundary between an intron sequence and exon sequence. During splicing, the nucleotide sequence is cut at said splice sites, i.e., the nucleotide sequence is cut at the boundary between an intron sequence and exon sequence.

A splice acceptor site is a splicing site that occurs between and intron and exon, i.e., splice site immediately upstream of an exonic sequence wherein the intron is upstream of the exonic sequence. A splice acceptor site is characterised by any splice site that comprises the dinucleotide “AG” upstream of the splice site (i.e., at the end of the intron sequence which is upstream of the exon). A cryptic splice acceptor site is the splice acceptor site of the cryptic exon. Splice acceptor site and cryptic splice acceptor site may be interchangeable herein. The term splice acceptor site may be used interchangeably with the term “3-splice site” or “3-ss”

A splice donor site is a splicing site that occurs between an exon and an intron, i.e., an exonic sequence wherein the exon is upstream of the intron. A splice donor site is characterised by any splice site that comprises the dinucleotide “GU” downstream of the splice site (i.e., at the start of the intron sequence which is downstream of the exon). A cryptic splice donor site is the splice donor site of the cryptic exon. Splice donor site and cryptic splice donor site may be interchangeable herein. The term splice donor site may be used interchangeably with the term “5-splice site” or “5-ss”

“Depletion of TDP-43” or “depleted of TDP-43” as described herein, may be defined as a cell of a cell or as an average (mean) of a population of cells, with at least 20% loss of TDP-43, or at least 25% loss, or preferably at least 50% loss of TDP-43 in the cell, preferably the nucleus, as compared to a healthy cell (or as an average (mean) of a population of healthy cells) of the same type. In some examples, the term “nuclear depletion of TDP-43” can be replaced with or is interchangeable with the term “absence of binding of TDP-43 to the TDP-43 binding region”, and the term “without nuclear depletion of TDP-43” can be replaced with or is interchangeable with the term “presence of binding of TDP-43 to the TDP-43 binding region. Depletion of TDP-43 can be determined by standard methods, such as western blotting. In other embodiments, depletion may be determined by determining the presence of a STMN2 cryptic splicing event (i.e., the presence of a STMN2 cryptic exon 2a as defined herein) in a cell transcript, which may be determined by RNA-sequencing. Depletion of TDP-43 refers to depletion of “normal” or wild-type TDP-43, and may not include pathological or mutated TDP-43. Pathological TDP-43 may be a hyper-phosphorylated, ubiquitinated or cleaved form of TDP-43, a TDP-43 form with decreased solubility, or a misfolded form of TDP-43, a mutant form of TDP-43, or a TDP-43 with altered cellular location.

The term “RNA-seq” referred to herein, otherwise known as “RNA sequencing”, refers to a next-generation sequencing technology which reveals the presence and quantity of RNA in a sample which can be used to analyse the cellular transcriptome.

“Capable of modulating splicing of a TDP-43 regulated cryptic exon” as described herein refers to a construct that corrects splicing by at least partially preventing inclusion of the TDP-43 regulated cryptic exon in the mature mRNA of the cell transcript, (e.g., by binding to the pre-mRNA which contains the TDP-43 regulated cryptic exon).

An “anti-sense oligonucleotide” or “ASO” described herein has its normal meaning in the art and refers to an isolated (i.e., stand-alone) synthetic single stranded string of nucleic acids, typically less than 30 nucleotides in length. ASOs are used in the art as therapeutics, e.g., for targeting mRNA. They bind complementarily (‘antisense’) through Watson-Crick base pairing to a defined part of a nucleotide sequence of the pre-messenger ribonucleic acid (pre-mRNA) or mature mRNA (‘sense’) to modulate mRNA function or splicing. ASO as described herein is distinct from a modified U7 snRNA constructs described herein which instead incorporate an antisense sequence within a modified U7 snRNA construct, e.g., comprising a modified Sm sequence, more preferably a smOPT sequence.

Unless context explicitly states otherwise, it is envisaged that any embodiment described herein may be combined with any other embodiment described herein. Similarly, the features of any dependent claim (i.e., representing preferred embodiments of the present invention) may be readily combined with the features of any of the independent claims or other dependent claim or embodiments, unless context clearly dictates otherwise.

Any genomic or chromosomal position described herein refers to the position on the human genome and associated transcriptome (hg38).

When ranges are used herein, all combinations and sub-combinations of ranges and specific embodiments therein are intended to be included. The term “about” or “˜” when referring to a number or a numerical range means that the number or numerical range referred to is an approximation within experimental variability (or within statistical experimental error), and thus the number or numerical range may vary. Typical experimental variabilities may stem from, for example, changes and adjustments necessary during scale-up from laboratory experimental and manufacturing settings to large scale.

It must be noted that as used herein and in the appended claims, the singular forms “a”, “an”, and “the” include plural referents unless the context clearly dictates otherwise.

Unless defined otherwise, all technical and scientific terms used herein have the same meanings as commonly understood by one of ordinary skill in the art to which this invention belongs. Abbreviations used herein have their conventional meaning within the chemical and biological arts, unless otherwise indicated.

### Construct

Disclosed herein is a modified U7 snRNA construct comprising
(i) an antisense sequence having between 16 to 30 nucleotides which is at least 90% complementary to a TDP-43 regulated cryptic exon sequence or flanking regions thereof, and
(ii) a sequence comprising a binding domain for a hnRNP protein, wherein the modified U7 snRNA construct is capable of modulating splicing of the TDP-43 regulated cryptic exon in a cell.

In some embodiments, the flanking regions described herein may be defined as 150 nucleotides upstream and downstream of the TDP-43 regulated cryptic exon, or 100 nucleotides upstream and downstream of the TDP-43 regulated cryptic exon, or 50 nucleotides upstream and downstream of the TDP-43 regulated cryptic exon, or 25 nucleotides upstream and downstream of the TDP-43 regulated cryptic exon. In some embodiments, the cryptic exon sequence or flanking regions thereof may be defined by a defined sequence for a particular TDP-43 cryptic exon (e.g., SEQ ID NO: 1, 2, 3,4, 7 or 9).

In some embodiments, the modified U7 snRNA construct comprises a transcription start site, e.g., in the form of an A nucleotide, at the start of the construct.

In some embodiments, the modified U7 snRNA construct comprises the sequence comprising a binding domain for a hnRNP protein downstream of the transcription start site, preferably immediately downstream of the transcription start site.

In some embodiments (and in the examples described herein), the modified U7 snRNA construct comprises the antisense sequence (i.e., which is at least 90% complementary to a TDP-43 regulated cryptic exon sequence or flanking regions thereof) downstream of the transcription start site, and preferably downstream of the transcription start site and binding sequence for the hnRNP protein. In alternative embodiments, the modified U7 snRNA construct comprises the antisense sequence (i.e., which is at least 90% complementary to a TDP-43 regulated cryptic exon sequence or flanking regions thereof) immediately downstream of the transcription start site, and preferably upstream of the sequence comprising the binding domain for the hnRNP protein.

In some embodiments, the modified U7 snRNA construct comprises a modified Sm sequence (i.e., the modified U7 snRNA is a U7 smOPT construct). Preferably, the modified Sm sequence is downstream of both the sequence comprising a binding domain for a hnRNP protein and the antisense sequence which is at least 90% complementary to a TDP-43 regulated cryptic exon sequence or flanking regions thereof. In some embodiments, the U7 snRNA construct comprises a modified Sm sequence that has at least 80% sequence identity, (i.e., for the same number of nucleotides), to SEQ ID NO 355: AAUUUUUGGAG, or at least 85% sequence identity, or at least 90% sequence identity, or at least 100% sequence identity to SEQ ID NO 355. In preferred embodiments, the modified U7 snRNA construct is a U7 smOPT construct. The U7 smOPT construct comprises the modified Sm sequence corresponding to SEQ ID NO 355. In some embodiments, the modified U7 snRNA construct comprises a 3′ hairpin sequence downstream of the modified Sm sequence. This may be any suitable hairpin sequence. In some embodiments, the 3′ hairpin sequence has a sequence that is at least 80%, or at least 85%, or at least 90%, or at least 95%, or at least 100% identical to CAGGUUUUCUGACUUCGGUCGGAAAACCCCU (SEQ ID NO: 356). The modified U7 snRNA construct does not comprise a wildtype Sm sequence (SEQ ID NO: 353) The modified U7 snRNA construct does not comprise a binding sequence to a histone-downstream element (HDE), i.e., the modified U7 snRNA construct does not comprise the sequence corresponding to SEQ ID NO: 354. In preferred embodiments, the sequence comprising the binding domain for the hnRNP protein and the antisense sequence which is at least 90% complementary to a TDP-43 regulated cryptic exon and flanking regions thereof is directly present in the modified U7 snRNA construct in place of the binding sequence for the histone-downstream element in wild-type U7 snRNA.

In some examples, the modified U7 snRNA construct comprises a sequence that is at least 80% identical to, or at least 85% identical to, or at least 90% identical to, or at least 95% identical to SEQ ID NO: 358, 360, 363, 365,367, 369, 371, 373, 375, 377, 379, 381, 383, 385 for a (i.e., for UNC13A), SEQ ID NO: 390, 392, 394, 396, 398, 400, 402, 404, 406 (i.e., for STMN2) and SEQ ID NO: 408, 410, 412, 414, 416, 418 (i.e., for INSR). Sequence identity is compared to a sequence with the same number of nucleotides.

### Antisense Sequence

The constructs described herein comprise an antisense sequence that is at least 90% complementary to a TDP-43 regulated cryptic exon or flanking regions thereof. In some embodiments, the antisense sequence is at least 91% complementary, or at least 92% complementary, or at least 93% complementary, or at least 94% complementary, or at least 95% complementary, or at least 96% complementary, or at least 97% complementary, or at least 98% complementary, or at least 99% complementary, or at least 100% complementary to a TDP-43 regulated cryptic exon or flanking regions thereof. In some embodiments, the TDP-43 regulated cryptic exon or flanking regions thereof may be defined by SEQ ID NO: 1, 2, 3, 4, 7 or 9 or SEQ ID NO: 448-453.

The flanking region of the TDP-43 regulated cryptic exon may be defined as the 150 nucleotides upstream and/or downstream of the cryptic exon (i.e., in intronic regions surrounding the cryptic exon sequence). In some embodiments, the flanking region may be the 100 nucleotides upstream and/or downstream of the cryptic exon, or up to 75 nucleotides upstream and/or downstream of the cryptic exon, or up to 50 nucleotides upstream and/or downstream of the cryptic exon, or up to 30 nucleotides upstream and/or downstream of the cryptic exon, or 25 nucleotides upstream and/or downstream of the cryptic exon. In some embodiments, the antisense sequence may partially overlap with the cryptic exon sequence (i.e., the antisense sequence is capable of binding to a part of the cryptic exon sequence and part of the flanking region thereof). In some embodiments, the antisense sequence is capable of binding to at least 5 nucleotides within the cryptic exon, or at least 10 nucleotides, or at least 15 nucleotides within the cryptic exon sequence. The cryptic exon sequence may be any cryptic exon sequence defined herein. In some embodiments, the antisense sequence may be capable of binding within the cryptic exon sequence. In some embodiments, the antisense sequence is at least 90%, or at least 91%, or at least 92%, or at least 93%, or at least 94%, or at least 95%, or at least 96%, or at least 97%, or at least 98%, or at least 99%, or 100% complementary to any one of SEQ ID NO: 5, 6 (short and long cryptic exon for UNC13A), SEQ ID NO 8 (cryptic exon of STMN2) or SEQ ID NO 10 (cryptic exon for INSR).

A “TDP-43 regulated cryptic exon” defined herein is a cryptic exon that is regulated by binding of TDP-43 to a TDP-43 binding region in close proximity to the cryptic exon, such that splicing of the cryptic exon is repressed. A TDP-43 regulated cryptic exon is therefore characterized as a cryptic exon that is present or increased relative to a healthy cell in the mature mRNA of a gene when there is depletion of TDP-43 in the cell and/or in the absence of TDP-43 binding, but is absent in the mature mRNA of a gene or decreased when there is no such depletion of TDP-43. A TDP-43 regulated cryptic exon is further characterized by a cryptic exon that comprises or is in close proximity to a TDP-43 binding region (defined below), wherein close proximity is defined as a region which is entirely within, partially overlaps, or is within 150 nucleotides of the cryptic exon sequence. In some embodiments, the TDP-43 binding region encompasses at least part of the cryptic exon sequence, and/or extends upstream or downstream of the cryptic exon sequence. In some embodiments, the TDP-binding region (or at least a part of the TDP-43 binding region) is within 150 nucleotides (i.e., upstream or downstream) of the cryptic exon, or within 100 nucleotides, or within 50 nucleotides, or within 25 nucleotides of the cryptic exon, or within the cryptic exon. In some embodiments, the TDP-43 binding region is upstream of the cryptic exon sequence, within the cryptic exon sequence, or downstream of the cryptic exon sequence or any combination thereof. Such cryptic exons are well-known in the art and can be readily identified in the art (e.g., by comparing the level of cryptic splicing in healthy or wildtype cells versus cells depleted by TDP-43 (i.e., in diseased cells or cells with TDP-43 knockdown or TDP-43 knockout). In some embodiments, the TDP-43 binding region comprises or is a TDP-binding motif. The TDP-43 binding motif may be as elsewhere described herein.

In some embodiments, the TDP-43 regulated cryptic exon is a cryptic exon within the following genes: AARS1, AC002310.11, AC008676.3, AC022387.2, ACTL6B, ADARBI, ADCY1, ADGRLI, AGK, AHNAK, AKT3, AL035461.3, AL360181.3, AP000662.4, ARAP3, ARHGAP22, ARHGAP23, ATAD5, ATG4B, ATP5MG, ATP8A2, ATXNI, C2orf81, CAMK2B, CAMTA1, CCDCl02B, CCDC33, CDHR2, CELF5, CEP290, CEP83, CHD8, CHFR, CRLS1, CTD-2162K18.4, CYFIP2, DACH2, DACT3-AS1, DAGLA, DELE1, DGKA, DLG5, DLGAP1, DNAJC12, DNMT3A, DOCK1, DPF1, DUXAP9, EIF2A, ELAVL3, EP400, EPB41L4A, EPS8L2, FADS2, FAM114A2, FAM156A, FIRRE, FKBP14-AS1, FRYL, G3BP1, GALNT12, GATA2, GPSM2, GREB1, GRIN2D, GSTCD, HAUS2, HDGFL2, ICA1, IGSF21, IK, IL15, INSR, INTS11, IQCE, IQCK, ISYNA1, ITGA7, ITPR3, KALRN, KCNQ2, KCNTI, KIAA1211, KIAA1217, KIF21A, KLC1, KNDC1, L3MBTL1, LINC01322, LINC01503, LINGO1, LRPIB, LRP8, LTBP2, MACRODI, MADD, MANBAL, MAP2K6, MBP, MCIR, MCM9, MED13L, MEIS2, MGAT5B, MIER3, MMAA, MRPL34, NBPF9, NIPSNAP3B, NTRK2, NUP188, PAOX, PATJ, PCDH1IX, PDCD6, PDE2A, PHF2, PLEKHG2, PLEKHM2, PRUNE2, PTPN13, PTPN21, PUDP, PUS7L, RBMXL1, RFLNA, RHOQ, RP1-138B7.8, RP11-108K14.8, RP11-411B6.6, RP11-47909.4, RP11-505D17.1, RP11-61L23.2, RP11-73M18.2, RP5-967N21.13, RSF1, SEC31B, SEPT7P2, SEPTIN6, SEPTIN7P2, SERGEF, SETD5, SGMSI, SIPAIL3, SLC24A3, SLC25A14, SLC2A11, SLC35G1, SLC41A2, SPATS2, SPINI, STMN2, STRA6, STXBP5L, SYNEI, SYNJ2, SYT7, TAF6, TAFA2, TEX9, TGFB3, THUMPD3-AS1, TMEM175, TMEM189, TPRAI, TRAPPC12, TRIO, TRRAP, TTC39C-AS1, TTTY14, TXLNGY, UNC13A, USP10, WARS2, WASL, WDR19, WWOX, ZBTB18, ZCCHC4, ZFAT, ZNF202, ZNF236, ZNF382, ZNF420, ZNF423, ZNF429, ZNF527, ZNF571-AS1, ZNF583, ZNF598, ZNF81, ZNF814, ZNF826P, ZRANB3.

In some embodiments, the TDP-43 regulated cryptic exon is selected from a UNC13A cryptic exon, a TDP-43 regulated STMN2 cryptic exon or a TDP-43 regulated INSR cryptic exon, a TDP-43 regulated ELAVL3 cryptic exon, a TDP-43 regulated G3BP1 cryptic exon, a TDP-43 regulated AARS1 cryptic exon, a TDP-43 regulated CELF5 cryptic exon, a CAMK2B cryptic exon, or an UNC13B cryptic exon, preferably wherein the antisense sequence comprises a sequence that is at least 90%, or at least 95%, or at least 100% complementary to any one of SEQ ID NO: 1, 2, 3, 4, 7, 9, 448-453.

In some examples described herein, the TDP-43 regulated cryptic exon is a TDP-43 regulated UNC13A cryptic exon, a TDP-43 regulated STMN2 cryptic exon or a TDP-43 regulated INSR cryptic exon. In some examples, the antisense sequence comprises a sequence that is at least 90%, or at least 95%, or at least 100% complementary to any one of SEQ ID NO: 1, 2, 3, 4, 7 or 9.

As defined herein, the TDP-43 binding region is defined as a sequence that is capable of binding to TDP-43. This term may be used interchangeably with the term “TDP-43 binding domain” or “TDP-43 binding site” and may encompass a sequence with a “TDP-43 binding motif”. The TDP-43 binding region is typically characterised or encompasses a “UG rich” sequence or region. In some embodiments, the “UG” rich region may be defined, and the TDP-43 binding region may comprise a region of at least 6 nucleotides, or preferably at least 10 nucleotides, or at least 20 nucleotides, with a statistically significant enrichment of UG dinucleotides and/or UGNNUG hexanucleotides, wherein N is A, U, C or G. In some embodiments, the TDP-43 binding region comprises a region of at least 6 nucleotides (e.g., 6 to 1000 nucleotides, or 6 to 150 nucleotides), with a statistically significant enrichment of UG dinucleotides and/or UGNNUG hexanucleotides, wherein N is A, U, C or G, wherein statistically significant enrichment is defined as a probability of less than 0.2% that a random sequence of nucleotides of equal length would feature an equal number of UG dinucleotides and/or UGNNUG hexanucleotides. In some embodiments, the statistically significant enrichment is defined as a probability of less than or equal to 0.15% that a random sequence of nucleotides of equal length would feature an equal number of UG dinucleotides and/or UGNNUG hexanucleotides, or less than or equal to 0.1%, or less than or equal to 0.05%, or less than or equal to 0.01%, or less than or equal to 0.003%, or equal or less than 0.001%, or equal or less than 0.0003%, or equal or less than 0.0001%, or equal or less than 1×10⁻⁵, or of less than or equal to 1×10⁶, or of less than or equal to 1×10⁻⁷, or of less than or equal to 1×10⁻⁸, or of less than or equal to 1×10⁻⁹, or less than or equal to 1×10⁻¹⁰. These definitions cover both short sequences or regions which are highly enriched for UG, and longer sequences which are broadly enriched for UG, both of which are shown to be preferentially bound by TDP-43. In some embodiments, the TDP-43 binding region comprises a sequence that is enriched with UG dinucleotides. In some embodiments, an enrichment of UG dinucleotides may be described as a TDP-binding motif and is defined as a sequence comprising at least 6 nucleotides with 100% UG dinucleotides (i.e., UGUGUG), or one or more region with at least 6 nucleotides with 100% UG dinucleotides. In some embodiments, an enrichment of UG dinucleotides is defined as a sequence comprising at least 8 nucleotides (or one or more region with at least 8 nucleotides) with at least 80% UG dinucleotides, or at least 85%, or at least 90%, or at least 95%, or 100% UG dinucleotides. In some embodiments, an enrichment of UG dinucleotides is defined as a sequence which comprises at least 10 nucleotides (or one or more region with at least 10 nucleotides) with at least 60% UG dinucleotides, or at least 65%, or at least 70%, or at least 75%, or at least 80%, or at least 85%, or at least 90%, or at least 95%, or at least 100% UG dinucleotides. In some embodiments, an enrichment of UG dinucleotides is defined as a sequence that comprises at least 15 nucleotides (or one or more region with at least 15 nucleotides) with at least 53% UG dinucleotides, or at least 60%, or at least 65%, or at least 70%, or at least 75%, or at least 80%, or at least 85%, or at least 90%, or at least 95%, or 100% UG dinucleotides). In some embodiments, the TDP-43 binding region comprises a sequence that comprises at least one UGUGUG motif, or at least one UGUGUGUGUG motif. However, while TDP-43 is capable of binding a large variety of different sequences that are UG-rich, the TDP-43 binding region does not have to bind a pure UG-repeat. This is in part due to the protein's lack of contact with some RNA residues within its binding footprint, and in part due to multivalent protein-protein interactions which enhance binding to large regions of UG-rich RNA. This means that in some embodiments, the TDP-43 binding region may not require any “pure” UG-repeats or motifs, such as the TDP-43 binding region in UNC13A. In some embodiments, the TDP-43 binding region may be a well-known, described annotated binding region. For example, the sequence characteristics which promote binding of TDP-43 are described in Lukavsky et al., 2013 (NSMB, 20, pages 1443-1449) which is incorporated herein by reference. The TDP-43 binding region may be or may have been previously identified by transcriptome mapping of TDP-43 on the human genome, for example, as determined by immunoprecipitation, for example, iCLIP (individual-nucleotide resolution UV Cross-Linking and Immunoprecipitation). In some embodiments, the antisense sequence may be selected to bind upstream or downstream of a TDP-43 motif, for example, within 40 nucleotides upstream or downstream of a TDP-43 motif, or within 20 nucleotides upstream or downstream of a TDP-43 motif. This is because the present construct works by bringing the hnRNP protein to where TDP-43 usually binds, and as a result, it may be beneficial to target the flanking regions of the TDP-43 motif.

The antisense sequence described herein may comprise or consist of from 16 to 30 nucleotides. In some embodiments, the antisense sequence is between 16 and 26 nucleotides, or between 17 and 23 nucleotides, or between 18 and 22 nucleotides. In some embodiments, the antisense sequence comprises or consists of 16 nucleotides, or 17 nucleotides, or 18 nucleotides, or 19 nucleotides, or 20 nucleotides, or 21 nucleotides, or 22 nucleotides, or 23 nucleotides, or 24 nucleotides, or 25 nucleotides, or 26 nucleotides, or 27 nucleotides, or 28 nucleotides, or 29 nucleotides or 30 nucleotides. In some embodiments, the antisense sequence comprises at least 16 nucleotides, or at least 17 nucleotides, or at least 18 nucleotides, or at least 19 nucleotides, or at least 20 nucleotides, or at least 21 nucleotides, or at least 22 nucleotides, or at least 23 nucleotides, or at least 24 nucleotides, or at least 25 nucleotides, or at least 26 nucleotides, or at least 27 nucleotides, or at least 28 nucleotides, or at least 29 nucleotides. In some embodiments, the antisense sequence comprises less than 30 nucleotides, or less than 29 nucleotides, or less than 28 nucleotides, or less than 27 nucleotides, or less than 26 nucleotides, or less than 25 nucleotides, or less than 24 nucleotides, or less than 23 nucleotides, or less than 22 nucleotides, or less than 21 nucleotides, or less than 20 nucleotides, or less than 19 nucleotides, or less than 18 nucleotides, or less than 17 nucleotides. The longer the antisense sequence, the more efficiently the modified U7 snRNA construct is found to bind and the more effective the construct is as a steric block, however, this comes with a trade-off of an increased tendency for off-target binding.

In some embodiments, the construct may comprise more than one antisense sequence, for example, two or more antisense sequences, that are at least 90% complementary to a TDP-43 regulated cryptic exon or flanking region thereof, or 95% complementary to a TDP-43 regulated cryptic exon or flanking region thereof, or 100% complementary to a TDP-43 regulated cryptic exon or flanking region thereof. These antisense sequences may be capable of binding to different splicing elements.

In some embodiments, the antisense sequence is capable of binding (i.e., at least 90%, or at least 95%, or 100% complementary) to a splicing element of the cryptic exon sequence, optionally wherein the antisense sequence is at least 90% complementary to one of SEQ ID NO: 11-40 or 454-471.

In some embodiments, the antisense sequence is capable of binding (i.e., at least partially) to a splicing element of the cryptic exon sequence, or two or more splicing elements of the cryptic exon sequence, preferably wherein one of the two or more splicing elements of the cryptic exon sequence is a TDP-43 binding region. In some embodiments, the antisense sequence may be capable of binding to a TDP-43 binding region and a splice site (e.g., a 5′ splice site or a 3′ splice-site). In some embodiments, the antisense sequence may be capable of binding to a TDP-43 binding region and an ESE. In an example, for an antisense sequence targeting the UNC13A cryptic exon, results are particularly good when the antisense sequence is capable of binding to the TDP-binding sequence and a 5′-splice site. In some embodiments, the splicing element is selected from a splice site, a TDP-43 binding region (e.g., a TDP-43 binding motif), or an exonic splice enhancer. In some embodiments, the antisense sequence is capable of binding, at least partially, to a splicing element of the cryptic exon sequence, but may also bind to a flanking region upstream or downstream of the splicing element. The flanking regions may include the 25 nucleotides upstream of downstream of the splicing element, optionally the 20 nucleotides upstream of downstream of the splicing element, optionally the 15 nucleotides upstream or downstream of the splicing element, or the 10 nucleotides upstream or downstream of the splicing element, or the 5 nucleotides upstream or downstream of the splicing element. In some embodiments, (e.g., for some embodiments where the antisense sequence is capable of binding to a TDP-43 binding region), the antisense sequence is capable of binding completely to or within the splicing element (i.e., within the TDP-43 binding region, or completely overlapping with the ESE).

In some embodiments, the portion of the antisense sequence that is capable of binding to the splicing element is closer to the 3′-end of the antisense sequence. In some embodiments, the portion of the antisense sequence that is capable of binding to the splicing element is within 7 nucleotides, or 6 nucleotides, or 5 nucleotides, or 4 nucleotides, or 3 nucleotides, or 2 nucleotides from the 3′ end of the antisense sequence. In some embodiments, the portion of the antisense sequence that is capable of binding to the splicing element is closer to the 5′-end of the antisense sequence. In some embodiments, the portion of the antisense sequence that is capable of binding to the splicing element is within 7 nucleotides, or 6 nucleotides, or 5 nucleotides, or 4 nucleotides, or 3 nucleotides, or 2 nucleotides from the 5′ end of the antisense sequence.

In some embodiments, the splicing element is a splice site, (i.e., the antisense sequence is capable of binding (in other words, overlaps with) a splice site of the cryptic exon, more particularly wherein the antisense sequence overlaps with at least one nucleotide upstream or downstream of the splice site). In some embodiments, the antisense sequence is capable of binding to at least 2 nucleotides, or at least 3 nucleotides, or at least 4 nucleotides, or at least 5 nucleotides, or at least 6 nucleotides, or at least 7 nucleotides, or at least 8 nucleotides upstream and/or downstream of the splice site.

In some embodiments, the antisense sequence is capable of binding to a splice site (i.e., and flanking regions thereof), preferably wherein the antisense sequence is at least 90% complementary, or at least 95% complementary, or at least 100% complementary to any one of SEQ ID NO 11, 19, 20, 21, 22, 31, 454, 458, 460, 463, 467 or 469.

In some embodiments, the splicing element may be a 3′-splice site (i.e., a splice acceptor site). In some embodiments, the antisense sequence is capable of binding (in other words, overlaps with) to the “ag” dinucleotide upstream of the splice acceptor site.

In some embodiments, the splicing element may be a 5′ splice site (i.e., a splice donor site). In some embodiments, the antisense sequence is capable of binding (in other words, overlaps with) the “gu” dinucleotide downstream of the splice donor site.

In some embodiments, the splicing element is a TDP-43 binding region, and the antisense sequence is capable of binding to at least a portion of the TDP-43 binding region. In some embodiments, the antisense sequence may bind to at least a portion of the TDP-43 binding region and a flanking region thereof (i.e., as defined as 20 nucleotides upstream or downstream of the TDP-43 binding region, optionally the 15 nucleotides upstream or downstream of the TDP-43 binding region, or the 10 nucleotides upstream or downstream of the TDP-43 binding region, or the 5 nucleotides upstream or downstream of the TDP-43 binding region). In some embodiments, the antisense sequence binds to at least 5 nucleotides, or least 7 nucleotides, or at least 10 nucleotides, or at least 15 nucleotides of the TDP-43 binding region, or completely overlaps with (i.e., is contained within) the TDP-43 binding region. In some embodiments, the TDP-43 binding region comprises a sequence of at least 6 nucleotides, or preferably at least 10 nucleotides, with a statistically significant enrichment of UG dinucleotides and/or UGNNUG hexanucleotides, wherein N is A, U, C or G, wherein statistically significant enrichment is defined as a probability of less than 0.2% that a random sequence of nucleotides of equal length would feature an equal number of UG dinucleotides and/or UGNNUG hexanucleotides, or preferably wherein statistically significant enrichment is defined as a probability of less than 0.05% that a random sequence of nucleotides of equal length would feature an equal number of UG dinucleotides and/or UGNNUG hexanucleotides. In other embodiments, the TDP-43 binding region may be as according to any other definition as described herein. In some embodiments, the TDP-43 binding region may comprise or is a TDP-43 binding motif as described herein. In some embodiments, the TDP-43 binding region or TDP-43 binding region and flanking region thereof is defined by SEQ ID NO: 12, 13, 23, 23, 24, 25, 26, 32, 33, 455, 456, 457, 459, 461, 462, 464, 465, 466, 468, 470 or 471, more preferably SEQ ID NO: SEQ ID NO: 12, 13, 23, 23, 24, 25, 26, 32, 33.

In some embodiments, the antisense sequence is capable of binding to one or more exonic splice enhancers (ESE) (i.e., and flanking regions thereof) (ESE) as defined by ESE finder 3.0, e.g., using the SR Protein matrix library. In some embodiments, the following thresholds are used when using ESE finder 3.0, when selecting the SR Protein matrix library, SRSF1-1.956, SRSF2-2.383, SRSF5 2.67 and SRSF6-2.676. The ESEs defined by ESE finder 3.0 may be as described in the reference “An increased specificity score matrix for the prediction of SF2 ASF-specific exonic splicing enhancers. Hum. Mol. Genet. 15 (16): 2490-2508, which is incorporated herein by reference. The ESE may be an SR protein binding site, i.e., selected from SRSF1, SRSF2, SRSF5 or SRSF6. In some embodiments, the ESE and flanking regions thereof is defined by SEQ ID NO: 14, 15, 16, 17, 18, 27, 28, 29, 30, 34, 35, 36, 37, 38, 39 or 40.

In some embodiments, the ESE may be a SRSF1 binding site. In some embodiments, the SRSF1 binding site may comprise a motif selected from CACACGA, CACACGU, CACACGG, CAGACGA, CAGACGU, CAGACGG, CACAGGA, CACAGGU, CACAGGG, CAGAGGA, CAGAGGU, CAGAGGG, CGCACGA, CGCACGU, CGCACGG, CGGACGA, CGGACGU, CGGACGG, CGCAGGA, CGCAGGU, CGCAGGG, CGGAGGA, CGGAGGU, CGGAGGG, CUCACGA, CUCACGU, CUCACGG, CUGACGA, CUGACGU, CUGACGG, CUCAGGA, CUCAGGU, CUCAGGG, CUGAGGA, CUGAGGU, CUGAGGG, CACCCGA, CACCCGU, CACCCGG, CAGCCGA, CAGCCGU, CAGCCGG, CACCGGA, CACCGGU, CACCGGG, CAGCGGA, CAGCGGU, CAGCGGG, CGCCCGA, CGCCCGU, CGCCCGG, CGGCCGA, CGGCCGU, CGGCCGG, CGCCGGA, CGCCGGU, CGCCGGG, CGGCGGA, CGGCGGU, CGGCGGG, CUCCCGA, CUCCCGU, CUCCCGG, CUGCCGA, CUGCCGU, CUGCCGG, CUCCGGA, CUCCGGU, CUCCGGG, CUGCGGA, CUGCGGU, or CUGCGGG.

In some embodiments, the ESE may be a SRSF2 binding site. In some embodiments, the SRSF2 binding site may comprise a motif selected from GGWWNCWG, GAWWNCWG, GGWWNGWG, GAWWNGWG where N is A, U, C or G and W is U or A, or wherein the SRSF2 binding site is GGCCNCUG, GACCNCUG, GGUCNCUG, GAUCNCUG, GGCUNCUG, GACUNCUG, GGUUNCUG, GAUUNCUG, GGCCNCUA, GACCNCUA, GGUCNCUA, GAUCNCUA, GGCUNCUA, GACUNCUA, GGUUNCUA, GAUUNCUA, GGCCNCCG, GACCNCCG, GGUCNCCG, GAUCNCCG, GGCUNCCG, GACUNCCG, GGUUNCCG, GAUUNCCG, GGCCNCCA, GACCNCCA, GGUCNCCA, GAUCNCCA, GGCUNCCA, GACUNCCA, GGUUNCCA, GAUUNCCA.

In some embodiments, the ESE may be a SRSF5 binding site. In some embodiments, the SRSF5 binding site may comprise a motif selected from UCWCWGG, CCWCWGG, UCWCWCG, CCWCWCG, UCWCWGC, CCWCWGC, UCWCWCC, CCWCWCC, UCWCWAG, CCWCWAG, UCWCWAG, CCWCWAG, UCWCWAC, CCWCWAC, UCWCWAC, CCWCWAC, where W is A or U.

In some embodiments, the ESE may be a SRSF6 binding site. In some embodiments, the SRSF6 binding site may comprise a motif selected from UGCGUC, CGCGUC, UACGUC, CACGUC, UGCAUC, CGCAUC, UACAUC, CACAUC, UGCGGC, CGCGGC, UACGGC, CACGGC, UGCAGC, CGCAGC, UACAGC, CACAGC, UGCGUA, CGCGUA, UACGUA, CACGUA, UGCAUA, CGCAUA, UACAUA, CACAUA, UGCGGA, CGCGGA, UACGGA, CACGGA, UGCAGA, CGCAGA, UACAGA, or CACAGA.

In some embodiments, the antisense sequence comprises or consists of a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 42-352. In some embodiments, the antisense sequence comprises a nucleotide sequence having at least 16 and 30 nucleotides that is at least 90% complementary to a TDP-43 regulated cryptic exon sequence, and wherein the nucleotide sequence comprises a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 42-352. In some embodiments, the nucleotide sequence may consist of 17, 18, 19, 20, 21, 22, 23, 24,25, 26, 27 28, 29 or 30 nucleotides.

In some embodiments, the antisense sequence comprises a sequence that is at least 80% identical, or at least 85% identical, or at least 90% identical, or at least 95% identical or at least 100% identical to any one of SEQ ID NO 420, 362, 364, 366, 368, 370, 372, 374, 382, 384, 391, 393, 395, 397, 399, 401, 403, 405, 407, 409, 411, 413, 415, 417 or 419, for a sequence with the same number of nucleotides. In some embodiments, the antisense sequence comprises sequence of at least 16 nucleotides (or 16 nucleotides) that is at least 80% identical, or at least 85% identical, or at least 90% identical, or at least 95% identical or at least 100% identical to at least a portion of any one of SEQ ID NO 420, 362, 364, 366, 368, 370, 372, 374, 382, 384, 391, 393, 395, 397, 399, 401, 403, 405, 407, 409, 411, 413, 415, 417 or 419, i.e., for the same number of nucleotides. In some embodiments, the antisense sequence comprises sequence of 17 nucleotides that is at least 80% identical, or at least 85% identical, or at least 90% identical, or at least 95% identical or at least 100% identical to a 17 nucleotide sequence of any one of SEQ ID NO 420, 362, 364, 366, 368, 370, 372, 374, 382, 384, 391, 393, 395, 397, 399, 401, 403, 405, 407, 409, 411, 413, 415, 417 or 419. In some embodiments, the antisense sequence comprises sequence of 18 nucleotides that is at least 80% identical, or at least 85% identical, or at least 90% identical, or at least 95% identical or at least 100% identical to an 18 nucleotide sequence of any one of SEQ ID NO 420, 362, 364, 366, 368, 370, 372, 374, 382, 384, 391, 393, 395, 397, 399, 401, 403, 405, 407, 409, 411, 413, 415, 417 or 419. In some embodiments, the antisense sequence comprises sequence of 19 nucleotides that is at least 80% identical, or at least 85% identical, or at least 90% identical, or at least 95% identical or at least 100% identical to a 19 nucleotide sequence of any one of SEQ ID NO 420, 362, 364, 366, 368, 370, 372, 374, 382, 384, 391, 393, 395, 397, 399, 401, 403, 405, 407, 409, 411, 413, 415, 417 or 419. In some embodiments, the antisense sequence comprises sequence of 20 nucleotides that is at least 80% identical, or at least 85% identical, or at least 90% identical, or at least 95% identical or at least 100% identical to a 20 nucleotide sequence of any one of SEQ ID NO 420, 362, 364, 366, 368, 370, 372, 374, 382, 384, 391, 393, 395, 397, 399, 401, 403, 405, 407, 409, 411, 413, 415, 417 or 419. In some embodiments, the antisense sequence comprises sequence of 21 nucleotides that is at least 80% identical, or at least 85% identical, or at least 90% identical, or at least 95% identical or at least 100% identical to a 21 nucleotide sequence of any one of SEQ ID NO 420, 362, 364, 366, 368, 370, 372, 374, 382, 384, 391, 393, 395, 397, 399, 401, 403, 405, 407, 409, 411, 413, 415, 417 or 419. In some embodiments, the antisense sequence comprises sequence of 22 nucleotides that is at least 80% identical, or at least 85% identical, or at least 90% identical, or at least 95% identical or at least 100% identical to a 22 nucleotide sequence of any one of SEQ ID NO 420, 362, 364, 366, 368, 370, 372, 374, 382, 384, 391, 393, 395, 397, 399, 401, 403, 405, 407, 409, 411, 413, 415, 417 or 419.

### UNC13A

In some embodiments, the TDP-43 regulated cryptic exon is an UNC13A cryptic exon. The TDP-43 regulated UNC13A cryptic exon is the cryptic exon between exons 20 and 21 in the human UNC13A gene. In some embodiments, the antisense sequence is at least 90% complementary (or at least 95%, or at least 100% complementary) to SEQ ID NO: 1 or SEQ ID NO: 2. In some embodiments, the antisense sequence is at least 90% complementary (or at least 95%, or at least 100% complementary) to SEQ ID NO: 3 or SEQ ID NO: 4. In some embodiments, the antisense sequence is at least 90% complementary (or at least 95%, or at least 100% complementary) to SEQ ID NO: 5. In some embodiments, the antisense sequence is at least 90% complementary (or at least 95%, or at least 100% complementary) to SEQ ID NO: 6.

In some embodiments, the antisense sequence comprises or consists of a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 103-260. In some embodiments, the antisense sequence comprises a nucleotide sequence having 16 and 30 nucleotides that is at least 90% complementary to a TDP-43 regulated cryptic exon sequence, and wherein the nucleotide sequence comprises a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 103-260. In some embodiments, the nucleotide sequence may consist of 17, 18, 19, 20, 21, 22, 23, 24,25, 26, 27 28, 29 or 30 nucleotides.

In some embodiments, the antisense sequence is capable of binding to a UNC13A splice site (i.e., and flanking regions thereof), preferably wherein the antisense sequence is at least 90% complementary, or at least 95% complementary, or at least 100% complementary to any one of SEQ ID NO 19, 20, 21 or 22.

In some embodiments, the antisense sequence is a 16 nucleotide sequence which is at least 90% complementary, or at least 95% complementary, or at least 100% complementary to any one of SEQ ID NO 19-22, or a 17 nucleotide sequence which is at least 90% complementary, or at least 95% complementary, or at least 100% complementary to any one of SEQ ID NO 19-22, a 18 nucleotide sequence which is at least 90% complementary, or at least 95% complementary, or at least 100% complementary to any one of SEQ ID NO 19-22, a 19 nucleotide sequence which is at least 90% complementary, or at least 95% complementary, or at least 100% complementary to any one of SEQ ID NO 19-22, or a 20 nucleotide sequence which is at least 90% complementary, or at least 95% complementary, or at least 100% complementary to any one of SEQ ID NO 19-22, a 21 nucleotide sequence which is at least 90% complementary, or at least 95% complementary, or at least 100% complementary to any one of SEQ ID NO 19-22, or a 22 nucleotide sequence which is at least 90% complementary, or at least 95% complementary, or at least 100% complementary to any one of SEQ ID NO 19-22. In some embodiments, the antisense sequence comprises or consists of a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 103-152. In some embodiments, the antisense sequence comprises a nucleotide sequence having 16 and 30 nucleotides that is at least 90% complementary to a TDP-43 regulated cryptic exon sequence, and wherein the nucleotide sequence comprises a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 103-152. In some embodiments, the nucleotide sequence may consist of 17, 18, 19, 20, 21, 22, 23, 24,25, 26, 27 28, 29 or 30 nucleotides.

In some embodiments, the antisense sequence is capable of binding to a UNC13A splice donor site (i.e., a 5′ splice site), i.e., and flanking regions thereof, preferably wherein the antisense sequence is at least 90% complementary, or at least 90% complementary, or at least 100% complementary to SEQ ID NO: 21-22. In some embodiments, the antisense sequence is capable of binding to one of more of the motifs GAUGG/G, AUGG/GU, UGG/GUG, GG/GUGA, G/GUGAG of the UNC13A cryptic exon and flanking regions, wherein/represents the cryptic exon/intron boundary of the UNC13A cryptic exon. In some embodiments, the antisense sequence comprises at least a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 136-152. In some embodiments, the antisense sequence comprises a nucleotide sequence having 16 and 30 nucleotides that is at least 90% complementary to a TDP-43 regulated cryptic exon sequence, and wherein the nucleotide sequence comprises a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 136-152. In some embodiments, the nucleotide sequence may consist of 17, 18, 19, 20, 21, 22, 23, 24,25, 26, 27 28, 29 or 30 nucleotides. In some embodiments, the antisense sequence is capable of binding to a UNC13A splice acceptor site (i.e., a 3′-splice site), i.e., and flanking regions thereof, preferably wherein the antisense sequence is at least 95% complementary, or at least 90% complementary, or at least 100% complementary to one of SEQ ID NO: 19 or SEQ ID NO: 20. In some embodiments, the antisense sequence is capable of binding to one of more of the motifs UCCAG/C, CCAG/CC, CAG/CCC, AG/CCCU or G/CCCUA the UNC13A cryptic exon and flanking regions, wherein/represents the intron/cryptic exon boundary of the UNC13A cryptic exon. In some embodiments, the antisense sequence is capable of binding to one of more of the motifs UCCAG/C, CCAG/CU, CAG/CUG, AG/CUGC, G/CUGCC in the UNC13A cryptic exon and flanking regions, wherein/represents the intron/cryptic exon boundary of the UNC13A cryptic exon. In some embodiments, the antisense sequence comprises or consists of a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 103-135. In some embodiments, the antisense sequence comprises a nucleotide sequence having 16 and 30 nucleotides that is at least 90% complementary to a TDP-43 regulated cryptic exon sequence, and wherein the nucleotide sequence comprises a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 103-135. In some embodiments, the nucleotide sequence may consist of 17, 18, 19, 20, 21, 22, 23, 24,25, 26, 27 28, 29 or 30 nucleotides.

In some embodiments, the antisense sequence is capable of at least partially binding to the TDP-43 binding region of the UNC13A cryptic exon, i.e., and/or flanking regions thereof, preferably wherein the antisense sequence is at least 90% complementary, or at least 95% complementary, or at least 100% complementary to any one of SEQ ID NO 23, 24, 25 or 26. In some embodiments, the antisense sequence is a 16 nucleotide sequence which is at least 90% complementary, or at least 95% complementary, or at least 100% complementary to SEQ ID SEQ ID NO23, 24, 25 or 26, or a 17 nucleotide sequence which is at least 90% complementary, or at least 95% complementary, or at least 100% complementary to any one of SEQ ID NO 23, 24, 25 or 26, a 18 nucleotide sequence which is at least 90% complementary, or at least 95% complementary, or at least 100% complementary to any one of SEQ ID NO 23, 24, 25 or 26, a 19 nucleotide sequence which is at least 90% complementary, or at least 95% complementary, or at least 100% complementary to any one of SEQ ID NO 23, 24, 25 or 26, or a 20 nucleotide sequence which is at least 90% complementary, or at least 95% complementary, or at least 100% complementary to any one of SEQ ID NO 23, 24, 25 or 26, a 21 nucleotide sequence which is at least 90% complementary, or at least 95% complementary, or at least 100% complementary to any one of SEQ ID NO 23, 24, 25 or 26, or a 22 nucleotide sequence which is at least 90% complementary, or at least 95% complementary, or at least 100% complementary to any one of SEQ ID NO 23, 24, 25 or 26. In some embodiments, the antisense sequence comprises or consists of a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 153-260. In some embodiments, the antisense sequence comprises a nucleotide sequence having 16 and 30 nucleotides that is at least 90% complementary to a TDP-43 regulated cryptic exon sequence, and wherein the nucleotide sequence comprises a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 153-260. In some embodiments, the nucleotide sequence may consist of 17, 18, 19, 20, 21, 22, 23, 24,25, 26, 27 28, 29 or 30 nucleotides.

In some embodiments, the antisense sequence is capable of at least partially binding to an exonic sequence enhancer in the UNC13A cryptic exon, defined by ESE finder 3.0 using the SR Protein matrix library, preferably wherein the antisense sequence is at least 90% complementary, or at least 95% complementary, or at least 100% complementary to any one of SEQ ID NO: 27, 28, 29 or 30. In some embodiments, the antisense sequence is a 16 nucleotide sequence which is at least 90% complementary, or at least 95% complementary, or at least 100% complementary to any one of SEQ ID NO 27, 28, 29 or 30, or a 17 nucleotide sequence which is at least 90% complementary, or at least 95% complementary, or at least 100% complementary to any one of SEQ ID NO 27, 28, 29 or 30, a 18 nucleotide sequence which is at least 90% complementary, or at least 95% complementary, or at least 100% complementary to any one of SEQ ID NO 27, 28, 29 or 30, a 19 nucleotide sequence which is at least 90% complementary, or at least 95% complementary, or at least 100% complementary to any one of SEQ ID NO 27, 28, 29 or 30, or a 20 nucleotide sequence which is at least 90% complementary, or at least 95% complementary, or at least 100% complementary to any one of SEQ ID NO 27, 28, 29 or 30, a 21 nucleotide sequence which is at least 90% complementary, or at least 95% complementary, or at least 100% complementary to any one of SEQ ID NO 27, 28, 29 or 30, or a 22 nucleotide sequence which is at least 90% complementary, or at least 95% complementary, or at least 100% complementary to any one of SEQ ID NO 27, 28, 29 or 30.

In some embodiments, the exonic sequence enhancer is an SRSF1 binding site and the antisense sequence is capable of binding (i.e., complementary to or overlapping with) the motif CUCAGGA within the UNC13A cryptic exon sequence, preferably wherein the antisense sequence is at least 90% complementary, or at least 95% complementary, or at least 100% complementary to SEQ ID NO: 27. In some embodiments, the exonic sequence enhancer is an SRSF2 binding site and the antisense sequence is capable of binding (i.e., i.e., complementary to or overlapping with) the motif GUUUCCUG within the UNC13A cryptic exon sequence, preferably wherein the antisense sequence is at least 90% complementary, or at least 95% complementary, or at least 100% complementary to SEQ ID NO: 28. In some embodiments, the exonic sequence enhancer is an SRSF5 binding site and the antisense sequence is capable of binding (i.e., complementary to or overlapping with) the motif ACUCAGG within the UNC13A cryptic exon sequence, preferably wherein the antisense sequence is at least 90% complementary, or at least 95% complementary, or at least 100% complementary to SEQ ID NO: 28. In some embodiments, the exonic sequence enhancer is an SRSF6 binding site and the antisense sequence is capable of binding (i.e., complementary to) the motif UGUGUC within the UNC13A cryptic exon sequence, preferably wherein the antisense sequence is at least 90% complementary, or at least 95% complementary, or at least 100% complementary to SEQ ID NO: 29. In some embodiments, the antisense sequence is capable of binding (i.e., complementary to or overlapping with) both the SRSF5 binding site (ACUCAGG), and the SRSF1 binding site (CUCAGGA) in the UNC13A cryptic exon, preferably wherein the antisense sequence is at least 90% complementary, or at least 95% complementary, or at least 100% complementary to SEQ ID NO: 28.

In some embodiments, the antisense sequence comprises a sequence that is at least 80% identical, or at least 85% identical, or at least 90% identical, or at least 95% identical or at least 100% identical to any one of SEQ ID NO 420, 362, 364, 366, 368, 370, 372, 374, 382, 384, for a sequence with the same number of nucleotides.

### STMN2

In some embodiments, the TDP-43 regulated cryptic exon is a STMN2 cryptic exon. The TDP-43 regulated STMN2 cryptic exon corresponds to exon 2a in the human STMN2 gene. In some embodiments, the antisense sequence is at least 90% complementary (or at least 95%, or at least 100% complementary) to SEQ ID NO: 7. In some embodiments, the TDP-43 regulated cryptic exon is a STMN2 cryptic exon. The TDP-43 regulated STMN2 cryptic exon corresponds to exon 2a in the human STMN2 gene. In some embodiments, the antisense sequence is at least 90% complementary (or at least 95%, or at least 100% complementary) to SEQ ID NO: 8.

In some embodiments, the antisense sequence is capable of binding to the STMN2 3′ splice site (i.e., splice acceptor site), i.e., and flanking regions thereof, preferably wherein the antisense sequence is at least 90% complementary, or at least 90% complementary, or at least 100% complementary to SEQ ID NO 11. In some embodiments, the antisense sequence is a 16 nucleotide sequence which is at least 90% complementary, or at least 95% complementary, or at least 100% complementary to of SEQ ID NO 11, or a 17 nucleotide sequence which is at least 90% complementary, or at least 95% complementary, or at least 100% complementary to SEQ ID NO 11, a 18 nucleotide sequence which is at least 90% complementary, or at least 95% complementary, or at least 100% complementary to SEQ ID NO 11, a 19 nucleotide sequence which is at least 90% complementary, or at least 95% complementary, or at least 100% complementary to any one of SEQ ID NO 11, or a 20 nucleotide sequence which is at least 90% complementary, or at least 95% complementary, or at least 100% complementary to SEQ ID NO 11, a 21 nucleotide sequence which is at least 90% complementary, or at least 95% complementary, or at least 100% complementary to SEQ ID NO 11, or a 22 nucleotide sequence which is at least 90% complementary, or at least 95% complementary, or at least 100% complementary to SEQ ID NO 11. In some embodiments, the antisense sequence is capable of binding to one of more of the motifs UGCAG/G, GCAG/GA, CAG/GAC, AG/GACU or G/GACUC in the STMN2 cryptic exon and flanking regions, wherein/represents the intron/cryptic exon boundary of the STMN2 cryptic exon. In some embodiments, the antisense sequence comprises or consists of a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 42-59. In some embodiments, the antisense sequence comprises a nucleotide sequence having 16 and 30 nucleotides that is at least 90% complementary to a TDP-43 regulated cryptic exon sequence, and wherein the nucleotide sequence comprises a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 42-59. In some embodiments, the nucleotide sequence may consist of 17, 18, 19, 20, 21, 22, 23, 24,25, 26, 27 28, 29 or 30 nucleotides.

In some embodiments, the antisense sequence is capable of at least partially binding to the TDP-43 binding region, or TDP-43 binding motif, of the STMN2 cryptic exon, i.e., and/or flanking regions thereof, preferably wherein the antisense sequence is at least 90% complementary, or at least 95% complementary, or at least 100% complementary to any one of SEQ ID NO: 12 or 13. In some embodiments, the antisense sequence is a 16 nucleotide sequence which is at least 90% complementary, or at least 95% complementary, or at least 100% complementary to any one of SEQ ID NO 12 or 13, or a 17 nucleotide sequence which is at least 90% complementary, or at least 95% complementary, or at least 100% complementary to any one of SEQ ID NO 12 or 13, a 18 nucleotide sequence which is at least 90% complementary, or at least 95% complementary, or at least 100% complementary to any one of SEQ ID NO 12 or 13, a 19 nucleotide sequence which is at least 90% complementary, or at least 95% complementary, or at least 100% complementary to any one of SEQ ID NO 12 or 13, or a 20 nucleotide sequence which is at least 90% complementary, or at least 95% complementary, or at least 100% complementary to any one of SEQ ID NO 12 or 13, a 21 nucleotide sequence which is at least 90% complementary, or at least 95% complementary, or at least 100% complementary to any one of SEQ ID NO 12 or 13, or a 22 nucleotide sequence which is at least 90% complementary, or at least 95% complementary, or at least 100% complementary to any one of SEQ ID NO 12 or 13. In some embodiments, the antisense sequence comprises or consists of a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 60-102. In some embodiments, the antisense sequence comprises a nucleotide sequence having 16 and 30 nucleotides that is at least 90% complementary to a TDP-43 regulated cryptic exon sequence, and wherein the nucleotide sequence comprises a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 60-102. In some embodiments, the nucleotide sequence may consist of 17, 18, 19, 20, 21, 22, 23, 24,25, 26, 27 28, 29 or 30 nucleotides.

In some embodiments, the antisense sequence is capable of at least partially binding to an exonic sequence enhancer in the STMN2 cryptic exon, i.e., and flanking regions thereof, defined by ESE finder 3.0, preferably wherein the antisense sequence is at least 90% complementary, or at least 95% complementary, or at least 100% complementary to any one of SEQ ID NO: 14, 15, 16, 17, or 18. In some embodiments, the antisense sequence is a 16 nucleotide sequence which is at least 90% complementary, or at least 95% complementary, or at least 100% complementary to any one of SEQ ID NO 14, 15, 16, 17, or 18, or a 17 nucleotide sequence which is at least 90% complementary, or at least 95% complementary, or at least 100% complementary to any one of SEQ ID NO 14, 15, 16, 17, or 18, a 18 nucleotide sequence which is at least 90% complementary, or at least 95% complementary, or at least 100% complementary to any one of SEQ ID NO 14, 15, 16, 17, or 18, a 19 nucleotide sequence which is at least 90% complementary, or at least 95% complementary, or at least 100% complementary to any one of SEQ ID NO 14, 15, 16, 17, or 18, or a 20 nucleotide sequence which is at least 90% complementary, or at least 95% complementary, or at least 100% complementary to any one of SEQ ID NO 14, 15, 16, 17, or 18, a 21 nucleotide sequence which is at least 90% complementary, or at least 95% complementary, or at least 100% complementary to any one of SEQ ID NO 14, 15, 16, 17, or 18, or a 22 nucleotide sequence which is at least 90% complementary, or at least 95% complementary, or at least 100% complementary to any one of SEQ ID NO 14, 15, 16, 17, or 18.

In some embodiments, the exonic sequence enhancer is an SRSF1 binding site and the antisense sequence is capable of binding (i.e., complementary to or overlapping with) the motif CAGAAGA within the STMN2 cryptic exon sequence, preferably wherein the antisense sequence is at least 90% complementary, or at least 95% complementary, or at least 100% complementary to SEQ ID NO:14. In some embodiments, the exonic sequence enhancer is an SRSF2 binding site and the antisense sequence is capable of binding (i.e., i.e., complementary to or overlapping with) the motif GGCUUGUG within the STMN2 cryptic exon sequence, preferably wherein the antisense sequence is at least 90% complementary, or at least 95% complementary, or at least 100% complementary to SEQ ID NO: 15. In some embodiments, the exonic sequence enhancer is an SRSF5 binding site and the antisense sequence is capable of binding (i.e., complementary to or overlapping with) the motif UGACAAG within the STMN2 cryptic exon sequence, preferably wherein the antisense sequence is at least 90% complementary, or at least 95% complementary, or at least 100% complementary to SEQ ID NO: 16. In some embodiments, the exonic sequence enhancer is an SRSF6 binding site and the antisense sequence is capable of binding (i.e., complementary to) the motif UGCGGC within the STMN2 cryptic exon sequence, preferably wherein the antisense sequence is at least 90% complementary, or at least 95% complementary, or at least 100% complementary to SEQ ID NO: 15. In some embodiments, the antisense sequence is capable of binding (i.e., complementary to or overlapping with) both the SRSF6 binding site (UGCGGC) and the SRSF2 binding site (GGCUUGUG) in the STMN2 cryptic exon, preferably wherein the antisense sequence is at least 90% complementary, or at least 95% complementary, or at least 100% complementary to SEQ ID NO: 15.

In some embodiments, the antisense sequence comprises a sequence that is at least 80% identical, or at least 85% identical, or at least 90% identical, or at least 95% identical or at least 100% identical to any one of SEQ ID NO 391, 393, 395, 397, 399, 401, 403, 405, 407, for a sequence with the same number of nucleotides.

### INSR

In some embodiments, the TDP-43 regulated cryptic exon is an INSR cryptic exon. The TDP-43 regulated INSR cryptic exon is between exon 6 and 7 in the human INSR gene. In some embodiments, the antisense sequence is at least 90% complementary (or at least 95%, or at least 100% complementary) to SEQ ID NO: 9 . . . . In some embodiments, the antisense sequence is at least 90% complementary (or at least 95%, or at least 100% complementary) to SEQ ID NO: 10.

In some embodiments, the antisense sequence is capable of binding to the INSR 3′ splice site, (i.e., the INSR splice acceptor site), i.e., and flanking regions thereof, preferably wherein the antisense sequence is at least 90% complementary, or at least 90% complementary, or at least 100% complementary to SEQ ID NO 31. In some embodiments, the antisense sequence is a 16 nucleotide sequence which is at least 90% complementary, or at least 95% complementary, or at least 100% complementary to of SEQ ID NO 31, or a 17 nucleotide sequence which is at least 90% complementary, or at least 95% complementary, or at least 100% complementary to SEQ ID NO 31, a 18 nucleotide sequence which is at least 90% complementary, or at least 95% complementary, or at least 100% complementary to SEQ ID NO 31, a 19 nucleotide sequence which is at least 90% complementary, or at least 95% complementary, or at least 100% complementary to any one of SEQ ID NO 31, or a 20 nucleotide sequence which is at least 90% complementary, or at least 95% complementary, or at least 100% complementary to SEQ ID NO 31, a 21 nucleotide sequence which is at least 90% complementary, or at least 95% complementary, or at least 100% complementary to SEQ ID NO 31, or a 22 nucleotide sequence which is at least 90% complementary, or at least 95% complementary, or at least 100% complementary to SEQ ID NO 31. In some embodiments, the antisense sequence is capable of binding to one of more of the motifs UAUAG/U, AUAG/UA, UAG/UAC, AG/UACC, G/UACCG in the INSR cryptic exon and flanking regions, wherein/represents the intron/cryptic exon boundary of the INSR cryptic exon. In some embodiments, the antisense sequence comprises or consists of a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 261-277. In some embodiments, the antisense sequence comprises a nucleotide sequence having 16 and 30 nucleotides that is at least 90% complementary to a TDP-43 regulated cryptic exon sequence, and wherein the nucleotide sequence comprises a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 261-277. In some embodiments, the nucleotide sequence may consist of 17, 18, 19, 20, 21, 22, 23, 24,25, 26, 27 28, 29 or 30 nucleotides.

In some embodiments, the antisense sequence is capable of at least partially binding to the TDP-43 binding region, or TDP-43 binding motif, of the INSR cryptic exon, i.e., and/or flanking regions thereof, preferably wherein the antisense sequence is at least 90% complementary, or at least 95% complementary, or at least 100% complementary to any one of SEQ ID NO: 32 or 33. In some embodiments, the antisense sequence is a 16 nucleotide sequence which is at least 90% complementary, or at least 95% complementary, or at least 100% complementary to any one of SEQ ID NO 32 or 33, or a 17 nucleotide sequence which is at least 90% complementary, or at least 95% complementary, or at least 100% complementary to any one of SEQ ID NO 32 or 33, a 18 nucleotide sequence which is at least 90% complementary, or at least 95% complementary, or at least 100% complementary to any one of SEQ ID NO 32 or 33, a 19 nucleotide sequence which is at least 90% complementary, or at least 95% complementary, or at least 100% complementary to any one of SEQ ID NO 32 or 33, or a 20 nucleotide sequence which is at least 90% complementary, or at least 95% complementary, or at least 100% complementary to any one of SEQ ID NO 32 or 33, a 21 nucleotide sequence which is at least 90% complementary, or at least 95% complementary, or at least 100% complementary to any one of SEQ ID NO 32 or 33, or a 22 nucleotide sequence which is at least 90% complementary, or at least 95% complementary, or at least 100% complementary to any one of SEQ ID NO 32 or 33. In some embodiments, the antisense sequence comprises or consists of a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 278-352. In some embodiments, the antisense sequence comprises a nucleotide sequence having 16 and 30 nucleotides that is at least 90% complementary to a TDP-43 regulated cryptic exon sequence, and wherein the nucleotide sequence comprises a 16 nucleotide portion that has at least 90% sequence identity, or at least 91% sequence identity, or at least 92% sequence identity, or at least sequence 93% sequence identity, or at least 94% sequence identity, or at least 95% sequence identity, or at least 96% sequence identity, or at least 97% sequence identity, or at least 98% sequence identity, or at least 99% sequence identity, or at least 100% sequence identity to one or more of SEQ ID NO 278-352. In some embodiments, the nucleotide sequence may consist of 17, 18, 19, 20, 21, 22, 23, 24,25, 26, 27 28, 29 or 30 nucleotides.

In some embodiments, the antisense sequence is capable of at least partially binding to an exonic sequence enhancer in the INSR cryptic exon, defined by ESE finder 3.0, preferably wherein the antisense sequence is at least 90% complementary, or at least 95% complementary, or at least 100% complementary to any one of SEQ ID NO: 34, 35, 36, 37, 38, 39 or 40. In some embodiments, the antisense sequence is a 16 nucleotide sequence which is at least 90% complementary, or at least 95% complementary, or at least 100% complementary to any one of SEQ ID NO 34, 35, 36, 37, 38, 39 or 40, or a 17 nucleotide sequence which is at least 90% complementary, or at least 95% complementary, or at least 100% complementary to any one of SEQ ID NO 34, 35, 36, 37, 38, 39 or 40, a 18 nucleotide sequence which is at least 90% complementary, or at least 95% complementary, or at least 100% complementary to any one of SEQ ID NO 34, 35, 36, 37, 38, 39 or 40, a 19 nucleotide sequence which is at least 90% complementary, or at least 95% complementary, or at least 100% complementary to any one of SEQ ID NO 34, 35, 36, 37, 38, 39 or 40, or a 20 nucleotide sequence which is at least 90% complementary, or at least 95% complementary, or at least 100% complementary to any one of SEQ ID NO 34, 35, 36, 37, 38, 39 or 40, a 21 nucleotide sequence which is at least 90% complementary, or at least 95% complementary, or at least 100% complementary to any one of SEQ ID NO 34, 35, 36, 37, 38, 39 or 40, or a 22 nucleotide sequence which is at least 90% complementary, or at least 95% complementary, or at least 100% complementary to any one of SEQ ID NO 34, 35, 36, 37, 38, 39 or 40

In some embodiments, the exonic sequence enhancer is an SRSF1 binding site and the antisense sequence is capable of binding (i.e., complementary to or overlapping with) the motif GACACCT or CTGAAGA within the INSR cryptic exon, preferably wherein the antisense sequence is at least 90% complementary, or at least 95% complementary, or at least 100% complementary to any one of SEQ ID NO: 34 or 35. In some embodiments, the exonic sequence enhancer is an SRSF2 binding site and the antisense sequence is capable of binding (i.e., i.e., complementary to or overlapping with) the motif GAAUGAUG or GGCUGAUG within the INSR cryptic exon sequence, preferably wherein the antisense sequence is at least 90% complementary, or at least 95% complementary, or at least 100% complementary to any one of SEQ ID NO: 36 or 37. In some embodiments, the exonic sequence enhancer is an SRSF5 binding site and the antisense sequence is capable of binding (i.e., complementary to or overlapping with) the motif AUACAAG within the INSR cryptic exon sequence, preferably wherein the antisense sequence is at least 90% complementary, or at least 95% complementary, or at least 100% complementary to SEQ ID NO: 38. In some embodiments, the exonic sequence enhancer is an SRSF6 binding site and the antisense sequence is capable of binding (i.e., complementary to) the motif UACGGG or UGUGUA within the INSR cryptic exon sequence, preferably wherein the antisense sequence is at least 90% complementary, or at least 95% complementary, or at least 100% complementary to any one of SEQ ID NO: 39 or 40.

In some embodiments, the antisense sequence comprises a sequence that is at least 80% identical, or at least 85% identical, or at least 90% identical, or at least 95% identical or at least 100% identical to any one of SEQ ID NO 409, 411, 413, 415, 417, 419, for a sequence with the same number of nucleotides.

Other TDP-43 regulated cryptic exons.

### ELAVL3

In some embodiments, the TDP-43 regulated cryptic exon is an ELAVL3 cryptic exon. In some embodiments, the antisense sequence is at least 90% complementary (or at least 95%, or at least 100% complementary) to SEQ ID NO: 448.

In some embodiments, the antisense sequence is capable of binding to the ELALV3 3′ splice site, (i.e., the ELAVL3 splice acceptor site), i.e., and flanking regions thereof, preferably wherein the antisense sequence is at least 90% complementary, or at least 90% complementary, or at least 100% complementary to SEQ ID NO 454.

In some embodiments, the antisense sequence is capable of binding to a ELAVL3 TDP-43 binding region i.e., and flanking regions thereof, preferably wherein the antisense sequence is at least 90% complementary, or at least 90% complementary, or at least 100% complementary to SEQ ID NO 455, 456 or 457.

### G3BP1

In some embodiments, the TDP-43 regulated cryptic exon is an G3BP1 cryptic exon. In some embodiments, the antisense sequence is at least 90% complementary (or at least 95%, or at least 100% complementary) to SEQ ID NO: 449.

In some embodiments, the antisense sequence is capable of binding to the G3BP13 3′ splice site, (i.e., the G3BP1 splice acceptor site), i.e., and flanking regions thereof, preferably wherein the antisense sequence is at least 90% complementary, or at least 90% complementary, or at least 100% complementary to SEQ ID NO 458.

In some embodiments, the antisense sequence is capable of binding to a G3BP1 TDP-43 binding region i.e., and flanking regions thereof, preferably wherein the antisense sequence is at least 90% complementary, or at least 90% complementary, or at least 100% complementary to SEQ ID NO 459

### AARS1

In some embodiments, the TDP-43 regulated cryptic exon is an AARS1. cryptic exon. In some embodiments, the antisense sequence is at least 90% complementary (or at least 95%, or at least 100% complementary) to SEQ ID NO: 450.

In some embodiments, the antisense sequence is capable of binding to the AARS1 3′ splice site, (i.e., the AARS1 splice acceptor site), i.e., and flanking regions thereof, preferably wherein the antisense sequence is at least 90% complementary, or at least 90% complementary, or at least 100% complementary to SEQ ID NO 460.

In some embodiments, the antisense sequence is capable of binding to a AARS1 TDP-43 binding region i.e., and flanking regions thereof, preferably wherein the antisense sequence is at least 90% complementary, or at least 90% complementary, or at least 100% complementary to SEQ ID NO 461 or 462.

### CELF5

In some embodiments, the TDP-43 regulated cryptic exon is an CELF5 cryptic exon. In some embodiments, the antisense sequence is at least 90% complementary (or at least 95%, or at least 100% complementary) to SEQ ID NO: 451.

In some embodiments, the antisense sequence is capable of binding to the CELF5 5′ splice site, (i.e., the CELF5 splice donor site), i.e., and flanking regions thereof, preferably wherein the antisense sequence is at least 90% complementary, or at least 90% complementary, or at least 100% complementary to SEQ ID NO 463.

In some embodiments, the antisense sequence is capable of binding to a CELF5 TDP-43 binding region i.e., and flanking regions thereof, preferably wherein the antisense sequence is at least 90% complementary, or at least 90% complementary, or at least 100% complementary to SEQ ID NO 464, 465, or 466.

### CAMK2B

In some embodiments, the TDP-43 regulated cryptic exon is an CAMK2B cryptic exon. In some embodiments, the antisense sequence is at least 90% complementary (or at least 95%, or at least 100% complementary) to SEQ ID NO: 452.

In some embodiments, the antisense sequence is capable of binding to the CAMK2B 5′ splice site, (i.e., the CAMK2B splice donor site), i.e., and flanking regions thereof, preferably wherein the antisense sequence is at least 90% complementary, or at least 90% complementary, or at least 100% complementary to SEQ ID NO 467.

In some embodiments, the antisense sequence is capable of binding to a CAMK2B TDP-43 binding region i.e., and flanking regions thereof, preferably wherein the antisense sequence is at least 90% complementary, or at least 90% complementary, or at least 100% complementary to SEQ ID NO 468.

### UNC13B

In some embodiments, the TDP-43 regulated cryptic exon is an UNC13B cryptic exon. In some embodiments, the antisense sequence is at least 90% complementary (or at least 95%, or at least 100% complementary) to SEQ ID NO: 453.

In some embodiments, the antisense sequence is capable of binding to the UNC13B 5′ splice site, (i.e., the UNC13B splice donor site), i.e., and flanking regions thereof, preferably wherein the antisense sequence is at least 90% complementary, or at least 90% complementary, or at least 100% complementary to SEQ ID NO 469.

In some embodiments, the antisense sequence is capable of binding to a UNC13B TDP-43 binding region i.e., and flanking regions thereof, preferably wherein the antisense sequence is at least 90% complementary, or at least 90% complementary, or at least 100% complementary to SEQ ID NO 470 or 471.

Sequence Complementary to a Binding Domain for a hnRNP Protein

The constructs described herein comprise a sequence comprising a binding domain for a hnRNP protein. The term binding domain may be used interchangeably with hnRNP binding site, hnRNP binding sequence or hnRNP binding domain. The sequence comprising a binding domain for a hnRNP protein may also be described as a hnRNP tail herein.

A hnRNP protein defined herein and as known in the art is a heterogeneous nuclear ribonucleoprotein. These are a family of RNA-binding proteins that participate in pre-mRNA processing. The definition of a hnRNP protein described herein is not intended to include or encompass the protein TDP-43.

In preferred embodiments, the hnRNP protein is a hnRNP protein comprising at least 2 RNA recognition motifs or quasi-RNA recognition motifs. In preferred embodiments, the hnRNP protein is a protein that is highly endogenously expressed in a human cell, more particularly a human cell nucleus. In some embodiments, highly endogenous expressed refers to any protein with a “high” protein expression or a “high” protein expression score in neuronal and/or glial cells in any part of the brain as defined by human protein atlas. (i.e., https://www.proteinatlas.org/). In some embodiments, the part of the brain may be selected from the basal ganglia, hippocampus, cerebellum, cerebral cortex, or a combination thereof. In some embodiments, the protein expression score in any part of the brain may be at least 100, or at least 150, or at least 200, or at least 250, or at least 300, or at least 350 nTPM in the brain, which may be determined in accordance with the consensus data set on human protein atlas, wherein nTPM refers to normalised transcript expression values per million.

In some embodiments, the hnRNP protein is not a hnRNP protein that has to form a tetramer (e.g., hnRNP C) and/or a hnRNP that functions by binding on both sides of the cryptic exon in order to have a repressive effect on splicing.

In some embodiments, the hnRNP protein is selected from a hnRNP A and hnRNP H protein, and the sequence comprising a binding domain comprises at least one binding motif for hnRNP A and hnRNP H respectively. These are found to more effectively correct splicing compared to other hnRNP proteins tested (e.g., hnRNP C or hnRNP L). In some embodiments, the hnRNP A protein is hnRNP A1 or hnRNP A2 . . . . In some examples, the hnRNP A protein in hnRNP A1. hnRNP A1 and hnRNP A2 are the most abundant hnRNPs with nearly identical functions and play important roles in regulating gene expression at multiple levels.

In some embodiments, the sequence comprising a binding domain for a hnRNP protein is from about 8 to 24 nucleotides, preferably from about 16 nucleotides to 22 nucleotides, or about 20 nucleotides. In some embodiments, the binding sequence for a hnRNP protein comprises at least 8, preferably at least 16 nucleotides, or at least 17 nucleotides (or 17 nucleotides), or at least 18 nucleotides (or 18 nucleotides), or at least 19 nucleotides (or 19 nucleotides), or at least 20 nucleotides (or 20 nucleotides). In some embodiments, the binding sequence comprises one, two or three binding motifs for a hnRNP protein. Example binding motifs are described below.

In some embodiments, the binding sequence is a binding sequence for hnRNP A (e.g., hnRNP A1 or hnRNP A2). The binding sequence for hnRNP A may be any sequence or comprise any motif known in the art to bind hnRNP A (e.g., hnRNP A1 or hnRNP A2). In some embodiments the binding sequence for hnRNP A (e.g., hnRNP A1 or hnRNP A2) may have been determined by immunoprecipitation of hnRNP A1 to the human transcriptome, e.g., using CLIP.

In some embodiments, the binding sequence for a hnRNP A protein (e.g., hnRNP A1 or hnRNP A2) comprises at least one or two motifs comprising UAGGG. In some embodiments, the binding sequence for hnRNP A1 comprises at least one motif according to WUAGGGWS, where W is A or U, and wherein S is C or G, and preferably wherein the binding sequence for hnRNP A1 comprises at least two motifs according to WUAGGGWS, where W is A or U, and wherein S is C or G. In some embodiments, the binding sequence for hnRNP A1 comprises at least one or two motifs selected from UAGGG (more preferably UUAGGG, more preferably UAGGGU or UAGGGA, and furthermore preferably UUAGGGUG), or ATAGGGA (more preferably ATAGGGAC). In some embodiments, the binding sequence is at least 80% identical, or at least 85% identical, or at least 90% identical, or at least 100% identical to SEQ ID NO: 361 or 389. In some embodiments, the binding sequence for hnRNP A2 comprises at last one or two motifs selected from UAGGG, GGUAGUAG, or AGGAUAGA.

In some embodiments, the binding sequence is a binding sequence for hnRNP H. hnRNP H may encompass both hnRNP H1 and hnRNP H2. The binding sequence for hnRNP H may be any sequence or motif known in the art to bind hnRNP H. In some embodiments the binding sequence for hnRNP H may have been determined by immunoprecipitation of hnRNP H to the human transcriptome, e.g., using CLIP. In some embodiments, the binding sequence for hnRNP H comprises one or two binding motifs comprising the motif GGGGA. In some embodiments, the binding sequence is at least 80% identical, or at least 85% identical, or at least 90% identical, or at least 100% identical to SEQ ID NO: 376 or 386.

### Function of Construct

The construct described herein is capable of modulating splicing of the TDP-43 regulated cryptic exon. In other words, the construct described herein is capable of correcting splicing and/or at least partially preventing inclusion of the TDP-43 regulated cryptic exon in mature RNA, such that a functional protein is produced.

The construct described herein is configured to recruit a hnRNP protein, preferably and endogenous hnRNP protein when present in a cell. The cell may be a cell that is depleted of TDP-43 protein (i.e., as compared to a healthy or wild-type cell), more preferably a cell that is depleted of TDP-43 protein in the nucleus. The recruitment of a hnRNP protein may at least partially compensate for the loss of TDP-43 in cells that are depleted of TDP-43, where the hnRNP recruitment of the hnRNP protein represses splicing of the TDP-43 regulated cryptic exon. In some embodiments, the antisense sequence may at least partially contribute to modulating splicing of the TDP-43 regulated cryptic exon by sterically blocking or masking splicing elements.

In some embodiments, the construct described herein completely rescue splicing (i.e., defined as 0% cryptic exon present in the mature mRNA product of the cell).

In some embodiments, the construct described herein partially rescue splicing i.e., defined as less than 90% cryptic exon present in mature mRNA products of the cell, or less than 80%, or less than 70%, or less than 60%, or less than 50%, or less than 40%, or less than 30%, or less than 20%, or less than 10%, or less than 5% present in mature mRNA products of the cell. This may be determined by RNA-sequencing, RT-qPCR, or RT-PCR. Even partial rescue of correct splicing has some therapeutic benefit and can be used to further understand the role of cryptic exons in TDP-43 pathology.

As defined herein, the cell may be any suitable cell. In preferred embodiments, the cell is a mammalian cell, more preferably a human cell. In preferred embodiments, the cell has nuclear depletion of TDP-43. In some embodiments, the cell is a brain cell. In some embodiments, the cell is a neuron or neuronal cell. In some embodiments, the cell is a microglial cell or astrocyte cell. In some embodiments, the cell is a muscle cell.

### Vector

Also described herein is a vector comprising the modified U7 snRNA construct described herein, or encoding for the modified U7 snRNA construct described herein. The vector encoding for the modified U7 snRNA construct described herein comprises a sequence that is the reverse complement of any modified U7 snRNA construct described herein. The vector typically comprises a promoter upstream of the modified U7 snRNA construct sequence, or upstream of the sequence encoding for the construct, wherein the promoter is a UsnRNA promoter. In some examples described herein, the vector additionally comprises one more expression cassette consisting of a CMV promoter, a blasticidin S deaminase cDNA and an SV40 polyadenylation signal downstream of the U7 snRNA expression cassette. This allows for the selection of cells that have taken up the vector.

The vectors described herein also comprise a 3′ box sequence downstream of the construct sequence or sequence encoding for the construct sequence. Any suitable 3′ box sequence may be used. In some examples, the vectors comprise a 3′ box sequence that has at least 80% sequence identity, or at least 85%, or at least 90%, or at least 95%, or at least 100% sequence identity to SEQ ID NO: 357

In some embodiments, the vector comprises an expression cassette, e.g., an inverted terminal repeat (ITR) cassette. In such systems the vector comprises a sequence encoding for the construct described herein and one or more inverted terminal repeat (ITR) sequences flanking the construct sequence. In some embodiments, the vector is a viral vector. The viral vector may be a human viral vector or a non-human viral vector (e.g., a primate vector). In some embodiments, the vector is a viral vector, such as an adeno-associated (AAV) vector, a retrovirus vector, a lentivirus vector or an adenovirus vector. The viral vector may be an RNA vector or a DNA vector.

### Pharmaceutical Composition

Also disclosed herein is a pharmaceutical composition comprising one or more of the modified U7 snRNA constructs disclosed herein and/or one or more of the vectors disclosed herein. In some embodiments, the pharmaceutical composition comprises two or more, modified U7 snRNA constructs or vectors as defined herein. The two or more constructs or vectors may be capable of binding to the same or different TDP-43 regulated cryptic exons. The two or more constructs or vectors may comprise different antisense sequences and/or different sequences that comprise a hnRNP binding domain. In some embodiments, the different antisense sequences may be capable of binding different splicing elements.

The pharmaceutical composition may further comprise a pharmaceutical excipient.

### Therapy and Medicaments

The modified U7 snRNA constructs of the present disclosure, the vectors of the present disclosure, or the pharmaceutical compositions of the present disclosure may be for use in therapy (i.e., as therapeutic agents for disease treatment). The therapeutic use of the constructs of the present disclosure may involve in modulation of splicing of endogenously existing pre-RNAs to at least partially prevent inclusion of a TDP-43 regulated cryptic exon in the mature RNA of the cell transcript. This provides protection from the disease since the absence of the TDP-43 regulated cryptic exon in the mature RNA transcript leads to the production of fully functional protein.

The modified U7 snRNA constructs of the present disclosure, the vectors of the present disclosure, or the pharmaceutical compositions of the present disclosure may be for use, or used, as a medicament, for example, in therapy.

The modified U7 snRNA constructs of the present disclosure, the vectors of the present disclosure, or the pharmaceutical compositions of the present disclosure may be for use in the treatment of a disease associated with TDP-43 pathology or dysfunction. In some embodiments, the disease is a neurodegenerative disease or a neuromuscular disease. In an embodiment, the neurodegenerative disorder is associated with reduced nuclear TDP-43. In an embodiment, the neurodegenerative disorder is caused by nucleus-cytoplasmic mislocalization of TDP-43. In an embodiment, the neurodegenerative disorder is associated with TDP-43 pathology (e.g., pathological TDP-43).

In an embodiment, the construct, vector, or pharmaceutical composition for use or the method of treating comprises first diagnosing a subject with a neurodegenerative disorder associated with TDP-43 pathology. In an embodiment, this is determined using a biomarker of TDP-43 pathology. In an embodiment, this may be determined by genetics, for example, a genetic mutation. In an embodiment, TDP-43 pathology associated with ALS may be determined if FUS and SOD1 mutations are not found in the subject. In an embodiment, TDP-pathology associated with FTD may be determined if C9orf72 or PGRN mutations are not found in the subject. In an embodiment, the biomarker of TDP-43 pathology may include mutant TDP-43. In some embodiments, TDP-43 pathology may be determined with TDP-43 phosphorylation. In some embodiments, TDP-43 pathology may be determined by expression of the STMN2 cryptic exon, which may be determined by RNA-seq. In an embodiment, the construct, vector, or pharmaceutical composition for use or the method of treating comprises first identifying in a subject whether they possess a SNP variant associated with rs12973192 and/or rs12608932 ahead of the method of treating. This may be determined by genomics.

In an embodiment, the disorder (i.e., neurodegenerative disorder) may be selected from ALS, frontotemporal dementia, Alzheimer's disease, Inclusion body myositis/myopathy (IBM), FOSMNN (Facial onset sensory and motor neuronopathy), Perry Syndrome, Limbic-Predominant Age-Related TDP-43 Encephalopathy (LATE) or a combination thereof.

In an embodiment, the neurodegenerative disorder is ALS (amyotrophic lateral sclerosis). ALS is a chronic and fatal form of motor neuron disease (MND) and may otherwise be referred to as MND, Charcot disease or Lou Gehrig's disease. In some embodiments, the ALS may be ALS is familial ALS or sporadic (idiopathic) ALS. Familial ALS (FALS) is ALS that runs in the family, and accounts for about 10% of ALS cases. Sporadic ALS is non-familial ALS. In an embodiment, the ALS may not be an ALS-FUS and ALS-SOD1 which are genetically-defined forms of ALS. The construct, vectors, or pharmaceutical compositions for use, or the method of treatment described herein, may ameliorate one or more symptoms associated with ALS. Symptoms of ALS may include fasciculation (muscle twitches); muscle cramps; tight and stiff muscles (spasticity), muscle weakness, slurred and nasal speech and a difficulty chewing or swallowing. ALS leads to progressive deterioration of muscle function and ultimately often leads to death due to respiratory failure. In an embodiment, the TDP-43 regulated cryptic exon is a UNC13A TDP-43 regulated cryptic exon and the neurodegenerative disorder is ALS. In another embodiment, the TDP-43 regulated cryptic exon is STMN2 TDP-43 regulated cryptic exon and the neurodegenerative disorder is ALS.

In an embodiment, the neurodegenerative disorder is frontotemporal dementia (FTD). Frontotemporal dementia is a type of dementia that affects the frontal and temporal lobes of the brain. The constructs, vectors, or pharmaceutical composition for use, or the method of treatment described herein, may ameliorate one or more symptoms associated with FTD. Symptoms of FTD may include personality and behavior changes, language problems, problems with mental abilities, memory problems and physical problems (e.g., difficulties with movement). The FTD may be characterized by frontotemporal lobar degeneration (FTLD). The FTLD may be FTLD-TDP, which is an FTLD associated with TDP-43 pathology. This may be characterized by ubiquitin and TDP-43 positive, tau negative, FUS negative inclusion bodies. The FTLD-TDP may be of Type A, Type B, Type C or Type D. Type A is a type of FTLD-TDP that presents with small neurites and neuronal cytoplasmic inclusion bodies in the upper (superficial) cortical layers. Bar-like neuronal intranuclear inclusions may also be seen, although comparatively fewer in number. Type B is a type of FTLD-TDP that presents with neuronal and glial cytoplasmic inclusions in both the upper (superficial) and lower (deep) cortical layers, and lower motor neurons. Neuronal intranuclear inclusions may be absent or are in comparatively small number. Type B may be associated with ALS and C9ORF92 mutations. Type C is a type of FTLD-TDP that presents long neuritic profiles found in the superficial cortical laminae. There may be comparatively few or no neuronal cytoplasmic inclusions, neuronal intranuclear inclusions or glial cytoplasmic inclusions. FTLD-TDP is often associated with semantic dementia. Type D is a type of FTLD-TDP that presents with neuronal intranuclear inclusion and dystrophic neurites. There may be no inclusions in the granule cell layer of the hippocampus. Type D may be associated with VCP mutations. In an embodiment, the FTLD may not be of type FTLD-FUS or FTLD-tau. In an embodiment, the TDP-43 regulated cryptic exon is a UNC13A TDP-43 regulated cryptic exon and the neurodegenerative disorder is FTD. In another embodiment, the TDP-43 regulated cryptic exon is STMN2 TDP-43 regulated cryptic exon and the neurodegenerative disorder is FTD.

Also disclosed herein is a method of treating a disease associated with TDP-43 dysfunction (e.g., a neurodegenerative disorder or a muscular disorder) the method comprising administering to a subject in need thereof a therapeutically effective amount of the construct, vector, or pharmaceutical composition disclosed herein.

The construct, vector or pharmaceutical composition described for use or in the methods of treatment herein can be used to prevent loss of and/or restore functionality of certain proteins that are regulated by TDP-43 splicing. In some embodiments, the construct, vector or pharmaceutical composition described for use or in the methods of treatment herein can be used to prevent loss of and/or restore functionality of genes containing a TDP-43 regulated cryptic exon. This may be any gene described herein and includes, for example, UNC13A, STMN2 or INSR.

The construct, vector or pharmaceutical composition for use, or when used as a medicament or used in a method of treatment as described herein may be administered to any suitable subject. In a preferred embodiment, the subject is human. In an embodiment, the subject possesses a SNP variant associated with rs12973192 and/or rs12608932. The human subject is any suitable age, for example, an infant (less than 1 year of age) a child (younger than 18 years of age) including adolescents (10 to 18 years of age inclusive), or adults (older than 18 years of age) including elderly subjects (older than 65 years of age).

The construct, vector or pharmaceutical composition for use, or when used as a medicament or used in a method of treatment as described herein may be administered using any suitable mode of administration.

### Methods of Use

The present disclosure provides methods for use of the constructs, vectors and pharmaceutical compositions of the present disclosure. These methods may be in vivo or in vitro methods.

The constructs, vectors and pharmaceutical compositions re may be used for regulating gene expression at multiple levels. Some aspects of the present disclosure provide methods for regulation gene expression in a cell comprising administering to the cell the construct, vector or pharmaceutical compositions described herein. In some embodiments, the gene expression is regulated at the transcription level, or post-transcription level, or translational level, or post-translational level.

Disclosed herein is a method of modulating splicing of a TDP-43 regulated cryptic exon, the method comprising delivering to a cell the construct described herein, the vector of described herein, or the pharmaceutical composition of described herein, wherein the method comprises contacting the construct with a cell to modulate splicing of the TDP-43 regulated cryptic exon.

Disclosed herein is a method of modulating splicing of the UNC13A cryptic exon, the method comprising delivering to a cell a construct described herein, the vector of described herein, or the pharmaceutical composition of described herein, each comprising an antisense sequence that is at least 90% complementary with SEQ ID NO: 1 or 2 wherein the method comprises contacting the construct with a cell to modulate splicing of the UNC13A cryptic exon. In some embodiments, the antisense sequence is at least 90% complementary with SEQ ID NO: 3 or 4.

Disclosed herein is a method of modulating splicing of the STMN2 cryptic exon 2a, the method comprising delivering to a cell a construct described herein, the vector of described herein, or the pharmaceutical composition of described herein, each comprising an antisense sequence that is at least 90% complementary with SEQ ID NO: 7, wherein the method comprises contacting the construct with a cell to modulate splicing of the STMN2 cryptic exon 2a.

Disclosed herein is a method of modulating splicing of the INSR cryptic exon, the method comprising delivering to a cell a construct described herein, the vector of described herein, or the pharmaceutical composition of described herein, each comprising an antisense sequence that is at least 90% complementary with SEQ ID NO: 9, wherein the method comprises contacting the construct with a cell to modulate splicing of the INSR cryptic exon 2a.

Disclosed herein is a method of preventing inclusion of the TDP-43 regulated cryptic exon in the mature mRNA of a cell transcript, the method comprising delivering to a cell the construct described herein, the vector of described herein, or the pharmaceutical composition of described herein, wherein the method comprises contacting the construct with a cell to prevent inclusion of the TDP-43 regulated cryptic exon in the mature mRNA of a cell transcript.

### Combined Vector

According to a sixth aspect of the invention, there is provided a combined vector comprising two or more of the constructs described herein or of the first aspect of the invention (i.e., in tandem, or one downstream of another, such that the combined vector comprises at least two constructs, each comprising one antisense sequence as defined herein and each comprising a sequence comprising a binding domains for a hnRNP protein as defined herein). In preferred embodiments, the two or more modified U7 snRNA constructs comprise different antisense sequences that are capable of binding to (i.e., they are at least 90%, or at least 95%, or 100% complementary to) different TDP-43 regulated cryptic exons described herein. In some embodiments, the combined vector may comprise three or more constructs as defined herein. In some embodiments, the combined construct comprises two or more antisense sequences that are complementary (i.e., at least 90% complementary, or at least 95% complementary, or 100% complementary) to two or more TDP-43 regulated cryptic exon sequences or flanking regions thereof. In some embodiments, the TDP-43 regulated cryptic exon is selected from one of the TDP-43 regulated cryptic exons defined herein. In some embodiments, each antisense sequence is a sequence that is complementary (i.e., 90%, 95% or 100% complementary) to SEQ ID NO: 1, 2, 3,4, 7, 9, or 448-453). In some embodiments, at least one of the antisense sequences, or each antisense sequences, is complementary to a TDP-43 binding region of the TDP-43 regulated cryptic exon, preferably wherein at least one of the antisense sequences, or each antisense sequence, is complementary (i.e., 90%, 95% or 100% complementary) to SEQ ID NO: 12, 23-26 or 32. In some embodiments, the combined vector comprises a construct as defined herein comprising an antisense sequence which is at least 90% complementary to a UNC13A TDP-43 regulated cryptic exon or flanking region thereof and a construct as defined herein comprising an antisense sequence which is at least 90% complementary to a STMN2 TDP-43 regulated cryptic exon or flanking region thereof. In some embodiments, the combined vector comprises a construct as defined herein comprising an antisense sequence which is at least 90% complementary to a UNC13A TDP-43 regulated cryptic exon or flanking region thereof and a construct as defined herein comprising an antisense sequence which is at least 90% complementary (or 95%, or 100% complementary) to a INSR TDP-43 regulated cryptic exon or flanking region thereof. In some embodiments, the combined vector comprises a construct as defined herein comprising an antisense sequence which is at least 90% complementary (or 95%, or 100% complementary) to a STMN2 TDP-43 regulated cryptic exon or flanking region thereof and a construct as defined herein comprising an antisense sequence which is at least 90% complementary (or 95%, or 100% complementary) to a INSR TDP-43 regulated cryptic exon or flanking region thereof. In some embodiments, the combined vector comprises a construct comprising an antisense sequence which is at least 90% complementary (or 95%, or 100% complementary) to a UNC13A TDP-43 regulated cryptic exon or flanking region thereof, a construct comprising an antisense sequence which is at least 90% complementary (or 95%, or 100% complementary) to a STMN2 TDP-43 regulated cryptic exon or flanking region thereof, and a construct comprising an antisense sequence which is at least 90% complementary (or 95%, or 100% complementary) to a INSR TDP-43 regulated cryptic exon or flanking region thereof.

In some embodiments, the combined vector comprises two or more constructs defined herein, wherein the two or more sequences comprising a binding domain for a hnRNP protein may be according to any sequence as described herein. In some embodiments, the two or more sequences comprising a binding domain for a hnRNP protein may be different or identical. In some embodiments, the two or more sequences comprising a binding domain for a hnRNP protein may be a binding domain for a hnRNP A or hnRNP H protein, and in some examples, a hnRNP A protein.

In some embodiments, the combined vector comprises two or more promoter sequences, wherein the two or more promoter sequences are upstream of each construct. The promoters may be any promoter sequence used in the art. In some embodiments, each of the two or more promoter sequences are the same or different. In some embodiments, the combined vector comprises two or more 3′ box sequences, wherein the two or more 3′ box sequences are downstream of each construct. The 3′ box sequences may be the same or different and may be any 3′ box sequence used in the art.

In some embodiments, the combined vector comprises two or more U7 cassettes, wherein each cassette comprises a promoter, a modified U7 snRNA construct as defined herein, and a 3′ box sequence, wherein the promoter is upstream of the modified U7 snRNA construct and the 3′ box sequence is downstream of the modified U7 snRNA construct. In some embodiments, the combined vector comprises a stuffer sequence between each of the two or more U7 cassettes. The stuffer sequences serve to space out the two promoters. The stuffer sequence may be any suitable stuffer sequence used in the art.

In some embodiments, the combined vector comprises (from upstream to downstream) at least:
A first promoter,
A first modified U7 modified RNA construct as defined herein,
A first 3′ box sequence,
A stuffer sequence,
A second promoter,
A second modified U7 modified RNA construct as defined herein, and
A second 3′ box sequence.

### EXAMPLES

The modified U7 snRNA constructs described in the Examples are all U7 smOPT constructs designed to target TDP-43 regulated cryptic exon sequences to restore correct splicing in TDP-43 depleted cells. The U7 smOPT constructs comprise (i) a binding sequence for a hnRNP, (ii) an antisense sequence designed to target a TDP-43 regulated cryptic exon and flanking regions thereof, and (iii) a modified Sm sequence (e.g., smOPT sequence).

### UNC13A

One such TDP-43 regulated cryptic exon is in the gene UNC13A, which is located between exons 20 and 21. SEQ ID NO 1-shows a portion of UNC13A transcribed pre mRNA intronic sequence including the cryptic exon sequence and flanking regions thereof, including the TDP-43 binding region in the proximity of the cryptic exon as determined by iCliP. The shorter cryptic exon sequence is in italics and the longer cryptic exon sequence is underlined. The lower-case bases denote the bases immediately flanking the splice donor site (gu) and the splice acceptor sites (ag). The ESE targets identified by ESE finder 3.0 are shown in bold.

UGGGAAGCCCACCUUGGCCUCCAGGUUGACUCUCACUACUCAUCAUCA
GGUUCUUCCUUCUAUUCCagCCCUAACCACUCAGGAUUGGGCCGUUUG
UGUCUGGGUAUGUCUCUUCCagCUGCCUGGGUUUCCUGGAAAGAACUC
UUAUCCCCAGGAACUAGUUUGUUGAAUAAAUGCUGGUGAAUGAAUGAA
UGAUUGAACAGAUGAAUGAGUGAUGAGUAGAUAAAAGGAUGGAUGGAG
AGAUGGguGAGUACAUGGAUGGAUAGAUGGAUGAGUUGGUGGGUAGAU
UCGUGGCUAGAUGGAUGAUGGAUGGAUGGACAGAUGGAUGGAUAUAUG
AUUGAACUAUUGAAAGUAUAGAUGUAUGGAUGGGUGAAUUUGGGGGUA
AUUGUUAGAUGAUGGAUGAGUAUAGAUGAAUGAUGGAUGGAUAACUUG
AUGAGUGGAUAGAUAGAUUGCUGGAUAGAUGAUUGACUGGGUGGAUAG
AUGAAAUGUUGGAUGAGCAGAUUAAGUUGUAUUGGAUGGGAUGGAUGG
AAGUGUGGUUGAGUUAUUAGAAGGAAGAUUGAGUAGAUAGGUGAAUUU
GUUGAUAGUCAGAUGGGUAGAUAGGUAGAUGGAUGGAUGGAUGGAUGG
AUGUAUAGGCAGAUGGACAAAUGGAUGAAUGGGUGGGUGGAUGAAUGG
AAGGAUGUGUGGUUGAACUAUUGCAAGUAUUGAUAAUUGGGUUCAUAA
UUUCUGAAUAUUUAGAUGGAUGGUUGUGAGUGGCUGGUGGACAGACGA
AAAAUGGAUGGUUGGAUAAAUUGAUGGGUGGAUGGAUGGUUGGUUGUA
UGAAAGAAUGAAUGAUUGGGUAGGUGGAUUAAGUUGCGGAUCAAUGUA
UGGGAUGGAUGAAUGGAUGGAUGGAUGGAUGUGUGGUUGAAUUACUGA
AAGGUUGGAAGAGUGGAUGGGUGAAAUUUGGGGUAGUUAGAUGGGUGG
GUGUGUGGAUGGAUAAAAGAGUAGAUGAAUG

The ESE targets correspond to binding sites for SR proteins, these motifs are as follows: SRSF5 (ACUCAGG), SRSF1 (CUCAGGA), SRSF6 (UGUGUC) and SRSF2 (GUUUCCUG).

SEQ ID NO: 1 is reproduced again below. Here, the SNPs are located at position rs 12973192 (i.e., within the UNC13A CE sequence), and rs12608932 (i.e., within the intronic region) are shown underlined and the TDP-43 binding region is shown in bold (i.e., as determined by iCLIP data).

UGGGAAGCCCACCUUGGCCUCCAGGUUGACUCUCACUACUCAUCAUCA
GGUUCUUCCUUCUAUUCCagCCCUAACCACUCAGGAUUGGGCCGUUUG
UGUCUGGGUAUGUCUCUUCCagCUGCCUGGGUUUCCUGGAAAGAACUC
UUAUCCCCAGGAACUAGUUUGUUGAAUAAAUGCUGGUGAAUGAAUGAA
UGAUUGAACAGAUGAAUGAGUGAUGAGUAGAUAAAAGGAUGGAUGGAG
AGAUGGguGAGUACAUGGAUGGAUAGAUGGAUGAGUUGGUGGGUAGAU
UCGUGGCUAGAUGGAUGAUGGAUGGAUGGACAGAUGGAUGGAUAUAUG
AUUGAACUAUUGAAAGUAUAGAUGUAUGGAUGGGUGAAUUUGGGGGUA
AUUGUUAGAUGAUGGAUGAGUAUAGAUGAAUGAUGGAUGGAUAACUUG
AUGAGUGGAUAGAUAGAUUGCUGGAUAGAUGAUUGACUGGGUGGAUAG
AUGAAAUGUUGGAUGAGCAGAUUAAGUUGUAUUGGAUGGGAUGGAUGG
AAGUGUGGUUGAGUUAUUAGAAGGAAGAUUGAGUAGAUAGGUGAAUUU
GUUGAUAGUCAGAUGGGUAGAUAGGUAGAUGGAUGGAUGGAUGGAUGG
AUGUAUAGGCAGAUGGACAAAUGGAUGAAUGGGUGGGUGGAUGAAUGG
AAGGAUGUGUGGUUGAACUAUUGCAAGUAUUGAUAAUUGGGUUCAUAA
UUUCUGAAUAUUUAGAUGGAUGGUUGUGAGUGGCUGGUGGACAGACGA
AAAAUGGAUGGUUGGAUAAAUUGAUGGGUGGAUGGAUGGUUGGUUGUA
UGAAAGAAUGAAUGAUUGGGUAGGUGGAUUAAGUUGCGGAUCAAUGUA
UGGGAUGGAUGAAUGGAUGGAUGGAUGGAUGUGUGGUUGAAUUACUGA
AAGGUUGGAAGAGUGGAUGGGUGAAAUUUGGGGUAGUUAGAUGGGUGG
GUGUGUGGAUGGAUAAAAGAGUAGAUGAAUG

The splice sites are defined as follows: Long cryptic acceptor is the phosphodiester bond between chr19: 17,642,591-17,642,592; the Short cryptic acceptor is the phosphodiester bond between chr19: 17,642,541-17,642,542 and the Cryptic donor is the phosphodiester bond between chr19: 17,642,413-17,642,414.

SEQ ID NO: 1 encompasses the minor allele of the SNP (i.e., the risk variant) or the major allele at rs12973192 and/or rs12608932, therefore SEQ ID NO: 1 also encompasses the sequence with SNP at these positions (e.g., wherein the G at rs12973192 is replaced with a C, defined by SEQ ID NO: 2).

SEQ ID NO: 3 shows a shorter portion of UNC13A transcribed pre mRNA intronic sequence including the cryptic exon sequence and flanking regions thereof.

UGGGAAGCCCACCUUGGCCUCCAGGUUGACUCUCACUACUCAUCAUCA
GGUUCUUCCUUCUAUUCCagCCCUAACCACUCAGGAUUGGGCCGUUUG
UGUCUGGGUAUGUCUCUUCCagCUGCCUGGGUUUCCUGGAAAGAACUC
UUAUCCCCAGGAACUAGUUUGUUGAAUAAAUGCUGGUGAAUGAAUGAA
UGAUUGAACAGAUGAAUGAGUGAUGAGUAGAUAAAAGGAUGGAUGGAG
AGAUGGguGAGUACAUGGAUGGAUAGAUGGAUGAGUUGGUGGGUAGAU
UCGUGGCUAGAUGGAUGAUGGAUGGAUGGACA

SEQ ID NO: 3 encompasses the minor allele of the SNP (i.e., the risk variant) or the major allele at rs12973192 and/or rs12608932, therefore SEQ ID NO: 3 also encompasses the sequence wherein the G at rs12973192 is replaced with a C. This is defined by SEQ ID NO: 4.

SEQ ID NO: 5 corresponds to the shorter UNC13A cryptic exon sequence transcribed UNC13A mRNA-cords chr19: 17642414-17,642,541.

SEQ ID NO: 5 has the sequence:

CUGCCUGGGUUUCCUGGAAAGAACUCUUAUCCCCAGGAACUAGUU
UGUUGAAUAAAUGCUGGUGAAUGAAUGAAUGAUUGAACAGAUGAA
UGAGUGAUGAGUAGAUAAAAGGAUGGAUGGAGAGAUGG.

SEQ ID NO: 5 encompasses minor allele of the SNP (i.e., the risk variant), or the major allele at rs12973192, therefore SEQ ID NO: 5 also encompasses the sequence wherein the G at rs12973192 is replaced with a C.

SEQ ID NO 6 corresponds to the longer UNC13A cryptic exon sequence in transcribed UNC13A mRNA-cords chr19: 17642414-17642591.

SEQ ID NO 6 has the sequence

CCCUAACCACUCAGGAUUGGGCCGUUUGUGUCUGGGUAUGUCUCU
UCCAGCUGCCUGGGUUUCCUGGAAAGAACUCUUAUCCCCAGGAAC
UAGUUUGUUGAAUAAAUGCUGGUGAAUGAAUGAAUGAUUGAACAG
AUGAAUGAGUGAUGAGUAGAUAAAAGGAUGGAUGGAGAGAUGG.

SEQ ID NO: 6 may encompass the risk variant of the SNP (i.e., minor allele), or the major allele at rs12973192, therefore SEQ ID NO: 6 also encompasses the sequence wherein the G at rs 12973192 is replaced with a C.

### STMN2

Another TDP-43 regulated cryptic exon is in the gene STMN2, corresponding to exon 2a of the STMN2 gene.

SEQ ID NO 7-shows a portion of STMN2 transcribed pre mRNA intronic sequence and part of the cryptic exon sequence 2a. The lower-case bases denote the bases immediately flanking the splice acceptor site (ag). The polyA site is shown underlined. The ESE targets identified by ESE finder 3.0 are shown in bold. The TDP-43 binding motif is shown underlined.

UGCCCCAUCACUCUCUCUUAAUU
GGAUUUUUAAAAUUAUAUUCAUA
UUGCagGACUCGGCAGAAGA
CCUUCGAGAGAAAGGUAGAAA
AUAAGAAUUUGGCUCUC
UGUGUGAGCAUGUGUGCGUGUGU
GCGAGAGAGAGAGACAGACAGCC
UGCCUAAGAAGAAAUGAAUGUGAA
UGCGGCUUGUGGCACAGU
UGACAAGGAUGAUAAAUC
AAUAAUGCAAGCUUACUAUCAUU
UAUGAAUAGCAAU
ACUGAAGAAAUUAAAACAAAAGA
UUGCUGUCUC

The ESE targets in the STMN2 cryptic exon and flanking regions thereof correspond to binding sites for SRSF1 (CAGAAGA), SRSF2 (GGCUUGUG), SRSF5 (UGACAAG) and SRSF6 (UGCGGC).

SEQ ID NO: 8 shows the STMN2 cryptic exon 2a. This has the genomic position. chr8: 79,616,822-79,617,048

GACUCGGCAGAAGACCUUCGAGAGAAAGGUA
GAAAAUAAGAAUUUGGCUCUCUGUGUGAGCA
UGUGUGCGUGUGUGCGAGAGAGAGAGACAGA
CAGCCUGCCUAAGAAGAAAUGAAUGUGAAUG
CGGCUUGUGGCACAGUUGACAAGGAUGAUAA
AUCAAUAAUGCAAGCUUACUAUCAUUUAUGA
AUAGCAAUACUGAAGAAAUUAAAACAAAAGA
UUGCUGUCUC

### INSR

SEQ ID NO: 9 shows a portion of INSR transcribed pre mRNA intronic sequence with cryptic exon corresponding to the genomic position chr 19, complement: 7169720-716983. The lower-case bases denote the bases immediately flanking the splice acceptor site (ag). The ESE targets identified by ESE finder 3.0 are shown in bold. The TDP-43 binding motif is shown underlined.

UAUUAAUAUUAUCACUAUGCUUA
CUGUGCCAUAUagUACCGGAUAC
GGGAUGAAGUCAUACAAGCACUG
AAUGAAUGGAUGAAUGAAUGAUG
GAUGAAUGGAUGACACCUUCUUA
UAUGUGUAUCAGGCUGAUGCUGA
AGACUUCAAAGUUGAGUAAAAUA
CCUAUGUCAGUC

The ESE targets correspond to binding sites for SRSF1 (GACACCT and CTGAAGA), SRSF2 (GAAUGAUG and GGCUGAUG), SRSF5 (AUACAAG) and SRSF6 (UACGGG and UGUGUA).

SEQ ID NO: 10 shows the INSR cryptic exon.

UACCGGAUACGGGAUGAAGUCAUACAAGCAC
UGAAUGAAUGGAUGAAUGAAUGAUGGAUGAA
UGGAUGACACCUUCUUAUAUGUGUAUCAGGC
UGAUGCUGAAGACUUCAAA

### ELALV3

SEQ ID NO: 448 shows a portion of ELAVL3 transcribed pre mRNA intronic sequence with cryptic exon corresponding to the genomic position chr19, complement: 11463496-11463662 The lower-case bases denote the bases immediately flanking the splice acceptor site (ag). The TDP-43 hindino region is shown underlined

CCCGGCCCAGGAUGACGUGCUUAUUAU
UUGACagGUGCAUGUGACACUGUGACU
CCGGCUGUGACCUGAUGGGGCCUCAGG
GAUGCGUCUGGCUCUGGCAGGAUGUUU
GUGUGUCACCGCGAUGUUGUGUGGGUG
UGUCUACCUGUGCCCUGCUCUGAGGGA
UUGAGUGUGAUAUCGUGUGUUUGUGCU
GCGCUGUGAUGG

### G3BP1

SEQ ID NO: 449 shows a portion of G3BP1 transcribed pre mRNA intronic sequence with cryptic exon corresponding to the genomic position chr5, complement: 151787765-151787794 The lower-case bases denote the bases immediately flanking the splice acceptor site (ag). The TDP-43 binding region is shown underlined.

GACCAGAACUAUUUUUUCCCUUACAC
CUUGACCagCUUGCAUAUUGGAUACC
ACAUGAUUAUCAG

### AARS1

SEQ ID NO: 450 shows a portion of AARS1 transcribed pre mRNA intronic sequence with cryptic exon corresponding to the genomic position chr 16, complement: 70272796-70272882 The lower-case bases denote the bases immediately flanking the splice acceptor site (ag). The TDP-43 binding region is shown underlined.

AUCUUUUGUGUGUGUGUGUGUGUGUG
UGUGUGUGUGUGUGUCACCCagGCUG
GAGUGCAGUGGCAUGAUCACAGCUCA
CUGCAGCCUCAACUUCCUGGGCUCAA
GUGAUCCUCUCCCGAGUAGCUGGGAC
UACAG

### CELF5

SEQ ID NO: 451 shows a portion of CELF5 transcribed pre mRNA intronic sequence with cryptic exon corresponding to the genomic position chr19, complement: 3278209-3278316.

The lower-case bases denote the bases immediately flanking the splice donor site (gu). The TDP-43 binding region is shown underlined.

GUAGCCCCUGGCUGUCCUUCAGAGGG
GGCACAGGUGGAGAAAGAGGCGCAGU
CCCUGGCUGUGGUCCCUGGAGUGGGU
AUACACGUGUGAGUGUGUGCAGAUGU
GGAGguGAGUAGGCAAGCGAAGUGUA
UGUGUGUGCAUGGAUGUAUUACAAGU
GUGUGCGUGUGGGUGAGUGUGCAUGU
CUGGGUGUGAGUGUGCCCGAGACUGC
AUGCAUGUGUGUGUGUGAGU

### CAMK2B

SEQ ID NO: 452 shows a portion of CAMK2B transcribed pre mRNA intronic sequence with cryptic exon corresponding to the genomic position chr7, complement: 44258490-44258514

The lower-case bases denote the bases immediately flanking the splice donor site (gu). The TDP-43 binding region is shown underlined.

CUGGACUGGGGCACCACUGGCUGGCgu
GAGUGCACAUGUGUGUGUUUAUGAGUG
UGGCUGUGAAAUGUGAGCACGUGCACC
UGUAUGUAUGUGUGUGGGUGUUUGCAC
GUGGGGGCGCGUGAGCACAUGAAAUCA
GGGUCCAUAUGGGUGGGGAUGUGCAUA
CAUGUGCAUGUGUAUUGUGUGAAUGUA
UGAGUGAGCGUGUGGAGGUGUGUGCAU
GUGG

### UNC13B

SEQ ID NO: 453 shows a portion of UNC13B transcribed pre mRNA intronic sequence with cryptic exon corresponding to the genomic position chr9, complement: 35,364,545-35,364,567. The lower-case bases denote the bases immediately flanking the splice donor site (gu). The TDP-43 binding region is shown underlined.

AAGAAAAGCGAGGAGCCCUUCAGguUG
UGCCUAUGACCCUUUGGGUCGUCCUUU
UUUGUCUAUUUCCCUCCCCUCCUUCCC
AAGCACUGUAUGUGUGUGUGUAUGUGU
GUGUGUGUGUGUACAUGCACAUGUGCG
UGCAUGAUCUGUGCCUCUGAGCUUUGG
CUCAUGCAGUCUAUUUUUCUGAAAAGC
AGUUUGUGUGCAUGC

### Example Target Sequences for Splicing Elements in TDP-43 Regulated Cryptic Exons

The following sequences comprising target sequences. The antisense sequences used in the constructs of the present invention may comprise sequences which are at least 90%, or at least 95%, or at least 100% complementary to these target sequences.

| SEQ ID | Target Sequence | Target site |
|---|---|---|
| SEQ ID NO: 11 | UAAAAUUAUAUUCAUAUUGCagGACUCGGCAGAA | STMN2 2a 3′ splice site |
| | GACCUU | and flanking regions |
| SEQ ID NO: 12 | GUAGAAAAUAAGAAUUUGGCUCUCUGUGUGAGCA | STMN2 2a TDP-43 |
| | UGUGUGCGUGUGUGCGAGAGAGAGAGACAGACA | binding region and |
| | | flanking regions |
| SEQ ID NO: 13 | UGUGUGAGCAUGUGUGCGUGUGU | STMN2 2a TDP-43 |
| | | binding region |
| SEQ ID NO: 14 | AUUCAUAUUGCAGGACUCGGCAGAAGACCUUCGA | STMN2 2a ESE and |
| | GAGAAAGGUAGAA | flanking region (SRSF1) |
| SEQ ID NO: 15 | UAAGAAGAAAUGAAUGUGAAUGCGGCUUGUGGC | STMN2 2a ESE and |
| | ACAGUUGACAAGGAUGAU | flanking region (SRSF2 |
| | | and SRSF6) |
| SEQ ID NO: 16 | AAUGCGGCUUGUGGCACAGUUGACAAGGAUGAUA | STMN2 2a ESE and |
| | AAUCAAUAAUGCA | flanking region (SRSF5) |
| SEQ ID NO: 17 | UGCGGCUUGUGGCACAGUUGACAA | STMN2 ESE target region |
| | | (i.e., comprising one or |
| | | more ESE sites identified |
| | | by ESE finder 3.0) |
| SEQ ID NO: 18 | UAAGAAGAAAUGAAUGUGAAUGCGGCUUGUGGC | STMN2 ESE target region |
| | ACAGUUGACAAGGAUGAUAAAUCAAUAAUGCA | and flanking region |
| | | thereof |
| SEQ ID NO: 19 | GGUUCUUCCUUCUAUUCCAGCCCUAACCACUCAG | UNC13A 1^{st} 3′ splice site |
| | GAUUGG | and flanking regions |
| SEQ ID NO: 20 | UGUCUGGGUAUGUCUCUUCCAGCUGCCUGGGUUU | UNC13A 3′ splice site and |
| | CCUGGA | flanking regions |
| SEQ ID NO: 21 | AAGGAUGGAUGGAGAGAUGGGUGAGUACAUGGA | UNC13A 5′ splice site and |
| | UGGAUAG | flanking regions |
| SEQ ID NO: 22 | AAGCAUGGAUGGAGAGAUGGGUGAGUACAUGGA | UNC13A 5′ splice site and |
| | UGGAUAG | flanking regions with CE |
| | | SNP |
| SEQ ID NO: 23 | UGAAUGAAUGAAUGAUUGAACAGAUGAAUGAGU | UNC13A TDP-43 binding |
| | GAUGAGUAGAUAAAAGGAUGGAUGGAGAGAUGG | region as determined by |
| | GUGAGUACAUGGAUGGAUAGAUGGAUGAGUUGG | iClip |
| | UGGGUAGAUUCGUGGCUAGAUGGAUGAUGGAUG | |
| | GAUGGACAGAUGGAUGGAUAUAUGAUUGAACUA | |
| | UUGAAAGUAUAGAUGUAUGGAUGGGUGAAUUUG | |
| | GGGGUAAUUGUUAGAUGAUGGAUGAGUAUAGAU | |
| | GAAUGAUGGAUGGAUAACUUGAUGAGUGGAUAG | |
| | AUAGAUUGCUGGAUAGAUGAUUGACUGGGUGGA | |
| | UAGAUGAAAUGUUGGAUGAGCAGAUUAAGUUGU | |
| | AUUGGAUGGGAUGGAUGGAAGUGUGGUUGAGUU | |
| | AUUAGAAGGAAGAUUGAGUAGAUAGGUGAAUUU | |
| | GUUGAUAGUCAGAUGGGUAGAUAGGUAGAUGGA | |
| | UGGAUGGAUGGAUGGAUGUAUAGGCAGAUGGAC | |
| | AAAUGGAUGAAUGGGUGGGUGGAUGAAUGGAAG | |
| | GAUGUGUGGUUGAACUAUUGCAAGUAUUGAUAA | |
| | UUGGGUUCAUAAUUUCUGAAUAUUUAGAUGGAU | |
| | GGUUGUGAGUGGCUGGUGGACAGACGAAAAAUG | |
| | GAUGGUUGGAUAAAUUGAUGGGUGGAUGGAUGG | |
| | UUGGUUGUAUGAAAGAAUGAAUGAUUGGGUAGG | |
| | UGGAUUAAGUUGCGGAUCAAUGUAUGGGAUGGA | |
| | UGAAUGGAUGGAUGGAUGGAUGUGUGGUUGAAU | |
| | UACUGAAAGGUUGGAAGAGUGGAUGGGUGAAAU | |
| | UUGGGGUAGUUAGAUGGGUGGGUGUGUGGAUGG | |
| | AUAAAAGAGUAGAUGAAUG | |
| SEQ ID NO: 24 | GUUUGUUGAAUAAAUGCUGGUGAAUGAAUGAAU | Shorter UNC13A TDP-43 |
| | GAUUGAACAGAUGAAUGAGUGAUGAGUAGAUAA | binding region and |
| | AAGGAUGGAUGGAGAGAUGGguGAGUACAUGGAU | flanking regions without |
| | GGAUAGAU | CE SNP |
| SEQ ID NO: 25 | GUUUGUUGAAUAAAUGCUGGUGAAUGAAUGAAU | UNC13A TDP-43 binding |
| | GAUUGAACAGAUGAAUGAGUGAUGAGUAGAUAA | region and flanking |
| | AAGCAUGGAUGGAGAGAUGGGUGAGUACAUGGA | regions with CE SNP |
| | UGGAUAGAU | |
| SEQ ID NO: 26 | GGAUGGAUGGAGAGAUGGGUGA | UNC13A TDP-43 shorter |
| | | binding region comprising |
| | | UG-rich motifs |
| SEQ ID NO: 27 | CUUCUAUUCCAGCCCUAACCACUCAGGAUUGGGC | UNC13A ESE and |
| | CGUUUGUGUCUGGG | flanking region (SRSF1 |
| | | and SRSF5) |
| SEQ ID NO: 28 | UGUCUCUUCCAGCUGCCUGGGUUUCCUGGAAAGA | UNC13A ESE and |
| | ACUCUUAUCCCCAG | flanking region (SRSF2) |
| SEQ ID NO: 29 | CCACUCAGGAUUGGGCCGUUUGUGUCUGGGUAUG | UNC13A ESE and |
| | UCUCUUCCAGCU | flanking region (SRSF6) |
| SEQ ID NO: 30 | ACUCAGGAUUGGGCCGUUUGUGUCUGGGUAUGUC | UNC13A target ESE |
| | UCUUCCAGCUGCCUGGGUUUCCUG | region (i.e., comprising |
| | | one or more ESE sites |
| | | identified by ESE finder |
| | | 3.0) |
| SEQ ID NO: 31 | CUAUGCUUACUGUGCCAUAUAGUACCGGAUACGG | INSR 3′ splice site and |
| | GAUGAA | flanking regions |
| SEQ ID NO: 32 | GGAUACGGGAUGAAGUCAUACAAGCACUGAAUGA | INSR TDP-43 binding |
| | AUGGAUGAAUGAAUGAUGGAUGAAUGGAUGACA | region and flanking |
| | CCUUCUUAUAUGUAUCAGGCUGAUGCUGAAGACG | regions |
| | GAUACGGGAUGAAGUCAUACAAGCAC | |
| SEQ ID NO: 33 | UGAAUGAAUGGAUGAAUGAAUGAUGGAUGAAUG | INSR TDP-43 binding |
| | GAUGACACCUUCUUAUAUGU | region |
| SEQ ID NO: 34 | UGAAUGAUGGAUGAAUGGAUGACACCUUCUUAUA | INSR ESE and flanking |
| | UGUGUAUCAGGCU | region (SRSF1) |
| SEQ ID NO: 35 | AUAUGUGUAUCAGGCUGAUGCUGAAGACUUCAAA | INSR ESE and flanking |
| | GUUGAGUAAAAUA | region (SRSF1) |
| SEQ ID NO: 36 | CACUGAAUGAAUGGAUGAAUGAAUGAUGGAUGA | INSR ESE and flanking |
| | AUGGAUGACACCUUC | region (SRSF2) |
| SEQ ID NO: 37 | ACCUUCUUAUAUGUGUAUCAGGCUGAUGCUGAAG | INSR ESE and flanking |
| | ACUUCAAAGUUGAGUAAAAUA | region (SRSF2) |
| SEQ ID NO: 38 | GUACCGGAUACGGGAUGAAGUCAUACAAGCACUG | INSR ESE and flanking |
| | AAUGAAUGGAUGAAU | region (SRSF5) |
| SEQ ID NO: 39 | CUGUGCCAUAUagUACCGGAUACGGGAUGAAGUC | INSR ESE and flanking |
| | AUACAAGCACUG | region (SRSF6) |
| SEQ ID NO: 40 | AUGGAUGACACCUUCUUAUAUGUGUAUCAGGCUG | INSR ESE and flanking |
| | AUGCUGAAGACU | region (SRSF6) |
| SEQ ID NO: 454 | GACGUGCUUAUUAUUUGACagGUGCAUGUGACAC | ELALV3 3 ′splice site |
| | UGUGACU | and flanking region |
| SEQ ID NO: 455 | ACGUGCUUAUUAUUUGACagGUGCAUGUGACACU | ELALV3 TDP-43 binding |
| | GUGACUCCGGCUGUGACCUGAUGGG | region and flanking region |
| | | thereof |
| SEQ ID NO: 456 | GGCCUCAGGGAUGCGUCUGGCUCUGGCAGGAUGU | ELALV3 TDP-43 binding |
| | UUGUGUGUCACCGCGAUGUUGUGUGGG | region and flanking region |
| | | thereof. |
| SEQ ID NO: 457 | UGUGUCACCGCGAUGUUGUGUGGGUGUGUCUACC | ELALV3 TDP-43 binding |
| | UGUGCCCUGCUCUGAGGGAUUGAGUGUG | region and flanking region |
| | | thereof. |
| SEQ ID NO: 458 | UUUUCCCUUACACCUUGACCagCUUGCAUAUUGGA | G3BP1 3′ splice site and |
| | UACCACA | flanking region thereof |
| SEQ ID NO: 459 | ACACCUUGACCagCUUGCAUAUUGGAUACCAC | G3BP1 TDP-43 binding |
| | | region |
| SEQ ID NO: 460 | UGUGUGUGUGUGUGUCACCCagGCUGGAGUGCAG | AARS1 3′ splice site and |
| | UGGCAUGA | flanking region thereof. |
| SEQ ID NO: 461 | AUCUUUUGUGUGUGUGUGUGUGUGUGUGUGUGU | AARS1 TDP-43 binding |
| | GUGUGUGUCACCCagGCUGGAGUGCAGUGGCAUG | region |
| | AUCACAGCUCACUGCAGCCUCAACUUCCU | |
| SEQ ID NO: 462 | AGUGAUCCUCUCCCGAGUAGCUGGGACUACAG | AARS1 TDP-43 binding |
| | | region |
| SEQ ID NO: 463 | GUGUGUGCAGAUGUGGAGguGAGUAGGCAAGCG | CELF5 5′ splice site and |
| | AAGUGUA | flanking region thereof. |
| SEQ ID NO: 464 | AGGCAAGCGAAGUGUAUGUGUGUGCAUGGAUGU | CELF5 TDP-43 binding |
| | AUUACAAGUGUGUGCGUGUGGGUGAGUG | region and flanking |
| | | regions thereof |
| SEQ ID NO: 465 | CAAGUGUGUGCGUGUGGGUGAGUGUGCAUGUC | CELF5 TDP-43 binding |
| | UGGGUGUGAGUGUGCCCGAGA | region and flanking |
| | | regions thereof |
| SEQ ID NO: 466 | AGUGUGCCCGAGACUGCAUGCAUGUGUGUGUGU | CELF5 TDP-43 binding |
| | GAGU | region and flanking |
| | | regions thereof |
| SEQ ID NO: 467 | GGGGCACCACUGGCUGGCguGAGUGCACAUGUG | CAMK2B 5′ splice site |
| | UGUGUUU | and flanking regions |
| | | thereof |
| SEQ ID NO: 468 | ACAUGUGCAUGUGUAUUGUGUGAAUGUAUGAGU | CAMK2B TDP-43 |
| | GAGCGUGUGGAGGUGUGUGCAUGUGG | binding region and |
| | | flanking regions thereof |
| SEQ ID NO: 469 | AAGAAAAGCGAGGAGCCCUUCAGguUGUGCCUAU | UNC13B 5′ splice site |
| | GACCCUUUGGG | and flanking regions |
| | | thereof |
| SEQ ID NO: 470 | UCCCAAGCACUGUAUGUGUGUGUGUAUGUGUGU | UNC13B TDP-43 binding |
| | GUGUGUGUGUACAUGCACAUGUGCGUGCAUGAU | region and flanking |
| | CUGUGCCUCUGAGCUUUGGCUCAUGC | regions thereof |
| SEQ ID NO: 471 | CAUGCAGUCUAUUUUUCUGAAAAGCAGUUUGUG | UNC13B TDP-43 binding |
| | UGCAUGC | region and flanking |
| | | regions thereof |

### Example Antisense Sequences for Splicing Elements in TDP-43 Regulated Cryptic Exons

Example antisense sequences that target the STMN2 3′ splice site/splice acceptor site

| | |
|---|---|
| SEQ ID NO: 42 | GUCUUCUGCCGAGUCC |
| SEQ ID NO: 43 | GUCUUCUGCCGAGUCc |
| SEQ ID NO: 44 | UCUUCUGCCGAGUCcU |
| SEQ ID NO: 45 | CUUCUGCCGAGUCcUG |
| SEQ ID NO: 46 | UUCUGCCGAGUCcUGC |
| SEQ ID NO: 47 | UCUGCCGAGUCcUGCA |
| SEQ ID NO: 48 | CUGCCGAGUCcUGCAA |
| SEQ ID NO: 49 | UGCCGAGUCcUGCAAU |
| SEQ ID NO: 50 | GCCGAGUCcUGCAAUA |
| SEQ ID NO: 51 | CCGAGUCcUGCAAUAU |
| SEQ ID NO: 52 | CGAGUCcUGCAAUAUG |
| SEQ ID NO: 53 | GAGUCcUGCAAUAUGA |
| SEQ ID NO: 54 | AGUCcUGCAAUAUGAA |
| SEQ ID NO: 55 | GCCcUGCAAUAUGAAU |
| SEQ ID NO: 56 | CCCUGCAAUAUGAAUA |
| SEQ ID NO: 57 | CcUGCAAUAUGAAUAU |
| SEQ ID NO: 58 | cUGCAAUAUGAAUAUA |
| SEQ ID NO: 59 | UGCAAUAUGAAUAUAA |

Example antisense sequences that target the STMN2 TDP-43 binding region and/or flanking regions thereof

| | |
|---|---|
| SEQ ID NO: 60 | UGUCUGUCUCUCUCUC |
| SEQ ID NO: 61 | GUCUGUCUCUCUCUCU |
| SEQ ID NO: 62 | UCUGUCUCUCUCUCUC |
| SEQ ID NO: 63 | CUGUCUCUCUCUCUCG |
| SEQ ID NO: 64 | UGUCUCUCUCUCUCGC |
| SEQ ID NO: 65 | GUCUCUCUCUCUCGCa |
| SEQ ID NO: 66 | UCUCUCUCUCUCGCac |
| SEQ ID NO: 67 | CUCUCUCUCUCGCaca |
| SEQ ID NO: 68 | UCUCUCUCUCGCacac |
| SEQ ID NO: 69 | CUCUCUCUCGCacaca |
| SEQ ID NO: 70 | UCUCUCUCGCacacac |
| SEQ ID NO: 71 | CUCUCUCGCacacacg |
| SEQ ID NO: 72 | UCUCUCGCacacacgc |
| SEQ ID NO: 73 | CUCUCGCacacacgca |
| SEQ ID NO: 74 | UCUCGCacacacgcac |
| SEQ ID NO: 75 | CUCGCacacacgcaca |
| SEQ ID NO: 76 | UCGCacacacgcacac |
| SEQ ID NO: 77 | CGCacacacgcacaca |
| SEQ ID NO: 78 | GCacacacgcacacaU |
| SEQ ID NO: 79 | CacacacgcacacaUg |
| SEQ ID NO: 80 | acacacgcacacaUgc |
| SEQ ID NO: 81 | cacacgcacacaUgcU |
| SEQ ID NO: 82 | acacgcacacaUgcUc |
| SEQ ID NO: 83 | cacgcacacaUgcUca |
| SEQ ID NO: 84 | acgcacacaUgcUcac |
| SEQ ID NO: 85 | cgcacacaUgcUcaca |
| SEQ ID NO: 86 | gcacacaUgcUcacac |
| SEQ ID NO: 87 | cacacaUgcUcacaca |
| SEQ ID NO: 88 | acacaUgcUcacacaG |
| SEQ ID NO: 89 | cacaUgcUcacacaGA |
| SEQ ID NO: 90 | acaUgcUcacacaGAG |
| SEQ ID NO: 91 | caUgcUcacacaGAGA |
| SEQ ID NO: 92 | aUgcUcacacaGAGAG |
| SEQ ID NO: 93 | UgcUcacacaGAGAGC |
| SEQ ID NO: 94 | gcUcacacaGAGAGCC |
| SEQ ID NO: 95 | cUcacacaGAGAGCCA |
| SEQ ID NO: 96 | UcacacaGAGAGCCAA |
| SEQ ID NO: 97 | cacacaGAGAGCCAAA |
| SEQ ID NO: 98 | acacaGAGAGCCAAAU |
| SEQ ID NO: 99 | cacaGAGAGCCAAAUU |
| SEQ ID NO: 100 | acaGAGAGCCAAAUUC |
| SEQ ID NO: 101 | caGAGAGCCAAAUUCU |
| SEQ ID NO: 102 | aGAGAGCCAAAUUCUU |

Example antisense sequences that target the first UNC13A 3′ splice site/splice acceptor site

| | |
|---|---|
| SEQ ID NO: 103 | CCUGAGUGGUUAGGGc |
| SEQ ID NO: 104 | CUGAGUGGUUAGGGcU |
| SEQ ID NO: 105 | UAGUGGUUAGGGcUG |
| SEQ ID NO: 106 | AGUGGUUAGGGcUGG |
| SEQ ID NO: 107 | GUGGUUAGGGcUGGA |
| SEQ ID NO: 108 | UGGUUAGGGcUGGAA |
| SEQ ID NO: 109 | GGUUAGGGcUGGAAU |
| SEQ ID NO: 110 | GUUAGGGcUGGAAUA |
| SEQ ID NO: 111 | UUAGGGcUGGAAUAG |
| SEQ ID NO: 112 | UAGGGcUGGAAUAGA |
| SEQ ID NO: 113 | AGGGcUGGAAUAGAA |
| SEQ ID NO: 114 | GGGcUGGAAUAGAAG |
| SEQ ID NO: 115 | GGcUGGAAUAGAAGG |
| SEQ ID NO: 116 | GcUGGAAUAGAAGGAA |
| SEQ ID NO: 117 | cUGGAAUAGAAGGAAU |
| SEQ ID NO: 118 | cUGGAAUAGAAGGAAUA |

Example antisense sequences that target the second UNC13A 3′ splice site/splice acceptor site

| | |
|---|---|
| SEQ ID NO: 119 | AGGAAACCCAGGCAGc |
| SEQ ID NO: 120 | GGAAACCCAGGCAGCU |
| SEQ ID NO: 121 | GAAACCCAGGCAGcUG |
| SEQ ID NO: 122 | AAACCCAGGCAGcUGG |
| SEQ ID NO: 123 | AACCCAGGCAGcUGGA |
| SEQ ID NO: 124 | ACCCAGGCAGcUGGAA |
| SEQ ID NO: 125 | CCCAGGCAGcUGGAAG |
| SEQ ID NO: 126 | CCAGGCAGcUGGAAGA |
| SEQ ID NO: 127 | CAGGCAGcUGGAAGAG |
| SEQ ID NO: 128 | AGGCAGcUGGAAGAGA |
| SEQ ID NO: 129 | GGCAGcUGGAAGAGAC |
| SEQ ID NO: 130 | GCAGcUGGAAGAGACA |
| SEQ ID NO: 131 | CAGcUGGAAGAGACAU |
| SEQ ID NO: 132 | AGcUGGAAGAGACAUA |
| SEQ ID NO: 133 | GcUGGAAGAGACAUAC |
| SEQ ID NO: 134 | cUGGAAGAGACAUACC |
| SEQ ID NO: 135 | UGGAAGAGACAUACCC |

Example antisense sequences that target the UNC13A 5′ splice site/splice donor site

| | |
|---|---|
| SEQ ID NO: 136 | UCCAUCCAUGUACUCa |
| SEQ ID NO: 137 | CCAUCCAUGUACUCac |
| SEQ ID NO: 138 | CAUCCAUGUACUCacC |
| SEQ ID NO: 139 | AUCCAUGUACUCacCC |
| SEQ ID NO: 140 | UCCAUGUACUCacCCA |
| SEQ ID NO: 141 | CCAUGUACUCacCCAU |
| SEQ ID NO: 142 | CAUGUACUCacCCAUC |
| SEQ ID NO: 143 | AUGUACUCacCCAUCU |
| SEQ ID NO: 144 | UGUACUCacCCAUCUC |
| SEQ ID NO: 145 | GUACUCacCCAUCUCU |
| SEQ ID NO: 146 | UACUCacCCAUCUCUC |
| SEQ ID NO: 147 | ACUCacCCAUCUCUCC |
| SEQ ID NO: 148 | CUCacCCAUCUCUCCA |
| SEQ ID NO: 149 | UCacCCAUCUCUCCAU |
| SEQ ID NO: 150 | CacCCAUCUCUCCAUC |
| SEQ ID NO: 151 | acCCAUCUCUCCAUCC |
| SEQ ID NO: 152 | cCCAUCUCUCCAUCCA |

Example antisense sequences that target the UNC13A TDP-43 binding region and/or flanking regions thereof.

| | |
|---|---|
| SEQ ID NO: 153 | AUCUAUCCAUCCAUGU |
| SEQ ID NO: 154 | UCUAUCCAUCCAUGUA |
| SEQ ID NO: 155 | CUAUCCAUCCAUGUAC |
| SEQ ID NO: 156 | UAUCCAUCCAUGUACU |
| SEQ ID NO: 157 | AUCCAUCCAUGUACUC |
| SEQ ID NO: 158 | UCCAUCCAUGUACUCA |
| SEQ ID NO: 159 | CCAUCCAUGUACUCAC |
| SEQ ID NO: 160 | CAUCCAUGUACUCAcC |
| SEQ ID NO: 161 | AUCCAUGUACUCAcCC |
| SEQ ID NO: 162 | UCCAUGUACUCAcCCA |
| SEQ ID NO: 163 | CCAUGUACUCAcCCAU |
| SEQ ID NO: 164 | AUGUACUCAcCCAUC |
| SEQ ID NO: 165 | UGUACUCAcCCAUCU |
| SEQ ID NO: 166 | GUACUCAcCCAUCUC |
| SEQ ID NO: 167 | UACUCAcCCAUCUCU |
| SEQ ID NO: 168 | ACUCAcCCAUCUCUC |
| SEQ ID NO: 169 | CUCAcCCAUCUCUCC |
| SEQ ID NO: 170 | UCAcCCAUCUCUCCA |
| SEQ ID NO: 171 | CAcCCAUCUCUCCAU |
| SEQ ID NO: 172 | AcCCAUCUCUCCAUC |
| SEQ ID NO: 173 | cCCAUCUCUCCAUCC |
| SEQ ID NO: 174 | CCAUCUCUCCAUCCA |
| SEQ ID NO: 175 | CAUCUCUCCAUCCAU |
| SEQ ID NO: 176 | AUCUCUCCAUCCAUC |
| SEQ ID NO: 177 | AUCUCUCCAUCCAUG |
| SEQ ID NO: 178 | UCUCUCCAUCCAUCC |
| SEQ ID NO: 179 | UCUCUCCAUCCAUGC |
| SEQ ID NO: 180 | CUCUCCAUCCAUCCU |
| SEQ ID NO: 181 | CUCUCCAUCCAUGCU |
| SEQ ID NO: 182 | UCUCCAUCCAUCCUU |
| SEQ ID NO: 183 | UCUCCAUCCAUGCUU |
| SEQ ID NO: 184 | CUCCAUCCAUCCUUU |
| SEQ ID NO: 185 | CUCCAUCCAUGCUUU |
| SEQ ID NO: 186 | UCCAUCCAUCCUUUU |
| SEQ ID NO: 187 | UCCAUCCAUGCUUUU |
| SEQ ID NO: 188 | CCAUCCAUCCUUUUA |
| SEQ ID NO: 189 | CCAUCCAUGCUUUUA |
| SEQ ID NO: 190 | CAUCCAUCCUUUUAU |
| SEQ ID NO: 191 | CAUCCAUGCUUUUAU |
| SEQ ID NO: 192 | AUCCAUCCUUUUAUC |
| SEQ ID NO: 193 | AUCCAUGCUUUUAUC |
| SEQ ID NO: 194 | UCCAUCCUUUUAUCU |
| SEQ ID NO: 195 | UCCAUGCUUUUAUCU |
| SEQ ID NO: 196 | CCAUCCUUUUAUCUA |
| SEQ ID NO: 197 | CCAUGCUUUUAUCUA |
| SEQ ID NO: 198 | CAUCCUUUUAUCUAC |
| SEQ ID NO: 199 | CAUGCUUUUAUCUAC |
| SEQ ID NO: 200 | AUCCUUUUAUCUACU |
| SEQ ID NO: 201 | AUGCUUUUAUCUACU |
| SEQ ID NO: 202 | UCCUUUUAUCUACUC |
| SEQ ID NO: 203 | UGCUUUUAUCUACUC |
| SEQ ID NO: 204 | CCUUUUAUCUACUCA |
| SEQ ID NO: 205 | GCUUUUAUCUACUCA |
| SEQ ID NO: 206 | CUUUUAUCUACUCAU |
| SEQ ID NO: 207 | UUUUAUCUACUCAUC |
| SEQ ID NO: 208 | UUUAUCUACUCAUCA |
| SEQ ID NO: 209 | UUAUCUACUCAUCAC |
| SEQ ID NO: 210 | UAUCUACUCAUCACU |
| SEQ ID NO: 211 | AUCUACUCAUCACUC |
| SEQ ID NO: 212 | UCUACUCAUCACUCA |
| SEQ ID NO: 213 | CUACUCAUCACUCAU |
| SEQ ID NO: 214 | UACUCAUCACUCAUU |
| SEQ ID NO: 215 | ACUCAUCACUCAUUC |
| SEQ ID NO: 216 | CUCAUCACUCAUUCA |
| SEQ ID NO: 217 | UCAUCACUCAUUCAU |
| SEQ ID NO: 218 | CAUCACUCAUUCAUC |
| SEQ ID NO: 219 | AUCACUCAUUCAUCU |
| SEQ ID NO: 220 | UCACUCAUUCAUCUG |
| SEQ ID NO: 221 | CACUCAUUCAUCUGU |
| SEQ ID NO: 222 | ACUCAUUCAUCUGUU |
| SEQ ID NO: 223 | CUCAUUCAUCUGUUC |
| SEQ ID NO: 224 | UCAUUCAUCUGUUCA |
| SEQ ID NO: 225 | CAUUCAUCUGUUCAA |
| SEQ ID NO: 226 | AUUCAUCUGUUCAAU |
| SEQ ID NO: 227 | UUCAUCUGUUCAAUC |
| SEQ ID NO: 228 | UCAUCUGUUCAAUCA |
| SEQ ID NO: 229 | CAUCUGUUCAAUCAU |
| SEQ ID NO: 230 | AUCUGUUCAAUCAUU |
| SEQ ID NO: 231 | UCUGUUCAAUCAUUC |
| SEQ ID NO: 232 | CUGUUCAAUCAUUCA |
| SEQ ID NO: 233 | UGUUCAAUCAUUCAU |
| SEQ ID NO: 234 | GUUCAAUCAUUCAUU |
| SEQ ID NO: 235 | UUCAAUCAUUCAUUC |
| SEQ ID NO: 236 | UCAAUCAUUCAUUCA |
| SEQ ID NO: 237 | CAAUCAUUCAUUCAU |
| SEQ ID NO: 238 | AAUCAUUCAUUCAUU |
| SEQ ID NO: 239 | AUCAUUCAUUCAUUC |
| SEQ ID NO: 240 | UCAUUCAUUCAUUCA |
| SEQ ID NO: 241 | CAUUCAUUCAUUCAC |
| SEQ ID NO: 242 | AUUCAUUCAUUCACC |
| SEQ ID NO: 243 | UUCAUUCAUUCACCA |
| SEQ ID NO: 244 | UCAUUCAUUCACCAG |
| SEQ ID NO: 245 | CAUUCAUUCACCAGC |
| SEQ ID NO: 246 | AUUCAUUCACCAGCA |
| SEQ ID NO: 247 | UUCAUUCACCAGCAU |
| SEQ ID NO: 248 | UCAUUCACCAGCAUU |
| SEQ ID NO: 249 | CAUUCACCAGCAUUU |
| SEQ ID NO: 250 | AUUCACCAGCAUUUA |
| SEQ ID NO: 251 | UUCACCAGCAUUUAU |
| SEQ ID NO: 252 | UCACCAGCAUUUAUU |
| SEQ ID NO: 253 | CACCAGCAUUUAUUC |
| SEQ ID NO: 254 | ACCAGCAUUUAUUCA |
| SEQ ID NO: 255 | CCAGCAUUUAUUCAA |
| SEQ ID NO: 256 | CAGCAUUUAUUCAAC |
| SEQ ID NO: 257 | AGCAUUUAUUCAACA |
| SEQ ID NO: 258 | GCAUUUAUUCAACAA |
| SEQ ID NO: 259 | CAUUUAUUCAACAAA |
| SEQ ID NO: 260 | AUUUAUUCAACAAAC |

Example antisense sequences that target the INSR 3′ splice site/splice acceptor site

| | |
|---|---|
| SEQ ID NO: 261 | AUCCCGUAUCCGGUAc |
| SEQ ID NO: 262 | UCCCGUAUCCGGUAcU |
| SEQ ID NO: 263 | CCCGUAUCCGGUAcUA |
| SEQ ID NO: 264 | CCGUAUCCGGUAcUAU |
| SEQ ID NO: 265 | CGUAUCCGGUAcUAUA |
| SEQ ID NO: 266 | GUAUCCGGUAcUAUAU |
| SEQ ID NO: 267 | UAUCCGGUAcUAUAUG |
| SEQ ID NO: 268 | AUCCGGUAcUAUAUGG |
| SEQ ID NO: 269 | UCCGGUAcUAUAUGGC |
| SEQ ID NO: 270 | CCGGUAcUAUAUGGCA |
| SEQ ID NO: 271 | CGGUAcUAUAUGGCAC |
| SEQ ID NO: 272 | GGUAcUAUAUGGCACA |
| SEQ ID NO: 273 | AUAcUAUAUGGCACAG |
| SEQ ID NO: 274 | UAcUAUAUGGCACAGU |
| SEQ ID NO: 275 | AcUAUAUGGCACAGUA |
| SEQ ID NO: 276 | cUAUAUGGCACAGUAA |
| SEQ ID NO: 277 | UAUAUGGCACAGUAAG |

Example antisense sequences that target the INSR TDP-43 binding region and/or flanking regions thereof.

| | |
|---|---|
| SEQ ID NO: 278 | GUCUUCAGCAUCAGCC |
| SEQ ID NO: 279 | UCUUCAGCAUCAGCCU |
| SEQ ID NO: 280 | CUUCAGCAUCAGCCUG |
| SEQ ID NO: 281 | UUCAGCAUCAGCCUGA |
| SEQ ID NO: 282 | UCAGCAUCAGCCUGAU |
| SEQ ID NO: 283 | CAGCAUCAGCCUGAUA |
| SEQ ID NO: 284 | AGCAUCAGCCUGAUAC |
| SEQ ID NO: 285 | GCAUCAGCCUGAUACA |
| SEQ ID NO: 286 | CAUCAGCCUGAUACAU |
| SEQ ID NO: 287 | AUCAGCCUGAUACAUA |
| SEQ ID NO: 288 | UCAGCCUGAUACAUAU |
| SEQ ID NO: 289 | CAGCCUGAUACAUAUA |
| SEQ ID NO: 290 | AGCCUGAUACAUAUAA |
| SEQ ID NO: 291 | GCCUGAUACAUAUAAG |
| SEQ ID NO: 292 | CCUGAUACAUAUAAGA |
| SEQ ID NO: 293 | CUGAUACAUAUAAGAA |
| SEQ ID NO: 294 | UGAUACAUAUAAGAAG |
| SEQ ID NO: 295 | GAUACAUAUAAGAAGG |
| SEQ ID NO: 296 | AUACAUAUAAGAAGGU |
| SEQ ID NO: 297 | UACAUAUAAGAAGGUG |
| SEQ ID NO: 298 | ACAUAUAAGAAGGUG |
| SEQ ID NO: 299 | CAUAUAAGAAGGUGU |
| SEQ ID NO: 300 | ACAUAUAAGAAGGUGU |
| SEQ ID NO: 301 | CAUAUAAGAAGGUGUC |
| SEQ ID NO: 302 | AUAUAAGAAGGUGUCA |
| SEQ ID NO: 303 | UAUAAGAAGGUGUCAU |
| SEQ ID NO: 304 | AUAAGAAGGUGUCAUC |
| SEQ ID NO: 305 | UAAGAAGGUGUCAUCC |
| SEQ ID NO: 306 | AAGAAGGUGUCAUCCA |
| SEQ ID NO: 307 | AGAAGGUGUCAUCCAU |
| SEQ ID NO: 308 | GAAGGUGUCAUCCAUU |
| SEQ ID NO: 309 | AAGGUGUCAUCCAUUC |
| SEQ ID NO: 310 | AGGUGUCAUCCAUUCA |
| SEQ ID NO: 311 | GGUGUCAUCCAUUCAU |
| SEQ ID NO: 312 | GUGUCAUCCAUUCAUC |
| SEQ ID NO: 313 | UGUCAUCCAUUCAUCC |
| SEQ ID NO: 314 | GUCAUCCAUUCAUCCA |
| SEQ ID NO: 315 | UCAUCCAUUCAUCCAU |
| SEQ ID NO: 316 | CAUCCAUUCAUCCAUC |
| SEQ ID NO: 317 | AUCCAUUCAUCCAUCA |
| SEQ ID NO: 318 | UCCAUUCAUCCAUCAU |
| SEQ ID NO: 319 | CCAUUCAUCCAUCAUU |
| SEQ ID NO: 320 | CAUUCAUCCAUCAUUC |
| SEQ ID NO: 321 | AUUCAUCCAUCAUUCA |
| SEQ ID NO: 322 | UUCAUCCAUCAUUCAU |
| SEQ ID NO: 323 | UCAUCCAUCAUUCAUU |
| SEQ ID NO: 324 | CAUCCAUCAUUCAUUC |
| SEQ ID NO: 325 | AUCCAUCAUUCAUUCA |
| SEQ ID NO: 326 | UCCAUCAUUCAUUCAU |
| SEQ ID NO: 327 | CCAUCAUUCAUUCAUC |
| SEQ ID NO: 328 | CAUCAUUCAUUCAUCC |
| SEQ ID NO: 329 | AUCAUUCAUUCAUCCA |
| SEQ ID NO: 330 | UCAUUCAUUCAUCCAU |
| SEQ ID NO: 331 | CAUUCAUUCAUCCAUU |
| SEQ ID NO: 332 | AUUCAUUCAUCCAUUC |
| SEQ ID NO: 333 | UUCAUUCAUCCAUUCA |
| SEQ ID NO: 334 | UCAUUCAUCCAUUCAU |
| SEQ ID NO: 335 | CAUUCAUCCAUUCAUU |
| SEQ ID NO: 336 | AUUCAUCCAUUCAUUC |
| SEQ ID NO: 337 | UUCAUCCAUUCAUUCA |
| SEQ ID NO: 338 | UCAUCCAUUCAUUCAG |
| SEQ ID NO: 339 | CAUCCAUUCAUUCAGU |
| SEQ ID NO: 340 | AUCCAUUCAUUCAGUG |
| SEQ ID NO: 341 | UCCAUUCAUUCAGUGC |
| SEQ ID NO: 342 | CCAUUCAUUCAGUGCU |
| SEQ ID NO: 343 | CAUUCAUUCAGUGCUU |
| SEQ ID NO: 344 | AUUCAUUCAGUGCUUG |
| SEQ ID NO: 345 | UUCAUUCAGUGCUUGU |
| SEQ ID NO: 346 | UCAUUCAGUGCUUGUA |
| SEQ ID NO: 347 | CAUUCAGUGCUUGUAU |
| SEQ ID NO: 348 | AUUCAGUGCUUGUAUG |
| SEQ ID NO: 349 | UUCAGUGCUUGUAUGA |
| SEQ ID NO: 350 | UCAGUGCUUGUAUGAC |
| SEQ ID NO: 351 | CAGUGCUUGUAUGACU |
| SEQ ID NO: 352 | AGUGCUUGUAUGACUU |

### Example U7 smOPT Sequences

### Example 1: UNC13A bifunctional construct

An example U7 SmOPT bifunctional construct designed to target the TDP-43 regulated cryptic exon of UNC13A comprised the following U7 smOPT snRNA sequence:

SEQ ID NO: 358

AAUAUGAUAGGGACUUAGGGUGUUCAUCUGUUCAAUCAUUCAUUC
AAUUUUUGGAGCAGGUUUUCUGACUUCGGUCGGAAAACCCCU

The U7 SmOPT core expression cassette comprising the above snRNA sequence, was generated by gene synthesis cloned either in pUC-Simple (General Biosystems) or in a pMK vector followed by a fl origin and a CMV promoter driving a Blasticidin resistance cDNA followed by an SV40 polyadylation signal (GeneArt, Life technologies):

The complete U7 SmOPT Cassette for Example 1 is as follows (SEQ ID NO: 359):

GAAUUCCAACAUAGGAGCUGUGAUUGGCUGUUUUCAGCCAAUCAG
CACUGACUCAUUUGCAUAGCCUUUACAAGCGGUCACAAACUCAAG
AAACGAGCGGUUUUAAUAGUCUUUUAGAAUAUUGUUUAUCGAACC
GAAUAAGGAACUGUGCUUUGUGAUUCACAUAUCAGUGGAGGGGUG
UGGAAAUGGCACCUUGAUCUCACCCUCAUCGAAAGUGGAGUUGAU
GUCCUUCCCUGGCUCGCUACAGAGGCCUUUCCGC [symbol] AUAUGAUAGG
GACUUAGGGUGUUCAUCUGUUCAAUCAUUCAUUCAAUUUUUGGAG
CAGGUUUUCUGACUUCGGUCGGAAAACCCCUCCCAAGUUAACUGG
UCUACAAUGAAAGCAAAACAGUUCUCUUCCCCGCUCCCCGGUGUG
UGAGAGGGGCUUUGAUCCUUCUCUGGUUUCCUAGGAAACGCGUAA
GCUU

This includes the following components:

Mouse U7 promoter (this initiates transcription; only UsnRNA promoters can drive expression of U snRNAs but promoters with different sequences different to the example sequence below can be used-SEQ ID NO: 41

AACAUAGGAGCUGUGAUUGGCUGUUUUCAGCCAAUCAGCACUGAC
UCAUUUGCAUAGCCUUUACAAGCGGUCACAAACUCAAGAAACGAG
CGGUUUUAAUAGUCUUUUAGAAUAUUGUUUAUCGAACCGAAUAAG
GAACUGUGCUUUGUGAUUCACAUAUCAGUGGAGGGGUGUGGAAAU
GGCACCUUGAUCUCACCCUCAUCGAAAGUGGAGUUGAUGUCCUUC
CCUGGCUCGCUACAGAGGCCUUUCCGC

Transcription Start site.

A (shown highlighted above, immediately before the underlined section)

hnRNP A1 binding sequence
SEQ ID NO: 361
UAUGAUAGGGACUUAGGGUG
Antisense Sequence
SEQ ID NO: 420
UUCAUCUGUUCAAUCAUUCAUUC
Modified Sm sequence.
SEQ ID NO: 355
AAUUUUUGGAG
3′ Hairpin
SEQ ID NO: 356
CAGGUUUUCUGACUUCGGUCGGAAAACCCCU
3′box (for 3′end formation of the snRNA)
SEQ ID NO: 357
GUCUACAAUGAAAG

The antisense sequence and hnRNP binding sequence replace the 5′ end of the unmodified (i.e., endogenous or wildtype) U7 snRNA that contacts the histone downstream element of replication-dependent histone pre-mRNAs through complimentary base-pairing.

The antisense sequence enables binding of the construct to the TDP-43 regulated exon, while the presence of the binding domain for hnRNP A1 is designed to recruits endogenous hnRNP A1 in the cell, fulfilling the role of TDP-43, to repress splicing of the cryptic exon sequence to prevent its inclusion in the mature mRNA product of UNC13A.

Other alternative examples for bifunctional U7 smOPT constructs targeting the UNC13A cryptic exon are described below. The same expression cassette was described above but differed from Example 1 either by nature of the hnRNP tail/binding sequence and/or the antisense sequence.

Example 1B: This Construct Similarly Comprises a Different Antisense Sequence Designed to target the TDP-43 binding region for the UNC13A cryptic exon (shown bold), and the same hnRNP A1 binding sequence as for Example 1 (shown in italics)

| SEQ ID NO: | | Sequence |
|---|---|---|
| 360 | U7 SmOPT | AAUAUGAUAGGGACUUAGGGUGAUCUGUU |
| | snRNA | CAAUCAUUCAUUCAAUUUUUGGAGCAGGU |
| | sequence | UUUCUGACUUCGGUCGGAAAACCCCU |
| 362 | Antisense | AUCUGUUCAAUCAUUCAUUC |
| | sequence | |
| | targeting | |
| | TDP-43 | |
| | binding | |
| | region | |

Example 1C: This construct similarly comprises a different antisense sequence designed to target the TDP-43 binding domain for the UNC13A cryptic exon (shown bold), and the same hnRNP A1 binding sequence as for Example 1 (shown in italics)

| SEQ ID NO: | | Sequence |
|---|---|---|
| 363 | U7 SmOPT | AAUAUGAUAGGGACUUAGGGUGUUCAUCUGUU |
| | snRNA | CAAUCAUUCAAUUUUUGGAGCAGGUUUUCUGA |
| | sequence | CUUCGGUCGGAAAACCCCU |
| 364 | Antisense | UUCAUCUGUUCAAUCAUUC |
| | sequence | |
| | targeting | |
| | a TDP-43 | |
| | binding | |
| | region | |

Example 1D: This construct similarly comprises a different antisense sequence designed to target the TDP-43 binding domain for the UNC13A cryptic exon (shown bold), and the same hnRNP A1 binding sequence as for Example 1 (shown in italics)

| SEQ ID NO: | | Sequence |
|---|---|---|
| 365 | U7 SmOPT | AAUAUGAUAGGGACUUAGGGUGACUCAUUCAU |
| | snRNA | CUGUUCAAUCAAUUUUUGGAGCAGGUUUUCUG |
| | sequence | ACUUCGGUCGGAAAACCCCU |
| 366 | Antisense | ACUCAUUCAUCUGUUCAAUC |
| | sequence | |
| | targeting | |
| | a TDP-43 | |
| | binding | |
| | region | |

Example 1E: This construct similarly comprises a different antisense sequence designed to target the TDP-43 binding domain for the UNC13A cryptic exon (shown bold), and the same hnRNP A1 binding sequence as for Example 1 (shown in italics)

| SEQ ID NO: | | Sequence |
|---|---|---|
| 367 | U7 SmOPT | AAUAUGAUAGGGACUUAGGGUGACUCAUCACU |
| | snRNA | CAUUCAUCUGAAUUUUUGGAGCAGGUUUUCUG |
| | sequence | ACUUCGGUCGGAAAACCCCU |
| 368 | Antisense | ACUCAUCACUCAUUCAUCUG |
| | sequence | |
| | targeting | |
| | a TDP-43 | |
| | binding | |
| | region | |

Example 1F: This construct similarly comprises a different antisense sequence designed to target the TDP-43 binding domain for the UNC13A cryptic exon (shown bold), and the same hnRNP A1 binding sequence as for Example 1 (shown in italics)

| SEQ ID NO: | | Sequence |
|---|---|---|
| 369 | U7 SmOPT | AAUAUGAUAGGGACUUAGGGUGUCUACUCAU |
| | snRNA | CACUCAUUCAUCAAUUUUUGGAGCAGGUUUU |
| | sequence | CUGACUUCGGUCGGAAAACCCCU |
| 370 | Antisense | UCUACUCAUCACUCAUUCAUC |
| | sequence | |
| | targeting | |
| | a TDP-43 | |
| | binding | |
| | region | |

Example 1G: This construct similarly comprises a different antisense sequence designed to target the TDP-43 binding domain for the UNC13A cryptic exon (shown bold), and the same hnRNP A1 binding sequence as for Example 1 (shown in italics)

| SEQ ID NO: | | Sequence |
|---|---|---|
| 371 | U7 SmOPT | AAUAUGAUAGGGACUUAGGGUGUAUCUACU |
| | snRNA | CAUCACUCAUUCAUCAAUUUUUGGAGCAGG |
| | sequence | UUUUCUGACUUCGGUCGGAAAACCCCU |
| 372 | Antisense | UAUCUACUCAUCACUCAUUCAUC |
| | sequence | |
| | targeting | |
| | a TDP-43 | |
| | binding | |
| | region | |

Example 1H: This construct comprises a different antisense sequence designed to target an ESE within the UNC13A cryptic exon (shown bold), and the same hnRNP A1 binding sequence as for Example 1 (shown in italics)

| SEQ ID NO: | | Sequence |
|---|---|---|
| 373 | U7 SmOPT | AAUAUGAUAGGGACUUAGGGUGCCCAGGCAG |
| | snRNA | CUGGAAGAGACAAUUUUUGGAGCAGGUUUUC |
| | sequence | UGACUUCGGUCGGAAAACCCCU |
| 374 | Antisense | CCCAGGCAGCUGGAAGAGAC |
| | sequence | |
| | targeting | |
| | a TDP-43 | |
| | binding | |
| | region | |

Example 1I: This construct comprises the same antisense sequence as Example 1 to target TDP-43 binding domain for the UNC13A cryptic exon (shown in bold), but instead comprises a different example hnRNP H binding sequence.

| SEQ ID NO: | | Sequence |
|---|---|---|
| 375 | U7 SmOPT | AAUGUGGGGACCUUGUGGGGACCU |
| | snRNA | UCAUCUGUUCAAUCAUUCAUUCAA |
| | sequence | UUUUUGGAGCAGGUUUUCUGACUU |
| | | CGGUCGGAAAACCCCU |
| 376 | hnRNP H | UGUGGGGACCUUGUGGGGACC |
| | binding | |
| | sequence | |

Example 1L: This construct comprises the same antisense sequence as Example 1 to target TDP-43 binding domain for the UNC13A cryptic exon (shown in bold), but instead comprises a different example hnRNP C binding sequence (shown in italics)

| SEQ ID NO: | | Sequence |
|---|---|---|
| 377 | U7 SmOPT | AAGUAUUUUUGUGAUUUUUGUGU |
| | snRNA | UCAUCUGUUCAAUCAUUCAUUCA |
| | sequence | AUUUUUGGAGCAGGUUUUCUGAC |
| | | UUCGGUCGGAAAACCCCU |
| 378 | hnRNP C | GUAUUUUUGUGAUUUUUGUG |
| | binding | |
| | sequence | |

Example 1M: This construct comprises the same antisense sequence as Example 1 to target TDP-43 binding domain for the UNC13A cryptic exon (shown in bold), but instead comprises a different example hnRNP L binding sequence (shown in italics)

| SEQ ID NO: | | |
|---|---|---|
| 379 | U7 SmOPT snRNA | |
| | sequence | |
| 380 | hnRNP L binding | UUCACACAUUAGAACACAACA |
| | sequence | |

Example 1N: This construct comprises a different antisense sequence to target a 3′ splice site for UNC13A (shown in bold) but comprises the same hnRNP A1 binding sequence as Example 1.

| SEQ ID NO: | | Sequence |
|---|---|---|
| 381 | U7 SmOPT | AAUAUGAUAGGGACUUAGGGUGUC |
| | snRNA | CUGAGUGGUUAGGGCUGGAAUAGA |
| | sequence | AUUUUUGGAGCAGGUUUUCUGACU |
| | | UCGGUCGGAAAACCCCU |
| 382 | Antisense | UCCUGAGUGGUUAGGGCUGGAAUA |
| | sequence | G |
| | targeting | |
| | a 3′ splice | |
| | site | |

Example 10: This construct comprises a different antisense sequence to target a 5′ splice site for UNC13A (shown in bold) but comprises the same hnRNP A1 binding sequence as Example 1. This sequence also overlaps with and targets the TDP-43 binding sequence.

| SEQ ID NO: | | Sequence |
|---|---|---|
| 383 | U7 SmOPT | AAUAUGAUAGGGACUUAGGGUGUC |
| | snRNA | CAUGUACUCACCCAUCUCAAUUUU |
| | sequence | UGGAGCAGGUUUUCUGACUUCGGU |
| | | CGGAAAACCCCU |
| 384 | Antisense | UCCAUGUACUCACCCAUCUC |
| | sequence | |
| | targeting | |
| | a 5′ splice | |
| | site | |

Example 1P: This construct comprises the same antisense sequence as Example 1 to target TDP-43 binding domain for the UNC13A cryptic exon (shown in bold) but comprises a different example hnRNP H binding sequence (shown in italics).

| SEQ ID NO: | | Sequence |
|---|---|---|
| 385 | U7 SmOPT | AAUGUGGGGACCAGGGGGAUUC |
| | snRNA | AUCUGUUCAAUCAUUCAUUCAA |
| | sequence | UUUUUGGAGCAGGUUUUCUGAC |
| | | UUCGGUCGGAAAACCCCU |
| 386 | hnRNP H | UGUGGGGACCAGGGGGA |
| | binding | |
| | site | |

Example 1Q: This construct comprises the same antisense sequence to target TDP-43 binding domain for the UNC13A cryptic exon (shown in bold) but comprises a different example hnRNP A1 binding sequence (shown in italics).

| SEQ ID NO: | | Sequence |
|---|---|---|
| 387 | U7 SmOPT | AAUAUGUUAGGGACUUAGGGAGUU |
| | snRNA | CAUCUGUUCAAUCAUUCAUUCAAU |
| | sequence | UUUUGGAGCAGGUUUUCUGACUUC |
| | | GGUCGGAAAACCCCU |
| 389 | hnRNP A1 | UAUGUUAGGGACUUAGGGAG |
| | binding | |
| | site | |

Example 1R: This comparative example construct comprises an antisense sequence designed to target the 5′ splice site and TDP-43 binding sequence of the UNC13A cryptic exon, but wherein the construct does not contain a hnRNP A1 binding sequence. The antisense sequence is shown in bold. This contains the same antisense sequence as Example 10.

| SEQ ID NO: | | Sequence |
|---|---|---|
| 472 | U7 SmOPT | AAUCCAUGUACUCACCCAUCU |
| | snRNA | CAAUUUUUGGAGCAGGUUUUC |
| | sequence | UGACUUCGGUCGGAAAACCCC |
| | | U |

### Example 2: STMN2 Bifunctional Construct

An example U7 SmOPT bifunctional construct designed to target the TDP-43 regulated cryptic exon of STMN2 (corresponding to exon 2a) comprised the following U7 smOPT snRNA sequence:

This contained the antisense sequence SEQ ID NO: 391 AUGCUCACACAGAGAGCCAAAUUC (shown above underlined) designed to target the TDP-43 binding domain for the UNC13A cryptic exon.

The construct also contained an example hnRNP A1 binding sequence (SEQ ID NO: 361, shown above in italics).

Other alternative examples for bifunctional U7 smOPT constructs targeting the STMN2 2a cryptic exon are described below.

Example 2B: This construct comprises a different antisense sequence designed to target the TDP-43 binding domain for the STMN2 cryptic exon (shown bold), and a hnRNP A1 binding sequence (shown in italics)

| SEQ ID NO: | | Sequence |
|---|---|---|
| 392 | U7 SmOPT | AAUAUGAUAGGGACUUAGGGUGGAG |
| | snRNA | AGCCAAAUUCUUAUUUUCAAUUUUU |
| | sequence | GGAGCAGGUUUUCUGACUUCGGUCG |
| | | GAAAACCCCU |
| 393 | Antisense | GAGAGCCAAAUUCUUAUUUUC |
| | sequence | |
| | targeting | |
| | a TDP-43 | |
| | binding | |
| | region | |

Example 2C: This construct comprises a different antisense sequence designed to target the TDP-43 binding domain for the STMN2 cryptic exon (shown bold), and a hnRNP A1 binding sequence (shown in italics)

| SEQ ID NO: | | Sequence |
|---|---|---|
| 394 | U7 SmOPT | AAUAUGAUAGGGACUUAGGGUGCA |
| | snRNA | CAGAGAGCCAAAUUCUUAAAUUUU |
| | sequence | UGGAGCAGGUUUUCUGACUUCGGU |
| | | CGGAAAACCCCU |
| 395 | Antisense | CACAGAGAGCCAAAUUCUUA |
| | sequence | |
| | targeting | |
| | a TDP-43 | |
| | binding | |
| | region | |

Example 2D: This construct comprises a different antisense sequence designed to target the TDP-43 binding domain for the STMN2 cryptic exon (shown bold), and a hnRNP A1 binding sequence (shown in italics)

| SEQ ID NO: | | Sequence |
|---|---|---|
| 396 | U7 SmOPT | AAUAUGAUAGGGACUUAGGGUGUG |
| | snRNA | CUCACACAGAGAGCCAAAUAAUUU |
| | sequence | UUGGAGCAGGUUUUCUGACUUCGG |
| | | UCGGAAAACCCCU |
| 397 | Antisense | UGCUCACACAGAGAGCCAAAU |
| | sequence | |
| | targeting | |
| | a TDP-43 | |
| | binding | |
| | region | |

Example 2E: This construct comprises a different antisense sequence designed to target the TDP-43 binding domain for the STMN2 cryptic exon (shown bold), and a hnRNP A1 binding sequence (shown in italics)

| SEQ ID NO: | | Sequence |
|---|---|---|
| 398 | U7 SmOPT | AAUAUGAUAGGGACUUAGGGUGCA |
| | snRNA | CAUGCUCACACAGAGAGCAAUUUU |
| | sequence | UGGAGCAGGUUUUCUGACUUCGGU |
| | | CGGAAAACCCCU |
| 399 | Antisense | CACAUGCUCACACAGAGAGC |
| | sequence | |
| | targeting | |
| | a TDP-43 | |
| | binding | |
| | region | |

Example 2F: This construct comprises a different antisense sequence designed to target the TDP-43 binding domain for the STMN2 cryptic exon (shown bold), and a hnRNP A1 binding sequence (shown in italics)

| SEQ ID NO: | | Sequence |
|---|---|---|
| 400 | U7 SmOPT | AAUAUGAUAGGGACUUAGGGUGACG |
| | snRNA | CACACAUGCUCACACAGAAUUUUUG |
| | sequence | GAGCAGGUUUUCUGACUUCGGUCGG |
| | | AAAACCCCU |
| 401 | Antisense | ACGCACACAUGCUCACACAG |
| | sequence | |
| | targeting | |
| | a TDP-43 | |
| | binding | |
| | region | |

Example 2G: This construct comprises a different antisense sequence designed to target the TDP-43 binding domain for the STMN2 cryptic exon (shown bold), and a hnRNP A1 binding sequence (shown in italics)

| SEQ ID NO: | | |
|---|---|---|
| 402 | U7 SmOPT | AAUAUGAUAGGGACUUAGGGUGCAC |
| | snRNA | ACACGCACACAUGCUCAAAUUUUUG |
| | sequence | GAGCAGGUUUUCUGACUUCGGUCGG |
| | | AAAACCCCU |
| 403 | Antisense | CACACACGCACACAUGCUCA |
| | sequence | |
| | targeting | |
| | a TDP-43 | |
| | binding | |
| | region | |

Example 2H: This construct comprises a different antisense sequence designed to target an ESE within the STMN2 cryptic exon (shown bold), and a hnRNP A1 binding sequence (shown in italics)

| SEQ ID NO: | | Sequence |
|---|---|---|
| 404 | U7 SmOPT | AAUAUGAUAGGGACUUAGGGUGCUG |
| | snRNA | UGCCACAAGCCGCAUUCAAUUUUUG |
| | sequence | GAGCAGGUUUUCUGACUUCGGUCGG |
| | | AAAACCCCU |
| 405 | Antisense | CUGUGCCACAAGCCGCAUUC |
| | sequence | |
| | targeting | |
| | a TDP-43 | |
| | binding | |
| | region | |

Example 21: This construct comprises a different antisense sequence designed to target the 3′ splice site of the STMN2 cryptic exon (shown bold), and a hnRNP A1 binding sequence (shown in italics)

| SEQ ID NO: | | Sequence |
|---|---|---|
| 406 | U7 SmOPT | AAUAUGAUAGGGACUUAGGGUG |
| | snRNA | CGAAGGUCUUCUGCCGAGUCCU |
| | sequence | GCAAUUUUUGGAGCAGGUUUUC |
| | | UGACUUCGGUCGGAAAACCCCU |
| 407 | Antisense | CGAAGGUCUUCUGCCGAGUCCU |
| | sequence | GC |
| | targeting | |
| | a 3′ splice | |
| | site | |

Example 2J: This comparative example construct comprises an antisense sequence designed to target the 3′ splice site of the STMN2 cryptic exon, but wherein the construct does not contain a hnRNP A1 binding sequence. The antisense sequence is shown in bold.

| SEQ ID NO: | | Sequence |
|---|---|---|
| 473 | U7 SmOPT | AACGAAGGUCUUCUGCCGAGUCCUGC |
| | snRNA | AAUUUUUGGAGCAGGUUUUCUGACUU |
| | sequence | CGGUCGGAAAACCCCU |

### Example 3: INSR Bifunctional Construct

The following U7 smOPT snRNA sequences were also designed to target the INSR TDP-43 regulated cryptic exon. Each sequence comprises an antisense sequence directed to the TDP-43 binding region or flanking region thereof (shown in bold), and a binding sequence for hnRNP A1 (SEQ ID NO: 361, shown in italics).

Example 3A

| SEQ ID NO: | | Sequence |
|---|---|---|
| 408 | U7 SmOPT | AAUAUGAUAGGGACUUAGGGUG |
| | snRNA | UCCGGUACUAUAUGGCACAGAA |
| | sequence | UUUUUGGAGCAGGUUUUCUGAC |
| | | UUCGGUCGGAAAACCCCU |
| 409 | Antisense | UCCGGUACUAUAUGGCACAG |
| | sequence | |
| | targeting | |
| | a TDP-43 | |
| | binding | |
| | region | |

Example 3B:

| SEQ ID NO: | | Sequence |
|---|---|---|
| 410 | U7 SmOPT | AUAUGAUAGGGACUUAG |
| | snRNA | GGUGCCCGUAUCCGGUA |
| | sequence | CUAUAUGAAUUUUUGGA |
| | | GCAGGUUUUCUGACUUC |
| | | GGUCGGAAAACCCCU |
| 411 | Antisense sequence | CCCGUAUCCGGUACUAU |
| | targeting a TDP-43 | AUG |
| | binding region | |

Example 3C:

| SEQ ID NO: | | Sequence |
|---|---|---|
| 412 | U7 SmOPT | AAUAUGAUAGGGACUUA |
| | snRNA | GGGUGCUUCAGCAUCAG |
| | sequence | CCUGAUACAAUUUUUGG |
| | | AGCAGGUUUUCUGACUU |
| | | CGGUCGGAAAACCCCU |
| 413 | Antisense | CUUCAGCAUCAGCCUGA |
| | sequence | UAC |
| | targeting | |
| | a TDP-43 | |
| | binding | |
| | region | |

Example 3D:

| SEQ ID NO: | | Sequence |
|---|---|---|
| 414 | U7 SmOPT | AAUAUGAUAGGGACUUAG |
| | snRNA | GGUGCCAUUCAUUCAGUG |
| | sequence | CUUGUAUGACAAUUUUUG |
| | | GAGCAGGUUUUCUGACUU |
| | | CGGUCGGAAAACCCCU |
| 415 | Antisense | CCAUUCAUUCAGUGCUUG |
| | sequence | UAUGAC |
| | targeting | |
| | a TDP-43 | |
| | binding | |
| | region | |

Example 3E:

| SEQ ID NO: | | Sequence |
|---|---|---|
| 416 | U7 SmOPT | AAUAUGAUAGGGACUUAGG |
| | snRNA | GUGGUGCUUGUAUGACUUC |
| | sequence | AUCCCGAAUUUUUGGAGCA |
| | | GGUUUUCUGACUUCGGUCG |
| | | GAAAACCCCU |
| 417 | Antisense | GUGCUUGUAUGACUUCAUC |
| | sequence | CCG |
| | targeting | |
| | a TDP-43 | |
| | binding | |
| | region | |

Example 3F:

| SEQ ID NO: | | Sequence |
|---|---|---|
| 418 | U7 SmOPT | AAUAUGAUAGGGACUUAGG |
| | snRNA | GUGGAAGGUGUCAUCCAUU |
| | sequence | CAUCCAAUUUUUGGAGCAG |
| | | GUUUUCUGACUUCGGUCGG |
| | | AAAACCCCU |
| 419 | Antisense | GAAGGUGUCAUCCAUUCAU |
| | sequence | CC |
| | targeting | |
| | a TDP-43 | |
| | binding | |
| | region | |

### Combined U7 Vector Construct Example

A combined U7 vector construct was designed with contains a U7 construct cassette corresponding to Example 10 which targets UNC13A, a U7 construct cassette corresponding to Example 2C which targets STMN2 and a U7 construct cassette corresponding to Example 3C which targets INSR, spaced by stuffer sequences (shown below in bold).

| SEQ | | |
|---|---|---|
| ID | | |
| NO: | | Sequence |
| 474 | Combined U7 | GAATTCCAACATAGGAGCTGTGATTG |
| | Construct | GCTGTTTTCAGCCAATCAGCACTGAC |
| | Vector | TCATTTGCATAGCCTTTACAAGCGGT |
| | | CACAAACTCAAGAAACGAGCGGTTTT |
| | | AATAGTCTTTTAGAATATTGTTTATC |
| | | GAACCGAATAAGGAACTGTGCTTTGT |
| | | GATTCACATATCAGTGGAGGGGTGTG |
| | | GAAATGGCACCTTGATCTCACCCTCA |
| | | TCGAAAGTGGAGTTGATGTCCTTCCC |
| | | TGGCTCGCTACAGAGGCCTTTCCGCA |
| | | ATATGATAGGGACTTAGGGTGTCCAT |
| | | GTACTCACCCATCTCAATTTTTGGAG |
| | | CAGGTTTTCTGACTTCGGTCGGAAAA |
| | | CCCCTCCCAAGTTAACTGGTCTACAA |
| | | TGAAAGCAAAACAGTTCTCTTCCCCG |
| | | CTCCCCGGTGTGTGAGAGGGGCTTTG |
| | | ATCCTTCTCTGGTTTCCTAGGAAACG |
| | | CGTACGCGCCCTGTAGCGGCGCATTA |
| | | AGCGCGGCGGGTGTGGTGGTTACGCG |
| | | CAGCGTGACCGCTACACTTGCCAGCG |
| | | CCCTAGCGCCCGCTCCTTTCGCTTTC |
| | | TTCCCTTCCTTTCTCGCCACGTTCGC |
| | | CGGCTTTCCCCGTCAAGCTCTAAATC |
| | | GGGGGCTCCCTTTAGGGTTCCGATTT |
| | | AGTGCTTTACGGCACCTCGACCCCAA |
| | | AAAACTTGATTAGGGTGATGGTTCAC |
| | | GTAGTGGGCCATCGCCCTGTCGACGA |
| | | ATTCCAACATAGGAGCTGTGATTGGC |
| | | TGTTTTCAGCCAATCAGCACTGACTC |
| | | ATTTGCATAGCCTTTACAAGCGGTCA |
| | | CAAACTCAAGAAACGAGCGGTTTTAA |
| | | TAGTCTTTTAGAATATTGTTTATCGA |
| | | ACCGAATAAGGAACTGTGCTTTGTGA |
| | | TTCACATATCAGTGGAGGGGTGTGGA |
| | | AATGGCACCTTGATCTCACCCTCATC |
| | | GAAAGTGGAGTTGATGTCCTTCCCTG |
| | | GCTCGCTACAGAGGCCTTTCCGCAAT |
| | | ATGATAGGGACTTAGGGTGCACAGAG |
| | | AGCCAAATTCTTAAATTTTTGGAGCA |
| | | GGTTTTCTGACTTCGGTCGGAAAACC |
| | | CCTCCCAAGTTAACTGGTCTACAATG |
| | | AAAGCAAAACAGTTCTCTTCCCCGCT |
| | | CCCCGGTGTGTGAGAGGGGCTTTGAT |
| | | CCTTCTCTGGTTTCCTAGGAAGCTTT |
| | | ACAATCAACAGCATCCCCATCTCTGA |
| | | AGACTACAGCGTCGCCAGCGCAGCTC |
| | | TCTCTAGCGACGGCCGCATCTTCACT |
| | | GGTGTCAATGTATATCATTTTACTGG |
| | | GGGACCTTGTGCAGAACTCGTGGTGC |
| | | TGGGCACTGCTGCTGCTGCGGCAGCT |
| | | GGCAACCTGACTTGTATCGTCGCGAT |
| | | CGGAAATGAGAACAGGGGCATCTTGA |
| | | GCCCCTGCGGACGGTGCCGACAGGTG |
| | | CTTCTCGATCTGCAGGATCCGAATTC |
| | | CAACATAGGAGCTGTGATTGGCTGTT |
| | | TTCAGCCAATCAGCACTGACTCATTT |
| | | GCATAGCCTTTACAAGCGGTCACAAA |
| | | CTCAAGAAACGAGCGGTTTTAATAGT |
| | | CTTTTAGAATATTGTTTATCGAACCG |
| | | AATAAGGAACTGTGCTTTGTGATTCA |
| | | CATATCAGTGGAGGGGTGTGGAAATG |
| | | GCACCTTGATCTCACCCTCATCGAAA |
| | | GTGGAGTTGATGTCCTTCCCTGGCTC |
| | | GCTACAGAGGCCTTTCCGCAATATGA |
| | | TAGGGACTTAGGGTGCCCGTATCCGG |
| | | TACTATATGAATTTTTGGAGCAGGTT |
| | | TTCTGACTTCGGTCGGAAAACCCCTC |
| | | CCAAGTTAACTGGTCTACAATGAAAG |
| | | CAAAACAGTTCTCTTCCCCGCTCCCC |
| | | GGTGTGTGAGAGGGGCTTTGATCCTT |
| | | CTCTGGTTTCCTAGGAAGATCTATGG |
| | | CAGCGGCCGCATGATATCTAGACTGG |
| | | CCTGGGCCTCATGGGCCTTCCGCTC |

### Results and Discussion

“Bifunctional” U7 smOPT targeting the UNC13A cryptic exon.

An example construct of the invention (corresponding to Example 1) was found to almost perfectly rescue of UNC13A splicing in electroporated SH-SY5Y cells with TDP-43 knockdown. As described above, the example construct comprised an anti-sense sequence that targeted the UNC13A cryptic exon within a TDP-43 binding region, (i.e., as determined by iCLIP data) upstream of the UNC13A 5′ donor splice site, while additionally comprising a high-affinity binding site for the splicing repressor hnRNP A1.

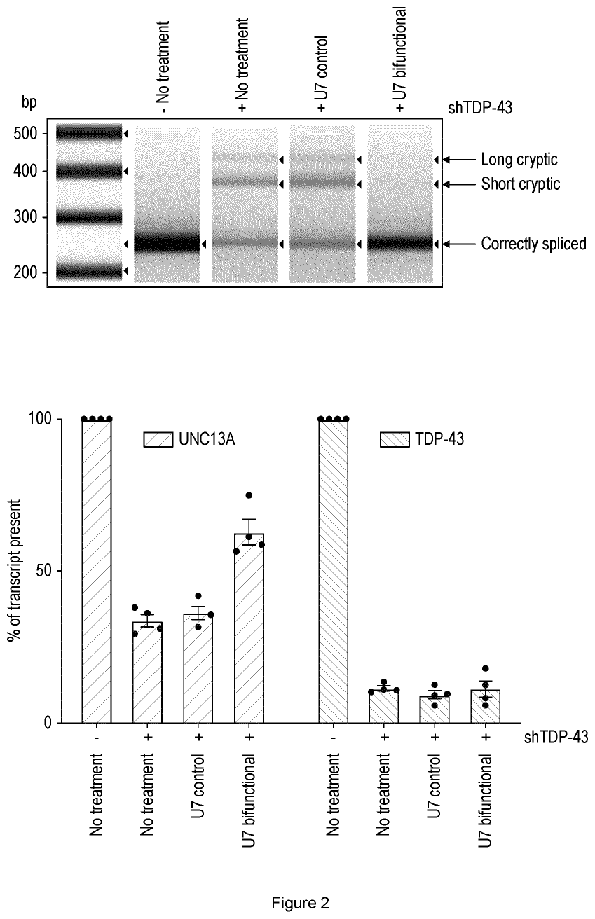

More specifically, SH-SY5Y cells with doxycycline-inducible TDP-43 knockdown were either electroporated with a U7 SmOPT control plasmid, or the UNC13A bi-functional U7 SmOPT construct, in the presence of TDP-43 shRNA. TDP-43 knockdown resulted in the appearance of UNC13A cryptic splicing. This was almost entirely rescued by expression of the bifunctional U7 SmOPT construct. FIG. 2 (top) shows almost complete disappearance of bands corresponding to cryptic splicing and the emergence of a stronger band corresponding to the correctly spliced mature mRNA product.

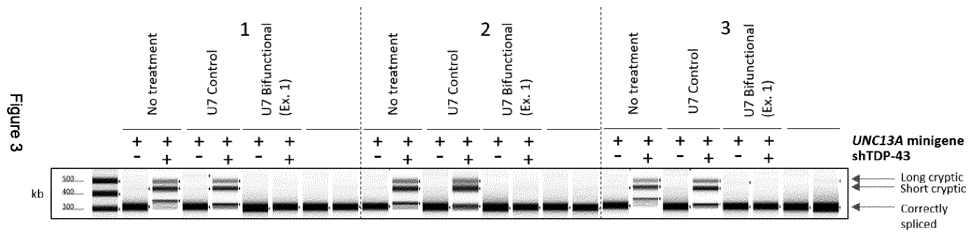

FIG. 3 instead shows the rescue of splicing in TDP-43 knockdown SK-N-DZ cells transfected with a UNC13A minigene and the Example 1 construct of the invention. This is demonstrated using RT-PCR. Again, FIG. 3 shows almost complete disappearance of bands corresponding to cryptic splicing and the emergence of a stronger band corresponding to the correctly spliced mature mRNA product for cells treated with the bifunctional U7 construct of the invention.

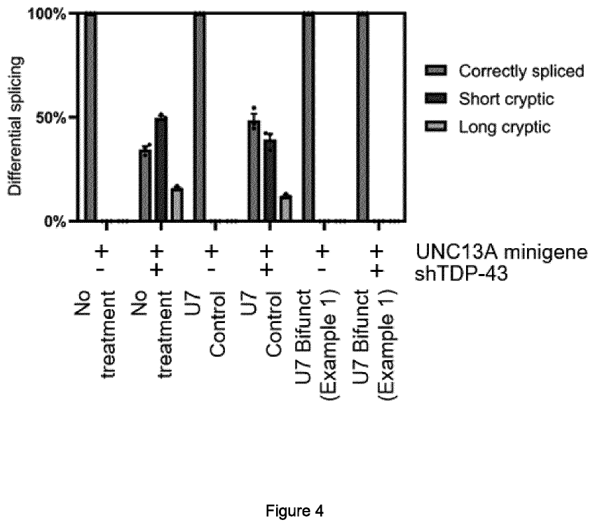

FIG. 4 shows the quantification of the correctly spliced mature RNA (far left bar), mature RNA comprising the short UNC13A cryptic exon (middle bar) and mature RNA comprising the long UNC13A cryptic exon (far right bar) in TDP-43 knockdown SK-N-DZ cells. This demonstrates that all or almost all of the mature mRNA product is correctly spliced in TDP-43 depleted cells treated with the construct of the invention.

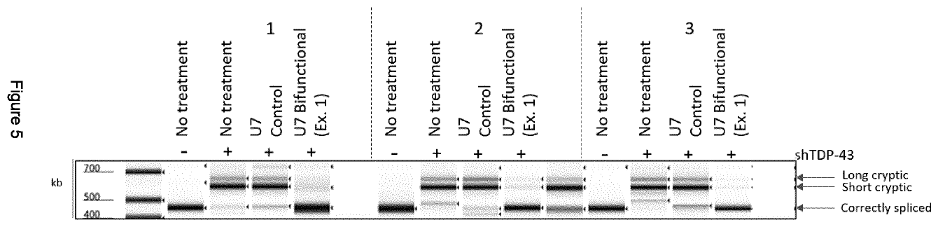

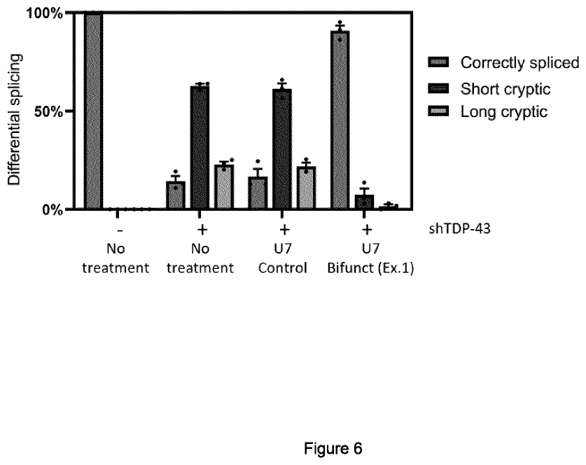

FIG. 5 shows the rescue of splicing by RT-PCR of SH-SY5Y cells with TDP-43 knockdown with mature RNA derived from endogenous UNC13A and electroporated with the Example 1 construct of the invention. FIG. 6 shows the % differential splicing of the correctly spliced mature RNA (far left bar), mature RNA comprising the short UNC13A cryptic exon (middle bar) and mature RNA comprising the long UNC13A cryptic exon (far right bar) in these cells. This demonstrates that the majority of the mature mRNA product is correctly spliced in TDP-43 depleted cells treated with the construct of the invention.

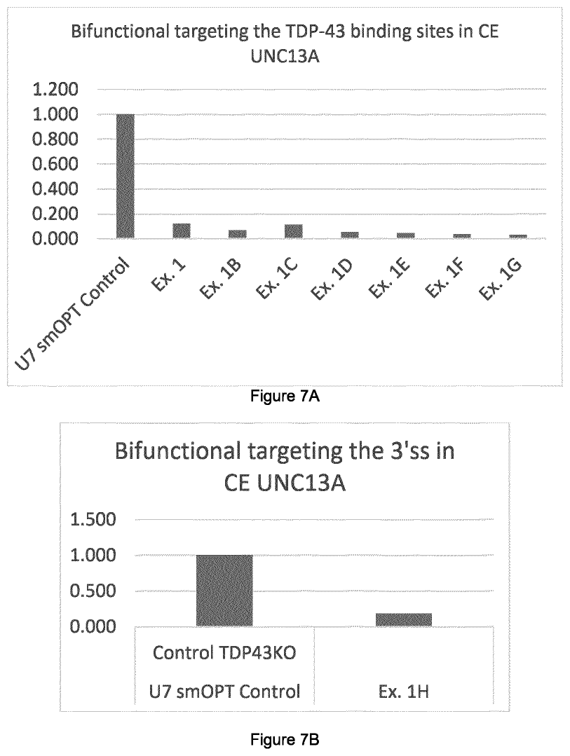

Further examples (Examples 1B-1G), along with Example 1, having different antisense sequences that targeted the TDP-43 binding region were next tested to see if they could also rescue UNC13A cryptic exon splicing. This experiment was performed by looking at splicing of the UNC13A minigene in 293T cells with TDP-43 inducible knockdown. FIG. 7A demonstrates that all of the tested constructs rescued splicing, as calculated by taking the ratios of cryptic exon containing to correctly spliced RNAs relative to control treated TDP-43 knockdown normalized to GAPDH mRNA in 293T cells. This demonstrates that the rescuing effect is not restricted to the targeting of certain sequences, although certain sequences were found to be more effective at correcting splicing than others. Interestingly, efficiency of cryptic exon repression appears to correlate with proximity to the TDP-43 motifs with Ex 1G being the most efficient in repressing cryptic exon inclusion.

The efficacy of bifunctional constructs targeting a different splicing element was also tested. Example 1H, which instead targets a different portion of the UNC13A cryptic exon and flanking region thereof, more particularly at a 3′ splice site. This construct was also shown to effectively rescue splicing (see FIG. 7B).

Bifunctional U7 smOPT targeting the STMN2 2a cryptic exon.

Treatment of TDP-43 depleted SH-SY5Y cells (i.e., treated with TDP-43 shRNA) with an example U7 smOPT bifunctional construct of the invention corresponding to Example 2, was also found to lead to partial rescue of correct splicing of the STMN2 cryptic exon. This suggests that constructs of the present invention and methods described herein may be used to target different TDP-43 regulated cryptic exons.

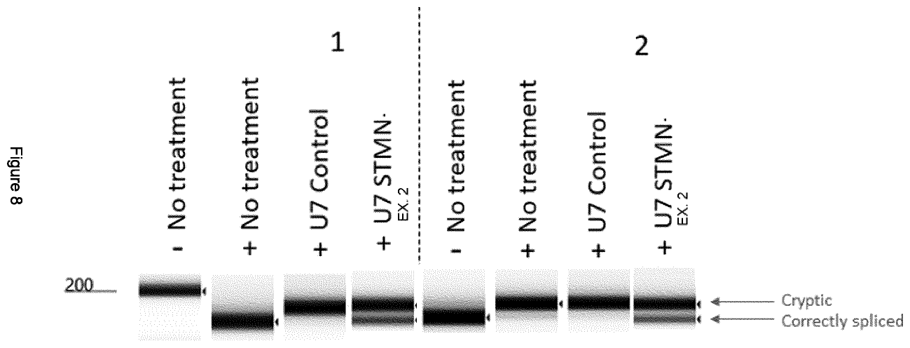

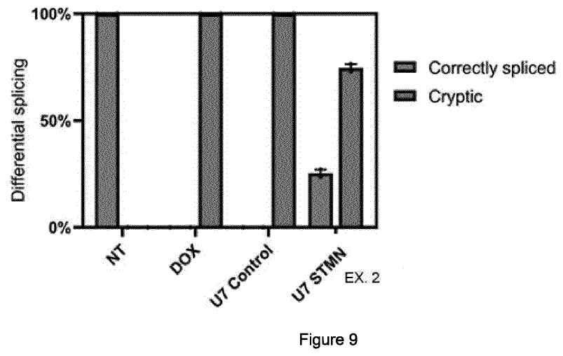

The rescue of splicing in STMN2 is demonstrated in FIG. 8, where a band corresponding to the correctly spliced mature mRNA STMN2 product is observed in cells treated with the U7 smOPT construct of the invention, but not for the U7 control. FIG. 9 shows the differential splicing of the correctly spliced mature RNA (left bar) compared with mature RNA containing the STMN2 cryptic exon as compared with no treatment, Dox or U7 control. TDP-43 knock-down completely eliminates correctly spliced and therefore functional STMN2, basically generating a full KO. Rescue of correct splicing to over 20% represents a strong improvement with likely strong functional benefits.

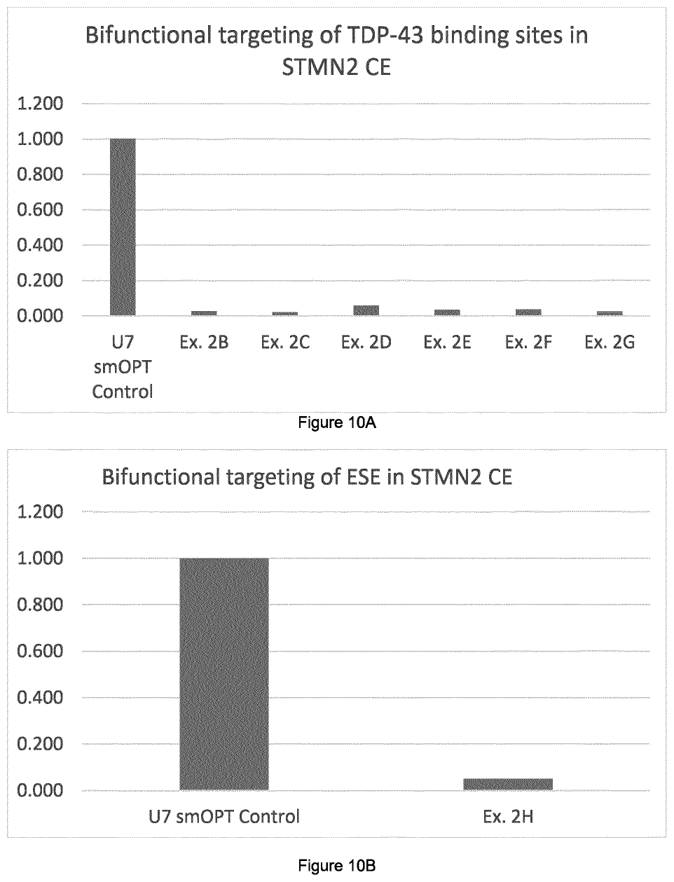

Further examples (Examples 2B-2G) having different antisense sequences that targeted the TDP-43 binding site were tested, along with Example 2, to see if they could rescue STMN2 2a cryptic exon splicing. The experiment was performed by looking at splicing of the STMN2 minigene in 293T cells with TDP-43 inducible knockdown. FIG. 10A demonstrates that all of the tested constructs rescued splicing, as calculated by taking the ratios of cryptic exon containing to correctly spliced RNAs relative to control treated TDP-43 knockdown normalized to GAPDH.

The efficacy of bifunctional constructs targeting a different antisense site to the TDP-43 binding site was also tested using the same set-up. Example 2H, which instead targets a different portion of the STMN2 cryptic exon and flanking region thereof, more particularly at an ESE site (as identified using ESE finder 3.0). This was also shown to effectively rescue splicing (see FIG. 10B).

Bifunctional U7 smOPT Targeting the INSR Cryptic Exon.

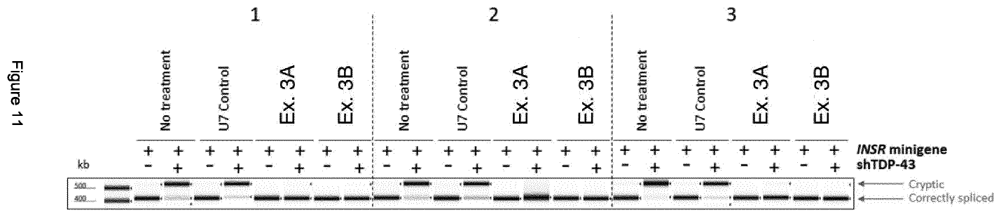

Next, it was also demonstrated that U7 smOPT constructs could also be used to rescue splicing of a third TDP-43 regulated cryptic exon corresponding to the TDP-43 regulated cryptic exon in the INSR gene. FIG. 11 shows the RT-PCR in SK-N-DZ cells with TDP-43 knockdown and transfected with a INSR minigene using Example constructs of the invention. As compared with the control, example constructs almost eliminated incorrect “cryptic” splicing, as demonstrated by the stronger band corresponding to the correctly spliced mature mRNA product.

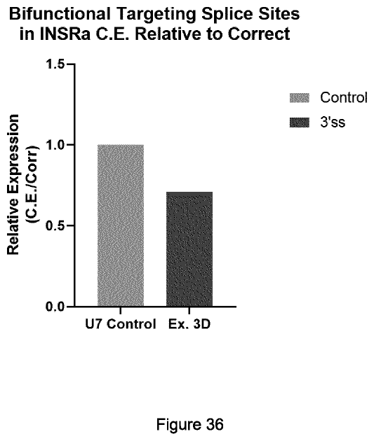

FIG. 36 further shows the ratio of cryptic exon included to total RT-qPCR levels of INSRa in cells treated with Example 3D which targets the 3′ splice site. Data is shown relative to ratio in non-targeting control (U7 Control) transfected 293T-2xTDP-shRNA cells containing INSRa minigene and under TDP-43 knockdown normalized to GAPDH mRNA.

Testing of U7 smOPT constructs having different hnRNP sequences for different hnRNP proteins

Preliminary studies were next conducted to determine whether the constructs could be used to recruit any suitable hnRNP protein and/or whether the recruitment of a specific hnRNP protein is important. Analogous bifunctional constructs to Example 1 were tested comprising different sequences for hnRNP binding proteins (i.e., according to Examples 1L-1M), hnRNP H, hnRNP C and hnRNP L. The experiment was performed by looking at splicing of the UNC13A minigene in 293T cells with TDP-43 inducible knockdown.

Constructs comprising a binding sequence for hnRNP A1 (i.e., Example 1) were found to be the most effective at rescuing splicing. This may reflect the higher levels of endogenous hnRNP A1 in the cell. hnRNP H (Example 1L) was also found to be efficient at rescuing splicing but was not as effective as hnRNP A1. While constructs with binding sequences for hnRNP C and hnRNP L showed partial rescue of splicing and were improved as compared with the U7 smOPT control. However, these hnRNP proteins were less efficient as compared with hnRNP A1 and hnRNP H. This may reflect the lower levels of hnRNP L as well as the requirement for hnRNP C to form tetramers and bind to both sides of an exon to induce exon skipping.

Comparison with U7 smOPT Constructs without a hnRNP Binding Tail.

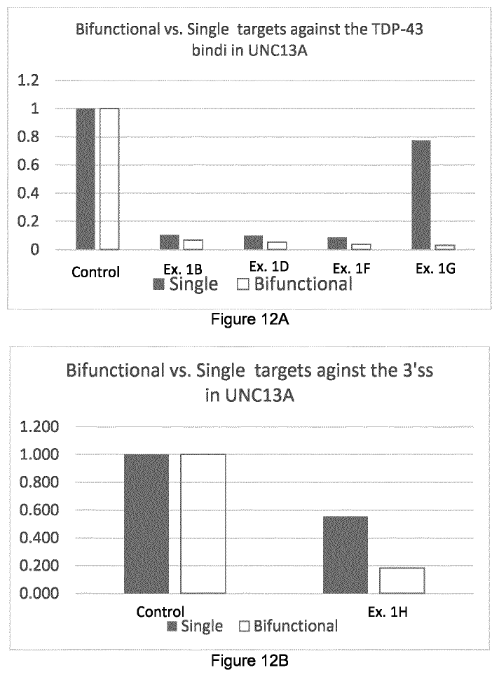

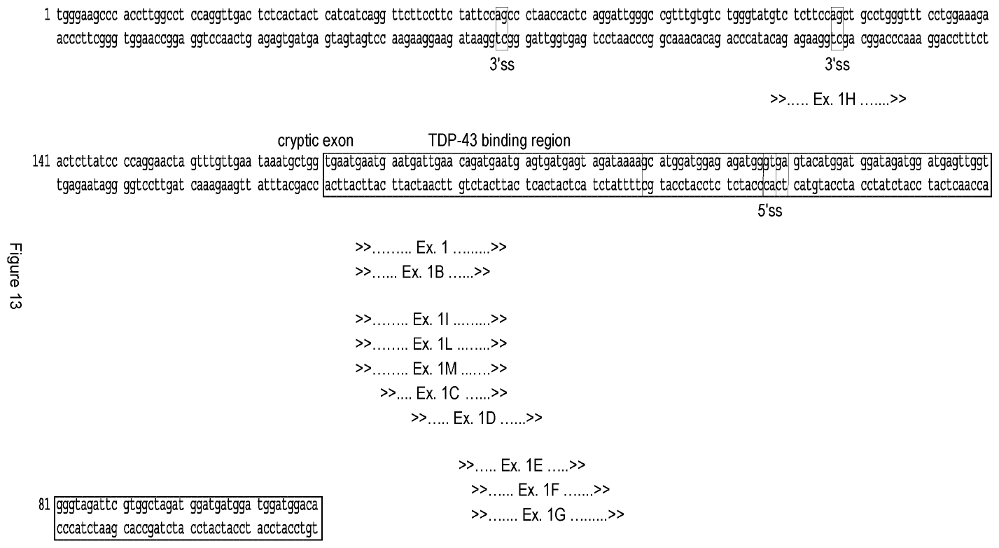

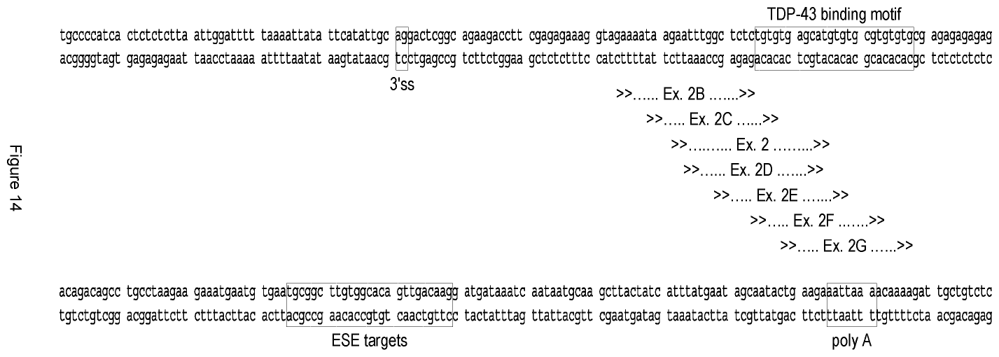

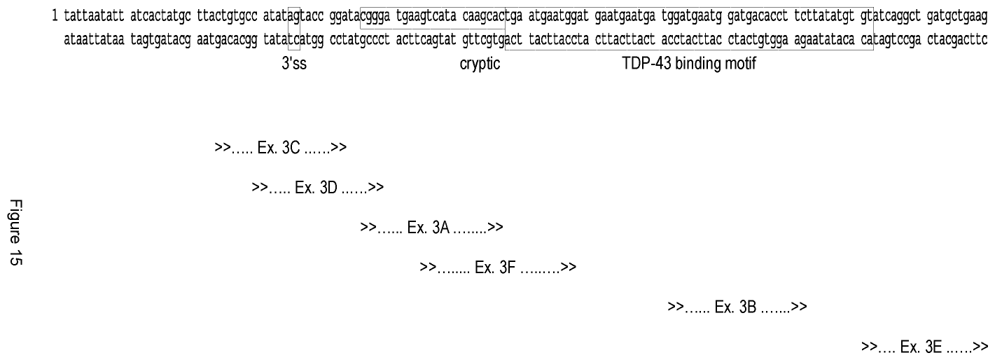

Next, “bifunctional” U7 smOPT constructs according to the present invention (i.e., comprising both a sequence comprising a hnRNP binding sequence and an antisense sequence complementary to UNC13A), were compared with analogous U7 smOPT constructs which contained the same antisense sequence which targeted the UNC13A cryptic exon, but which lacked the hnRNP binding tail/sequence. The experiment was performed by looking at splicing of the UNC13A minigene in 293T cells with TDP-43 inducible knockdown. As can be seen from FIGS. 12A and 12B, the “bifunctional” constructs of the present invention were significantly more effective than those which just contained the antisense sequence. This indicates that endogenous hnRNP proteins are being actively recruited to the pre-mRNA and fulfilling the role of TDP-43, in order to restore correct splicing.

### Further Exemplification

### Minigene Data

Comparison of bifunctional constructs of the invention versus comparative monofunctional constructs (i.e., where bifunctional constructs comprise an antisense sequence for the TDP-43 regulated cryptic exon and a binding sequence for a hnRNP protein, while the comparative “monofunctional” U7 constructs comprise an antisense sequence for the TDP-43 regulated cryptic exon but not a binding sequence for a hnRNP protein.

### STMN2

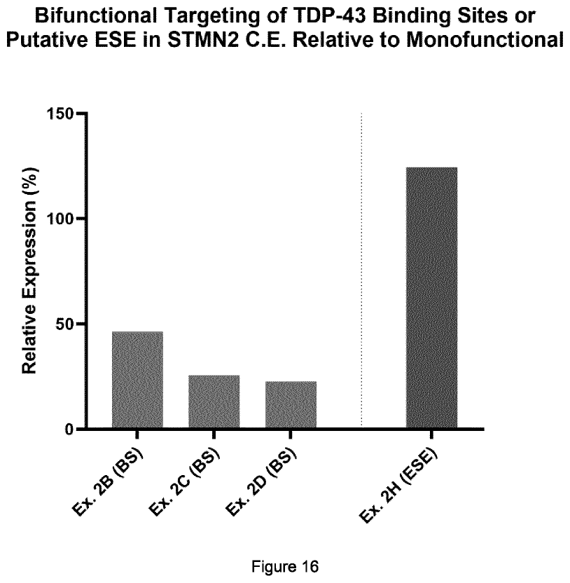

FIG. 16 shows the ratio of correctly spliced RT-qPCR levels of STMN2 mRNA from a bifunctional approach relative to the ratio obtained with a comparative monofunctional approach comprising the same antisense target which targets either a TDP-43 binding site (BS) or a putative ESE (ESE). Data is shown relative to the ratio in non-targeting control (U7 Control) transfected 293T-2xTDP-shRNA cells containing STMN2 minigene and under TDP-43 knockdown normalized to GDPDH mRNA. It is demonstrated that the bifunctional construct of the invention reduces C.E./Corr more effectively than a monofunctional approach when targeting the TDP-43 binding sequence. This provides further evidence that the bifunctional approach is more effective than a monofunctional approach when targeting both the TDP-43 binding site.

### UNC13A

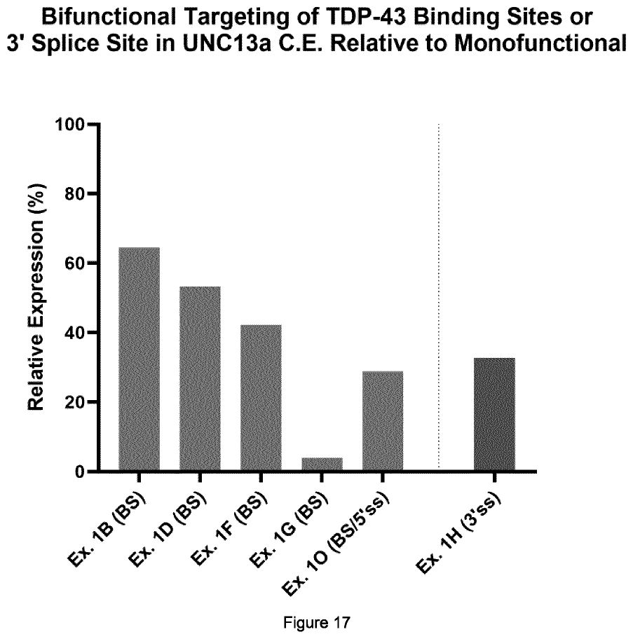

FIG. 17 shows the ratio of correctly spliced RT-qPCR levels of UNC13A mRNA from a bifunctional approach of the invention relative to the ratio obtained with a comparative monofunctional approach comprising the same antisense sequence which targets either a TDP-43 binding site (BS) or a 3′-splice site (3′ss). It is demonstrated that the bifunctional construct of the invention reduces C.E./Corr more effectively than a monofunctional approach when targeting the TDP-43 binding sequence and a 3′-splice site. This provides further evidence that the bifunctional approach is more effective than a monofunctional approach when seeking to rescue splicing of TDP-43 regulated CEs.

### Comparison of Bifunctional U7 Constructs Targeting TDP-43 Binding Sequences Versus Other Splice Elements

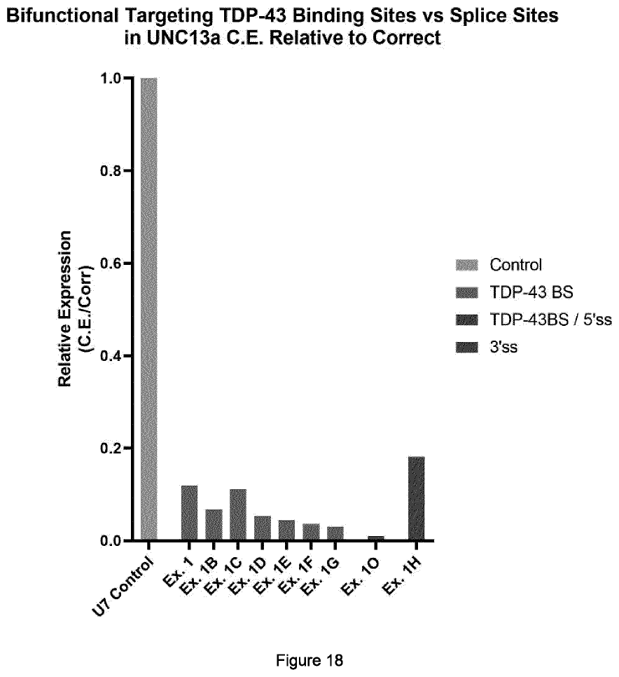

FIG. 18 shows the ratio of cryptic exon included to correctly spliced RT-qPCR levels of UNC13A mRNA comparing bifunctional approach targeting TDP-43 binding site (TDP-43 BS, Examples 1, 1B, 1C, 1D, 1E, IF or 1G) or 5′ splice site/TDP-43 BS (5′ss/TDP-43 BS, Example 10) to 3′ splice site (3′ss, Example 1H). Data is shown relative to ratio in non-targeting control (U7 Control) transfected 293T-2xTDP-shRNA cells containing UNC13A minigene and under TDP-43 knockdown normalized to GAPDH mRNA. The graph demonstrates that constructs targeting the TDP-43 binding site are more effective than a construct which targets the 3′-splice site. Of note, the construct which targets the TDP-43 binding site overlapping with the 5′-splice site (Example 10) was found to be particularly effective.

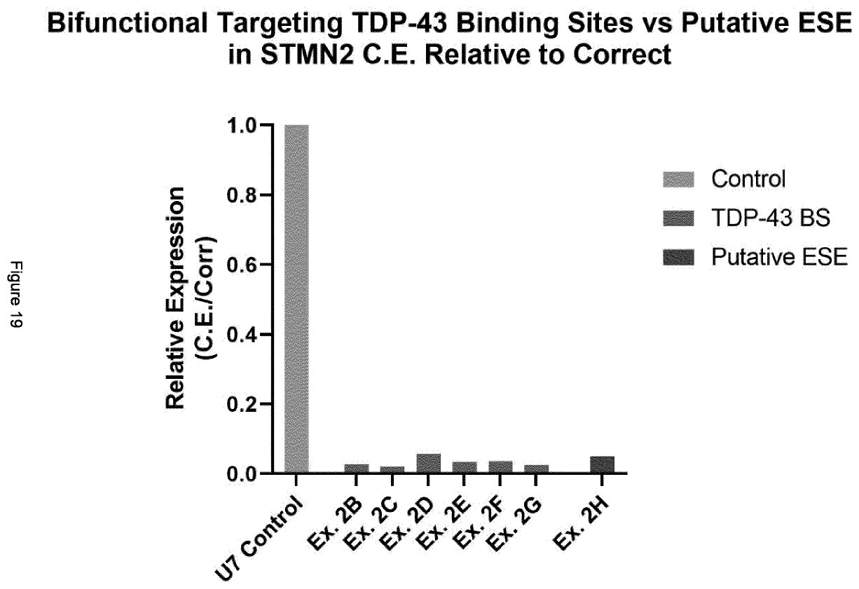

FIG. 19 shows the ratio of cryptic exon included to correctly spliced RT-qPCR levels of STMN2 mRNA comparing bifunctional approach targeting TDP-43 binding site (TDP-43 BS, Examples 2B-2G) to putative ESE (Example 2H). Data is shown relative to the ratio in non-targeting control (U7 Control) transfected 293T-2xTDP-shRNA cells containing STMN2 minigene and under TDP-43 knockdown normalized to GDPDH mRNA. The graph demonstrates that constructs targeting the TDP-43 binding site are in general more effective than constructs which targets the exonic splice enhancer (Example 2H) in the STMN2 CE.

### Data for Endogenous Testing in SH-SY5Y Cells

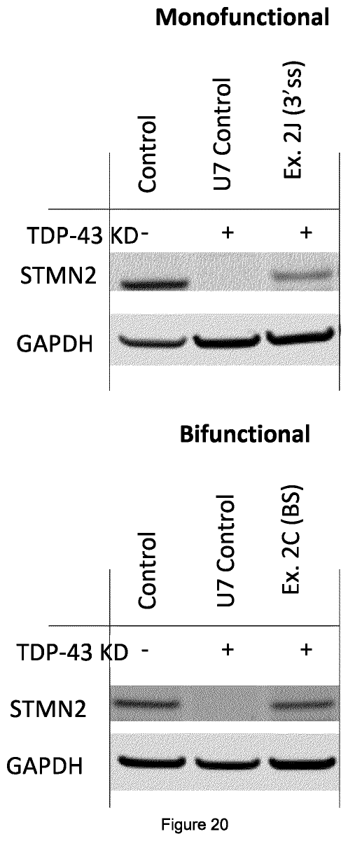

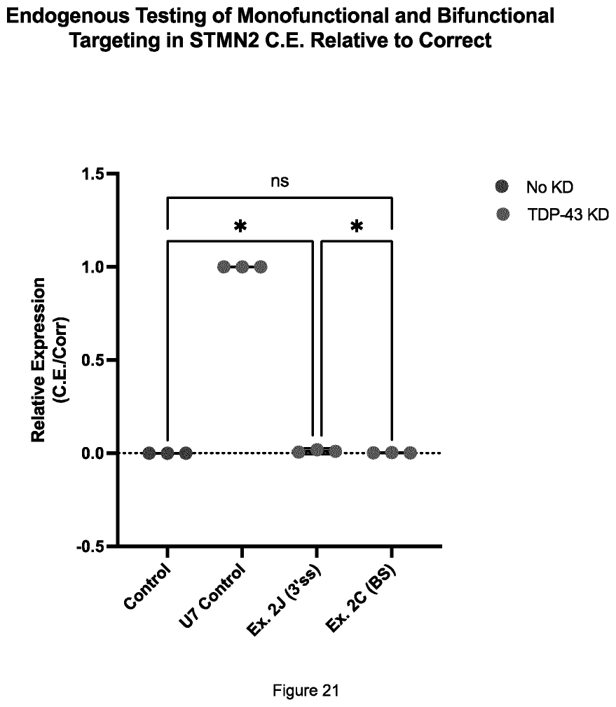

FIGS. 20 and 21 show that STMN2 levels are rescued using constructs of the invention to target the STMN2 cryptic exon in SH-SY5Y cells. The data further demonstrates that a bifunctional approach is more effective than a monofunctional approach at rescuing correct STMN2 mRNA and protein as evidenced by comparing constructs Example 2C and comparative Example 2J.

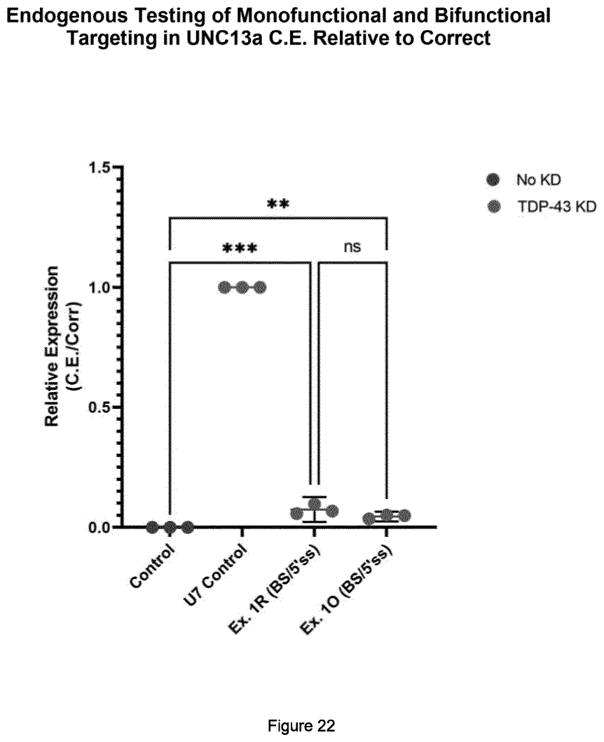

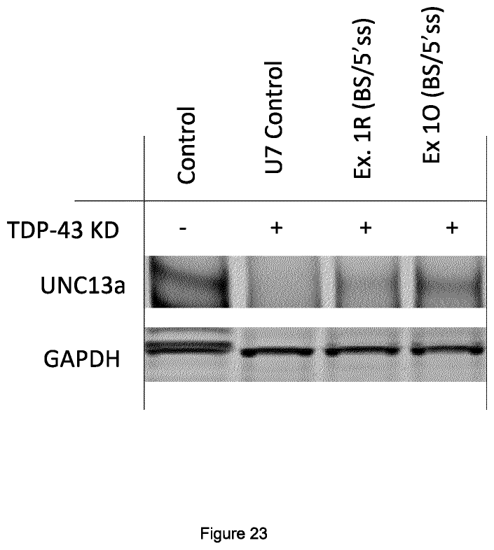

FIGS. 22 and 23 analogously show that UNC13A levels are rescued using constructs of the invention to target the UNC13A cryptic exon in SH-SY5Y cells. The data further demonstrates that a bifunctional approach is more effective at rescuing UNC13A at a protein level as evidenced by comparing the bifunctional construct Example 10 with a comparative monofunctional construct absent of hnRNP A1 binding sequence (Example 1R)

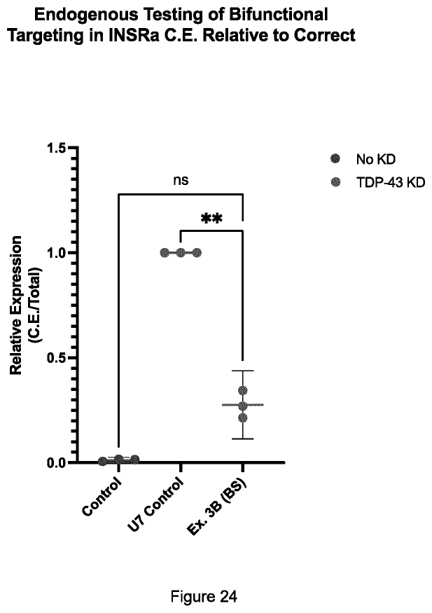

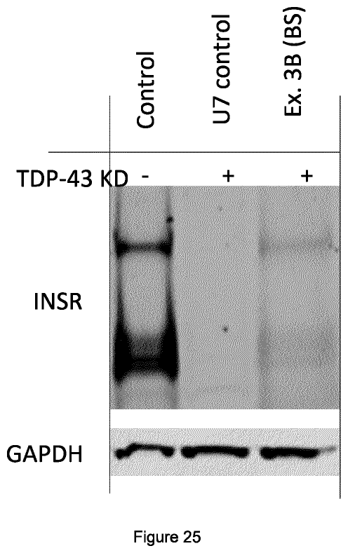

FIGS. 24 and 25 show that the bifunctional construct Example 3B targeting the INSR cryptic exon can also partially rescue and suppress TDP-43 regulated INSRa cryptic exon inclusion in SH-SY5Y cells.

### I3Neuron Data

Successful RNA and protein rescue was also demonstrated in i3Neurons using a U7 constructs of the disclosure to correct mis-splicing of the TDP-43 regulated cryptic exons UNC13A, STMN2 and INSR. Human iPSC-derived cortical neurons (i3Neurons) expressing a U7 constructs of the disclosure were cultured. TDP-43 knockdown was achieved by treating the cells with Halo-Protac (300 nM). RNA and protein were harvested on day 11.

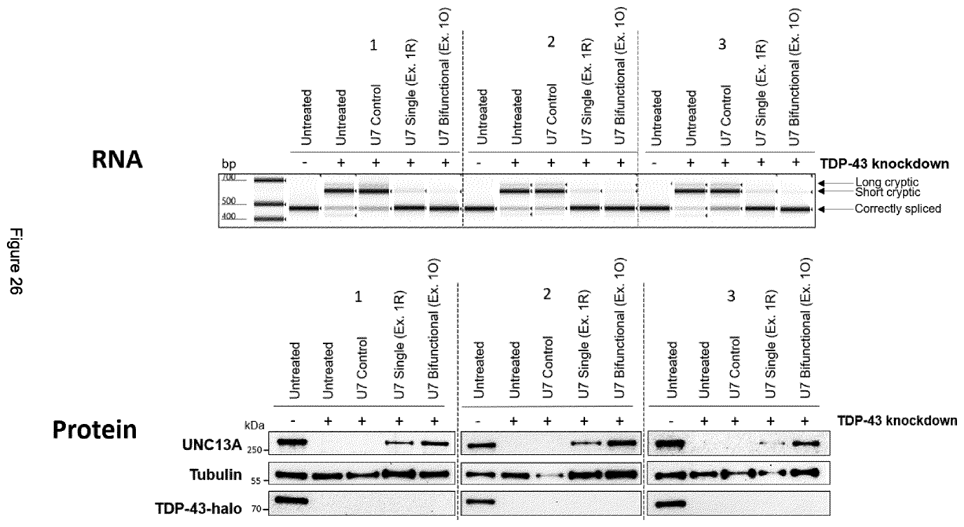

FIG. 26 Top) shows RT-PCR analysis of UNC13A splicing between exons 19 and 22 shows a rescue in splicing with Example 10. FIG. 26 Bottom) shows western blot analysis of UNC13A levels following treatment with Example 10. Also shown is a comparative U7 construct (Example 1R) containing an antisense sequence targeting the 5′ splice site, but without a hnRNP binding sequence. Rescue of splicing is more effective with the bifunctional construct than the comparative monofunctional construct.

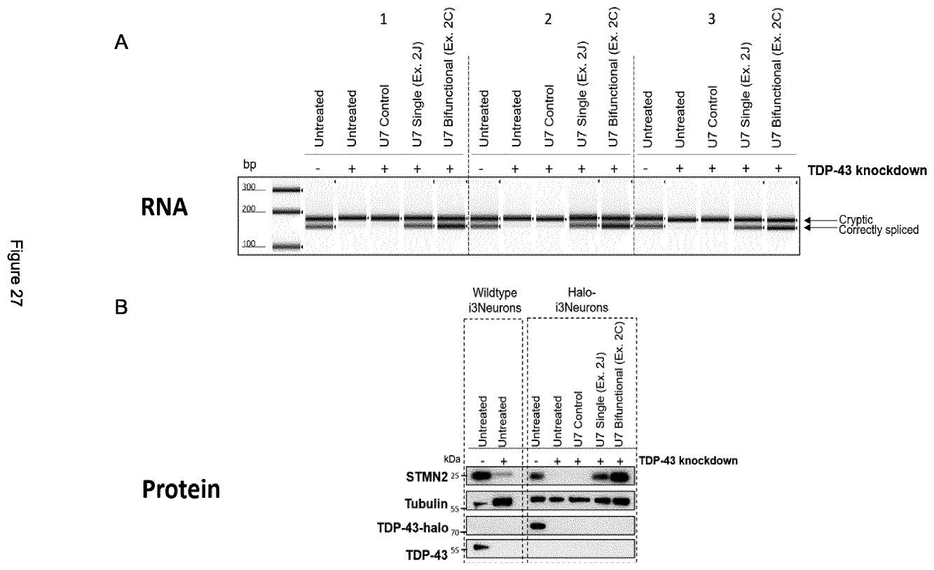

FIG. 27 Top) shows the three-primer RT-PCR analysis of STMN2 splicing at between exons 1 and 2 shows a rescue in splicing with Example 2C. FIG. 27 Bottom) Western blot analysis of STMN2 levels following treatment with Example 2C. Also shown is a comparative U7 construct (Example 2J) containing an antisense sequence targeting the 3′-splice site, but without a hnRNP binding sequence. Rescue of splicing is more effective with the bifunctional construct than the comparative monofunctional construct.

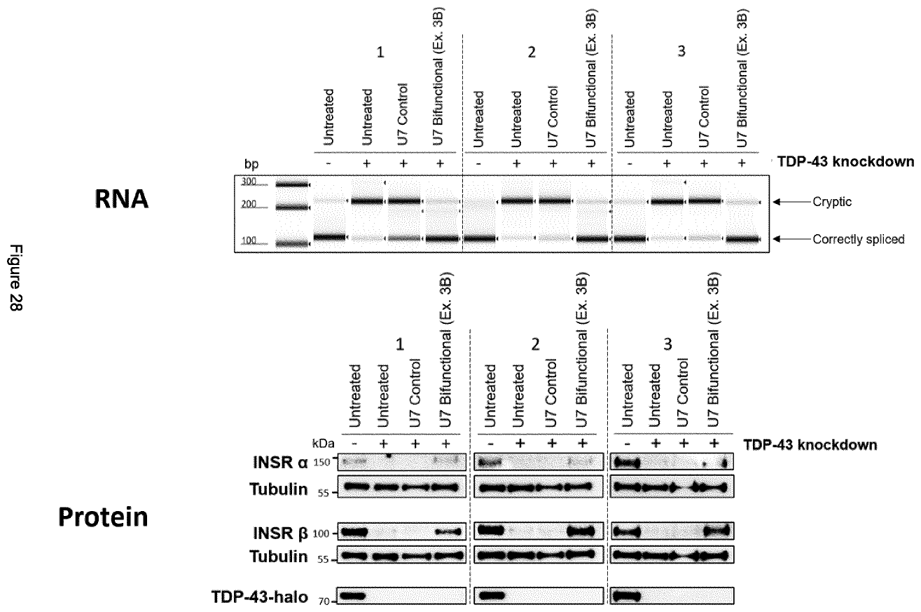

FIG. 28 shows RNA protein rescue of INSR mis-splicing using an INSR-targeting construct of the invention (Example 3B). FIG. 28 Top) shows RT-PCR analysis of INSR splicing at between exons 6 and 7 shows a rescue in splicing with the U7 bifunctional construct. FIG. 28 Bottom) shows Western blot analysis of INSR levels following treatment with the U7 Bifunctional construct, which shows rescue of INSR protein.

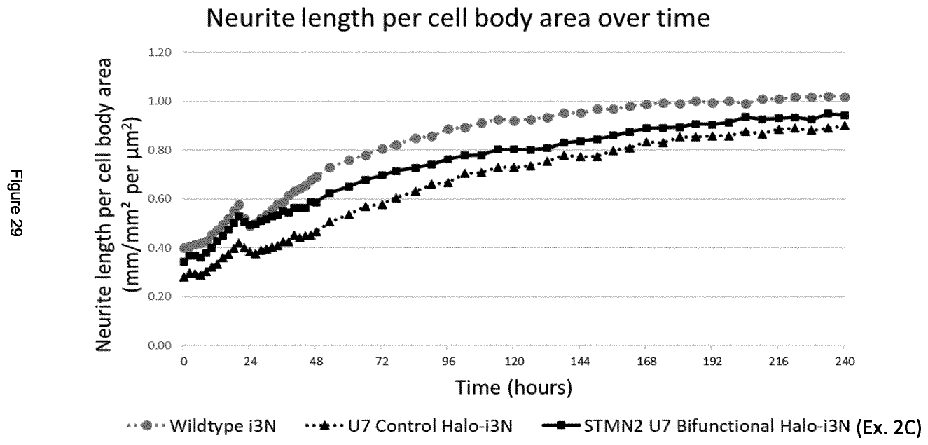

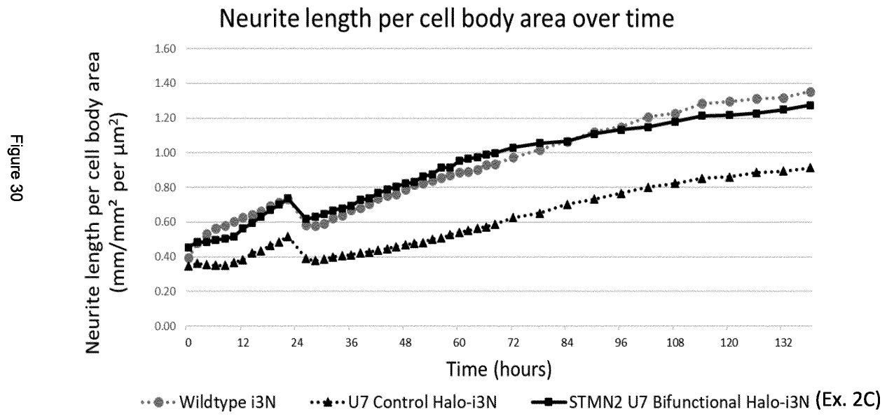

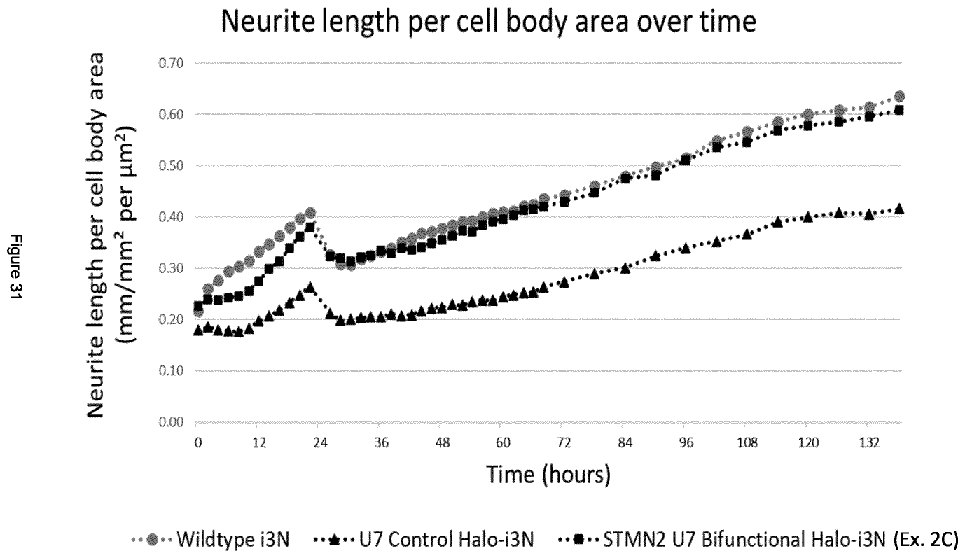

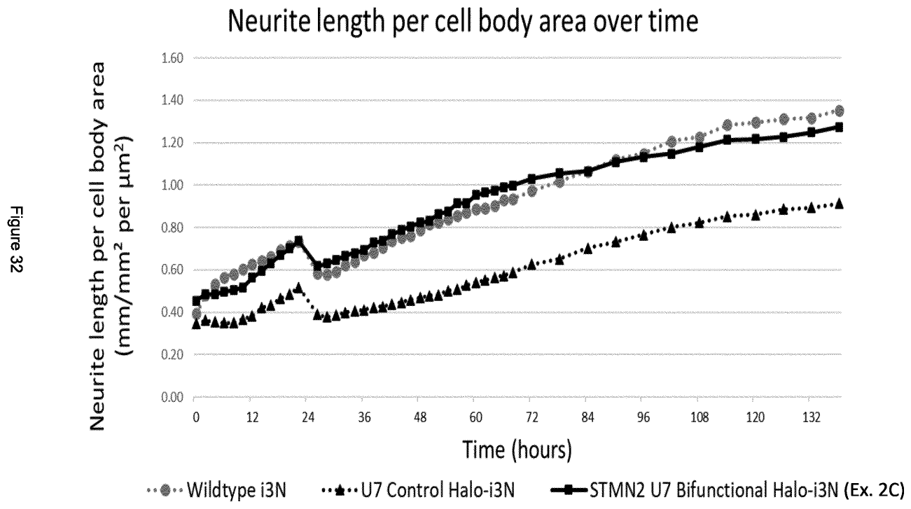

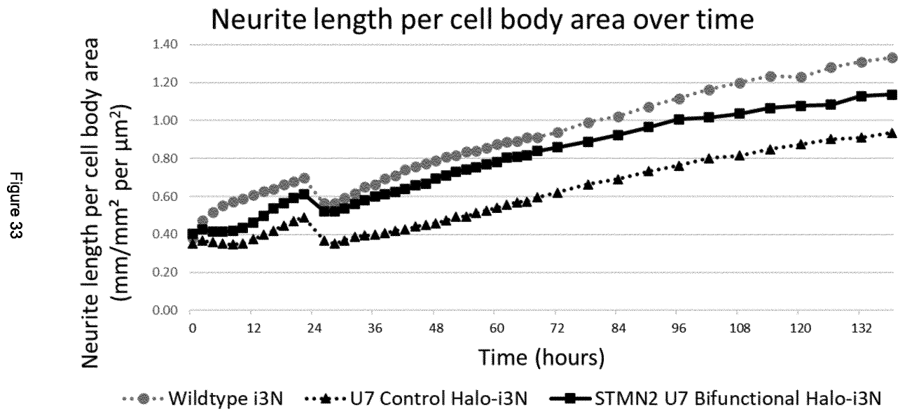

Rescue of reduced neurite outgrowth phenotype in i3 Neurons was also demonstrated using a construct of the disclosure (Example 2C), which targeted STMN2. FIGS. 29-33 show that neurite outgrowth of i3 Neurons is impaired by TDP-43 depletion and rescued by the STMN2-targeting U7 construct of the disclosure. After three days of neuronal induction media, human iPSC-derived cortical neurons (13Neurons) expressing a non-targeting Control U7 construct and an Example construct of the disclosure (Example 2C) were plated alongside wildtype i3Neurons in a 96-well plate. TDP-43 knockdown was achieved in the Control U7 and the Example construct of the disclosure by treating the cells with Halo-Protac (300 nM) from day 1 of induction media. The i3Neurons were longitudinally imaged for several days using an IncuCyte (Sartorius) imaging and analysis system, with eight technical replicates for each condition. Experiments were also performed to determine neurite outgrowth and cell body area were calculated. Five independent differentiations were performed and plotted on separate graphs, shown in FIGS. 29-34. Neurite length, normalised for cell body area, is reduced in TDP-43 depleted i3 Neurons expressing the Control U7, but is rescued in those expressing the STMN2-targeting construct of the disclosure (i.e., , corresponding to Example 2C)

### Combined Construct Vector

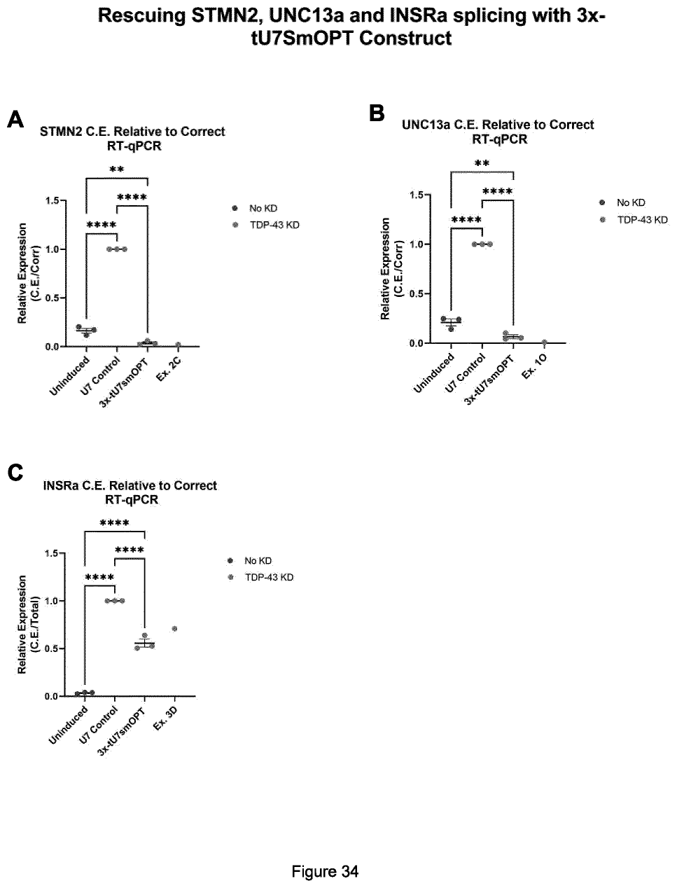

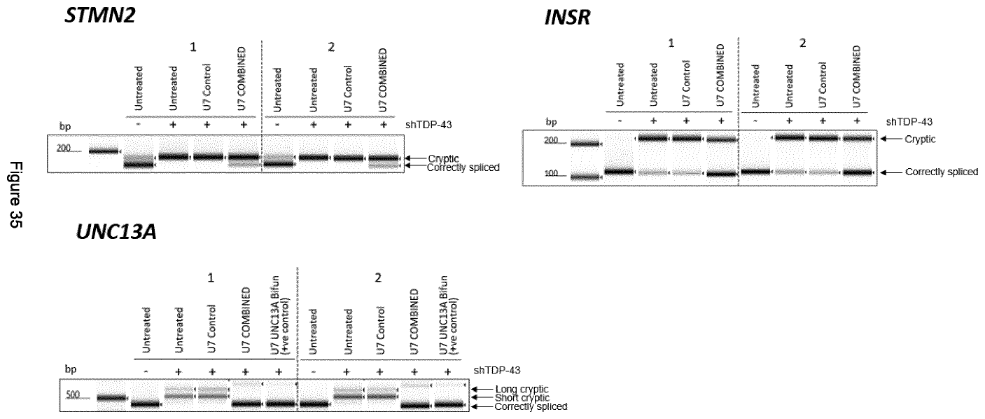

An example “multiple” construct vector was designed, which comprised three separate constructs in tandem targeting 3 different TDP-43 regulated exons: UNC13A, STMN2 and INSR. This construct is referred to herein as “3x-U7SmOPT” or “U7 Combined”. FIG. 34 shows the ratio of cryptic exon included to correctly spliced or total RT-qPCR levels of STMN2 (A), UNC13A (B) and INSR (C) mRNA in 293T-2xTDP-shRNA cells transfected with an STMN2 and an UNC13A minigene upon transfection with non-targeting control (Uninduced and U7 Control) or pMA-3x-U7SmOPT (3x-tU7SmOPT). The 3x-tU7SmOPT construct contains three U7s in tandem (Ex. 2C, Ex. 10 and Ex. 3D) and is compared to CE/Correct ratios obtained upon transfection with individual constructs corresponding to Ex. 2C, Ex. 10 and Ex. 3D alone. Data are presented as mean±SD relative to the ratio in non-targeting control and analyzed using ordinary one-way ANOVA with Tukey's multiple comparison test (*p<0.05, **p<0.01, ***p<0.001, ****p<0.0001). FIG. 35 shows RNA rescue of STMN2, and INSR mis-splicing using the U7 combined construct vector in SH-SY5Y neuronal cells. TDP-43 inducible shRNA knockdown SH-SY5Y cells were left untreated or treated with doxycycline 0.025 μg/mL for 5 days. The cells were then electroporated with 2 μg of U7 DNA constructs with Ingenio Electroporation Kit (Mirus) using the A-023 setting on an Amaxa II nucleofector (Lonza). The cells were then left untreated or treated with 1 μg/mL doxycycline for 5 further days before RNA extraction on day 10. RT-PCR analysis of STMN2, INSR, and UN (13A splicing shows a rescue in splicing of all three genes using the combined triple U7 construct. The positive control demonstrated good electroporation efficiency. PCR products were resolved on a TapeStation 4200 (Agilent). The combined construct vector showed similar suppression of 3 TDP-43 regulated exons, UNC13A, INSR and STMN2, as compared to individual construct transfection.

### CONCLUSIONS

Crucially, it has been demonstrated that replacing the natural antisense sequence of a modified U7 snRNA (U7 SmOPT) with constructs comprising both (i) an antisense sequence targeting a TDP-43 regulated cryptic exon and ii) a binding sequence for an alternative hnRNP splicing repressor, can effectively restore or rescue normal splicing in TDP-43 depleted cells. This has been demonstrated (i) for a number of TDP-43 regulated cryptic exons, including for UNC13A, STMN2 and INSR, (ii) for a wide range of antisense sequences that target different splicing elements, and (iii) with constructs that recruit different hnRNP proteins. In particular, constructs that recruited hnRNP A1 and hnRNP H, more particularly hnRNP A1, were found to be the most effective.

The bifunctional approach enables the TDP-43 cryptic exon sequence to be targeted using an antisense sequence, while recruiting an endogenous hnRNP to the cryptic site. Recruitment of hnRNP protein fulfils the repressive role of TDP-43 in the cell, leading to correct splicing. This approach is demonstrated to be more effective at correct splicing (e.g., as compared to a monofunctional approach) and is considered more robust than an approach that simply targets the cryptic exon or its splicing elements. The present inventors also found that constructs comprising antisense sequences which targeted the TDP-43 binding sites could be more effective than constructs comprising antisense sequences which targeted other splice elements, and that efficiency is even improved when sequences are targeted that contain TDP-43 binding sites as well as other splice elements (e.g splice sites)

The constructs of the present invention are also improved over alternative gene therapy approaches, such as antisense oligonucleotides, since ASOs are sensitive to degradation. As a result, ASO approaches would be less suitable as a therapy since they would need to be repeatedly delivered intrathecally and their distribution within the CNS is suboptimal. In contrast, U7 smOPT snRNA constructs can be delivered in vivo with vectorisation precluding the requirement for continuous oligo injections.

The present invention can therefore be used to further probe and understand the role of TDP-43 regulated cryptic exons in disease, and provide promising therapeutics for diseases associated with TDP-43 pathology.

The present inventors have also uniquely demonstrated a combined vector approach, which comprises two or more of the constructs of the invention (i.e., in tandem). Different from any prior approach, this combined construct vector targets different cryptic exons in different genes. The result is unexpected considering the combined construct comprises multiple identical promoters. This approach would not be expected to yield such a similar efficiency due to promoter competition and promoter interference. Indeed, it would be expected from previous literature that multiple promoters on one plasmid would have a different outcome to multiple plasmids with one promoter. While transcriptional interference can be prevented by cloning them in divergent orientation, this is not possible with three promoters where one promoter set will be in convergent position resulting in potential transcriptional interference.

Further advantages of the present invention are summarised in the statement of invention section.

### Materials and Methods

### Cloning of U7 Constructs Targeting Cryptic Exons

The U7 SmOPT expression cassettes containing the antisense sequence to the histone downstream element were ordered as gene synthesis either in pUC Simple (General Biosystems) or pMK (GeneArt, Life Technologies). To generate the constructs targeting cryptic exons, these constructs were digested with Stul and HindIII (New England Biolabs). DNA strings with 15 bp overhangs upstream and downstream of the Stul, HindIII cleavage sites containing the U7 SmOPT sequence with hnRNP binding and antisense sequences were designed as described below and cloned into the Stul and HindIII digested U7SmOPT plasmid using InFusion Snap Assembly EcoDry Master Mix (Takara) according to the manufacturer's instructions.

Design principle of strings cloned into Stul-HindIII digested U7 SmOPT cassettes: 15 bp overhangs required for the InFusion Snap Assembly reaction are underlined, Stul and HindIII sites are shown in bold. x=hnRNP tail (e.g., SEQ ID NO: 361 UAUGAUAGGGACUUAGGGUG), y=antisense sequence (e.g. SEQ ID NO: 420 UUCAUCUGUUCAAUCAUUCAUUC), SmOPT sequence is indicated in italics.

GGCUCGCUACAGAGGCCUUUCCGCAA-X-Y-AAUUUUUGGAG
CAGGUUUUCUGACUUCGGUCGGAAAACCCCUCCCAAGUUAAC
UGGUCUACAAUGAAAGCAAAACAGUUCUCUUCCCCGCUCCCC
GGUGUGUGAGAGGGGCUUUGAUCCUUCUCUGGUUUCCUAGGA
AACGCGUAAGCUUGUAUUCAGAU
(e.g., SEQ ID NO-421, shown with example
hnRNP and antisense sequences according to
Example 1).

### UNC13A and STMN Minigenes

The UNC13A minigene is described in Brown A.-L. et al, Nature, volume 603, pages131-137 (2022). The STMN2 minigene was generated by gene synthesis. A fragment containing exon 1 and the first 300 bp of intronic sequence followed by the cryptic exon 2a preceded by 300 bp intron 1 sequence and followed by 200 bp intronic sequence followed by exon 2 preceded by 200 bp intronic sequence and followed by 200 bp intronic sequence, followed by exon 3 preceeded by 200 bp intronic sequence was synthesized by GeneArt (Life Technologies). This fragment was cloned between the BamHI and Xhol sites of pcDNA3.1 (+).

Generation of Inducible TDP-43 Knockdown 293T Cells 293T cells were cultured in DMEM/F12 medium (Gibco) with 10% tetracyclein-free FBS and 1% Penicillin/Streptomycin. Inducible 293T TDP-43 knockdown cells were generated by transfecting 80% confluent cells in a well of a 6-well plate with AAVS1-SA-puro-EF1-hspCas9 (System Biosciences) targeting the AAVS1 locus (SEQ ID NO: 388 ggggccactagggacaggat) and pAAVSI-puro 2x TDP-43 shRNA in a 1:3 ratio. pAAVSI-puro 2x TDP-43 shRNA was generated by cloning a gene synthetised fragment containing two tet-operator containing 7SK/H1 hybrid promoters expressing each one TDP-43 shRNA (target 1: SEQ ID NO: 446 GAGACTTGGTGGTGCATAA, target 2: SEQ ID NO: 422 GGAGAGGACTTGATCATTA) into the BstB1 and Sall sites of pAAV-Puro_siKD (Bertero A., et al. Current Protocols in Stem Cell Biology, 44, 5C.4.1-5C.4.48. doi: 10.1002/cpsc.45). 24 hours post transfection, cells were split into a T150 plate and subjected to selection with 0.75 ug/ml Puromycin (Gibco) for seven days, followed by a 4-day selection with 1.5 ug/ml puromycin. Single colonies were picked, expanded. The inducible TDP-43 knockdown clone was identified by qRT-PCR. Cells were induced with 1 μg/ml doxycycline for 2 days, followed by RNA isolation using the Direct-zol RNA Miniprep Plus kit (Zymo Research) and TDP-43 knockdown was validated by assessment of TDP-43 mRNA levels by comparing induced and uninduced cells by qRT-PCR with Mesa Green qPCR MasterMix (Eurogentec) according to the manufacturer's instructions using 40 ng of cDNA and 0.6 uM f.c. primers sybr TDP-43 fwd: SEQ ID NO: 423 AACCGAACAGGACCTGAAAGAG and sybr TDP-43 rev: SEQ ID NO: 424 CAGTCACACCATCGTCCATCTATC and sybr beta-actin fwd: SEQ ID NO: 425 TCCATCATGAAGTGTGACGT and sybr beta-actin rev SEQ ID NO: 447 TACTCCTGCTTGCTGATCCAC in a total volume of 20 ul using a RotorGene Q (Qiagen).

### Testing of U7SmOPT Constructs on UNC13A and STMN2 Minigenes in TDP-43 Inducible Knockdown 293T Cells

To examine the efficiency of the U7 constructs on cryptic exon splicing for STMN2 and UNC13A, 80% confluent 293T-2xTDP-shRNA cells in 6-well plates were transfected with 200 ng of STMN2 or UNC13A minigenes and 1800 ng U7SmOPT-CMV-BSD plasmids using Mirus TransIT-LT1 (Mirus Bio) according to the manufacturer's instructions. 24 hours post-transfection, cells were split 1:1 and induced with 1 ug/ml doxycycline (Sigma Aldrich). 72 hours post transfection cells were harvested and RNA was isolated using the Absolutely RNA Miniprep Kit (Agilent technologies) according to the manufacturer's instructions. RNA was reverse transcribed to cDNA using the High-capacity RNA-to-cDNA kit (Applied Biosystems). TDP-43 mRNA levels as well as the ratio of cryptic to correctly spliced levels of STMN2 and UNC13A were assessed by RT-qPCR using 20 ul final volume of PowerUp™ SYBR™ Green Master Mix (ThermoFisher) with 40 ng of cDNA, 0.3 uM f.c. primers
hTDP-43 qPCR f: TCATCCCCAAGCCATTCAGG (SEQ ID NO: 426),
hTDP-43 qPCR r: TGCTTAGGTTCGGCATTGGA (SEQ ID NO: 427),
GADPH fwd: CCAGAACATCATCCCTGCCT (SEQ ID NO: 428),
GAPDH rev: SEQ ID NO: 429 GGTCAGGTCCACCACTGACA,
UNC13A Corr f: SEQ ID NO: 430 ACCTGTCTGCATGAGAACCT,
UNC13A Cryptic f: SEQ ID NO: 431 ATGGATGGAGAGATGGAACCT,
UNC13A r: SEQ ID NO: 432 GGGCTGTCTCATCGTAGTAAAC,
STMN2 Corr f: SEQ ID NO: 433 GCTAAAACAGCAATGGCCTAC,
STMN2 Corr r: SEQ ID NO: 434 TTGCTTCACTTCCATATCATCG,
STMN2 Cryptic f: SEQ ID NO: 435 GCTAAAACAGCAATGGGACTC,
STMN2 Cryptic r: SEQ ID NO: 436 GCAGGCTGTCTGTCTCTCTC on a Rotor-Gene Q using the fast cycling mode according to the manufacturer's instruction.

### INSR Minigene

The INSR minigene was generated via PCR of the genomic region of interest, containing exon 6, intron 6 including the cryptic exon 6a, and exon 7, using Q5 polymerase, followed by Gibson assembly into a suitable linearised vector featuring a CMV promoter and an SV40 poly A signal.

### Generation of Inducible TDP-43 Knockdown in SH-SY5Y and SK-N-DZ Cells

SH-SY5Y and SK-N-DZ cells were transduced with SmartVector lentivirus (V3IHSHEG_6494503) containing a doxycycline-inducible shRNA cassette for TDP-43. Transduced cells were selected with puromycin (1 μg/mL) for one week.

### Testing of U7SmOPT Constructs on UNC13A and INSR Minigenes in TDP-43 Inducible Knockdown SK-N-DZ Cells

TDP-43 inducible knockdown SK-N-DZ cells were left untreated or treated with doxycycline 1 μg/mL for 3 days. The cells were then transfected with total 1 μg of DNA with a ratio of minigene to U7smOPT of 1:3 using Lipofectamine3000 (Thermofisher Scientific) and then left untreated or treated with doxycycline for 3 further days before RNA extraction on day 6. Reverse transcription was performed with RervertAid (Thermo Scientific) and cDNA was amplified by PCR with minigene-specific primers 5′-TCCTCACTCTCTGACGAGG-3′ (SEQ ID NO: 437) and 5′-CATGGCGGTCGACCTAG-3′ (SEQ ID NO: 438) for the UNC13A minigene, and primers 5′-TACCATCCACTCGACACACC-3′ (SEQ ID NO: 439) and 5′-AGTCAGTCAAGCTAGCAGAGG-3′ (SEQ ID NO: 440) for the INSR minigene. PCR products were resolved on a TapeStation 4200 (Agilent) and bands were quantified with TapeStation Systems Software v3.2 (Agilent).

### Testing of U7SmOPT Constructs on Rescue of Endogenous UNC13A and STMN2 in TDP-43 Inducible Knockdown SH-SY5Y Cells

TDP-43 inducible knockdown SH-SY5Y cells were left untreated or treated with doxycycline 0.025 μg/mL for 5 days. The cells were then electroporated with 2 μg of U7SmOPT DNA with the Ingenio Electroporation Kit (Mirus) using the A-023 setting on an Amaxa II nucleofector (Lonza). The cells were then left untreated or treated with doxycycline for 5 further days with 1 μg/mL doxycycline with a PBS wash the day after electroporation before RNA extraction on day 10. Reverse transcription was performed with RervertAid (Thermo Scientific) and cDNA was amplified by PCR with primers 5′-GACATCAAATCCCGCGTGAA-3′ (SEQ ID NO: 441) and 5′-CATTGATGTTGGCGAGCAGG-3′ (SEQ ID NO: 442) for UNC13A, and primers 5′-GCTCTCTCCGCTGCTGTAG-3′ (SEQ ID NO: 443), 5′-CGAGGTTCCGGGTAAAAGCA-3′ (SEQ ID NO: 444), and 5′-CTGTCTCTCTCTCTCGCACA-3′ (SEQ ID NO: 445) for STMN2. PCR products were resolved on a TapeStation 4200 (Agilent) and bands were quantified with TapeStation Systems Software v3.2 (Agilent).

### pL.VX-EFla-mCherryT2a-BSD-U7smOPT Cloning and Virus Production

For endogenous testing in SH-SY5Y cells, U7smOPT strings were cloned into the Clal sites of a pLVX-EFla-mCherry T2A-BSD vector. pL VX-EFla-mCherry T2A-BSD was generated by cloning a gene synthesized string containing the mCherry T2A-BSD ORF between the EcoRI and MluI sites of pL Vx-EF1a-IRES-Puro (Clonetech Laboratories, Takara Bio) using In-Fusion Snap Assembly EcoDry (Takara Bio) following the manufacturer's instructions. The U7smOPT strings were PCR amplified from their respective U7smOPT-CMV-BSD construct with additional 15 bp overhangs using CloneAmp HiFi PCR (Clonetech Laboratories, Takara Bio) following manufacturer's instructions with use of 0.3 uM of primers LV inf pLVX Cla f: AGATCCAGTTTATCGATACCAACATAGGAGCTGTGATTGG (SEQ ID NO: 475) and LV inf pL VX Cla r:

ATGAATTACTCATCGGCGAGAAAGGAAGGGAAGAAAGC (SEQ ID NO: 476) and 100 ng of template plasmid. This was then cloned into the pLVX-EFla-mCherryT2A-BSD backbone digested with Clal (New England BioLabs) using In-Fusion Snap Assembly EcoDry (Takara Bio) following the manufacturer's instructions.

21 μg of the cloned pLVX-EFla-mCherryT2A-BSD-U7smOPT and 30 ul Trans-Lentiviral Packaging Mix (Dharmacon) was transfected using Lipofectamine 2000 (Invitrogen) following manufacturer's instructions into >80% confluent HEK293T cells (Takara Bio) cultured in T-150 flasks using DMEM/F12 medium (Gibco) with 10% tetracycline-free FBS and 1% Penicillin/Streptomycin. Medium exchange was performed 24 hours post-transfection and 35 ml supernatant was harvested, filtered with 0.45 um SFCA filter (Thermo Scientific), and supplemented with LentiX Concentrator (1:4, Takara Bio) for the following two days. The mixture was then incubated overnight at 4° C., centrifuged at 1,500×g for 45 minutes at 4° C., resuspended in a total of 2 ml PBS, and aliquoted before flash freezing in LN2 and storing at-70° C. Virus titer was estimated to be at least 1×107 IFU/ml using Lenti-X GoStix (Takara Bio) prior to freezing aliquots.

mCherry TSA-BSD-ORF-
(SEQ ID NO: 477)
TATTTCCGGTGAATTCGCCGCCACCATGGTTTCCAAGGGCGAAGAG
GACAACATGGCCATCATCAAAGAATTCATGCGGTTCAAGGTGCAC
ATGGAAGGCAGCGTGAACGGCCACGAGTTCGAGATTGAAGGCGAA
GGCGAGGGCAGACCTTACGAGGGAACACAGACCGCCAAGCTGAAA
GTGACCAAAGGCGGCCCTCTGCCTTTTGCCTGGGACATTCTGAGC
CCTCAGTTTATGTACGGCAGCAAGGCCTACGTGAAGCACCCCGCC
GATATTCCCGACTACCTGAAGCTGAGCTTCCCCGAGGGCTTCAAG
TGGGAGAGAGTGATGAACTTCGAGGACGGCGGCGTGGTCACCGTG
ACTCAAGATAGCTCTCTGCAGGACGGCGAGTTCATCTACAAAGTG
AAGCTGCGGGGCACCAACTTTCCCTCTGATGGCCCCGTGATGCAG
AAAAAGACCATGGGCTGGGAAGCCAGCAGCGAGAGAATGTACCCT
GAAGATGGCGCCCTGAAGGGCGAGATCAAGCAGCGGCTGAAACTG
AAGGATGGCGGCCACTACGACGCCGAAGTGAAAACCACCTACAAG
GCCAAGAAACCCGTGCAGCTGCCTGGCGCCTACAACGTGAACATC
AAGCTGGACATCACCAGCCACAACGAGGACTACACCATCGTGGAA
CAGTACGAGAGAGCCGAAGGCAGACACAGCACAGGCGGAATGGAC
GAGCTGTACAAAGGCTCTGGCGAAGGCCGTGGCAGCCTGCTTACA
TGCGGAGATGTGGAAGAGAACCCCGGACCTATGGCCAAGCCTCTG
AGCCAAGAGGAAAGCACCCTGATCGAAAGAGCCACCGCCACAATC
AACAGCATCCCCATCAGCGAGGATTACAGCGTGGCCTCTGCTGCC
CTCAGCTCCGATGGCAGAATCTTCACAGGCGTGAACGTGTACCAC
TTCACCGGCGGACCTTGTGCCGAACTGGTGGTTCTTGGAACAGCT
GCCGCTGCCGCAGCCGGCAATCTGACATGTATTGTGGCCATCGGC
AACGAGAACCGGGGCATCCTTAGTCCTTGCGGCAGATGCAGACAG
GTGCTGCTGGATCTGCACCCTGGCATCAAGGCCATTGTGAAGGAC
TCTGACGGCCAGCCTACAGCCGTGGGAATTAGAGAGCTGCTGCCT
AGCGGCTATGTGTGGGAGGGATGAACGCGTCTGGAACAAT

Generation of i3Neuron-Halo Line

The human iPSC cell line with doxycycline inducible expression of NGN2 was obtained from Michael Ward, NIH (Tian et al., 2019) and maintained following a published protocol (Fernandopulle et al., 2018). The endogenous copy of Tardbp was tagged with the HaloTag using CRISPR-Cas12 genome editing. iPSCs were nucleofected with a 4-D nucleofector (Amaxa) with the P3 Primary Cell 4-D Nucleofector kit (Amaxa V4XP-3024). One million cells were nucleofected with ribonucleoprotein complexes formed of 5 mL of Tardbp targeting crRNA (Integrated DNA Technologies 100 mM) and 20 mg of recombinant Cas12a (IDT 10001272) and 10 mg of Homology Directed Repair template (Addgene plasmid 178131). Cells were plated in Geltrex (ThermoFisher Scientific, A1413202) coated dishes in E8 Flex media (ThermoFisher Scientific, A2858501) with 1x RevitaCell (ThermoFisher Scientific, A2644501) and 1 mM HDR enhancer V2 (Integrated DNA Technologies) and maintained in a 5% CO2 incubator at 32° C. for 24 hours. After 24 hours media was changed on cells to E8 Flex without RevitaCell or HDR enhancer and maintained in a 5% CO2 incubator at 37° C. iPSCs were expanded and single cell plated on to Geltrex coated 96 well plates. Genomic DNA was harvested from single cell colonies and their genotype was determined by PCR amplification with primers Halo_Geno_Forl and Halo_Geno_Rev1 followed by analysis with agarose gel electrophoresis.

Tardbp crRNA: SEQ ID NO: 478

/A1TR1/rUrArArUrUrUrCrUrArCrUrCrUrUrGrUr
ArGrArUrGrGrArArArArGrUrArArArArGrArUrGrU
rCrUrGrArArU/A1TR2/
Halo_Geno_For1:
(SEQ ID NO: 479)
5′-CTGGCGAGGCATCACATTTT-3′
Halo_Geno_Rev1:
(SEQ ID NO: 480)
5′-CGTTCTCATCTTCGGTTACCC-3′

Generation of iPSC Lines with Stable Expression of U7s

To achieve stable expression of the U7 constructs, they were delivered to iPSCs by lentiviral transduction. 50 mL of concentrated virus was delivered to 250,000 iPSCs in suspension in E8 Flex media (ThermoFisher Scientific, A2858501) with 10 mg/mL polybrene (hexadimethrine bromide, Sigma H9268) into one well of a 12-well plate following an accutase split. Cells were plated and cultured overnight. The following morning, cells were washed with PBS and media was changed to E8 Flex. Two days after lentiviral delivery, cells were selected for 48 hours with 10 mg/mL blasticidin (Sigma, SBR000221ML) iPSCs were then expanded 1-2 days before initiating neuronal differentiation. Transduction efficiency was confirmed using the fluorescence marker.

iPSC-Derived i3Neuron Differentiation and Culture

The WTC11 human iPSCs used in this study were previously engineered to express mouse or human neurogenin-2 (NGN2) under a doxycycline-inducible promoter, as well as an enzymatically dead Cas9 (+/−CAG-dCas9-BFP-KRAB) (Fernandopulle et al., 2018). These were integrated at the AAVS1 safe harbour and the CLYBL promoter safe harbour, respectively.

To initiate neuronal differentiation, 2.5 million iPSCs per 10 cm plate were single-cell plated using accutase on day 0 and re-plated onto Geltrex-coated tissue culture dishes in N2 differentiation media containing: knockout DMEM/F12 media (Life Technologies Corporation, cat. no. 12660012) with N₂ supplement (Life Technologies Corporation, cat. no. 17502048), 1× GlutaMAX (ThermoFisher Scientific, cat. no. 35050061), 1× MEM nonessential amino acids (NEAA) (ThermoFisher Scientific, cat. no. 11140050), 10 mM ROCK inhibitor (Y-27632; Selleckchem, cat. no. S1049) and 2 mg/ml doxycycline (Clontech, cat. no. 631311). Media was changed daily during this stage.

On day 3, pre-neuron cells were replated onto dishes coated with freshly made 100 mg/mL poly-D-lysine (Sigma, P7886) overnight and 10 mg/mL laminin (Thermo, cat no. 23017015) overnight in either 96-well plates (12,500-25,000 cells per well) for IncuCyte experiments, or 12-well dishes (500,000 cells per well) for RNA and protein extraction in i3Neuron Culture Media: BrainPhys media (Stemcell Technologies, cat. no. 05790) supplemented with 1x B27 Plus Supplement (ThermoFisher Scientific, cat. no. A3582801), 10 ng/ml BDNF (PeproTech, cat. no. 450-02), 10 ng/ml NT-3 (PeproTech, cat. no. 450-03), 1 mg/ml mouse laminin (Sigma, cat. no. L2020-1 MG), and 2 mg/ml doxycycline (Clontech, cat. no. 631311). On the day of plating, 1x RevitaCell (Thermo, cat no. A2644501) was added to the media. 24 hours after plating, media was fully replaced to remove RevitaCell. Following this, i3Neurons were then fed twice a week by half-media changes.

### RNA Extraction and RT-PCR

RNA was extracted from i3Neurons on day 11 and SH-SY5Y cells on day 10 using the RNeasy kit (Qiagen) or from i3Neurons on day 7 after the initiation of differentiation using a Direct-zol RNA miniprep kit (Zymo Research R2052) following the manufacturer's protocol including the on-column DNA digestion step. RNA concentrations were measured by Nanodrop and 500-1,000 ng of RNA was used for reverse transcription. First strand cDNA synthesis was performed using RevertAid (Thermo K1622) using random hexamer primers and following the manufacturer's protocol including all optional steps. cDNA was amplified by PCR with primers as described above for UNC13A and STMN2 (SEQ ID NO: 441-445), and the following primers for INSR for: 5′-AACGACATTGCCCTGAAGAC-3′ (SEQ ID NO: 481) INSR rev: 5′-CCAGTACGGCTCCCATCT-3′ (SEQ ID NO: 482). PCR products were resolved on a TapeStation 4200 (Agilent).

### Western Blot

i3Neurons were lysed directly on day 11 in the sample loading buffer (Thermo NP0008). Lysates were heated at 95° C. for 5 min with 100 mM DTT. Lysates were passed through a QIAshredder (Qiagen) to shear DNA. Lysates were resolved on 4-12% Bis-Tris Gels (Thermo) and transferred to 0.45 μm PVDF (Millipore) membranes. After blocking with 5% milk, blots were probed with antibodies (Rb anti-UNC13A (Synaptic Systems 126 103) 1:2,000; Rb anti-STMN2 (ProteinTech 10586-1-AP) 1:1,000; Rb anti-INSR-a (Cell Signaling Technology #74118 clone D3U7I) 1:1,000; Rb anti-INSR-B (Cell Signaling Technology #3025 clone 4B8) 1:1,000; Rat anti-Tubulin (Millipore MAB1864 clone YL1/2) 1:5,000, Mouse anti-TDP-43 (abcam ab 104223 clone 3H8) 1:5,000) at 4° C. overnight. After washing, blots were probed with HRP-conjugated secondary antibodies (Goat anti-Rabbit HRP (Bio-Rad 1706515) 1:10,000; Goat anti-Mouse HRP (Bio-Rad 1706516) 1:10,000; Rabbit anti-Rat HRP (Dako P0450) 1:10,000) and developed with Chemiluminescent substrate (Merck Millipore WBKLS0500) on a ChemiDoc Imaging System (Bio-Rad).

### Generation of Inducible TDP-43 Knockdown in SH-SY5Y Neuronal Cells

SH-SY5Y cells were transduced with SmartVector lentivirus (V3IHSHEG_6494503) containing a doxycycline-inducible shRNA cassette for TDP-43. Transduced cells were selected with puromycin (1 μg/mL) for one week.

Testing of the Combined Triple U7 Construct on Rescue of Endogenous UNC13A, STMN2, and INSR Splicing in SH-SY5Y Cells with Inducible TDP-43 Knockdown

TDP-43 inducible knockdown SH-SY5Y cells were left untreated or treated with doxycycline 0.025 μg/mL for 5 days. The cells were then electroporated with 2 μg of the combined triple U7 construct and a non-targeting U7 control with the Ingenio Electroporation Kit (Mirus) using the A-023 setting on an Amaxa II nucleofector (Lonza). An UN (13A-targeting U7 Bifunctional construct known to successfully rescue splicing was included as an experimental condition to demonstrate electroporation efficiency. The cells were then left untreated or treated with doxycycline for 5 further days with 1 μg/mL doxycycline before RNA extraction on day 10.

Neurite Outgrowth Experiment in i3Neurons

After three days of induction media, i3Neurons stably expressing a non-targeting Control U7 construct and a STMN2-targeting Bifunctional U7 construct were plated alongside wildtype i3 Neurons in i3Neuron Culture Media with RevitaCell in a 96-well plate coated with poly-D-lysine and laminin, as described previously. For each condition, two cell densities were plated (12,500 and 25,000 cells), and each density was plated in eight wells, serving as eight technical replicates per condition. TDP-43 knockdown was achieved in the Control U7 and STMN2 Bifunctional U7 conditions by treating the cells with Halo-Protac3 (Promega, GA3110, 300 nM) from day 1 of induction media. The 96-well plate was then placed in an IncuCyte (Sartorius) longitudinal imaging and analysis system for several days. The IncuCyte machine was set-up to capture four images per well every 2 hours initially, after which the frequency was increased to every 6 hours. 24 hours after plating, a full media change was performed to remove RevitaCell. Following this, half-media changes were performed twice a week. Using the cell body and neurite masks, respectively, cell body area and neurite outgrowth were calculated for each condition. Neurite length was normalised to cell body area and plotted over time. Five independent differentiations were performed, with data from each differentiation plotted on a separate graph.

### Endogenous Testing of U7smOPT in TDP-43 Inducible Knockdown SH-SY5Y Cells

˜2.5 million inducible TDP-43 knockdown SH-SY5Y cells in 5 ml DMEM/F12 medium (Gibco) supplemented with 10% tetracycline-free FBS, 1% Penicillin/Streptomycin and 4 ug/ml Polybrene (SantaCruz) in T-25 flasks were transduced with 100 ul of lentivirus for 24 hours. Stable clones were selected using 2 ug/ml Blasticidin (Gibco) for 4 days followed by 2 days 1 ug/ml Puromycin (Gibco) to reselect stable clones expressing the TDP-43 shRNA cassettes. Two T-25 flasks, one for protein and one for RNA isolation, were seeded for each line and TDP-43 knockdown was induced the next day by 0.1 ug/ml doxycycline for 5 days and another 5 days of 1 ug/ml doxycycline.

### Western Blot

Total protein was harvested using cold lysis buffer [Pierce RIPA Buffer (Thermo Scientific), 2X Halt Protease Inhibitor Cocktail 100x (Thermo Scientific) 1:50, 2M MnSO4 (1:500), Cyanase Nuclease (1:1000, SERVA)] and equal amount of 2xLDL [50% NuPage LDS Sample Buffer (Invitrogen) and 50% DTT] was then added before denaturing samples for 10 minutes at 70° C. Identical quantities of denatured protein lysate were run on NuPage 4-12% Bis-Tris Gel (Invitrogen) for STMN2, and NuPage 3-8% Tris Acetate Gel (Invitrogen) for UNC13A and INSRa. Gels were then transferred in Nitrocellulose membranes (Invitrogen) and incubated in Antigen Pretreatment solution from SuperSignal™ Western Blot Enhancer Kit (Thermo Scientific) according to manufacturer instruction to enhance protein bands. After blocking with Fish Serum Blocking Buffer (Thermo Scientific), membranes were incubated at 4° C. overnight in mouse monoclonal GAPDH (1:1000, SantaCruz) and rabbit polyclonal STMN2 antibody (1:1000, Proteintech), mouse monoclonal STMN2 (1:500, R&D Systems), rabbit polyclonal Munc13-1 antibody (1:1000, Synaptic Systems) or rabbit monoclonal INSRa antibody (1:1000, Cell Signaling Technology) diluted in Primary Antibody Diluent from SuperSignal™ Western Blot Enhancer Kit (Thermo Scientific). Next, they were washed with 1x TBST and incubated with donkey anti-rabbit and anti-mouse secondary antibodies (1: 10′000, Li-Cor) for 2 hours at room temperature and washed again. Finally, membranes were imaged with Odyssey CLx imaging system (Li-Cor) and protein bands quantified using Image Studio Lite (Li-Cor) by analyzing pixel density, and protein levels were normalized to GAPDH.

### RT-qPCR

RNA was extracted from SH-SY5Y cells using the Absolutely RNA Miniprep Kit (Agilent technologies) according to the manufacturer's protocol and first strand cDNA synthesis was performed with High-capcity RNA-to-cDNA kit (Applied Biosystems) or LunaScript RT SuperMix Kit (New England BioLabs). The ratio of cryptic to correctly spliced levels of STMN2 and UNC13A, cryptic to total levels of INSR as well as TDP-43 mRNA levels normalized to GAPDH were assessed by RT-qPCR using 20 ul final volume of PowerUp™ SYBR™ Green Master Mix (ThermoFisher) with 40 ng of cDNA, 0.3 uM f.c. using the primers outlined above (SEQ ID NO: 426-436) for GAPDH, UNC13A, STMN2 and hTDP-43 and the following primers for INSR

Total INSR f:
(SEQ ID NO: 483)
TGGGACCGCTTTACGCTTC,
total INSR r:
(SEQ ID NO: 484)
GAGACTGGCTGACTCGTTGAC,
CE INSR f:
(SEQ ID NO: 485)
CTCTGGGACTGGAGCAAAC,
CE INSR r:
(SEQ ID NO: 486)
CATCCCGTATCCGGTAAGG,

on a Rotor-Gene Q using the fast cycling mode according to the manufacturer's instruction.
Testing of the Combined 3x-U7SmOPT Vector Construct on STMN2 and UNC13a Minigenes in TDP-43 Inducible Knockdown 293T Cells

A pMA-3x-U7smOPT vector containing three U7 cassettes against STMN2, UNC13a and INSR was ordered as gene synthesis. To examine the efficiency of the 3x-U7 constructs on cryptic exon splicing for STMN2 and UNC13A, 80% confluent 293T-2xTDP-shRNA cells in 6-well plates were transfected with 200 ng of STMN2 and 200 ng UNC13A minigenes, and 1800 ng of pMA-3x-U7smOPT plasmids using Mirus TransIT-LT1 (Mirus Bio) according to the manufacturer's instructions. 24 hours post-transfection, cells were split 1:1 and induced with 1 ug/ml doxycycline (Sigma Aldrich). 72 hours post transfection cells were harvested and RNA was isolated using the Absolutely RNA Miniprep Kit (Agilent technologies) according to the manufacturer's instructions. RNA was reverse transcribed to cDNA using the LunaScript RT SuperMix Kit (New England BioLabs). TDP-43 mRNA levels as well as the ratio of cryptic to correctly spliced levels of STMN2 and UNC13A, and ratio of cryptic to total levels of INSRa were assessed by RT-qPCR using 20 ul final volume of PowerUp™ SYBR™ Green Master Mix (ThermoFisher) with 40 ng of cDNA, 0.3 uM f.c. using the primers outlined above (SEQ ID NO: 426-436) for GAPDH, UNC13A, STMN2 and hTDP-43 and SEQ ID NO: 483-486 for INSR.

1. A modified U7 snRNA construct comprising
(i) an antisense sequence having between 16 to 30 nucleotides which is at least 90% complementary to a TDP-43 regulated cryptic exon sequence or flanking regions thereof, and
(ii) a sequence comprising a binding domain for a hnRNP protein,
wherein the modified U7 snRNA construct is capable of modulating splicing of the TDP-43 regulated cryptic exon in a cell.

2. The modified U7 snRNA construct of claim 1, wherein the cryptic exon sequence is present in one of following genes: UNC13A, STMN2, INSR, ELAVL3, G3BP1, AARS1, CELF5, CAMK2B or UNC13B, optionally wherein the cryptic exon sequence is present in UNC13A, STMN2 or INSR.

3. The modified U7 snRNA construct of claim 1, wherein the antisense sequence is 100% complementary to the TDP-43 regulated cryptic exon sequence or flanking regions thereof.

4. The modified U7 snRNA construct of claim 1, wherein the binding domain is for a hnRNP A or hnRNP H protein.

5. The modified U7 snRNA construct of claim 1, wherein the hnRNP protein is hnRNP A1, or wherein the hnRNP protein is hnRNP A1 and the sequence comprising the binding domain for the hnRNP A1 protein comprises at least one motif corresponding to WUAGGGWS wherein W is A or U and S is G or C, or wherein the hnRNP protein is hnRNP A1 and the hnRNP A1 comprises two motifs corresponding to WUAGGGWS, or wherein the hnRNP protein is hnRNP A1, the hnRNP A1 comprises two motifs corresponding to WUAGGGWS, and the sequence that comprises the binding domain for the hnRNP A1 protein has at least 80% sequence identity to SEQ ID NO: 361.

6. The modified U7 snRNA construct of claim 1, wherein the antisense sequence is between 16 and 26 nucleotides, more or wherein the antisense sequence is between 17 and 23 nucleotides, and more or wherein the antisense sequence is between 18 and 22 nucleotides.

7. The modified U7 snRNA construct of claim 1, wherein the antisense sequence is capable of binding to a splicing element of the cryptic exon sequence, optionally wherein the antisense sequence is at least 90% complementary to one of SEQ ID NO: 11-40

8. The modified U7 snRNA construct of claim 1, wherein the antisense sequence is capable of binding to a TDP-43 binding region of the TDP-43 regulated cryptic exon sequence.

9. The modified U7 snRNA construct of claim 1, wherein the TDP-43 binding domain is any sequence of at least 6 nucleotides, or wherein the TDP-43 binding domain is any sequence of at least 10 nucleotides, with a statistically significant enrichment of UG dinucleotides and/or UGNNUG hexanucleotides, wherein Nis A, U, C or G, wherein statistically significant enrichment is defined as a probability of less than 0.2% that a random sequence of nucleotides of equal length would feature an equal number of UG dinucleotides and/or UGNNUG hexanucleotides, or wherein statistically significant enrichment is defined as a probability of less than 0.05% that a random sequence of nucleotides of equal length would feature an equal number of UG dinucleotides and/or UGNNUG hexanucleotides

10. The modified U7 snRNA construct of claim 1, wherein the antisense sequence is capable of binding to a splice donor site of the cryptic exon sequence, a splice acceptor site of the cryptic exon sequence, or one or more exonic splicing enhancers (ESE) of the cryptic exon sequence as defined by ESE finder 3.0

11. The modified U7 snRNA construct of claim 1, wherein the modified U7 snRNA construct is a U7 smOPT construct.

12. The modified U7 snRNA construct of claim 1, wherein the TDP-43 regulated cryptic exon sequence is a UNC13A cryptic exon, and the antisense sequence is at least 90% complementary to SEQ ID NO: 1 or 2, optionally at least 90% complementary to SEQ ID NO: 3 or 4.

13. The modified U7 snRNA construct of claim 12, wherein the antisense sequence is capable of binding to a TDP-43 binding region and/or flanking regions of the UNC13A cryptic exon, or wherein the antisense sequence is capable of binding to a TDP-43 binding region and/or flanking regions of the UNC13A cryptic exon and is at least 90% complementary to any one of SEQ ID NO: 23-26.

14. The modified U7 snRNA construct of claim 12, wherein the antisense sequence is capable of binding to
(i) a splice site of the UNC13A cryptic exon, or a splice site of the UNC13A cryptic exon wherein the antisense sequence is capable of binding to any one of SEQ ID NO: 19, SEQ ID NO: 20 or SEQ ID NO: 21 or 22, or
(ii) one or more exonic splice enhancer(s) (ESE) in the UNC13A cryptic exon or flanking regions thereof as defined by ESE finder 3.0, or one or more ESE in the UNC13A cryptic exon or flanking regions thereof as defined by ESE finder 3.0 wherein the antisense sequence is at least 90% complementary to any one of SEQ ID NO: 27, SEQ ID NO: 28 or SEQ ID NO: 29.

15. The modified U7 snRNA construct of claim 1, wherein the TDP-43 regulated cryptic exon sequence is a STMN2 cryptic exon, or wherein the TDP-43 regulated cryptic exon sequence is a STMN2 cryptic exon and the antisense sequence is at least 90% complementary to SEQ ID NO: 7.

16. The modified U7 snRNA construct of claim 15, wherein the antisense sequence is capable of binding to a TDP-43 binding region and/or flanking regions thereof of the STMN2 cryptic exon, or wherein the antisense sequence is capable of binding to a TDP-43 binding region and/or flanking regions thereof of the STMN2 cryptic exon and the antisense sequence is at least 90% complementary to SEQ ID NO: 12.

17. The modified U7 snRNA construct of claim 15, wherein the antisense sequence is capable of binding to
(a) the 3′-splice site of the STMN2 cryptic exon, or the 3′-splice site of the STMN2 cryptic exon, wherein the antisense sequence is at least 90% complementary to SEQ ID NO: 11 or
(b) one or more exonic splice enhancer(s) (ESE in the STMN2 cryptic exon or flanking regions thereof, as defined by ESE finder 3.0, or one or more ESE in the STMN2 cryptic exon or flanking regions thereof, as defined by ESE finder 3.0, wherein the antisense sequence is at least 90% complementary to any one of SEQ ID NO: 14-16.

18. The modified U7 snRNA construct of claim 1, wherein the TDP-43 regulated cryptic exon sequence is the INSR cryptic exon, or wherein the TDP-43 regulated cryptic exon sequence is the INSR cryptic exon and the antisense sequence is at least 90% complementary to SEQ ID NO: 9.

19. The modified U7 snRNA construct of claim 18, wherein the antisense sequence is capable of binding to
(a) a TDP-43 binding region and/or flanking regions thereof of the INSR cryptic exon, or a TDP-43 binding region and/or flanking regions thereof of the INSR cryptic exon, wherein the antisense sequence is at least 90% complementary to SEQ ID NO: 32,
(b) a 3′-splice site of the INSR cryptic exon, or a 3′-splice site of the INSR cryptic exon, wherein the antisense sequence is at least 90% complementary to SEQ ID NO: 31, or
(c) one or more ESE in the INSR cryptic exon or flanking regions thereof, or one or more ESE in the INSR cryptic exon or flanking regions thereof, wherein the antisense sequence is at least 90% complementary to any one of SEQ ID NO: 34-40.

20. The modified U7 snRNA construct of claim 1, wherein the antisense sequence a 16 nucleotide sequence with at least 90% sequence identity to SEQ ID NO 42-352 and/or wherein the antisense sequence comprises at least a 16 nucleotide sequence which has at least 90% sequence identity with at least a portion of SEQ ID NO: 420, 362, 364, 366, 368, 370, 372, 374, 382, 384, 391, 393, 395, 397, 399, 401, 403, 405, 407, 409, 411, 413, 415, 417 or 419 for the same number of nucleotides.

21. A vector that comprises or encodes for the modified U7 snRNA construct of claim 1, or a vector that comprises or encodes for the modified U7 snRNA construct, wherein the vector is a viral vector.

22. A combined vector that comprises two or more modified U7 snRNA constructs of claim 1, or a combined vector that comprises two or more of the modified U7 snRNA constructs, wherein the two or more modified U7 snRNA constructs comprise different antisense sequences that are capable of binding to different TDP-43 regulated cryptic exons.

23. A pharmaceutical composition comprising one or more of the constructs of claim 1, a vector that comprises or encodes for the construct, or one or more combined vectors that comprises or encodes two or more of the constructs.

24. The construct of claim 1, a vector that comprises or encodes for the construct, a combined vector that comprises two or more of the constructs, or a pharmaceutical composition comprising one or more of the constructs, the vector, or the one or more combined vectors, for use in therapy.

25. The construct of claim 1, a vector that comprises or encodes for the construct, a combined vector that comprises two or more of the constructs, or a pharmaceutical composition comprising one or more of the constructs, the vector, or the one or more combined vectors, for use in the treatment of a disease characterised by TDP-43 dysfunction.

26. A method of modulating splicing of a TDP-43 regulated cryptic exon, the method comprising delivering to a cell the construct of claim 1, a vector that comprises or encodes for the construct, a combined vector that comprises two or more of the constructs, or a pharmaceutical composition comprising one or more of the constructs, the vector, or the one or more combined vectors, wherein the method comprises contacting the construct with a cell to modulate splicing of the TDP-43 regulated cryptic exon.

27. The construct of claim 1, a vector that comprises or encodes for the construct, a combined vector that comprises two or more of the constructs, or a pharmaceutical composition comprising one or more of the constructs, the vector, or the one or more combined vectors, for use in the treatment of a disease characterised by TDP-43 dysfunction, wherein the disease is a neurodegenerative or muscular disease, or wherein the disease is selected from the group consisting of Amyotrophic lateral sclerosis (ALS) and frontotemporal dementia (FTD), Alzheimer's disease, Inclusion body myositis/myopathy (IBM), FOSMNN (Facial onset sensory and motor neuronopathy), and Perry Syndrome, Limbic-Predominant Age-Related TDP-43 Encephalopathy (LATE), or a combination thereof.


SEQUENCE LISTING (486 sequences)

SEQ ID NO: 1 - RNA, 991 nt, Homo sapiens
  source 1..991: mol_type = mRNA
tgggaagcccaccttggcctccaggttgactctcactactcatcatcaggttcttccttctattccagccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaaggatggatggagagatgggtgagtacatggatggatagatggatgagttggtgggtagattcgtggctagatggatgatggatggatggacagatggatggatatatgattgaactattgaaagtatagatgtatggatgggtgaatttgggggtaattgttagatgatggatgagtatagatgaatgatggatggataacttgatgagtggatagatagattgctggatagatgattgactgggtggatagatgaaatgttggatgagcagattaagttgtattggatgggatggatggaagtgtggttgagttattagaaggaagattgagtagataggtgaatttgttgatagtcagatgggtagataggtagatggatggatggatggatggatgtataggcagatggacaaatggatgaatgggtgggtggatgaatggaaggatgtgtggttgaactattgcaagtattgataattgggttcataatttctgaatatttagatggatggttgtgagtggctggtggacagacgaaaaatggatggttggataaattgatgggtggatggatggttggttgtatgaaagaatgaatgattgggtaggtggattaagttgcggatcaatgtatgggatggatgaatggatggatggatggatgtgtggttgaattactgaaaggttggaagagtggatgggtgaaatttggggtagttagatgggtgggtgtgtggatggataaaagagtagatgaatg

SEQ ID NO: 2 - RNA, 991 nt, Homo sapiens
  source 1..991: mol_type = mRNA
tgggaagcccaccttggcctccaggttgactctcactactcatcatcaggttcttccttctattccagccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaagcatggatggagagatgggtgagtacatggatggatagatggatgagttggtgggtagattcgtggctagatggatgatggatggatggacagatggatggatatatgattgaactattgaaagtatagatgtatggatgggtgaatttgggggtaattgttagatgatggatgagtatagatgaatgatggatggataacttgatgagtggatagatagattgctggatagatgattgactgggtggatagatgaaatgttggatgagcagattaagttgtattggatgggatggatggaagtgtggttgagttattagaaggaagattgagtagataggtgaatttgttgatagtcagatgggtagataggtagatggatggatggatggatggatgtataggcagatggacaaatggatgaatgggtgggtggatgaatggaaggatgtgtggttgaactattgcaagtattgataattgggttcataatttctgaatatttagatggatggttgtgagtggctggtggacagacgaaaaatggatggttggataaattgatgggtggatggatggttggttgtatgaaagaatgaatgattgggtaggtggattaagttgcggatcaatgtatgggatggatgaatggatggatggatggatgtgtggttgaattactgaaaggttggaagagtggatgggtgaaatttggggtagttagatgggtgggtgtgtggatggataaaagagtagatgaatg

SEQ ID NO: 3 - RNA, 320 nt, Homo sapiens
  source 1..320: mol_type = mRNA
tgggaagcccaccttggcctccaggttgactctcactactcatcatcaggttcttccttctattccagccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaaggatggatggagagatgggtgagtacatggatggatagatggatgagttggtgggtagattcgtggctagatggatgatggatggatggaca

SEQ ID NO: 4 - RNA, 320 nt, Homo sapiens
  source 1..320: mol_type = mRNA
tgggaagcccaccttggcctccaggttgactctcactactcatcatcaggttcttccttctattccagccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaagcatggatggagagatgggtgagtacatggatggatagatggatgagttggtgggtagattcgtggctagatggatgatggatggatggaca

SEQ ID NO: 5 - RNA, 128 nt, Homo sapiens
  source 1..128: mol_type = mRNA
ctgcctgggtttcctggaaagaactcttatccccaggaactagtttgttgaataaatgctggtgaatgaatgaatgattgaacagatgaatgagtgatgagtagataaaaggatggatggagagatgg

SEQ ID NO: 6 - RNA, 178 nt, Homo sapiens
  source 1..178: mol_type = mRNA
ccctaaccactcaggattgggccgtttgtgtctgggtatgtctcttccagctgcctgggtttcctggaaagaactcttatccccaggaactagtttgttgaataaatgctggtgaatgaatgaatgattgaacagatgaatgagtgatgagtagataaaaggatggatggagagatgg

SEQ ID NO: 7 - RNA, 279 nt, Homo sapiens
  source 1..279: mol_type = mRNA
tgccccatcactctctcttaattggatttttaaaattatattcatattgcaggactcggcagaagaccttcgagagaaaggtagaaaataagaatttggctctctgtgtgagcatgtgtgcgtgtgtgcgagagagagagacagacagcctgcctaagaagaaatgaatgtgaatgcggcttgtggcacagttgacaaggatgataaatcaataatgcaagcttactatcatttatgaatagcaatactgaagaaattaaaacaaaagattgctgtctc

SEQ ID NO: 8 - RNA, 227 nt, Homo sapiens
  source 1..227: mol_type = mRNA
gactcggcagaagaccttcgagagaaaggtagaaaataagaatttggctctctgtgtgagcatgtgtgcgtgtgtgcgagagagagagacagacagcctgcctaagaagaaatgaatgtgaatgcggcttgtggcacagttgacaaggatgataaatcaataatgcaagcttactatcatttatgaatagcaatactgaagaaattaaaacaaaagattgctgtctc

SEQ ID NO: 9 - RNA, 173 nt, Homo sapiens
  source 1..173: mol_type = mRNA
tattaatattatcactatgcttactgtgccatatagtaccggatacgggatgaagtcatacaagcactgaatgaatggatgaatgaatgatggatgaatggatgacaccttcttatatgtgtatcaggctgatgctgaagacttcaaagttgagtaaaatacctatgtcagtc

SEQ ID NO: 10 - RNA, 112 nt, Homo sapiens
  source 1..112: mol_type = mRNA
taccggatacgggatgaagtcatacaagcactgaatgaatggatgaatgaatgatggatgaatggatgacaccttcttatatgtgtatcaggctgatgctgaagacttcaaa

SEQ ID NO: 11 - RNA, 40 nt, Homo sapiens
  source 1..40: mol_type = mRNA
taaaattatattcatattgcaggactcggcagaagacctt

SEQ ID NO: 12 - RNA, 67 nt, Homo sapiens
  source 1..67: mol_type = mRNA
gtagaaaataagaatttggctctctgtgtgagcatgtgtgcgtgtgtgcgagagagagagacagaca

SEQ ID NO: 13 - RNA, 23 nt, Homo sapiens
  source 1..23: mol_type = mRNA
tgtgtgagcatgtgtgcgtgtgt

SEQ ID NO: 14 - RNA, 47 nt, Homo sapiens
  source 1..47: mol_type = mRNA
attcatattgcaggactcggcagaagaccttcgagagaaaggtagaa

SEQ ID NO: 15 - RNA, 51 nt, Homo sapiens
  source 1..51: mol_type = mRNA
taagaagaaatgaatgtgaatgcggcttgtggcacagttgacaaggatgat

SEQ ID NO: 16 - RNA, 47 nt, Homo sapiens
  source 1..47: mol_type = mRNA
aatgcggcttgtggcacagttgacaaggatgataaatcaataatgca

SEQ ID NO: 17 - RNA, 24 nt, Homo sapiens
  source 1..24: mol_type = mRNA
tgcggcttgtggcacagttgacaa

SEQ ID NO: 18 - RNA, 65 nt, Homo sapiens
  source 1..65: mol_type = mRNA
taagaagaaatgaatgtgaatgcggcttgtggcacagttgacaaggatgataaatcaataatgca

SEQ ID NO: 19 - RNA, 40 nt, Homo sapiens
  source 1..40: mol_type = mRNA
ggttcttccttctattccagccctaaccactcaggattgg

SEQ ID NO: 20 - RNA, 40 nt, Homo sapiens
  source 1..40: mol_type = mRNA
tgtctgggtatgtctcttccagctgcctgggtttcctgga

SEQ ID NO: 21 - RNA, 40 nt, Homo sapiens
  source 1..40: mol_type = mRNA
aaggatggatggagagatgggtgagtacatggatggatag

SEQ ID NO: 22 - RNA, 40 nt, Homo sapiens
  source 1..40: mol_type = mRNA
aagcatggatggagagatgggtgagtacatggatggatag

SEQ ID NO: 23 - RNA, 811 nt, Homo sapiens
  source 1..811: mol_type = mRNA
tgaatgaatgaatgattgaacagatgaatgagtgatgagtagataaaaggatggatggagagatgggtgagtacatggatggatagatggatgagttggtgggtagattcgtggctagatggatgatggatggatggacagatggatggatatatgattgaactattgaaagtatagatgtatggatgggtgaatttgggggtaattgttagatgatggatgagtatagatgaatgatggatggataacttgatgagtggatagatagattgctggatagatgattgactgggtggatagatgaaatgttggatgagcagattaagttgtattggatgggatggatggaagtgtggttgagttattagaaggaagattgagtagataggtgaatttgttgatagtcagatgggtagataggtagatggatggatggatggatggatgtataggcagatggacaaatggatgaatgggtgggtggatgaatggaaggatgtgtggttgaactattgcaagtattgataattgggttcataatttctgaatatttagatggatggttgtgagtggctggtggacagacgaaaaatggatggttggataaattgatgggtggatggatggttggttgtatgaaagaatgaatgattgggtaggtggattaagttgcggatcaatgtatgggatggatgaatggatggatggatggatgtgtggttgaattactgaaaggttggaagagtggatgggtgaaatttggggtagttagatgggtgggtgtgtggatggataaaagagtagatgaatg

SEQ ID NO: 24 - RNA, 108 nt, Homo sapiens
  source 1..108: mol_type = mRNA
gtttgttgaataaatgctggtgaatgaatgaatgattgaacagatgaatgagtgatgagtagataaaaggatggatggagagatgggtgagtacatggatggatagat

SEQ ID NO: 25 - RNA, 108 nt, Homo sapiens
  source 1..108: mol_type = mRNA
gtttgttgaataaatgctggtgaatgaatgaatgattgaacagatgaatgagtgatgagtagataaaagcatggatggagagatgggtgagtacatggatggatagat

SEQ ID NO: 26 - RNA, 22 nt, Homo sapiens
  source 1..22: mol_type = mRNA
ggatggatggagagatgggtga

SEQ ID NO: 27 - RNA, 48 nt, Homo sapiens
  source 1..48: mol_type = mRNA
cttctattccagccctaaccactcaggattgggccgtttgtgtctggg

SEQ ID NO: 28 - RNA, 48 nt, Homo sapiens
  source 1..48: mol_type = mRNA
tgtctcttccagctgcctgggtttcctggaaagaactcttatccccag

SEQ ID NO: 29 - RNA, 46 nt, Homo sapiens
  source 1..46: mol_type = mRNA
ccactcaggattgggccgtttgtgtctgggtatgtctcttccagct

SEQ ID NO: 30 - RNA, 58 nt, Homo sapiens
  source 1..58: mol_type = mRNA
actcaggattgggccgtttgtgtctgggtatgtctcttccagctgcctgggtttcctg

SEQ ID NO: 31 - RNA, 40 nt, Homo sapiens
  source 1..40: mol_type = mRNA
ctatgcttactgtgccatatagtaccggatacgggatgaa

SEQ ID NO: 32 - RNA, 127 nt, Homo sapiens
  source 1..127: mol_type = mRNA
ggatacgggatgaagtcatacaagcactgaatgaatggatgaatgaatgatggatgaatggatgacaccttcttatatgtatcaggctgatgctgaagacggatacgggatgaagtcatacaagcac

SEQ ID NO: 33 - RNA, 53 nt, Homo sapiens
  source 1..53: mol_type = mRNA
tgaatgaatggatgaatgaatgatggatgaatggatgacaccttcttatatgt

SEQ ID NO: 34 - RNA, 47 nt, Homo sapiens
  source 1..47: mol_type = mRNA
tgaatgatggatgaatggatgacaccttcttatatgtgtatcaggct

SEQ ID NO: 35 - RNA, 47 nt, Homo sapiens
  source 1..47: mol_type = mRNA
atatgtgtatcaggctgatgctgaagacttcaaagttgagtaaaata

SEQ ID NO: 36 - RNA, 48 nt, Homo sapiens
  source 1..48: mol_type = mRNA
cactgaatgaatggatgaatgaatgatggatgaatggatgacaccttc

SEQ ID NO: 37 - RNA, 55 nt, Homo sapiens
  source 1..55: mol_type = mRNA
accttcttatatgtgtatcaggctgatgctgaagacttcaaagttgagtaaaata

SEQ ID NO: 38 - RNA, 49 nt, Homo sapiens
  source 1..49: mol_type = mRNA
gtaccggatacgggatgaagtcatacaagcactgaatgaatggatgaat

SEQ ID NO: 39 - RNA, 46 nt, Homo sapiens
  source 1..46: mol_type = mRNA
ctgtgccatatagtaccggatacgggatgaagtcatacaagcactg

SEQ ID NO: 40 - RNA, 46 nt, Homo sapiens
  source 1..46: mol_type = mRNA
atggatgacaccttcttatatgtgtatcaggctgatgctgaagact

SEQ ID NO: 41 - RNA, 252 nt, synthetic construct
  source 1..252: mol_type = other RNA
aacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagaggcctttccgc

SEQ ID NO: 42 - RNA, 16 nt, synthetic construct
  source 1..16: mol_type = other RNA
gtcttctgccgagtcc

SEQ ID NO: 43 - RNA, 16 nt, synthetic construct
  source 1..16: mol_type = other RNA
gtcttctgccgagtcc

SEQ ID NO: 44 - RNA, 16 nt, synthetic construct
  source 1..16: mol_type = other RNA
tcttctgccgagtcct

SEQ ID NO: 45 - RNA, 16 nt, synthetic construct
  source 1..16: mol_type = other RNA
cttctgccgagtcctg

SEQ ID NO: 46 - RNA, 16 nt, synthetic construct
  source 1..16: mol_type = other RNA
ttctgccgagtcctgc

SEQ ID NO: 47 - RNA, 16 nt, synthetic construct
  source 1..16: mol_type = other RNA
tctgccgagtcctgca

SEQ ID NO: 48 - RNA, 16 nt, synthetic construct
  source 1..16: mol_type = other RNA
ctgccgagtcctgcaa

SEQ ID NO: 49 - RNA, 16 nt, synthetic construct
  source 1..16: mol_type = other RNA
tgccgagtcctgcaat

SEQ ID NO: 50 - RNA, 16 nt, synthetic construct
  source 1..16: mol_type = other RNA
gccgagtcctgcaata

SEQ ID NO: 51 - RNA, 16 nt, synthetic construct
  source 1..16: mol_type = other RNA
ccgagtcctgcaatat

SEQ ID NO: 52 - RNA, 16 nt, synthetic construct
  source 1..16: mol_type = other RNA
cgagtcctgcaatatg

SEQ ID NO: 53 - RNA, 16 nt, synthetic construct
  source 1..16: mol_type = other RNA
gagtcctgcaatatga

SEQ ID NO: 54 - RNA, 16 nt, synthetic construct
  source 1..16: mol_type = other RNA
agtcctgcaatatgaa

SEQ ID NO: 55 - RNA, 16 nt, synthetic construct
  source 1..16: mol_type = other RNA
gccctgcaatatgaat

SEQ ID NO: 56 - RNA, 16 nt, synthetic construct
  source 1..16: mol_type = other RNA
ccctgcaatatgaata

SEQ ID NO: 57 - RNA, 16 nt, synthetic construct
  source 1..16: mol_type = other RNA
cctgcaatatgaatat

SEQ ID NO: 58 - RNA, 16 nt, synthetic construct
  source 1..16: mol_type = other RNA
ctgcaatatgaatata

SEQ ID NO: 59 - RNA, 16 nt, synthetic construct
  source 1..16: mol_type = other RNA
tgcaatatgaatataa

SEQ ID NO: 60 - RNA, 16 nt, synthetic construct
  source 1..16: mol_type = other RNA
tgtctgtctctctctc

SEQ ID NO: 61 - RNA, 16 nt, synthetic construct
  source 1..16: mol_type = other RNA
gtctgtctctctctct

SEQ ID NO: 62 - RNA, 16 nt, synthetic construct
  source 1..16: mol_type = other RNA
tctgtctctctctctc

SEQ ID NO: 63 - RNA, 16 nt, synthetic construct
  source 1..16: mol_type = other RNA
ctgtctctctctctcg

SEQ ID NO: 64 - RNA, 16 nt, synthetic construct
  source 1..16: mol_type = other RNA
tgtctctctctctcgc

SEQ ID NO: 65 - RNA, 16 nt, synthetic construct
  source 1..16: mol_type = other RNA
gtctctctctctcgca

SEQ ID NO: 66 - RNA, 16 nt, synthetic construct
  source 1..16: mol_type = other RNA
tctctctctctcgcac

SEQ ID NO: 67 - RNA, 16 nt, synthetic construct
  source 1..16: mol_type = other RNA
ctctctctctcgcaca

SEQ ID NO: 68 - RNA, 16 nt, synthetic construct
  source 1..16: mol_type = other RNA
tctctctctcgcacac

SEQ ID NO: 69 - RNA, 16 nt, synthetic construct
  source 1..16: mol_type = other RNA
ctctctctcgcacaca

SEQ ID NO: 70 - RNA, 16 nt, synthetic construct
  source 1..16: mol_type = other RNA
tctctctcgcacacac

SEQ ID NO: 71 - RNA, 16 nt, synthetic construct
  source 1..16: mol_type = other RNA
tctctctcgcacacac

SEQ ID NO: 72 - RNA, 16 nt, synthetic construct
  source 1..16: mol_type = other RNA
tctctcgcacacacgc

SEQ ID NO: 73 - RNA, 16 nt, synthetic construct
  source 1..16: mol_type = other RNA
ctctcgcacacacgca

SEQ ID NO: 74 - RNA, 16 nt, synthetic construct
  source 1..16: mol_type = other RNA
tctcgcacacacgcac

SEQ ID NO: 75 - RNA, 16 nt, synthetic construct
  source 1..16: mol_type = other RNA
ctcgcacacacgcaca

SEQ ID NO: 76 - RNA, 16 nt, synthetic construct
  source 1..16: mol_type = other RNA
tcgcacacacgcacac

SEQ ID NO: 77 - RNA, 16 nt, synthetic construct
  source 1..16: mol_type = other RNA
cgcacacacgcacaca

SEQ ID NO: 78 - RNA, 16 nt, synthetic construct
  source 1..16: mol_type = other RNA
gcacacacgcacacat

SEQ ID NO: 79 - RNA, 16 nt, synthetic construct
  source 1..16: mol_type = other RNA
cacacacgcacacatg

SEQ ID NO: 80 - RNA, 16 nt, synthetic construct
  source 1..16: mol_type = other RNA
cacacacgcacacatg

SEQ ID NO: 81 - RNA, 16 nt, synthetic construct
  source 1..16: mol_type = other RNA
cacacgcacacatgct

SEQ ID NO: 82 - RNA, 16 nt, synthetic construct
  source 1..16: mol_type = other RNA
acacgcacacatgctc

SEQ ID NO: 83 - RNA, 16 nt, synthetic construct
  source 1..16: mol_type = other RNA
cacgcacacatgctca

SEQ ID NO: 84 - RNA, 16 nt, synthetic construct
  source 1..16: mol_type = other RNA
acgcacacatgctcac

SEQ ID NO: 85 - RNA, 16 nt, synthetic construct
  source 1..16: mol_type = other RNA
cgcacacatgctcaca

SEQ ID NO: 86 - RNA, 16 nt, synthetic construct
  source 1..16: mol_type = other RNA
gcacacatgctcacac

SEQ ID NO: 87 - RNA, 16 nt, synthetic construct
  source 1..16: mol_type = other RNA
cacacatgctcacaca

SEQ ID NO: 88 - RNA, 16 nt, synthetic construct
  source 1..16: mol_type = other RNA
acacatgctcacacag

SEQ ID NO: 89 - RNA, 16 nt, synthetic construct
  source 1..16: mol_type = other RNA
cacatgctcacacaga

SEQ ID NO: 90 - RNA, 16 nt, synthetic construct
  source 1..16: mol_type = other RNA
acatgctcacacagag

SEQ ID NO: 91 - RNA, 16 nt, synthetic construct
  source 1..16: mol_type = other RNA
catgctcacacagaga

SEQ ID NO: 92 - RNA, 16 nt, synthetic construct
  source 1..16: mol_type = other RNA
atgctcacacagagag

SEQ ID NO: 93 - RNA, 16 nt, synthetic construct
  source 1..16: mol_type = other RNA
tgctcacacagagagc

SEQ ID NO: 94 - RNA, 16 nt, synthetic construct
  source 1..16: mol_type = other RNA
gctcacacagagagcc

SEQ ID NO: 95 - RNA, 16 nt, synthetic construct
  source 1..16: mol_type = other RNA
ctcacacagagagcca

SEQ ID NO: 96 - RNA, 16 nt, synthetic construct
  source 1..16: mol_type = other RNA
tcacacagagagccaa

SEQ ID NO: 97 - RNA, 16 nt, synthetic construct
  source 1..16: mol_type = other RNA
cacacagagagccaaa

SEQ ID NO: 98 - RNA, 16 nt, synthetic construct
  source 1..16: mol_type = other RNA
acacagagagccaaat

SEQ ID NO: 99 - RNA, 16 nt, synthetic construct
  source 1..16: mol_type = other RNA
cacagagagccaaatt

SEQ ID NO: 100 - RNA, 16 nt, synthetic construct
  source 1..16: mol_type = other RNA
acagagagccaaattc

SEQ ID NO: 101 - RNA, 16 nt, synthetic construct
  source 1..16: mol_type = other RNA
cagagagccaaattct

SEQ ID NO: 102 - RNA, 16 nt, synthetic construct
  source 1..16: mol_type = other RNA
cagagagccaaattct

SEQ ID NO: 103 - RNA, 16 nt, synthetic construct
  source 1..16: mol_type = other RNA
cctgagtggttagggc

SEQ ID NO: 104 - RNA, 16 nt, synthetic construct
  source 1..16: mol_type = other RNA
ctgagtggttagggct

SEQ ID NO: 105 - RNA, 15 nt, synthetic construct
  source 1..15: mol_type = other RNA
tagtggttagggctg

SEQ ID NO: 106 - RNA, 15 nt, synthetic construct
  source 1..15: mol_type = other RNA
agtggttagggctgg

SEQ ID NO: 107 - RNA, 15 nt, synthetic construct
  source 1..15: mol_type = other RNA
gtggttagggctgga

SEQ ID NO: 108 - RNA, 15 nt, synthetic construct
  source 1..15: mol_type = other RNA
tggttagggctggaa

SEQ ID NO: 109 - RNA, 15 nt, synthetic construct
  source 1..15: mol_type = other RNA
tggttagggctggaa

SEQ ID NO: 110 - RNA, 15 nt, synthetic construct
  source 1..15: mol_type = other RNA
gttagggctggaata

SEQ ID NO: 111 - RNA, 15 nt, synthetic construct
  source 1..15: mol_type = other RNA
ttagggctggaatag

SEQ ID NO: 112 - RNA, 15 nt, synthetic construct
  source 1..15: mol_type = other RNA
tagggctggaataga

SEQ ID NO: 113 - RNA, 15 nt, synthetic construct
  source 1..15: mol_type = other RNA
agggctggaatagaa

SEQ ID NO: 114 - RNA, 15 nt, synthetic construct
  source 1..15: mol_type = other RNA
gggctggaatagaag

SEQ ID NO: 115 - RNA, 15 nt, synthetic construct
  source 1..15: mol_type = other RNA
ggctggaatagaagg

SEQ ID NO: 116 - RNA, 16 nt, synthetic construct
  source 1..16: mol_type = other RNA
gctggaatagaaggaa

SEQ ID NO: 117 - RNA, 16 nt, synthetic construct
  source 1..16: mol_type = other RNA
ctggaatagaaggaat

SEQ ID NO: 118 - RNA, 17 nt, synthetic construct
  source 1..17: mol_type = other RNA
ctggaatagaaggaata

SEQ ID NO: 119 - RNA, 16 nt, synthetic construct
  source 1..16: mol_type = other RNA
aggaaacccaggcagc

SEQ ID NO: 120 - RNA, 16 nt, synthetic construct
  source 1..16: mol_type = other RNA
ggaaacccaggcagct

SEQ ID NO: 121 - RNA, 16 nt, synthetic construct
  source 1..16: mol_type = other RNA
gaaacccaggcagctg

SEQ ID NO: 122 - RNA, 16 nt, synthetic construct
  source 1..16: mol_type = other RNA
aaacccaggcagctgg

SEQ ID NO: 123 - RNA, 16 nt, synthetic construct
  source 1..16: mol_type = other RNA
aacccaggcagctgga

SEQ ID NO: 124 - RNA, 16 nt, synthetic construct
  source 1..16: mol_type = other RNA
acccaggcagctggaa

SEQ ID NO: 125 - RNA, 16 nt, synthetic construct
  source 1..16: mol_type = other RNA
cccaggcagctggaag

SEQ ID NO: 126 - RNA, 16 nt, synthetic construct
  source 1..16: mol_type = other RNA
ccaggcagctggaaga

SEQ ID NO: 127 - RNA, 16 nt, synthetic construct
  source 1..16: mol_type = other RNA
caggcagctggaagag

SEQ ID NO: 128 - RNA, 16 nt, synthetic construct
  source 1..16: mol_type = other RNA
aggcagctggaagaga

SEQ ID NO: 129 - RNA, 16 nt, synthetic construct
  source 1..16: mol_type = other RNA
aggcagctggaagaga

SEQ ID NO: 130 - RNA, 16 nt, synthetic construct
  source 1..16: mol_type = other RNA
gcagctggaagagaca

SEQ ID NO: 131 - RNA, 16 nt, synthetic construct
  source 1..16: mol_type = other RNA
cagctggaagagacat

SEQ ID NO: 132 - RNA, 16 nt, synthetic construct
  source 1..16: mol_type = other RNA
agctggaagagacata

SEQ ID NO: 133 - RNA, 16 nt, synthetic construct
  source 1..16: mol_type = other RNA
gctggaagagacatac

SEQ ID NO: 134 - RNA, 16 nt, synthetic construct
  source 1..16: mol_type = other RNA
ctggaagagacatacc

SEQ ID NO: 135 - RNA, 16 nt, synthetic construct
  source 1..16: mol_type = other RNA
tggaagagacataccc

SEQ ID NO: 136 - RNA, 16 nt, synthetic construct
  source 1..16: mol_type = other RNA
tccatccatgtactca

SEQ ID NO: 137 - RNA, 16 nt, synthetic construct
  source 1..16: mol_type = other RNA
ccatccatgtactcac

SEQ ID NO: 138 - RNA, 16 nt, synthetic construct
  source 1..16: mol_type = other RNA
ccatccatgtactcac

SEQ ID NO: 139 - RNA, 16 nt, synthetic construct
  source 1..16: mol_type = other RNA
atccatgtactcaccc

SEQ ID NO: 140 - RNA, 16 nt, synthetic construct
  source 1..16: mol_type = other RNA
atccatgtactcaccc

SEQ ID NO: 141 - RNA, 16 nt, synthetic construct
  source 1..16: mol_type = other RNA
ccatgtactcacccat

SEQ ID NO: 142 - RNA, 16 nt, synthetic construct
  source 1..16: mol_type = other RNA
catgtactcacccatc

SEQ ID NO: 143 - RNA, 16 nt, synthetic construct
  source 1..16: mol_type = other RNA
atgtactcacccatct

SEQ ID NO: 144 - RNA, 16 nt, synthetic construct
  source 1..16: mol_type = other RNA
tgtactcacccatctc

SEQ ID NO: 145 - RNA, 16 nt, synthetic construct
  source 1..16: mol_type = other RNA
gtactcacccatctct

SEQ ID NO: 146 - RNA, 16 nt, synthetic construct
  source 1..16: mol_type = other RNA
tactcacccatctctc

SEQ ID NO: 147 - RNA, 16 nt, synthetic construct
  source 1..16: mol_type = other RNA
actcacccatctctcc

SEQ ID NO: 148 - RNA, 16 nt, synthetic construct
  source 1..16: mol_type = other RNA
ctcacccatctctcca

SEQ ID NO: 149 - RNA, 16 nt, synthetic construct
  source 1..16: mol_type = other RNA
tcacccatctctccat

SEQ ID NO: 150 - RNA, 16 nt, synthetic construct
  source 1..16: mol_type = other RNA
cacccatctctccatc

SEQ ID NO: 151 - RNA, 16 nt, synthetic construct
  source 1..16: mol_type = other RNA
cacccatctctccatc

SEQ ID NO: 152 - RNA, 16 nt, synthetic construct
  source 1..16: mol_type = other RNA
cccatctctccatcca

SEQ ID NO: 153 - RNA, 16 nt, synthetic construct
  source 1..16: mol_type = other RNA
atctatccatccatgt

SEQ ID NO: 154 - RNA, 16 nt, synthetic construct
  source 1..16: mol_type = other RNA
tctatccatccatgta

SEQ ID NO: 155 - RNA, 16 nt, synthetic construct
  source 1..16: mol_type = other RNA
ctatccatccatgtac

SEQ ID NO: 156 - RNA, 16 nt, synthetic construct
  source 1..16: mol_type = other RNA
tatccatccatgtact

SEQ ID NO: 157 - RNA, 16 nt, synthetic construct
  source 1..16: mol_type = other RNA
atccatccatgtactc

SEQ ID NO: 158 - RNA, 16 nt, synthetic construct
  source 1..16: mol_type = other RNA
tccatccatgtactca

SEQ ID NO: 159 - RNA, 16 nt, synthetic construct
  source 1..16: mol_type = other RNA
ccatccatgtactcac

SEQ ID NO: 160 - RNA, 16 nt, synthetic construct
  source 1..16: mol_type = other RNA
catccatgtactcacc

SEQ ID NO: 161 - RNA, 16 nt, synthetic construct
  source 1..16: mol_type = other RNA
atccatgtactcaccc

SEQ ID NO: 162 - RNA, 16 nt, synthetic construct
  source 1..16: mol_type = other RNA
tccatgtactcaccca

SEQ ID NO: 163 - RNA, 16 nt, synthetic construct
  source 1..16: mol_type = other RNA
tccatgtactcaccca

SEQ ID NO: 164 - RNA, 15 nt, synthetic construct
  source 1..15: mol_type = other RNA
atgtactcacccatc

SEQ ID NO: 165 - RNA, 15 nt, synthetic construct
  source 1..15: mol_type = other RNA
tgtactcacccatct

SEQ ID NO: 166 - RNA, 15 nt, synthetic construct
  source 1..15: mol_type = other RNA
gtactcacccatctc

SEQ ID NO: 167 - RNA, 15 nt, synthetic construct
  source 1..15: mol_type = other RNA
tactcacccatctct

SEQ ID NO: 168 - RNA, 15 nt, synthetic construct
  source 1..15: mol_type = other RNA
actcacccatctctc

SEQ ID NO: 169 - RNA, 15 nt, synthetic construct
  source 1..15: mol_type = other RNA
ctcacccatctctcc

SEQ ID NO: 170 - RNA, 15 nt, synthetic construct
  source 1..15: mol_type = other RNA
tcacccatctctcca

SEQ ID NO: 171 - RNA, 15 nt, synthetic construct
  source 1..15: mol_type = other RNA
cacccatctctccat

SEQ ID NO: 172 - RNA, 15 nt, synthetic construct
  source 1..15: mol_type = other RNA
acccatctctccatc

SEQ ID NO: 173 - RNA, 15 nt, synthetic construct
  source 1..15: mol_type = other RNA
cccatctctccatcc

SEQ ID NO: 174 - RNA, 15 nt, synthetic construct
  source 1..15: mol_type = other RNA
ccatctctccatcca

SEQ ID NO: 175 - RNA, 15 nt, synthetic construct
  source 1..15: mol_type = other RNA
catctctccatccat

SEQ ID NO: 176 - RNA, 15 nt, synthetic construct
  source 1..15: mol_type = other RNA
atctctccatccatc

SEQ ID NO: 177 - RNA, 15 nt, synthetic construct
  source 1..15: mol_type = other RNA
atctctccatccatg

SEQ ID NO: 178 - RNA, 15 nt, synthetic construct
  source 1..15: mol_type = other RNA
tctctccatccatcc

SEQ ID NO: 179 - RNA, 15 nt, synthetic construct
  source 1..15: mol_type = other RNA
tctctccatccatgc

SEQ ID NO: 180 - RNA, 15 nt, synthetic construct
  source 1..15: mol_type = other RNA
ctctccatccatcct

SEQ ID NO: 181 - RNA, 15 nt, synthetic construct
  source 1..15: mol_type = other RNA
ctctccatccatgct

SEQ ID NO: 182 - RNA, 15 nt, synthetic construct
  source 1..15: mol_type = other RNA
tctccatccatcctt

SEQ ID NO: 183 - RNA, 15 nt, synthetic construct
  source 1..15: mol_type = other RNA
tctccatccatgctt

SEQ ID NO: 184 - RNA, 15 nt, synthetic construct
  source 1..15: mol_type = other RNA
ctccatccatccttt

SEQ ID NO: 185 - RNA, 15 nt, synthetic construct
  source 1..15: mol_type = other RNA
ctccatccatgcttt

SEQ ID NO: 186 - RNA, 15 nt, synthetic construct
  source 1..15: mol_type = other RNA
tccatccatcctttt

SEQ ID NO: 187 - RNA, 15 nt, synthetic construct
  source 1..15: mol_type = other RNA
tccatccatgctttt

SEQ ID NO: 188 - RNA, 15 nt, synthetic construct
  source 1..15: mol_type = other RNA
ccatccatcctttta

SEQ ID NO: 189 - RNA, 15 nt, synthetic construct
  source 1..15: mol_type = other RNA
ccatccatgctttta

SEQ ID NO: 190 - RNA, 15 nt, synthetic construct
  source 1..15: mol_type = other RNA
catccatccttttat

SEQ ID NO: 191 - RNA, 15 nt, synthetic construct
  source 1..15: mol_type = other RNA
catccatgcttttat

SEQ ID NO: 192 - RNA, 15 nt, synthetic construct
  source 1..15: mol_type = other RNA
atccatccttttatc

SEQ ID NO: 193 - RNA, 15 nt, synthetic construct
  source 1..15: mol_type = other RNA
atccatgcttttatc

SEQ ID NO: 194 - RNA, 15 nt, synthetic construct
  source 1..15: mol_type = other RNA
tccatccttttatct

SEQ ID NO: 195 - RNA, 15 nt, synthetic construct
  source 1..15: mol_type = other RNA
tccatgcttttatct

SEQ ID NO: 196 - RNA, 15 nt, synthetic construct
  source 1..15: mol_type = other RNA
ccatccttttatcta

SEQ ID NO: 197 - RNA, 15 nt, synthetic construct
  source 1..15: mol_type = other RNA
ccatgcttttatcta

SEQ ID NO: 198 - RNA, 15 nt, synthetic construct
  source 1..15: mol_type = other RNA
catccttttatctac

SEQ ID NO: 199 - RNA, 15 nt, synthetic construct
  source 1..15: mol_type = other RNA
catgcttttatctac

SEQ ID NO: 200 - RNA, 15 nt, synthetic construct
  source 1..15: mol_type = other RNA
atccttttatctact

SEQ ID NO: 201 - RNA, 15 nt, synthetic construct
  source 1..15: mol_type = other RNA
atgcttttatctact

SEQ ID NO: 202 - RNA, 15 nt, synthetic construct
  source 1..15: mol_type = other RNA
tccttttatctactc

SEQ ID NO: 203 - RNA, 15 nt, synthetic construct
  source 1..15: mol_type = other RNA
tgcttttatctactc

SEQ ID NO: 204 - RNA, 15 nt, synthetic construct
  source 1..15: mol_type = other RNA
ccttttatctactca

SEQ ID NO: 205 - RNA, 15 nt, synthetic construct
  source 1..15: mol_type = other RNA
gcttttatctactca

SEQ ID NO: 206 - RNA, 15 nt, synthetic construct
  source 1..15: mol_type = other RNA
cttttatctactcat

SEQ ID NO: 207 - RNA, 15 nt, synthetic construct
  source 1..15: mol_type = other RNA
ttttatctactcatc

SEQ ID NO: 208 - RNA, 15 nt, synthetic construct
  source 1..15: mol_type = other RNA
tttatctactcatca

SEQ ID NO: 209 - RNA, 15 nt, synthetic construct
  source 1..15: mol_type = other RNA
ttatctactcatcac

SEQ ID NO: 210 - RNA, 15 nt, synthetic construct
  source 1..15: mol_type = other RNA
tatctactcatcact

SEQ ID NO: 211 - RNA, 15 nt, synthetic construct
  source 1..15: mol_type = other RNA
atctactcatcactc

SEQ ID NO: 212 - RNA, 15 nt, synthetic construct
  source 1..15: mol_type = other RNA
tctactcatcactca

SEQ ID NO: 213 - RNA, 15 nt, synthetic construct
  source 1..15: mol_type = other RNA
ctactcatcactcat

SEQ ID NO: 214 - RNA, 15 nt, synthetic construct
  source 1..15: mol_type = other RNA
tactcatcactcatt

SEQ ID NO: 215 - RNA, 15 nt, synthetic construct
  source 1..15: mol_type = other RNA
actcatcactcattc

SEQ ID NO: 216 - RNA, 15 nt, synthetic construct
  source 1..15: mol_type = other RNA
ctcatcactcattca

SEQ ID NO: 217 - RNA, 15 nt, synthetic construct
  source 1..15: mol_type = other RNA
tcatcactcattcat

SEQ ID NO: 218 - RNA, 15 nt, synthetic construct
  source 1..15: mol_type = other RNA
catcactcattcatc

SEQ ID NO: 219 - RNA, 15 nt, synthetic construct
  source 1..15: mol_type = other RNA
atcactcattcatct

SEQ ID NO: 220 - RNA, 15 nt, synthetic construct
  source 1..15: mol_type = other RNA
tcactcattcatctg

SEQ ID NO: 221 - RNA, 15 nt, synthetic construct
  source 1..15: mol_type = other RNA
cactcattcatctgt

SEQ ID NO: 222 - RNA, 15 nt, synthetic construct
  source 1..15: mol_type = other RNA
actcattcatctgtt

SEQ ID NO: 223 - RNA, 15 nt, synthetic construct
  source 1..15: mol_type = other RNA
ctcattcatctgttc

SEQ ID NO: 224 - RNA, 15 nt, synthetic construct
  source 1..15: mol_type = other RNA
tcattcatctgttca

SEQ ID NO: 225 - RNA, 15 nt, synthetic construct
  source 1..15: mol_type = other RNA
cattcatctgttcaa

SEQ ID NO: 226 - RNA, 15 nt, synthetic construct
  source 1..15: mol_type = other RNA
attcatctgttcaat

SEQ ID NO: 227 - RNA, 15 nt, synthetic construct
  source 1..15: mol_type = other RNA
ttcatctgttcaatc

SEQ ID NO: 228 - RNA, 15 nt, synthetic construct
  source 1..15: mol_type = other RNA
tcatctgttcaatca

SEQ ID NO: 229 - RNA, 15 nt, synthetic construct
  source 1..15: mol_type = other RNA
catctgttcaatcat

SEQ ID NO: 230 - RNA, 15 nt, synthetic construct
  source 1..15: mol_type = other RNA
atctgttcaatcatt

SEQ ID NO: 231 - RNA, 15 nt, synthetic construct
  source 1..15: mol_type = other RNA
tctgttcaatcattc

SEQ ID NO: 232 - RNA, 15 nt, synthetic construct
  source 1..15: mol_type = other RNA
ctgttcaatcattca

SEQ ID NO: 233 - RNA, 15 nt, synthetic construct
  source 1..15: mol_type = other RNA
tgttcaatcattcat

SEQ ID NO: 234 - RNA, 15 nt, synthetic construct
  source 1..15: mol_type = other RNA
gttcaatcattcatt

SEQ ID NO: 235 - RNA, 15 nt, synthetic construct
  source 1..15: mol_type = other RNA
ttcaatcattcattc

SEQ ID NO: 236 - RNA, 15 nt, synthetic construct
  source 1..15: mol_type = other RNA
tcaatcattcattca

SEQ ID NO: 237 - RNA, 15 nt, synthetic construct
  source 1..15: mol_type = other RNA
caatcattcattcat

SEQ ID NO: 238 - RNA, 15 nt, synthetic construct
  source 1..15: mol_type = other RNA
aatcattcattcatt

SEQ ID NO: 239 - RNA, 15 nt, synthetic construct
  source 1..15: mol_type = other RNA
atcattcattcattc

SEQ ID NO: 240 - RNA, 15 nt, synthetic construct
  source 1..15: mol_type = other RNA
tcattcattcattca

SEQ ID NO: 241 - RNA, 15 nt, synthetic construct
  source 1..15: mol_type = other RNA
cattcattcattcac

SEQ ID NO: 242 - RNA, 15 nt, synthetic construct
  source 1..15: mol_type = other RNA
attcattcattcacc

SEQ ID NO: 243 - RNA, 15 nt, synthetic construct
  source 1..15: mol_type = other RNA
ttcattcattcacca

SEQ ID NO: 244 - RNA, 15 nt, synthetic construct
  source 1..15: mol_type = other RNA
tcattcattcaccag

SEQ ID NO: 245 - RNA, 15 nt, synthetic construct
  source 1..15: mol_type = other RNA
cattcattcaccagc

SEQ ID NO: 246 - RNA, 15 nt, synthetic construct
  source 1..15: mol_type = other RNA
attcattcaccagca

SEQ ID NO: 247 - RNA, 15 nt, synthetic construct
  source 1..15: mol_type = other RNA
ttcattcaccagcat

SEQ ID NO: 248 - RNA, 15 nt, synthetic construct
  source 1..15: mol_type = other RNA
tcattcaccagcatt

SEQ ID NO: 249 - RNA, 15 nt, synthetic construct
  source 1..15: mol_type = other RNA
cattcaccagcattt

SEQ ID NO: 250 - RNA, 15 nt, synthetic construct
  source 1..15: mol_type = other RNA
attcaccagcattta

SEQ ID NO: 251 - RNA, 15 nt, synthetic construct
  source 1..15: mol_type = other RNA
ttcaccagcatttat

SEQ ID NO: 252 - RNA, 15 nt, synthetic construct
  source 1..15: mol_type = other RNA
tcaccagcatttatt

SEQ ID NO: 253 - RNA, 15 nt, synthetic construct
  source 1..15: mol_type = other RNA
caccagcatttattc

SEQ ID NO: 254 - RNA, 15 nt, synthetic construct
  source 1..15: mol_type = other RNA
accagcatttattca

SEQ ID NO: 255 - RNA, 15 nt, synthetic construct
  source 1..15: mol_type = other RNA
ccagcatttattcaa

SEQ ID NO: 256 - RNA, 15 nt, synthetic construct
  source 1..15: mol_type = other RNA
cagcatttattcaac

SEQ ID NO: 257 - RNA, 15 nt, synthetic construct
  source 1..15: mol_type = other RNA
agcatttattcaaca

SEQ ID NO: 258 - RNA, 15 nt, synthetic construct
  source 1..15: mol_type = other RNA
gcatttattcaacaa

SEQ ID NO: 259 - RNA, 15 nt, synthetic construct
  source 1..15: mol_type = other RNA
catttattcaacaaa

SEQ ID NO: 260 - RNA, 15 nt, synthetic construct
  source 1..15: mol_type = other RNA
atttattcaacaaac

SEQ ID NO: 261 - RNA, 16 nt, synthetic construct
  source 1..16: mol_type = other RNA
atcccgtatccggtac

SEQ ID NO: 262 - RNA, 16 nt, synthetic construct
  source 1..16: mol_type = other RNA
tcccgtatccggtact

SEQ ID NO: 263 - RNA, 16 nt, synthetic construct
  source 1..16: mol_type = other RNA
cccgtatccggtacta

SEQ ID NO: 264 - RNA, 16 nt, synthetic construct
  source 1..16: mol_type = other RNA
ccgtatccggtactat

SEQ ID NO: 265 - RNA, 16 nt, synthetic construct
  source 1..16: mol_type = other RNA
cgtatccggtactata

SEQ ID NO: 266 - RNA, 16 nt, synthetic construct
  source 1..16: mol_type = other RNA
gtatccggtactatat

SEQ ID NO: 267 - RNA, 16 nt, synthetic construct
  source 1..16: mol_type = other RNA
tatccggtactatatg

SEQ ID NO: 268 - RNA, 16 nt, synthetic construct
  source 1..16: mol_type = other RNA
atccggtactatatgg

SEQ ID NO: 269 - RNA, 16 nt, synthetic construct
  source 1..16: mol_type = other RNA
tccggtactatatggc

SEQ ID NO: 270 - RNA, 16 nt, synthetic construct
  source 1..16: mol_type = other RNA
ccggtactatatggca

SEQ ID NO: 271 - RNA, 16 nt, synthetic construct
  source 1..16: mol_type = other RNA
cggtactatatggcac

SEQ ID NO: 272 - RNA, 16 nt, synthetic construct
  source 1..16: mol_type = other RNA
cggtactatatggcac

SEQ ID NO: 273 - RNA, 16 nt, synthetic construct
  source 1..16: mol_type = other RNA
atactatatggcacag

SEQ ID NO: 274 - RNA, 16 nt, synthetic construct
  source 1..16: mol_type = other RNA
tactatatggcacagt

SEQ ID NO: 275 - RNA, 16 nt, synthetic construct
  source 1..16: mol_type = other RNA
actatatggcacagta

SEQ ID NO: 276 - RNA, 16 nt, synthetic construct
  source 1..16: mol_type = other RNA
ctatatggcacagtaa

SEQ ID NO: 277 - RNA, 16 nt, synthetic construct
  source 1..16: mol_type = other RNA
tatatggcacagtaag

SEQ ID NO: 278 - RNA, 16 nt, synthetic construct
  source 1..16: mol_type = other RNA
gtcttcagcatcagcc

SEQ ID NO: 279 - RNA, 16 nt, synthetic construct
  source 1..16: mol_type = other RNA
tcttcagcatcagcct

SEQ ID NO: 280 - RNA, 16 nt, synthetic construct
  source 1..16: mol_type = other RNA
cttcagcatcagcctg

SEQ ID NO: 281 - RNA, 16 nt, synthetic construct
  source 1..16: mol_type = other RNA
ttcagcatcagcctga

SEQ ID NO: 282 - RNA, 16 nt, synthetic construct
  source 1..16: mol_type = other RNA
tcagcatcagcctgat

SEQ ID NO: 283 - RNA, 16 nt, synthetic construct
  source 1..16: mol_type = other RNA
cagcatcagcctgata

SEQ ID NO: 284 - RNA, 16 nt, synthetic construct
  source 1..16: mol_type = other RNA
agcatcagcctgatac

SEQ ID NO: 285 - RNA, 16 nt, synthetic construct
  source 1..16: mol_type = other RNA
gcatcagcctgataca

SEQ ID NO: 286 - RNA, 16 nt, synthetic construct
  source 1..16: mol_type = other RNA
catcagcctgatacat

SEQ ID NO: 287 - RNA, 16 nt, synthetic construct
  source 1..16: mol_type = other RNA
atcagcctgatacata

SEQ ID NO: 288 - RNA, 16 nt, synthetic construct
  source 1..16: mol_type = other RNA
tcagcctgatacatat

SEQ ID NO: 289 - RNA, 16 nt, synthetic construct
  source 1..16: mol_type = other RNA
cagcctgatacatata

SEQ ID NO: 290 - RNA, 16 nt, synthetic construct
  source 1..16: mol_type = other RNA
agcctgatacatataa

SEQ ID NO: 291 - RNA, 16 nt, synthetic construct
  source 1..16: mol_type = other RNA
gcctgatacatataag

SEQ ID NO: 292 - RNA, 16 nt, synthetic construct
  source 1..16: mol_type = other RNA
cctgatacatataaga

SEQ ID NO: 293 - RNA, 16 nt, synthetic construct
  source 1..16: mol_type = other RNA
ctgatacatataagaa

SEQ ID NO: 294 - RNA, 16 nt, synthetic construct
  source 1..16: mol_type = other RNA
tgatacatataagaag

SEQ ID NO: 295 - RNA, 16 nt, synthetic construct
  source 1..16: mol_type = other RNA
gatacatataagaagg

SEQ ID NO: 296 - RNA, 16 nt, synthetic construct
  source 1..16: mol_type = other RNA
atacatataagaaggt

SEQ ID NO: 297 - RNA, 16 nt, synthetic construct
  source 1..16: mol_type = other RNA
tacatataagaaggtg

SEQ ID NO: 298 - RNA, 15 nt, synthetic construct
  source 1..15: mol_type = other RNA
acatataagaaggtg

SEQ ID NO: 299 - RNA, 15 nt, synthetic construct
  source 1..15: mol_type = other RNA
catataagaaggtgt

SEQ ID NO: 300 - RNA, 16 nt, synthetic construct
  source 1..16: mol_type = other RNA
acatataagaaggtgt

SEQ ID NO: 301 - RNA, 16 nt, synthetic construct
  source 1..16: mol_type = other RNA
catataagaaggtgtc

SEQ ID NO: 302 - RNA, 16 nt, synthetic construct
  source 1..16: mol_type = other RNA
atataagaaggtgtca

SEQ ID NO: 303 - RNA, 16 nt, synthetic construct
  source 1..16: mol_type = other RNA
tataagaaggtgtcat

SEQ ID NO: 304 - RNA, 16 nt, synthetic construct
  source 1..16: mol_type = other RNA
ataagaaggtgtcatc

SEQ ID NO: 305 - RNA, 16 nt, synthetic construct
  source 1..16: mol_type = other RNA
taagaaggtgtcatcc

SEQ ID NO: 306 - RNA, 16 nt, synthetic construct
  source 1..16: mol_type = other RNA
aagaaggtgtcatcca

SEQ ID NO: 307 - RNA, 16 nt, synthetic construct
  source 1..16: mol_type = other RNA
agaaggtgtcatccat

SEQ ID NO: 308 - RNA, 16 nt, synthetic construct
  source 1..16: mol_type = other RNA
gaaggtgtcatccatt

SEQ ID NO: 309 - RNA, 16 nt, synthetic construct
  source 1..16: mol_type = other RNA
aaggtgtcatccattc

SEQ ID NO: 310 - RNA, 16 nt, synthetic construct
  source 1..16: mol_type = other RNA
aggtgtcatccattca

SEQ ID NO: 311 - RNA, 16 nt, synthetic construct
  source 1..16: mol_type = other RNA
ggtgtcatccattcat

SEQ ID NO: 312 - RNA, 16 nt, synthetic construct
  source 1..16: mol_type = other RNA
gtgtcatccattcatc

SEQ ID NO: 313 - RNA, 16 nt, synthetic construct
  source 1..16: mol_type = other RNA
tgtcatccattcatcc

SEQ ID NO: 314 - RNA, 16 nt, synthetic construct
  source 1..16: mol_type = other RNA
gtcatccattcatcca

SEQ ID NO: 315 - RNA, 16 nt, synthetic construct
  source 1..16: mol_type = other RNA
tcatccattcatccat

SEQ ID NO: 316 - RNA, 16 nt, synthetic construct
  source 1..16: mol_type = other RNA
catccattcatccatc

SEQ ID NO: 317 - RNA, 16 nt, synthetic construct
  source 1..16: mol_type = other RNA
atccattcatccatca

SEQ ID NO: 318 - RNA, 16 nt, synthetic construct
  source 1..16: mol_type = other RNA
atccattcatccatca

SEQ ID NO: 319 - RNA, 16 nt, synthetic construct
  source 1..16: mol_type = other RNA
ccattcatccatcatt

SEQ ID NO: 320 - RNA, 16 nt, synthetic construct
  source 1..16: mol_type = other RNA
cattcatccatcattc

SEQ ID NO: 321 - RNA, 16 nt, synthetic construct
  source 1..16: mol_type = other RNA
cattcatccatcattc

SEQ ID NO: 322 - RNA, 16 nt, synthetic construct
  source 1..16: mol_type = other RNA
ttcatccatcattcat

SEQ ID NO: 323 - RNA, 16 nt, synthetic construct
  source 1..16: mol_type = other RNA
tcatccatcattcatt

SEQ ID NO: 324 - RNA, 16 nt, synthetic construct
  source 1..16: mol_type = other RNA
catccatcattcattc

SEQ ID NO: 325 - RNA, 16 nt, synthetic construct
  source 1..16: mol_type = other RNA
atccatcattcattca

SEQ ID NO: 326 - RNA, 16 nt, synthetic construct
  source 1..16: mol_type = other RNA
tccatcattcattcat

SEQ ID NO: 327 - RNA, 16 nt, synthetic construct
  source 1..16: mol_type = other RNA
ccatcattcattcatc

SEQ ID NO: 328 - RNA, 16 nt, synthetic construct
  source 1..16: mol_type = other RNA
catcattcattcatcc

SEQ ID NO: 329 - RNA, 16 nt, synthetic construct
  source 1..16: mol_type = other RNA
atcattcattcatcca

SEQ ID NO: 330 - RNA, 16 nt, synthetic construct
  source 1..16: mol_type = other RNA
tcattcattcatccat

SEQ ID NO: 331 - RNA, 16 nt, synthetic construct
  source 1..16: mol_type = other RNA
cattcattcatccatt

SEQ ID NO: 332 - RNA, 16 nt, synthetic construct
  source 1..16: mol_type = other RNA
attcattcatccattc

SEQ ID NO: 333 - RNA, 16 nt, synthetic construct
  source 1..16: mol_type = other RNA
ttcattcatccattca

SEQ ID NO: 334 - RNA, 16 nt, synthetic construct
  source 1..16: mol_type = other RNA
tcattcatccattcat

SEQ ID NO: 335 - RNA, 16 nt, synthetic construct
  source 1..16: mol_type = other RNA
cattcatccattcatt

SEQ ID NO: 336 - RNA, 16 nt, synthetic construct
  source 1..16: mol_type = other RNA
attcatccattcattc

SEQ ID NO: 337 - RNA, 16 nt, synthetic construct
  source 1..16: mol_type = other RNA
ttcatccattcattca

SEQ ID NO: 338 - RNA, 16 nt, synthetic construct
  source 1..16: mol_type = other RNA
tcatccattcattcag

SEQ ID NO: 339 - RNA, 16 nt, synthetic construct
  source 1..16: mol_type = other RNA
catccattcattcagt

SEQ ID NO: 340 - RNA, 16 nt, synthetic construct
  source 1..16: mol_type = other RNA
atccattcattcagtg

SEQ ID NO: 341 - RNA, 16 nt, synthetic construct
  source 1..16: mol_type = other RNA
tccattcattcagtgc

SEQ ID NO: 342 - RNA, 16 nt, synthetic construct
  source 1..16: mol_type = other RNA
ccattcattcagtgct

SEQ ID NO: 343 - RNA, 16 nt, synthetic construct
  source 1..16: mol_type = other RNA
cattcattcagtgctt

SEQ ID NO: 344 - RNA, 16 nt, synthetic construct
  source 1..16: mol_type = other RNA
attcattcagtgcttg

SEQ ID NO: 345 - RNA, 16 nt, synthetic construct
  source 1..16: mol_type = other RNA
ttcattcagtgcttgt

SEQ ID NO: 346 - RNA, 16 nt, synthetic construct
  source 1..16: mol_type = other RNA
tcattcagtgcttgta

SEQ ID NO: 347 - RNA, 16 nt, synthetic construct
  source 1..16: mol_type = other RNA
cattcagtgcttgtat

SEQ ID NO: 348 - RNA, 16 nt, synthetic construct
  source 1..16: mol_type = other RNA
attcagtgcttgtatg

SEQ ID NO: 349 - RNA, 16 nt, synthetic construct
  source 1..16: mol_type = other RNA
ttcagtgcttgtatga

SEQ ID NO: 350 - RNA, 16 nt, synthetic construct
  source 1..16: mol_type = other RNA
tcagtgcttgtatgac

SEQ ID NO: 351 - RNA, 16 nt, synthetic construct
  source 1..16: mol_type = other RNA
cagtgcttgtatgact

SEQ ID NO: 352 - RNA, 16 nt, synthetic construct
  source 1..16: mol_type = other RNA
agtgcttgtatgactt

SEQ ID NO: 353 - RNA, 10 nt, synthetic construct
  source 1..10: mol_type = other RNA
atttgtctag

SEQ ID NO: 354 - RNA, 20 nt, synthetic construct
  source 1..20: mol_type = other RNA
aagtgttacagctcttttag

SEQ ID NO: 355 - RNA, 11 nt, synthetic construct
  source 1..11: mol_type = other RNA
aatttttggag

SEQ ID NO: 356 - RNA, 31 nt, synthetic construct
  source 1..31: mol_type = other RNA
caggttttctgacttcggtcggaaaacccct

SEQ ID NO: 357 - RNA, 14 nt, synthetic construct
  source 1..14: mol_type = other RNA
gtctacaatgaaag

SEQ ID NO: 358 - RNA, 87 nt, synthetic construct
  source 1..87: mol_type = other RNA
aatatgatagggacttagggtgttcatctgttcaatcattcattcaatttttggagcaggttttctgacttcggtcggaaaacccct

SEQ ID NO: 359 - RNA, 454 nt, synthetic construct
  source 1..454: mol_type = other RNA
gaattccaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagaggcctttccgcaatatgatagggacttagggtgttcatctgttcaatcattcattcaatttttggagcaggttttctgacttcggtcggaaaacccctcccaagttaactggtctacaatgaaagcaaaacagttctcttccccgctccccggtgtgtgagaggggctttgatccttctctggtttcctaggaaacgcgtaagctt

SEQ ID NO: 360 - RNA, 84 nt, synthetic construct
  source 1..84: mol_type = other RNA
aatatgatagggacttagggtgatctgttcaatcattcattcaatttttggagcaggttttctgacttcggtcggaaaacccct

SEQ ID NO: 361 - RNA, 20 nt, synthetic construct
  source 1..20: mol_type = other RNA
tatgatagggacttagggtg

SEQ ID NO: 362 - RNA, 20 nt, synthetic construct
  source 1..20: mol_type = other RNA
atctgttcaatcattcattc

SEQ ID NO: 363 - RNA, 83 nt, synthetic construct
  source 1..83: mol_type = other RNA
aatatgatagggacttagggtgttcatctgttcaatcattcaatttttggagcaggttttctgacttcggtcggaaaacccct

SEQ ID NO: 364 - RNA, 19 nt, synthetic construct
  source 1..19: mol_type = other RNA
ttcatctgttcaatcattc

SEQ ID NO: 365 - RNA, 84 nt, synthetic construct
  source 1..84: mol_type = other RNA
aatatgatagggacttagggtgactcattcatctgttcaatcaatttttggagcaggttttctgacttcggtcggaaaacccct

SEQ ID NO: 366 - RNA, 20 nt, synthetic construct
  source 1..20: mol_type = other RNA
actcattcatctgttcaatc

SEQ ID NO: 367 - RNA, 84 nt, synthetic construct
  source 1..84: mol_type = other RNA
aatatgatagggacttagggtgactcatcactcattcatctgaatttttggagcaggttttctgacttcggtcggaaaacccct

SEQ ID NO: 368 - RNA, 20 nt, synthetic construct
  source 1..20: mol_type = other RNA
actcatcactcattcatctg

SEQ ID NO: 369 - RNA, 85 nt, synthetic construct
  source 1..85: mol_type = other RNA
aatatgatagggacttagggtgtctactcatcactcattcatcaatttttggagcaggttttctgacttcggtcggaaaacccct

SEQ ID NO: 370 - RNA, 21 nt, synthetic construct
  source 1..21: mol_type = other RNA
tctactcatcactcattcatc

SEQ ID NO: 371 - RNA, 87 nt, synthetic construct
  source 1..87: mol_type = other RNA
aatatgatagggacttagggtgtatctactcatcactcattcatcaatttttggagcaggttttctgacttcggtcggaaaacccct

SEQ ID NO: 372 - RNA, 23 nt, synthetic construct
  source 1..23: mol_type = other RNA
tatctactcatcactcattcatc

SEQ ID NO: 373 - RNA, 84 nt, synthetic construct
  source 1..84: mol_type = other RNA
aatatgatagggacttagggtgcccaggcagctggaagagacaatttttggagcaggttttctgacttcggtcggaaaacccct

SEQ ID NO: 374 - RNA, 20 nt, synthetic construct
  source 1..20: mol_type = other RNA
cccaggcagctggaagagac

SEQ ID NO: 375 - RNA, 88 nt, synthetic construct
  source 1..88: mol_type = other RNA
aatgtggggaccttgtggggaccttcatctgttcaatcattcattcaatttttggagcaggttttctgacttcggtcggaaaacccct

SEQ ID NO: 376 - RNA, 21 nt, synthetic construct
  source 1..21: mol_type = other RNA
tgtggggaccttgtggggacc

SEQ ID NO: 377 - RNA, 87 nt, synthetic construct
  source 1..87: mol_type = other RNA
aagtatttttgtgatttttgtgttcatctgttcaatcattcattcaatttttggagcaggttttctgacttcggtcggaaaacccct

SEQ ID NO: 378 - RNA, 20 nt, synthetic construct
  source 1..20: mol_type = other RNA
gtatttttgtgatttttgtg

SEQ ID NO: 379 - RNA, 88 nt, synthetic construct
  source 1..88: mol_type = other RNA
aattcacacattagaacacaacattcatctgttcaatcattcattcaatttttggagcaggttttctgacttcggtcggaaaacccct

SEQ ID NO: 380 - RNA, 21 nt, synthetic construct
  source 1..21: mol_type = other RNA
ttcacacattagaacacaaca

SEQ ID NO: 381 - RNA, 89 nt, synthetic construct
  source 1..89: mol_type = other RNA
aatatgatagggacttagggtgtcctgagtggttagggctggaatagaatttttggagcaggttttctgacttcggtcggaaaacccct

SEQ ID NO: 382 - RNA, 25 nt, synthetic construct
  source 1..25: mol_type = other RNA
tcctgagtggttagggctggaatag

SEQ ID NO: 383 - RNA, 84 nt, synthetic construct
  source 1..84: mol_type = other RNA
aatatgatagggacttagggtgtccatgtactcacccatctcaatttttggagcaggttttctgacttcggtcggaaaacccct

SEQ ID NO: 384 - RNA, 20 nt, synthetic construct
  source 1..20: mol_type = other RNA
tccatgtactcacccatctc

SEQ ID NO: 385 - RNA, 84 nt, synthetic construct
  source 1..84: mol_type = other RNA
aatgtggggaccagggggattcatctgttcaatcattcattcaatttttggagcaggttttctgacttcggtcggaaaacccct

SEQ ID NO: 386 - RNA, 17 nt, synthetic construct
  source 1..17: mol_type = other RNA
tgtggggaccaggggga

SEQ ID NO: 387 - RNA, 87 nt, synthetic construct
  source 1..87: mol_type = other RNA
aatatgttagggacttagggagttcatctgttcaatcattcattcaatttttggagcaggttttctgacttcggtcggaaaacccct

SEQ ID NO: 388 - RNA, 20 nt, synthetic construct
  source 1..20: mol_type = other RNA
ggggccactagggacaggat

SEQ ID NO: 389 - RNA, 20 nt, synthetic construct
  source 1..20: mol_type = other RNA
tatgttagggacttagggag

SEQ ID NO: 390 - RNA, 88 nt, synthetic construct
  source 1..88: mol_type = other RNA
aatatgatagggacttagggtgatgctcacacagagagccaaattcaatttttggagcaggttttctgacttcggtcggaaaacccct

SEQ ID NO: 391 - RNA, 24 nt, synthetic construct
  source 1..24: mol_type = other RNA
atgctcacacagagagccaaattc

SEQ ID NO: 392 - RNA, 85 nt, synthetic construct
  source 1..85: mol_type = other RNA
aatatgatagggacttagggtggagagccaaattcttattttcaatttttggagcaggttttctgacttcggtcggaaaacccct

SEQ ID NO: 393 - RNA, 21 nt, synthetic construct
  source 1..21: mol_type = other RNA
gagagccaaattcttattttc

SEQ ID NO: 394 - RNA, 84 nt, synthetic construct
  source 1..84: mol_type = other RNA
aatatgatagggacttagggtgcacagagagccaaattcttaaatttttggagcaggttttctgacttcggtcggaaaacccct

SEQ ID NO: 395 - RNA, 20 nt, synthetic construct
  source 1..20: mol_type = other RNA
cacagagagccaaattctta

SEQ ID NO: 396 - RNA, 85 nt, synthetic construct
  source 1..85: mol_type = other RNA
aatatgatagggacttagggtgtgctcacacagagagccaaataatttttggagcaggttttctgacttcggtcggaaaacccct

SEQ ID NO: 397 - RNA, 21 nt, synthetic construct
  source 1..21: mol_type = other RNA
tgctcacacagagagccaaat

SEQ ID NO: 398 - RNA, 84 nt, synthetic construct
  source 1..84: mol_type = other RNA
aatatgatagggacttagggtgcacatgctcacacagagagcaatttttggagcaggttttctgacttcggtcggaaaacccct

SEQ ID NO: 399 - RNA, 20 nt, synthetic construct
  source 1..20: mol_type = other RNA
cacatgctcacacagagagc

SEQ ID NO: 400 - RNA, 84 nt, synthetic construct
  source 1..84: mol_type = other RNA
aatatgatagggacttagggtgacgcacacatgctcacacagaatttttggagcaggttttctgacttcggtcggaaaacccct

SEQ ID NO: 401 - RNA, 20 nt, synthetic construct
  source 1..20: mol_type = other RNA
acgcacacatgctcacacag

SEQ ID NO: 402 - RNA, 84 nt, synthetic construct
  source 1..84: mol_type = other RNA
aatatgatagggacttagggtgcacacacgcacacatgctcaaatttttggagcaggttttctgacttcggtcggaaaacccct

SEQ ID NO: 403 - RNA, 20 nt, synthetic construct
  source 1..20: mol_type = other RNA
cacacacgcacacatgctca

SEQ ID NO: 404 - RNA, 84 nt, synthetic construct
  source 1..84: mol_type = other RNA
aatatgatagggacttagggtgctgtgccacaagccgcattcaatttttggagcaggttttctgacttcggtcggaaaacccct

SEQ ID NO: 405 - RNA, 20 nt, synthetic construct
  source 1..20: mol_type = other RNA
ctgtgccacaagccgcattc

SEQ ID NO: 406 - RNA, 88 nt, synthetic construct
  source 1..88: mol_type = other RNA
aatatgatagggacttagggtgcgaaggtcttctgccgagtcctgcaatttttggagcaggttttctgacttcggtcggaaaacccct

SEQ ID NO: 407 - RNA, 24 nt, synthetic construct
  source 1..24: mol_type = other RNA
cgaaggtcttctgccgagtcctgc

SEQ ID NO: 408 - RNA, 84 nt, synthetic construct
  source 1..84: mol_type = other RNA
aatatgatagggacttagggtgtccggtactatatggcacagaatttttggagcaggttttctgacttcggtcggaaaacccct

SEQ ID NO: 409 - RNA, 20 nt, synthetic construct
  source 1..20: mol_type = other RNA
tccggtactatatggcacag

SEQ ID NO: 410 - RNA, 84 nt, synthetic construct
  source 1..84: mol_type = other RNA
aatatgatagggacttagggtgcccgtatccggtactatatgaatttttggagcaggttttctgacttcggtcggaaaacccct

SEQ ID NO: 411 - RNA, 20 nt, synthetic construct
  source 1..20: mol_type = other RNA
cccgtatccggtactatatg

SEQ ID NO: 412 - RNA, 84 nt, synthetic construct
  source 1..84: mol_type = other RNA
aatatgatagggacttagggtgcttcagcatcagcctgatacaatttttggagcaggttttctgacttcggtcggaaaacccct

SEQ ID NO: 413 - RNA, 20 nt, synthetic construct
  source 1..20: mol_type = other RNA
cttcagcatcagcctgatac

SEQ ID NO: 414 - RNA, 88 nt, synthetic construct
  source 1..88: mol_type = other RNA
aatatgatagggacttagggtgccattcattcagtgcttgtatgacaatttttggagcaggttttctgacttcggtcggaaaacccct

SEQ ID NO: 415 - RNA, 24 nt, synthetic construct
  source 1..24: mol_type = other RNA
ccattcattcagtgcttgtatgac

SEQ ID NO: 416 - RNA, 86 nt, synthetic construct
  source 1..86: mol_type = other RNA
aatatgatagggacttagggtggtgcttgtatgacttcatcccgaatttttggagcaggttttctgacttcggtcggaaaacccct

SEQ ID NO: 417 - RNA, 22 nt, synthetic construct
  source 1..22: mol_type = other RNA
gtgcttgtatgacttcatcccg

SEQ ID NO: 418 - RNA, 85 nt, synthetic construct
  source 1..85: mol_type = other RNA
aatatgatagggacttagggtggaaggtgtcatccattcatccaatttttggagcaggttttctgacttcggtcggaaaacccct

SEQ ID NO: 419 - RNA, 21 nt, synthetic construct
  source 1..21: mol_type = other RNA
gaaggtgtcatccattcatcc

SEQ ID NO: 420 - RNA, 23 nt, synthetic construct
  source 1..23: mol_type = other RNA
ttcatctgttcaatcattcattc

SEQ ID NO: 421 - RNA, 229 nt, synthetic construct
  source 1..229: mol_type = other RNA
ggctcgctacagaggcctttccgcaatatgatagggacttagggtgttcatctgttcaatcattcattcaatttttggagcaggttttctgacttcggtcggaaaacccctcccaagttaactggtctacaatgaaagcaaaacagttctcttccccgctccccggtgtgtgagaggggctttgatccttctctggtttcctaggaaacgcgtaagcttgtattcagat

SEQ ID NO: 422 - RNA, 19 nt, synthetic construct
  source 1..19: mol_type = other RNA
ggagaggacttgatcatta

SEQ ID NO: 423 - RNA, 22 nt, synthetic construct
  source 1..22: mol_type = other RNA
aaccgaacaggacctgaaagag

SEQ ID NO: 424 - RNA, 24 nt, synthetic construct
  source 1..24: mol_type = other RNA
cagtcacaccatcgtccatctatc

SEQ ID NO: 425 - RNA, 20 nt, synthetic construct
  source 1..20: mol_type = other RNA
tccatcatgaagtgtgacgt

SEQ ID NO: 426 - RNA, 20 nt, synthetic construct
  source 1..20: mol_type = other RNA
tcatccccaagccattcagg

SEQ ID NO: 427 - RNA, 20 nt, synthetic construct
  source 1..20: mol_type = other RNA
tgcttaggttcggcattgga

SEQ ID NO: 428 - RNA, 20 nt, synthetic construct
  source 1..20: mol_type = other RNA
ccagaacatcatccctgcct

SEQ ID NO: 429 - RNA, 20 nt, synthetic construct
  source 1..20: mol_type = other RNA
ggtcaggtccaccactgaca

SEQ ID NO: 430 - RNA, 20 nt, synthetic construct
  source 1..20: mol_type = other RNA
acctgtctgcatgagaacct

SEQ ID NO: 431 - RNA, 21 nt, synthetic construct
  source 1..21: mol_type = other RNA
atggatggagagatggaacct

SEQ ID NO: 432 - RNA, 22 nt, synthetic construct
  source 1..22: mol_type = other RNA
gggctgtctcatcgtagtaaac

SEQ ID NO: 433 - RNA, 21 nt, synthetic construct
  source 1..21: mol_type = other RNA
gctaaaacagcaatggcctac

SEQ ID NO: 434 - RNA, 22 nt, synthetic construct
  source 1..22: mol_type = other RNA
ttgcttcacttccatatcatcg

SEQ ID NO: 435 - RNA, 21 nt, synthetic construct
  source 1..21: mol_type = other RNA
gctaaaacagcaatgggactc

SEQ ID NO: 436 - RNA, 20 nt, synthetic construct
  source 1..20: mol_type = other RNA
gcaggctgtctgtctctctc

SEQ ID NO: 437 - RNA, 19 nt, synthetic construct
  source 1..19: mol_type = other RNA
tcctcactctctgacgagg

SEQ ID NO: 438 - RNA, 17 nt, synthetic construct
  source 1..17: mol_type = other RNA
catggcggtcgacctag

SEQ ID NO: 439 - RNA, 20 nt, synthetic construct
  source 1..20: mol_type = other RNA
taccatccactcgacacacc

SEQ ID NO: 440 - RNA, 21 nt, synthetic construct
  source 1..21: mol_type = other RNA
agtcagtcaagctagcagagg

SEQ ID NO: 441 - RNA, 20 nt, synthetic construct
  source 1..20: mol_type = other RNA
gacatcaaatcccgcgtgaa

SEQ ID NO: 442 - RNA, 20 nt, synthetic construct
  source 1..20: mol_type = other RNA
cattgatgttggcgagcagg

SEQ ID NO: 443 - RNA, 19 nt, synthetic construct
  source 1..19: mol_type = other RNA
gctctctccgctgctgtag

SEQ ID NO: 444 - RNA, 20 nt, synthetic construct
  source 1..20: mol_type = other RNA
cgaggttccgggtaaaagca

SEQ ID NO: 445 - RNA, 20 nt, synthetic construct
  source 1..20: mol_type = other RNA
ctgtctctctctctcgcaca

SEQ ID NO: 446 - RNA, 19 nt, synthetic construct
  source 1..19: mol_type = other RNA
gagacttggtggtgcataa

SEQ ID NO: 447 - RNA, 21 nt, synthetic construct
  source 1..21: mol_type = other RNA
tactcctgcttgctgatccac

SEQ ID NO: 448 - RNA, 201 nt, Homo sapiens
  source 1..201: mol_type = mRNA
cccggcccaggatgacgtgcttattatttgacaggtgcatgtgacactgtgactccggctgtgacctgatggggcctcagggatgcgtctggctctggcaggatgtttgtgtgtcaccgcgatgttgtgtgggtgtgtctacctgtgccctgctctgagggattgagtgtgatatcgtgtgtttgtgctgcgctgtgatgg

SEQ ID NO: 449 - RNA, 65 nt, Homo sapiens
  source 1..65: mol_type = mRNA
gaccagaactattttttcccttacaccttgaccagcttgcatattggataccacatgattatcag

SEQ ID NO: 450 - RNA, 135 nt, Homo sapiens
  source 1..135: mol_type = mRNA
atcttttgtgtgtgtgtgtgtgtgtgtgtgtgtgtgtgtgtcacccaggctggagtgcagtggcatgatcacagctcactgcagcctcaacttcctgggctcaagtgatcctctcccgagtagctgggactacag

SEQ ID NO: 451 - RNA, 228 nt, Homo sapiens
  source 1..228: mol_type = other RNA
gtagcccctggctgtccttcagagggggcacaggtggagaaagaggcgcagtccctggctgtggtccctggagtgggtatacacgtgtgagtgtgtgcagatgtggaggtgagtaggcaagcgaagtgtatgtgtgtgcatggatgtattacaagtgtgtgcgtgtgggtgagtgtgcatgtctgggtgtgagtgtgcccgagactgcatgcatgtgtgtgtgtgagt

SEQ ID NO: 452 - RNA, 220 nt, Homo sapiens
  source 1..220: mol_type = mRNA
ctggactggggcaccactggctggcgtgagtgcacatgtgtgtgtttatgagtgtggctgtgaaatgtgagcacgtgcacctgtatgtatgtgtgtgggtgtttgcacgtgggggcgcgtgagcacatgaaatcagggtccatatgggtggggatgtgcatacatgtgcatgtgtattgtgtgaatgtatgagtgagcgtgtggaggtgtgtgcatgtgg

SEQ ID NO: 453 - RNA, 204 nt, Homo sapiens
  source 1..204: mol_type = mRNA
aagaaaagcgaggagcccttcaggttgtgcctatgaccctttgggtcgtccttttttgtctatttccctcccctccttcccaagcactgtatgtgtgtgtgtatgtgtgtgtgtgtgtgtacatgcacatgtgcgtgcatgatctgtgcctctgagctttggctcatgcagtctatttttctgaaaagcagtttgtgtgcatgc

SEQ ID NO: 454 - RNA, 41 nt, Homo sapiens
  source 1..41: mol_type = mRNA
gacgtgcttattatttgacaggtgcatgtgacactgtgact

SEQ ID NO: 455 - RNA, 59 nt, Homo sapiens
  source 1..59: mol_type = mRNA
acgtgcttattatttgacaggtgcatgtgacactgtgactccggctgtgacctgatggg

SEQ ID NO: 456 - RNA, 61 nt, Homo sapiens
  source 1..61: mol_type = mRNA
ggcctcagggatgcgtctggctctggcaggatgtttgtgtgtcaccgcgatgttgtgtggg

SEQ ID NO: 457 - RNA, 62 nt, Homo sapiens
  source 1..62: mol_type = mRNA
tgtgtcaccgcgatgttgtgtgggtgtgtctacctgtgccctgctctgagggattgagtgtg

SEQ ID NO: 458 - RNA, 42 nt, Homo sapiens
  source 1..42: mol_type = mRNA
ttttcccttacaccttgaccagcttgcatattggataccaca

SEQ ID NO: 459 - RNA, 32 nt, Homo sapiens
  source 1..32: mol_type = mRNA
acaccttgaccagcttgcatattggataccac

SEQ ID NO: 460 - RNA, 42 nt, Homo sapiens
  source 1..42: mol_type = mRNA
tgtgtgtgtgtgtgtcacccaggctggagtgcagtggcatga

SEQ ID NO: 461 - RNA, 96 nt, Homo sapiens
  source 1..96: mol_type = mRNA
atcttttgtgtgtgtgtgtgtgtgtgtgtgtgtgtgtgtgtcacccaggctggagtgcagtggcatgatcacagctcactgcagcctcaacttcct

SEQ ID NO: 462 - RNA, 32 nt, Homo sapiens
  source 1..32: mol_type = mRNA
agtgatcctctcccgagtagctgggactacag

SEQ ID NO: 463 - RNA, 40 nt, Homo sapiens
  source 1..40: mol_type = mRNA
gtgtgtgcagatgtggaggtgagtaggcaagcgaagtgta

SEQ ID NO: 464 - RNA, 61 nt, Homo sapiens
  source 1..61: mol_type = mRNA
aggcaagcgaagtgtatgtgtgtgcatggatgtattacaagtgtgtgcgtgtgggtgagtg

SEQ ID NO: 465 - RNA, 53 nt, Homo sapiens
  source 1..53: mol_type = mRNA
caagtgtgtgcgtgtgggtgagtgtgcatgtctgggtgtgagtgtgcccgaga

SEQ ID NO: 466 - RNA, 37 nt, Homo sapiens
  source 1..37: mol_type = mRNA
agtgtgcccgagactgcatgcatgtgtgtgtgtgagt

SEQ ID NO: 467 - RNA, 40 nt, Homo sapiens
  source 1..40: mol_type = mRNA
ggggcaccactggctggcgtgagtgcacatgtgtgtgttt

SEQ ID NO: 468 - RNA, 59 nt, Homo sapiens
  source 1..59: mol_type = mRNA
acatgtgcatgtgtattgtgtgaatgtatgagtgagcgtgtggaggtgtgtgcatgtgg

SEQ ID NO: 469 - RNA, 45 nt, Homo sapiens
  source 1..45: mol_type = mRNA
aagaaaagcgaggagcccttcaggttgtgcctatgaccctttggg

SEQ ID NO: 470 - RNA, 92 nt, Homo sapiens
  source 1..92: mol_type = mRNA
tcccaagcactgtatgtgtgtgtgtatgtgtgtgtgtgtgtgtacatgcacatgtgcgtgcatgatctgtgcctctgagctttggctcatgc

SEQ ID NO: 471 - RNA, 40 nt, Homo sapiens
  source 1..40: mol_type = mRNA
catgcagtctatttttctgaaaagcagtttgtgtgcatgc

SEQ ID NO: 472 - RNA, 64 nt, synthetic construct
  source 1..64: mol_type = other RNA
aatccatgtactcacccatctcaatttttggagcaggttttctgacttcggtcggaaaacccct

SEQ ID NO: 473 - RNA, 68 nt, synthetic construct
  source 1..68: mol_type = other RNA
aacgaaggtcttctgccgagtcctgcaatttttggagcaggttttctgacttcggtcggaaaacccct

SEQ ID NO: 474 - RNA, 1897 nt, synthetic construct
  source 1..1897: mol_type = other RNA
gaattccaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagaggcctttccgcaatatgatagggacttagggtgtccatgtactcacccatctcaatttttggagcaggttttctgacttcggtcggaaaacccctcccaagttaactggtctacaatgaaagcaaaacagttctcttccccgctccccggtgtgtgagaggggctttgatccttctctggtttcctaggaaacgcgtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgtcgacgaattccaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagaggcctttccgcaatatgatagggacttagggtgcacagagagccaaattcttaaatttttggagcaggttttctgacttcggtcggaaaacccctcccaagttaactggtctacaatgaaagcaaaacagttctcttccccgctccccggtgtgtgagaggggctttgatccttctctggtttcctaggaagctttacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcaggatccgaattccaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagaggcctttccgcaatatgatagggacttagggtgcccgtatccggtactatatgaatttttggagcaggttttctgacttcggtcggaaaacccctcccaagttaactggtctacaatgaaagcaaaacagttctcttccccgctccccggtgtgtgagaggggctttgatccttctctggtttcctaggaagatctatggcagcggccgcatgatatctagactggcctgggcctcatgggccttccgctc

SEQ ID NO: 475 - RNA, 40 nt, synthetic construct
  source 1..40: mol_type = other RNA
agatccagtttatcgataccaacataggagctgtgattgg

SEQ ID NO: 476 - RNA, 40 nt, synthetic construct; identical to SEQ ID NO: 475
  source 1..40: mol_type = other RNA

SEQ ID NO: 477 - RNA, 1211 nt, synthetic construct
  source 1..1211: mol_type = other RNA
tatttccggtgaattcgccgccaccatggtttccaagggcgaagaggacaacatggccatcatcaaagaattcatgcggttcaaggtgcacatggaaggcagcgtgaacggccacgagttcgagattgaaggcgaaggcgagggcagaccttacgagggaacacagaccgccaagctgaaagtgaccaaaggcggccctctgccttttgcctgggacattctgagccctcagtttatgtacggcagcaaggcctacgtgaagcaccccgccgatattcccgactacctgaagctgagcttccccgagggcttcaagtgggagagagtgatgaacttcgaggacggcggcgtggtcaccgtgactcaagatagctctctgcaggacggcgagttcatctacaaagtgaagctgcggggcaccaactttccctctgatggccccgtgatgcagaaaaagaccatgggctgggaagccagcagcgagagaatgtaccctgaagatggcgccctgaagggcgagatcaagcagcggctgaaactgaaggatggcggccactacgacgccgaagtgaaaaccacctacaaggccaagaaacccgtgcagctgcctggcgcctacaacgtgaacatcaagctggacatcaccagccacaacgaggactacaccatcgtggaacagtacgagagagccgaaggcagacacagcacaggcggaatggacgagctgtacaaaggctctggcgaaggccgtggcagcctgcttacatgcggagatgtggaagagaaccccggacctatggccaagcctctgagccaagaggaaagcaccctgatcgaaagagccaccgccacaatcaacagcatccccatcagcgaggattacagcgtggcctctgctgccctcagctccgatggcagaatcttcacaggcgtgaacgtgtaccacttcaccggcggaccttgtgccgaactggtggttcttggaacagctgccgctgccgcagccggcaatctgacatgtattgtggccatcggcaacgagaaccggggcatccttagtccttgcggcagatgcagacaggtgctgctggatctgcaccctggcatcaaggccattgtgaaggactctgacggccagcctacagccgtgggaattagagagctgctgcctagcggctatgtgtgggagggatgaacgcgtctggaacaat

SEQ ID NO: 478 - RNA, 43 nt, synthetic construct
  source 1..43: mol_type = other RNA
taatttctactcttgtagatggaaaagtaaaagatgtctgaat

SEQ ID NO: 479 - RNA, 20 nt, synthetic construct
  source 1..20: mol_type = other RNA
ctggcgaggcatcacatttt

SEQ ID NO: 480 - RNA, 21 nt, synthetic construct
  source 1..21: mol_type = other RNA
cgttctcatcttcggttaccc

SEQ ID NO: 481 - RNA, 20 nt, synthetic construct
  source 1..20: mol_type = other RNA
aacgacattgccctgaagac

SEQ ID NO: 482 - RNA, 18 nt, synthetic construct
  source 1..18: mol_type = other RNA
ccagtacggctcccatct

SEQ ID NO: 483 - RNA, 19 nt, synthetic construct
  source 1..19: mol_type = other RNA
tgggaccgctttacgcttc

SEQ ID NO: 484 - RNA, 21 nt, synthetic construct
  source 1..21: mol_type = other RNA
gagactggctgactcgttgac

SEQ ID NO: 485 - RNA, 19 nt, synthetic construct
  source 1..19: mol_type = other RNA
ctctgggactggagcaaac

SEQ ID NO: 486 - RNA, 19 nt, synthetic construct
  source 1..19: mol_type = other RNA
catcccgtatccggtaagg